(19) United States
(12) Patent Application Publication
(10) Pub. No.: US 2023/0227406 A1
(43) Pub. Date: Jul. 20, 2023

(54) MODULATORS OF E3 LIGASES

(71) Applicant: Rekindle Therapeutics Inc., Cambridge, MA (US)

(72) Inventors: Arthur F. Kluge, Gainesville, FL (US); Soumya Ray, Quincy, MA (US); Suresh K. Jain, West Roxbury, MA (US); Detlev Biniszkiewicz, Boston, MA (US); Ekambar R. Kandimalla, Hopkinton, MA (US); Joshua Grady, Arlington, MA (US)

(21) Appl. No.: 18/009,972

(22) PCT Filed: Jun. 14, 2021
(86) PCT No.: PCT/US2021/037294

§ 371 Date: Dec. 12, 2022

Related U.S. Application Data
(60) Provisional application No. 63/038,732, filed on Jun. 12, 2020.
(60) Provisional application No. 63/038,730, filed on Jun. 12, 2020.

(51) Int. Cl. C07D 209/12; C07D 209/30; C07D 417/12; A61P 35/00

(52) U.S. Cl. CPC C07D 209/12; C07D 209/30; C07D 417/12; A61P 35/00

(57) ABSTRACT

Provided herein are methods, oligonucleotides, and compositions for modulating levels of at least one target protein in a cell, e.g., by reducing levels of the mRNA of the target protein or inhibiting translation of the mRNA. In some embodiments, the target protein can be the ubiquitin ligase polypeptide, including, but not limited to WWP1, WWP2, and NEDD4. Also provided herein are methods, oligonucleotides, and compositions for treating, ameliorating, delaying or reducing a symptom of a cancer. The present disclosure also relates to compounds, methods and compositions for modulating at least one target protein selected from ubiquitin protein ligase. Also provided herein are compounds, methods, and compositions for treating, ameliorating, delaying or reducing a symptom of a cancer or a viral infection. In particular, the present disclosure is directed to specific small-molecule modulators of HECT domain E3 ligases that are useful in the therapy and diagnosis of cancer and viral infection.

### CROSS-REFERENCE TO RELATED APPLICATION

This application claims priority to and the benefit of U.S. Provisional Patent Application No. 63/038,732, filed on Jun. 12, 2020, and U.S. Provisional Patent Application No. 63/038,730, filed on Jun. 12, 2020, the entire contents of each of which are incorporated by reference herein for all purposes.

### SEQUENCE LISTING

The instant application contains a Sequence Listing which has been submitted electronically in ASCII format and is hereby incorporated by reference in its entirety. Said ASCII copy, created on Jun. 14, 2021, is named REK-002WO_SL.txt and is 135,730 bytes in size.

### FIELD

The present disclosure relates to compounds, methods and compositions for modulating at least one target protein selected from ubiquitin protein ligases in a cell. In particular, the present disclosure is directed to small-molecule modulators of HECT domain E3 ligases that are useful in the therapy and diagnosis of cancer and viral infection.

### BACKGROUND

Ubiquitination is a critical post-translational protein modification (PTM) that governs practically all aspects of cellular function. The ubiquitination of the protein is accomplished by an E3 ubiquitin ligase that binds to a protein and adds ubiquitin molecules to the protein.

An increasing number of studies have showed that E3 ubiquitin ligases (E3s) are important enzymes in the process of ubiquitination that primarily determine substrate specificity and thus need to be tightly controlled (R. J. Mayer, Protein Degradation: The Ubiquitin-Proteasome System and Disease, Volume 4, WileyVCH, 2007). For example, the ubiquitination of protein substrates by HECT domain E3 ligases is known to affect protein trafficking and protein degradation/stabilization; and, as a consequence, HECT domain E3 ligases control numerous eukaryotic cellular processes (Buetow and Huang, Nature, 2016, 17, 626). Inhibition of these ligases is a therapeutic strategy to modulate the activity of proteins that are directly linked to cancer (Chan, et al., Mol Cell. 2017, 66, 345) and to viral replication (Shepley-McTaggart, et al. J. Biol. Chem. 2020, 295, 4604).

HECT domain E3 ligases control hallmarks of cancers by regulating the ubiquitination levels (Bernassola et al., Trends in Biochemical Sciences, 2019, 44, 12, 1057). Therefore, modulating interactions between E3 ubiquitin ligases and their substrates that play roles in cancer formation and progression would be a key target for developing anti-cancer drugs. For example, pathways relating to checkpoint inhibitors (e.g. PDL-1) can be regulated through WWP1-dependent sorting.

In addition, abnormal expression and dysfunction of HECT E3s have been shown in many different cancers (Weber et al., Front Physiol., 2019, 10, 370). To illustrate, WWP1 is an oncogenic factor that has been shown to be amplified or mutated in multiple cancer types (Chen et al., Oncogene, 2007, 26, 2386). The mutation spectrum is interesting as most known cancer mutations are copy number gains and a smaller number are copy number losses/deletions. Understanding the nature of these gain of function mutations (GoFs) could pave the way for developing new anti-cancer drugs.

Currently, most antiviral therapeutics are directed to targeting viral-specific proteins. On the other hand, targeting human host proteins that are essential for viral budding has been shown as a potentially better approach (Remdesivir by Gilead Sciences, Inc.). For example, WWP1 E3 Ligase has been found to modify the proline-rich Pro-Pro-x-Tyr (PPxY) motif on the viral protein to enhance virus budding (J Virol, 91 (20), 2008, 12-17, 2017). Thus, strategies to develop a new class of modulators of viral PPxY-host E3 Ligase interactions could be used as antiviral therapeutics.

Therefore, an effective method to inhibit a target ubiquitin ligase protein which causes or contributes to a disease would have significant diagnostic and therapeutic impacts. Antisense technology is emerging as an effective means for reducing the expression of specific gene products and may therefore prove to be uniquely useful in a number of therapeutic, diagnostic, and research applications. In addition, an ongoing need exists in the art for effective small molecule therapeutics across disease indications. The present disclosure provides useful compositions and methods for modulating levels of E3 ubiquitin ligases by using antisense technology and small molecule modulators to modulate HECT domain E3 ligases.

### SUMMARY

### Antisense Oligonucleotides

Provided herein is an antisense oligonucleotide comprising a nucleobase sequence complementary to at least 12 contiguous nucleotides in SEQ ID NO: 541 (or a variant thereof) selected from the group consisting of: 1-45, 61-97, 173-192, 205-224, 235-272, 426-445, 524-573, 611-630, 647-715, 790-809, 851-870, 994-1024, 1057-1076, 1134-1188, 1276-1295, 1307-1326, 1373-1392, 1470-1489, 1599-1632, 1780-1799, 1817-1846, 1918-1937, 2225-2244, 2316-2335, 2367-2386, 2407-2457, 2651-2670, and 2761-2780. In some embodiments, the complementary region of the antisense oligonucleotide is at least 80%, at least 85%, at least 90%, at least 95%, at least 96%, at least 97%, at least 98%, at least 99%, or at least 100% complementary to SEQ ID NO: 541 (or a variant thereof).

In some embodiments, the antisense oligonucleotide comprises a nucleobase sequence complementary to at least 12 contiguous nucleotides in a HECT domain E3 ligase selected from the group consisting of: 1-45, 61-97, 173-192, 205-224, 235-272, 426-445, 524-573, 611-630, 647-715, 790-809, 851-870, 994-1024, 1057-1076, 1134-1188, 1276-1295, 1307-1326, 1373-1392, 1470-1489, 1599-1632, 1780-1799, 1817-1846, 1918-1937, 2225-2244, 2316-2335, 2367-2386, 2407-2457, 2651-2670, and 2761-2780.

In some embodiments, the nucleobase sequence is (i) 12 nucleosides, (ii) 12-30 nucleosides, (iii) 15-30 linked nucleosides or (iv) at least 15 nucleosides.

Provided herein is an antisense oligonucleotide comprising at least 12 linked nucleosides having a nucleobase sequence of any one of SEQ ID NOS: 1-44 and 532-540, or a variant sequence at least about 90% identical thereto.

In some embodiments, the length of the antisense oligonucleotide is (i) 12 nucleosides, (ii) 12-30 nucleosides or (iii) 15-30 linked nucleosides.

In some embodiments, the antisense oligonucleotide comprises a nucleobase sequence complementary that is complimentary to at least 8 contiguous nucleotides at a nucleotide position in SEQ ID NO: 541 selected from: 1-20, 21-39, 26-45, 61-80, 78-97, 173-192, 205-224, 235-254, 253-272, 426-445, 524-543, 534-553, 554-573, 611-630, 647-666, 671-690, 696-715, 790-809, 851-870, 994-1013, 1005-1024, 1057-1076, 1134-1153, 1142-1161, 1153-1172, 1169-1188, 1276-1295, 1307-1326, 1373-1392, 1470-1489, 1599-1618, 1613-1632, 1780-1799, 1817-1836, 1827-1846, 1918-1937, 2225-2244, 2316-2335, 2367-2386, 2407-2426, 2426-2445, 2438-2457, 2651-2670, and 2761-2780.

In some embodiments, the antisense oligonucleotide comprises a nucleobase sequence complementary that is complimentary to at least 9 contiguous nucleotides at a nucleotide position in SEQ ID NO: 541 selected from: 1-20, 21-39, 26-45, 61-80, 78-97, 173-192, 205-224, 235-254, 253-272, 426-445, 524-543, 534-553, 554-573, 611-630, 647-666, 671-690, 696-715, 790-809, 851-870, 994-1013, 1005-1024, 1057-1076, 1134-1153, 1142-1161, 1153-1172, 1169-1188, 1276-1295, 1307-1326, 1373-1392, 1470-1489, 1599-1618, 1613-1632, 1780-1799, 1817-1836, 1827-1846, 1918-1937, 2225-2244, 2316-2335, 2367-2386, 2407-2426, 2426-2445, 2438-2457, 2651-2670, and 2761-2780.

In some embodiments, the antisense oligonucleotide comprises a nucleobase sequence complementary that is complimentary to at least 10 contiguous nucleotides at a nucleotide position in SEQ ID NO: 541 selected from: 1-20, 21-39, 26-45, 61-80, 78-97, 173-192, 205-224, 235-254, 253-272, 426-445, 524-543, 534-553, 554-573, 611-630, 647-666, 671-690, 696-715, 790-809, 851-870, 994-1013, 1005-1024, 1057-1076, 1134-1153, 1142-1161, 1153-1172, 1169-1188, 1276-1295, 1307-1326, 1373-1392, 1470-1489, 1599-1618, 1613-1632, 1780-1799, 1817-1836, 1827-1846, 1918-1937, 2225-2244, 2316-2335, 2367-2386, 2407-2426, 2426-2445, 2438-2457, 2651-2670, and 2761-2780.

In some embodiments, the antisense oligonucleotide comprises a nucleobase sequence complementary that is complimentary to at least 11 contiguous nucleotides at a nucleotide position in SEQ ID NO: 541 selected from: 1-20, 21-39, 26-45, 61-80, 78-97, 173-192, 205-224, 235-254, 253-272, 426-445, 524-543, 534-553, 554-573, 611-630, 647-666, 671-690, 696-715, 790-809, 851-870, 994-1013, 1005-1024, 1057-1076, 1134-1153, 1142-1161, 1153-1172, 1169-1188, 1276-1295, 1307-1326, 1373-1392, 1470-1489, 1599-1618, 1613-1632, 1780-1799, 1817-1836, 1827-1846, 1918-1937, 2225-2244, 2316-2335, 2367-2386, 2407-2426, 2426-2445, 2438-2457, 2651-2670, and 2761-2780.

In some embodiments, the antisense oligonucleotide comprises a nucleobase sequence complementary that is complimentary to at least 12 contiguous nucleotides at a nucleotide position in SEQ ID NO: 541 selected from: 1-20, 21-39, 26-45, 61-80, 78-97, 173-192, 205-224, 235-254, 253-272, 426-445, 524-543, 534-553, 554-573, 611-630, 647-666, 671-690, 696-715, 790-809, 851-870, 994-1013, 1005-1024, 1057-1076, 1134-1153, 1142-1161, 1153-1172, 1169-1188, 1276-1295, 1307-1326, 1373-1392, 1470-1489, 1599-1618, 1613-1632, 1780-1799, 1817-1836, 1827-1846, 1918-1937, 2225-2244, 2316-2335, 2367-2386, 2407-2426, 2426-2445, 2438-2457, 2651-2670, and 2761-2780.

In some embodiments, the antisense oligonucleotide comprises a nucleobase sequence complementary that is complimentary to at least 13 contiguous nucleotides at a nucleotide position in SEQ ID NO: 541 selected from: 1-20, 21-39, 26-45, 61-80, 78-97, 173-192, 205-224, 235-254, 253-272, 426-445, 524-543, 534-553, 554-573, 611-630, 647-666, 671-690, 696-715, 790-809, 851-870, 994-1013, 1005-1024, 1057-1076, 1134-1153, 1142-1161, 1153-1172, 1169-1188, 1276-1295, 1307-1326, 1373-1392, 1470-1489, 1599-1618, 1613-1632, 1780-1799, 1817-1836, 1827-1846, 1918-1937, 2225-2244, 2316-2335, 2367-2386, 2407-2426, 2426-2445, 2438-2457, 2651-2670, and 2761-2780.

In some embodiments, the antisense oligonucleotide comprises a nucleobase sequence complementary that is complimentary to at least 14 contiguous nucleotides at a nucleotide position in SEQ ID NO: 541 selected from: 1-20, 21-39, 26-45, 61-80, 78-97, 173-192, 205-224, 235-254, 253-272, 426-445, 524-543, 534-553, 554-573, 611-630, 647-666, 671-690, 696-715, 790-809, 851-870, 994-1013, 1005-1024, 1057-1076, 1134-1153, 1142-1161, 1153-1172, 1169-1188, 1276-1295, 1307-1326, 1373-1392, 1470-1489, 1599-1618, 1613-1632, 1780-1799, 1817-1836, 1827-1846, 1918-1937, 2225-2244, 2316-2335, 2367-2386, 2407-2426, 2426-2445, 2438-2457, 2651-2670, and 2761-2780.

In some embodiments, the antisense oligonucleotide comprises a nucleobase sequence complementary that is complimentary to at least 15 contiguous nucleotides at a nucleotide position in SEQ ID NO: 541 selected from: 1-20, 21-39, 26-45, 61-80, 78-97, 173-192, 205-224, 235-254, 253-272, 426-445, 524-543, 534-553, 554-573, 611-630, 647-666, 671-690, 696-715, 790-809, 851-870, 994-1013, 1005-1024, 1057-1076, 1134-1153, 1142-1161, 1153-1172, 1169-1188, 1276-1295, 1307-1326, 1373-1392, 1470-1489, 1599-1618, 1613-1632, 1780-1799, 1817-1836, 1827-1846, 1918-1937, 2225-2244, 2316-2335, 2367-2386, 2407-2426, 2426-2445, 2438-2457, 2651-2670, and 2761-2780.

Provided herein is an antisense oligonucleotide comprising 15 to 30 linked nucleosides having a nucleobase sequence or chemical modification thereof comprising a complementary region comprising at least 12 nucleobases to a target region of equal length of a WWP1 transcript, wherein the nucleobase sequence comprises SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41, 42, 43, or 44, or a variant sequence at least about 90% identical thereto.

In some embodiments, the complementary region of the antisense oligonucleotide is at least 80%, at least 85%, at least 90%, at least 95%, or at least 100% complementary to the target region, as measured over the complementary region or the entire length of the oligonucleotide.

In some embodiments, the oligonucleotide comprises a chemical modification.

In some embodiments, the antisense oligonucleotide is capable of modulating translation, expression or activity of WWP1 in a mammalian cell. In some embodiments, the modulation is inhibition or down-regulation.

In some embodiments, the antisense oligonucleotide inhibits or down-regulate translation, expression or activity of WWP1 in a mammalian cell.

In some embodiments, the nucleobases are independently selected from adenine, thymine, cytosine, guanine, uracil and 5-methylcytosine.

In some embodiments, the antisense oligonucleotide inhibits or down-regulate translation, expression or activity of WWP1 in a mammalian cell.

In some embodiments, the nucleobases complementary to a target region is contiguous.

In some embodiments, the antisense oligonucleotide is a modified oligonucleotide.

In some embodiments, the modified oligonucleotide is a gapmer.

In some embodiments, the modified oligonucleotide comprises at least one modified internucleoside linkage and/or at least one modified sugar moiety and/or at least one modified nucleobase.

In some embodiments, the at least one modified internucleoside linkage is a phosphorothioate internucleoside linkage, the at least one modified sugar is a bicyclic sugar or 2′-O-methyoxyethyl, and the at least one modified nucleobase is a 5 methylcytosine.

In some embodiments, the modified internucleoside linkage comprises a phosphorothioate internucleoside linkage.

In some embodiments, the modified oligonucleotide is a chimeric oligonucleotide.

In some embodiments, the modified sugar moiety comprises a 2′-substituted sugar moiety, wherein the 2′-substituent is selected from among: 2′-OMe, 2′-F, and 2′-MOE.

In some embodiments, the 2′-modification is 2′-OR1, wherein R1 is optionally substituted C1-C6 aliphatic.

In some embodiments, the 2′-modification is 2′-OCH2CH2OMe.

In some embodiments, the modified sugar moiety comprises a bicyclic sugar moiety.

In some embodiments, the bicyclic sugar moiety comprises LNA or cEt.

Provided herein, a pharmaceutical composition comprising at least one antisense oligonucleotide and a pharmaceutically acceptable carrier, in an amount effective to inhibit or down-regulate translation, expression or activity of WWP1 in a mammalian cell.

In some embodiments, the pharmaceutical composition according further comprises one or more vaccines, antigens, antibodies, cytotoxic agents, chemotherapeutic agents, kinase inhibitors, allergens, antibiotics, agonist, antagonist, antisense oligonucleotides, ribozymes, RNAi molecules, siRNA molecules, miRNA molecules, aptamers, proteins, gene therapy vectors, DNA vaccines, adjuvants, co-stimulatory molecules or combinations thereof.

In some embodiments, the pharmaceutical composition comprises two or more antisense oligonucleotides targeting different regions of WWP1.

Provided herein, a method for inhibiting WWP1 pre-RNA, WWP1 mRNA or protein expression, the method comprising contacting a cell with pharmaceutical composition comprising at least one antisense oligonucleotide and a pharmaceutically acceptable carrier, in an amount effective to inhibit or down-regulate translation, expression or activity of WWP1 in a mammalian cell.

In some embodiments, the method of inhibiting the synthesis of WWP1 in a cell that expresses WWP1, comprising contacting the cell with the pharmaceutical composition comprising at least one antisense oligonucleotide and a pharmaceutically acceptable carrier, in an amount effective to inhibit or down-regulate translation, expression or activity of WWP1 in a mammalian cell.

Provided herein, a method of enhancing apoptosis in a cell expressing WWP1, comprising contacting a pharmaceutical composition comprising at least one antisense oligonucleotide and a pharmaceutically acceptable carrier, in an amount effective to inhibit or down-regulate translation, expression or activity of WWP1 in a mammalian cell.

Provided herein, a method of inhibiting the growth of a cancer cell expressing WWP1, comprising contacting the cell with a pharmaceutical composition comprising at least one antisense oligonucleotide and a pharmaceutically acceptable carrier, in an amount effective to inhibit or down-regulate translation, expression or activity of WWP1 in a mammalian cell.

Provided herein, a method for the treatment of a disease, disorder, or condition associated with WWP1 in a mammal in need thereof, the method comprising administering a pharmaceutical composition comprising at least one antisense oligonucleotide and a pharmaceutically acceptable carrier, in an amount effective to inhibit or down-regulate translation, expression or activity of WWP1 in a mammalian cell.

Provided herein, a method for treating a cancer in a human, comprising administering to the mammal a therapeutically effective amount of a pharmaceutical composition comprising at least one antisense oligonucleotide and a pharmaceutically acceptable carrier, in an amount effective to inhibit or down-regulate translation, expression or activity of WWP1 in a mammalian cell.

In some embodiments, the pharmaceutical composition is administered in combination with one or more vaccines, antigens, antibodies, cytotoxic agents, allergens, antibiotics, antisense oligonucleotides, Toll-like receptor antagonists, peptides, proteins, gene therapy vectors, DNA vaccines, adjuvants, or kinase inhibitors.

In some embodiments, the administering of the pharmaceutical composition is daily.

In some embodiments, wherein the route of administration of the pharmaceutical composition is selected from, intrathecal, parenteral, intramuscular, subcutaneous, intraperitoneal, intravenous, mucosal delivery, oral, sublingual, transdermal, topical, inhalation, intranasal, aerosol, intraocular, intratracheal, intrarectal, vaginal, gene gun, dermal patch, eye drop or mouthwash.

Provided herein, a pharmaceutical composition comprising at least two antisense oligonucleotides comprising 12 to 15, 12 to 30 or 15 to 30 linked nucleosides having a nucleobase sequence or chemical modification thereof comprising a complementary region comprising at least 12 nucleobases to a target region of equal length of a WWP1 and/or WWP2 and/or NEDD4.

In some embodiments, the antisense oligonucleotides in the pharmaceutical composition are chemically conjugated or co-formulated.

In some embodiments, the target region of each oligonucleotide in the pharmaceutical composition is different.

Provided herein, an antisense oligonucleotide comprising 15 to 30 linked nucleosides having a nucleobase sequence or chemical modification thereof comprising a complementary region comprising at least 12 contiguous nucleobases complementary to a target region of equal length of a HECT domain E3 ligase transcript.

In some embodiments, the complementary region of the antisense oligonucleotide is at least 80%, at least 85%, at least 90%, at least 95%, or at least 100% complementary to the target region, as measured over the complementary region or the entire length of the oligonucleotide.

In some embodiments, the antisense oligonucleotide inhibits or down-regulates translation, expression or activity of HECT domain E3 ligase transcript in a mammalian cell.

In one embodiment, the HECT domain E3 ligase is selected from ITCH, SMURF1, SMURF2, WWP1, WWP2, NEDD4, NEDD4-2, HECW1, HECW2, HERC1, HERC2, HERC3, HERC4, HERC5, HERC6, E6AP, HUWE1, HACE1, TRIP12, UBR5, UBE3B, UBE3C, HECTD1, HECTD2, HECTD3, HECTD4, G2E3, AREL1. Preferably, the HECT domain E3 ligase is selected from WWP1, WWP2, and NEDD4.

Provided herein, a pharmaceutical composition comprising at least one antisense oligonucleotide comprising 15 to 30 linked nucleosides having a nucleobase sequence or chemical modification thereof comprising a complementary region comprising at least 12 contiguous nucleobases complementary to a target region of equal length of a HECT domain E3 ligase transcript and a pharmaceutically acceptable carrier, in an amount effective to inhibit or down-regulate translation, expression or activity of HECT domain E3 ligase in a mammalian cell.

The present disclosure also relates to a method of inhibiting budding and/or the growth, and/or the proliferation, and/or the infectivity, of an enveloped virus, said method comprises administering an effective amount of an antisense oligonucleotide comprising 15 to 30 linked nucleosides having a nucleobase sequence or chemical modification thereof comprising a complementary region comprising at least 12 contiguous nucleobases complementary to a target region of equal length of a HECT domain E3 ligase transcript to a mammal infected with said virus or at risk of infection with said virus. In one embodiment, the mammal is a mammal infected with said virus. In one embodiment, said mammal is a non-human mammal. In another embodiment, said mammal is a human.

In some embodiments, the antisense oligonucleotide decrease/prevent virus budding/egress.

In some embodiments, the virus is a member of a family selected from the group consisting of Herpesviridae, Poxviridae, Hepadnaviridae, Coronaviridae, Flaviviridae, Togaviridae, Retroviridae, Orthomyxoviridae, Arenaviridae, Bunyaviridae, Filoviridae, Paramyxoviridae, and Rhabdoviridae. In one embodiment, said virus is selected from the group consisting of Herpes simplex, type 1, Herpes simplex, type 2, Varicella-zoster virus, Epstein-Barr virus, Human cytomegalovirus, Human herpesvirus, Smallpox, Hepatitis B virus, Severe acute respiratory syndrome virus, Hepatitis C virus, yellow fever virus, dengue virus, West Nile virus, TBE virus, Zika virus, Rubella virus, Human immunodeficiency virus (HIV), Influenza virus, Lassa virus, Crimean-Congo, hemorrhagic fever virus, Hantaan virus, Ebola virus, Marburg virus, Measles virus, Mumps virus, Parainfluenza virus, Respiratory syncytial virus, Rabies virus, and Hepatitis D virus (HDV).

In a preferred embodiment, said virus is selected from SARS-CoV, MERS-CoV, 229E, NL63, OC43, and HKU1. Particularly, said virus is SARS-CoV-2.

In some embodiments, the present disclosure is directed to a method that ameliorates one of more symptoms of a pathology caused by said virus and/or slows or prevents infection of said mammal by said virus.

In some embodiments, the present disclosure provides a method of treating cancer in a patient need thereof, comprising: administering to said patient a pharmaceutical composition comprising at least one antisense oligonucleotide comprising 12-30 or 15 to 30 linked nucleosides having a nucleobase sequence or chemical modification thereof comprising a complementary region comprising at least 12 contiguous nucleobases complementary to a target region of equal length of a HECT domain E3 ligase transcript and a pharmaceutically acceptable carrier, in an amount effective to inhibit or down-regulate translation, expression or activity of HECT domain E3 ligase in a mammalian cell.

In some embodiments, the pharmaceutical composition further comprises one or more vaccines, antigens, antibodies, cytotoxic agents, chemotherapeutic agents, kinase inhibitors, allergens, antibiotics, agonist, antagonist, antisense oligonucleotides, ribozymes, RNAi molecules, siRNA molecules, miRNA molecules, aptamers, proteins, gene therapy vectors, DNA vaccines, adjuvants, co-stimulatory molecules or combinations thereof.

In one embodiment, the pharmaceutical composition further comprises a therapeutically effective amount of at least one other therapeutic agent or composition thereof selected from the group consisting of a corticosteroid, an anti-inflammatory signal transduction modulator, bronchodilator, an anticholinergic, a mucolytic agent, hypertonic saline or mixtures thereof.

The present disclosure also relates to a method of enhancing apoptosis in a cell expressing HECT domain E3 ligase, comprising contacting a pharmaceutical composition comprising at least one antisense oligonucleotide comprising 15 to 30 linked nucleosides having a nucleobase sequence or chemical modification thereof comprising a complementary region comprising at least 12 contiguous nucleobases complementary to a target region of equal length of a HECT domain E3 ligase transcript and a pharmaceutically acceptable carrier, in an amount effective to inhibit or down-regulate translation, expression or activity of HECT domain E3 ligase in a mammalian cell.

In some embodiments, the route of administration of the pharmaceutical composition is selected from, intrathecal, parenteral, intramuscular, subcutaneous, intraperitoneal, intravenous, mucosal delivery, oral, sublingual, transdermal, topical, inhalation, intranasal, aerosol, intraocular, intratracheal, intrarectal, vaginal, gene gun, dermal patch, eye drop or mouthwash.

The present disclosure also provides a method of treating a disease or disorder associated with PD-L1, the method comprising administering a pharmaceutical composition comprising at least one antisense oligonucleotide comprising 15 to 30 linked nucleosides having a nucleobase sequence or chemical modification thereof comprising a complementary region comprising at least 12 contiguous nucleobases complementary to a target region of equal length of a HECT domain E3 ligase transcript and a pharmaceutically acceptable carrier, in an amount effective to inhibit or down-regulate translation, expression or activity of HECT domain E3 ligase in a mammalian cell. In some embodiments, said antisense oligonucleotides modulate the stability and/or the half-life and/or exosomal excretion and/or the cell surface expression of PD-L1. In one embodiment, said antisense oligonucleotides reduce PD-L1 half-life. In another embodiment, said antisense oligonucleotides down-regulate PD-L1 on the tumor cell surface. In one embodiment, said antisense oligonucleotides prevent exosomal excretion of PD-L1 in the tumor microenvironment.

In some embodiments, the present disclosure is directed to method of modulating of PD-L1 receptors on tumor cell surface, comprising administering to a mammal a therapeutically effective amount of a pharmaceutical composition comprising at least one said antisense oligonucleotides.

The present disclosure also provides a method of treating cancer in a patient in need thereof, comprising: administering to said patient a therapeutically effective amount of an antisense oligonucleotide as described herein, a small molecule as described herein, or a pharmaceutical composition of either.

The present disclosure also provides a method for modulating or inhibiting the activity of WWP1 and/or WWP2 and/or NEDD4, comprising: contacting a cell with an antisense oligonucleotide as described herein, a small molecule as described herein, or a pharmaceutical composition of either.

The present disclosure also provides a method of enhancing apoptosis in a cell expressing WWP1 and/or WWP2 and/or NEDD4, comprising: contacting the cell with an antisense oligonucleotide as described herein, a small molecule as described herein, or a pharmaceutical composition of either.

The present disclosure also provides a method of inhibiting the growth of a cancer cell expressing WWP1 and/or WWP2 and/or NEDD4, comprising: contacting the cell with an antisense oligonucleotide as described herein, a small molecule as described herein, or a pharmaceutical composition of either.

The present disclosure also provides a method for the treatment of a disease, disorder, or condition associated with WWP1 and/or WWP2 and/or NEDD4 in a mammal in need thereof, the method comprising: administering an antisense oligonucleotide as described herein, a small molecule as described herein, or a pharmaceutical composition of either.

The present disclosure also provides a method for treating a cancer in a human, comprising: administering a therapeutically effective amount of an antisense oligonucleotide as described herein, a small molecule as described herein, or a pharmaceutical composition of either.

The present disclosure also provides a method of inhibiting the growth of a human prostate cancer cell, comprising: contacting the cell with an antisense oligonucleotide as described herein, a small molecule as described herein, or a pharmaceutical composition of either.

In some embodiments, the route of administration of the compound or pharmaceutical composition is selected from, intrathecal, parenteral, intramuscular, subcutaneous, intraperitoneal, intravenous, mucosal delivery, oral, sublingual, transdermal, topical, inhalation, intranasal, aerosol, intraocular, intratracheal, intrarectal, vaginal, gene gun, dermal patch, eye drop or mouthwash.

In some embodiments, the methods further comprise administration of one or more vaccines, antigens, antibodies, cytotoxic agents, chemotherapeutic agents, kinase inhibitors, allergens, antibiotics, agonist, antagonist, antisense oligonucleotides, ribozymes, RNAi molecules, siRNA molecules, miRNA molecules, aptamers, proteins, gene therapy vectors, DNA vaccines, adjuvants, co-stimulatory molecules or combinations thereof.

The present disclosure is also directed to a method of identifying E3 ligase gain of function mutations in cancer, the method comprising,
(i) Identifying mutations of E3 ligases via genomics and bioinformatics tools;
(ii) Inhibiting identified mutations with at least one agent selected from small molecules, anti-sense oligonucleotides, and/or siRNAs;
(iii) Identifying the gain of function mutations if the inhibition of the said mutation triggers tumor suppressive activity.

In some embodiments, the present disclosure provides a method of identifying E3 ligase gain of function mutations in cancer, the method comprising,
(i) Identifying mutations of E3 ligases via genomics and bioinformatics tools;
(ii) Inhibiting identified mutations with at least one antisense oligonucleotide comprising 15 to 30 linked nucleosides having a nucleobase sequence or chemical modification thereof comprising a complementary region comprising at least 12 contiguous nucleobases complementary to a target region of equal length of a HECT domain E3 ligase transcript;
(iii) Identifying the gain of function mutations if the inhibition of the said mutation triggers tumor suppressive activity.

In some embodiments, the present disclosure provides a method of treating autoimmune disease in a patient in need thereof, comprising: administering to said patient a therapeutically effective amount of the antisense oligonucleotide of any one of claims 35-54 or the pharmaceutical composition of any one of claims 55-60.

In some embodiments, the autoimmune disease is selected from the group consisting of: psoriasis, rheumatoid arthritis, systemic lupus erythematosus, ulcerative colitis, Crohn's disease, transplant rejection, immune disorder associated with graft transplantation rejection, benign lymphocytic angiitis, lupus erythematosus, Hashimoto's thyroiditis, primary myxedema, Graves's disease, pernicious anemia, autoimmune atrophic gastritis, Addison's disease, insulin dependent diabetes mellitis, Good pasture's syndrome, myasthenia gravis, pemphigus, sympathetic ophthalmia, autoimmune uveitis, autoimmune hemolytic anemia, idiopathic thrombocytopenia, primary biliary cirrhosis, chronic hepatitis, ulcerates colitis, Sjogren's syndrome, Wegener's sarcoidosis, antiphospholipid syndrome, inflammatory myopathy, polyarteritis, rheumatic disease, polymyositis, scleroderma, mixed connective tissue disease, inflammatory rheumatism, degenerative rheumatism, extra-articular rheumatism, collagen disease, chronic polyarthritis, psoriasis arthropathica, ankylosing spondylitis, juvenile rheumatoid arthritis, periarthritis humeroscapularis, panarteriitis nodosa, progressive systemic scleroderma, arthritis urica, dermatomyositis, muscular rheumatism, myositis, myogelosis, and chondrocalcinosis, thyroiditis, allergic oedema, granulomas, Alzheimer's disease, Parkinson's disease, multiple sclerosis, or amyotrophic lateral sclerosis (ALS).

In some embodiments, the autoimmune disease is psoriasis. In some embodiments, the autoimmune disease is rheumatoid arthritis. In some embodiments, the autoimmune disease is systemic lupus erythematosus. In some embodiments, the autoimmune disease is ulcerative colitis. In some embodiments, the autoimmune disease is Crohn's disease. In some embodiments, the autoimmune disease is transplant rejection. In some embodiments, the autoimmune disease is immune disorder associated with graft transplantation rejection. In some embodiments, the autoimmune disease is benign lymphocytic angiitis. In some embodiments, the autoimmune disease is lupus erythematosus. In some embodiments, the autoimmune disease is Hashimoto's thyroiditis. In some embodiments, the autoimmune disease is primary myxedema. In some embodiments, the autoimmune disease is Graves's disease. In some embodiments, the autoimmune disease is pernicious anemia. In some embodiments, the autoimmune disease is autoimmune atrophic gastritis. In some embodiments, the autoimmune disease is Addison's disease. In some embodiments, the autoimmune disease is insulin dependent diabetes mellitis. In some embodiments, the autoimmune disease is Good pasture's syndrome. In some embodiments, the autoimmune disease is myasthenia gravis. In some embodiments, the autoimmune disease is pemphigus. In some embodiments, the autoimmune disease is sympathetic ophthalmia. In some embodiments, the autoimmune disease is autoimmune uveitis. In some embodiments, the autoimmune disease is autoimmune hemolytic anemia. In some embodiments, the autoimmune disease is idiopathic thrombocytopenia. In some embodiments, the autoimmune disease is primary biliary cirrhosis. In some embodiments, the autoimmune disease is chronic hepatitis. In some embodiments, the autoimmune disease is ulcerates colitis. In some embodiments, the autoimmune disease is Sjogren's syndrome. In some embodiments, the autoimmune disease is Wegener's sarcoidosis. In some embodiments, the autoimmune disease is antiphospholipid syndrome. In some embodiments, the autoimmune disease is inflammatory myopathy. In some embodiments, the autoimmune disease is polyarteritis. In some embodiments, the autoimmune disease is rheumatic disease. In some embodiments, the autoimmune disease is polymyositis. In some embodiments, the autoimmune disease is scleroderma. In some embodiments, the autoimmune disease is mixed connective tissue disease. In some embodiments, the autoimmune disease is inflammatory rheumatism. In some embodiments, the autoimmune disease is degenerative rheumatism. In some embodiments, the autoimmune disease is extra-articular rheumatism. In some embodiments, the autoimmune disease is collagen disease. In some embodiments, the autoimmune disease is chronic polyarthritis. In some embodiments, the autoimmune disease is psoriasis arthropathica. In some embodiments, the autoimmune disease is ankylosing spondylitis. In some embodiments, the autoimmune disease is juvenile rheumatoid arthritis. In some embodiments, the autoimmune disease is periarthritis humeroscapularis. In some embodiments, the autoimmune disease is panarteriitis nodosa. In some embodiments, the autoimmune disease is progressive systemic scleroderma. In some embodiments, the autoimmune disease is arthritis urica. In some embodiments, the autoimmune disease is dermatomyositis. In some embodiments, the autoimmune disease is muscular rheumatism. In some embodiments, the autoimmune disease is myositis. In some embodiments, the autoimmune disease is myogelosis. In some embodiments, the autoimmune disease is and chondrocalcinosis. In some embodiments, the autoimmune disease is thyroiditis. In some embodiments, the autoimmune disease is allergic oedema. In some embodiments, the autoimmune disease is granulomas. In some embodiments, the autoimmune disease is Alzheimer's disease. In some embodiments, the autoimmune disease is Parkinson's disease. In some embodiments, the autoimmune disease is multiple sclerosis. In some embodiments, the autoimmune disease is or amyotrophic lateral sclerosis (ALS).

In some embodiments, the present disclosure provides a method of upregulating PD-L1, comprising: administering to a patient in need thereof a therapeutically effective amount of an antisense oligonucleotide as described herein or the pharmaceutical composition as described herein.

In some embodiments, the present disclosure relates to a method of screening for a therapeutic agent for treating a proliferative disease or disorder in an individual in need thereof, the method comprising:
(i) Contacting a cell with at least one test agent selected from small molecules, anti-sense oligonucleotides, and/or siRNAs;
(ii) Detecting inhibition of gain of function mutations of E3 ligases as compared to a control;
(iii) Identifying the test agent as a therapeutic agent if the test agent promotes tumor suppressive activity.

### Small Molecule Modulators

The present disclosure relates to a compound of formula I:

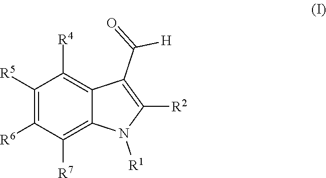

or a pharmaceutically acceptable salt or solvate thereof, wherein R¹, R², R⁴, R⁵, R⁶, and R⁷ are hereafter defined.

The present disclosure also relates to a compound of formula II:

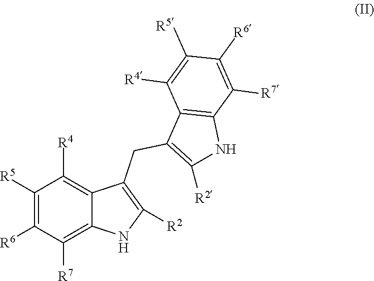

or a pharmaceutically acceptable salt or solvate thereof, wherein R², R⁴, R⁵, R⁶, R⁷, R^{2′}, R^{4′}, R^{5′}, R^{6′}, and R7′ are hereafter defined.

In one embodiment, the compound described in the present disclosure is selected from the compounds listed in Table 1 hereafter.

The present disclosure also relates to a pharmaceutical composition comprising a compound according to the present disclosure and at least one pharmaceutically acceptable excipient.

In one embodiment, the compound described in the present disclosure is selected from the group consisting of: 6-ethynyl-1-phenyl-1H-indole-3-carbaldehyde; N-(2-cyanopyridin-4-yl)-3-formyl-1-phenyl-1H-indole-5-carboxamide; bis(4-fluoro-1H-indol-3-yl)methane; bis(5-fluoro-1H-indol-3-yl)methane; 3-((1H-indol-3-yl)methyl)-1H-indole-6-carboxylic acid; 3-((1H-indol-3-yl)methyl)-1H-indole-5-carboxylic acid; 3-((1H-indol-3-yl)methyl)-N-(prop-2-yn-1-yl)-1H-indole-6-carboxamide; 3-((1H-indol-3-yl)methyl)-2-methyl-N-(prop-2-yn-1-yl)-1H-indole-6-carboxamide; 3-((1H-indol-3-yl)methyl)-N-(2-hydroxyethyl)-N-(prop-2-yn-1-yl)-1H-indole-6-carboxamide, and pharmaceutically acceptable salts or solvates thereof.

In one embodiment, the compound of formula I or II modulates activity of at least one HECT domain E3 ligase in a mammalian cell. In preferred embodiments, modulation is inhibition.

In one embodiment, the HECT domain E3 ligase is selected from ITCH, SMURF1, SMURF2, WWP1, WWP2, NEDD4, NEDD4-2, HECW1, HECW2, HERC1, HERC2, HERC3, HERC4, HERC5, HERC6, E6AP, HUWE1, HACE1, TRIP12, UBR5, UBE3B, UBE3C, HECTD1, HECTD2, HECTD3, HECTD4, G2E3, AREL1. Preferably, the HECT domain E3 ligase is selected from WWP1, WWP2, and NEDD4.

The present disclosure further relates to a method of modulating HECT domain E3 ligase in a patient in need thereof, comprising: administering to said patient an effective amount of a compound of formula I or II.

The present disclosure also relates to a method of inhibiting HECT domain E3 ligase in a cell or in a patient in need thereof, comprising: administering to said cell or said patient an effective amount of a compound of formula I or II.

The present disclosure also relates to a method of inhibiting budding and/or the growth, and/or the proliferation, and/or the infectivity, of an enveloped virus, said method comprising contacting said virus with an effective amount of a compound of formula I or II. In some embodiments, the method comprises administering said compound to a mammal infected with said virus or at risk of infection with said virus. In one embodiment, the mammal is a mammal infected with said virus. In one embodiment, said mammal is a non-human mammal. In another embodiment, said mammal is a human.

In some embodiments, the compounds of formula I and II decrease/prevent virus budding/egress.

In some embodiments, the compounds of formula I and II modulates/inhibits the interaction between viral the proline-rich Pro-Pro-x-Tyr (PPxY) motif and host HECT domain E3 ligases in a mammal.

The present disclosure also relates to method of modulating/inhibiting the interaction between viral the proline-rich Pro-Pro-x-Tyr (PPxY) motif and host HECT domain E3 ligases in a mammal need thereof, comprising: administering to said mammal an effective amount of a compound of formula I or II.

In some embodiments, the virus is a member of a family selected from the group consisting of Herpesviridae, Poxviridae, Hepadnaviridae, Coronaviridae, Flaviviridae, Togaviridae, Retroviridae, Orthomyxoviridae, Arenaviridae, Bunyaviridae, Filoviridae, Paramyxoviridae, and Rhabdoviridae. In one embodiment, said virus is selected from the group consisting of Herpes simplex, type 1, Herpes simplex, type 2, Varicella-zoster virus, Epstein-Barr virus, Human cytomegalovirus, Human herpesvirus, Smallpox, Hepatitis B virus, Severe acute respiratory syndrome virus, Hepatitis C virus, yellow fever virus, dengue virus, West Nile virus, TBE virus, Zika virus, Rubella virus, Human immunodeficiency virus (HIV), Influenza virus, Lassa virus, Crimean-Congo, hemorrhagic fever virus, Hantaan virus, Ebola virus, Marburg virus, Measles virus, Mumps virus, Parainfluenza virus, Respiratory syncytial virus, Rabies virus, and Hepatitis D virus (HDV).

In a preferred embodiment, said virus is selected from SARS-CoV, MERS-CoV, 229E, NL63, OC43, and HKU1. Particularly, said virus is SARS-CoV-2.

In some embodiments, the present disclosure is directed to a method that ameliorates one of more symptoms of a pathology caused by said virus and/or slows or prevents infection of said mammal by said virus.

In some embodiments, the present disclosure provides a method of treating cancer in a patient in need thereof, comprising administering to said patient an effective amount of a compound of formula I or II.

This present disclosure further relates to a pharmaceutical composition comprising at least one compound of formula I or II and at least one pharmaceutically acceptable excipient.

In some embodiments, the pharmaceutical composition comprise at least one compound of formula I or II and at least one pharmaceutically acceptable excipient, in an amount effective to inhibit the activity of at least one type of HECT domain E3 ligases in a mammalian cell.

In some embodiments, a pharmaceutical composition described herein further comprises one or more vaccines, antigens, antibodies, cytotoxic agents, chemotherapeutic agents, kinase inhibitors, allergens, antibiotics, agonist, antagonist, antisense oligonucleotides, ribozymes, RNAi molecules, siRNA molecules, miRNA molecules, aptamers, proteins, gene therapy vectors, DNA vaccines, adjuvants, co-stimulatory molecules or combinations thereof.

In one embodiment, the pharmaceutical composition further comprises a therapeutically effective amount of at least one other therapeutic agent or composition thereof selected from the group consisting of a corticosteroid, an anti-inflammatory signal transduction modulator, bronchodilator, an anticholinergic, a mucolytic agent, hypertonic saline or mixtures thereof.

This present disclosure also provides a method for modulating the activity of WWP1 and/or WWP2 and/or NEDD4, comprising contacting a cell with pharmaceutical composition comprising at least one compound of formula I or II.

This present disclosure also provides a method for inhibiting the activity of WWP1 and/or WWP2 and/or NEDD4, comprising contacting a cell with pharmaceutical composition comprising at least one compound of formula I or II.

This present disclosure also relates to a method of enhancing apoptosis in a cell expressing WWP1 and/or WWP2 and/or NEDD4, comprising contacting a pharmaceutical composition comprising at least one compound of formula I or II.

This present disclosure further relates to a method of inhibiting the growth of a cancer cell expressing WWP1 and/or WWP2 and/or NEDD4, comprising contacting the cell with a pharmaceutical composition comprising at least one compound of formula I or II.

This present disclosure is further directed to a method for the treatment of a disease, disorder, or condition associated with WWP1 and/or WWP2 and/or NEDD4 in a mammal in need thereof, the method comprising administering a pharmaceutical composition comprising at least one compound of formula I or II.

This present disclosure is also directed to a method for treating a cancer in a human, comprising administering to a mammal a therapeutically effective amount of a pharmaceutical composition comprising at least one compound of formula I or II.

This present disclosure further provides a method of inhibiting the growth of a human prostate cancer cell, comprising contacting the cell with a pharmaceutical composition comprising at least one compound of formula I or II.

In some embodiments, the route of administration of the pharmaceutical composition is selected from, intrathecal, parenteral, intramuscular, subcutaneous, intraperitoneal, intravenous, mucosal delivery, oral, sublingual, transdermal, topical, inhalation, intranasal, aerosol, intraocular, intratracheal, intrarectal, vaginal, gene gun, dermal patch, eye drop or mouthwash.

This present disclosure also provides a method of inhibiting the growth of a human prostate cancer cell, comprising contacting the cell with a pharmaceutical composition comprising at least one compound of formula I or II.

In some embodiments, the compounds of formula I and II binds to a HECT domain E3 ligase through non-covalent bonds.

In some embodiments, the compounds of formula I and II binds to a WWP1 docking site.

In some embodiments, docking site comprise protein residues including Try656 and Try628.

In some embodiments, the compounds of formula I and II inhibits the growth of human prostate cancer cell.

This present disclosure also provides a method of treating a disease or disorder associated with PD-L1, the method comprising administering an inhibitor/modulator of HECT domain E3 ligases.

This present disclosure further provides a method of treating a disease or disorder associated with PD-L1, the method comprising administering an small molecule inhibitor of the interaction between PD-L1 and HECT domain E3 ligases.

In some embodiments, the compounds of formula I and II modulates the stability and/or the half-life and/or exosomal excretion and/or the cell surface expression of PD-L1. In one embodiment, the compounds of formula I and II reduce PD-L1 half-life. In another embodiment, the compounds of formula I and II down-regulate PD-L1 on the tumor cell surface. In one embodiment, the compounds of formula I and II prevents exosomal excretion of PD-L1 in the tumor microenvironment.

In some embodiments, the present disclosure is directed to method of modulating of PD-L1 receptors on tumor cell surface, comprising administering to a mammal a therapeutically effective amount of a pharmaceutical composition comprising at least one compound of formula I or II.

This present disclosure is also directed to a method of identifying E3 ligase gain of function mutations in cancer, the method comprising,
i. Identifying mutations of E3 ligases via genomics and bioinformatics tools;
ii. Inhibiting identified mutations with at least one agent selected from small molecules, anti-sense oligonucleotides, and/or siRNAs;
iii. Identifying the gain of function mutations if the inhibition of the said mutation triggers tumor suppressive activity.

In some embodiments, this present disclosure provides a method of identifying E3 ligase gain of function mutations in cancer, the method comprising,
i. Identifying mutations of E3 ligases via genomics and bioinformatics tools;
ii. Inhibiting identified mutations with at least one compound of formula I or II;
iii. Identifying the gain of function mutations if the inhibition of the said mutation triggers tumor suppressive activity.

In some embodiments, this present disclosure relates to a method of screening for a therapeutic agent for treating a proliferative disease or disorder in an individual in need thereof, the method comprising:

Contacting a cell with at least one test agent selected from small molecules, anti-sense oligonucleotides, and/or siRNAs;

Detecting inhibition of gain of function mutations of E3 ligases as compared to a control;

Identifying the test agent as a therapeutic agent if the test agent promotes tumor suppressive activity.

### INCORPORATION BY REFERENCE

The entire disclosures of all patent and non-patent publications cited herein are each incorporated by reference in their entireties for all purposes.

### BRIEF DESCRIPTION OF THE DRAWINGS

FIG. 1 depicts molecular modeling showing binding mode for compound 9 in the pocket of WWP1. The docked pose for the compound 9 shows clear interactions between the molecule and protein residues including Try656 and Try628. These aromatic residues are critical for the binding.

FIG. 2 depicts lead small molecule candidates inhibiting WWP1 autoubiquitination. WWP1 will auto-ubiquitinate in the presence of E1 enzyme, E2 enzyme, ATP, ubiquitin, and a buffer system. On a SDS PAGE gel, this reaction is visualized by the disappearance of free ubiquitin and the formation of a ubiquitin smear at a high molecular weight. In the presence of low (IC50) and high (5×IC50) concentrations of inhibitor, to varying degrees, lead candidates from the small molecule program inhibited WWP1 auto ubiquitination compared to a DMSO only control.

FIG. 3 depicts ubiquitin chain forming ability dictating WWP1 autoubiquitination kinetics. WWP1 rapidly auto-ubiquitinates in the presence of Wt ubiquitin, and to a lesser degree but still completely, in the presence of K63-only and K48-only ubiquitin. WWP1 largely does not auto-ubiquitinate in the presence of K27-only ubiquitin.

FIG. 4 depicts that the WWP1 substrate PTEN is preferentially ubiquitinated using K27-only (K27O) or non-chain forming ubiquitin (NoK). During an in vitro ubiquitination reaction containing the substrate PTEN and wild type ubiquitin, WWP1 will preferentially autoubiquitinate. Presumably WWP1 forms K63 and K48 chains in this reaction; however, in the presence of K27 only ubiquitin, PTEN substrate ubiquitin is preferred. PTEN is readily ubiquitinated with non-chain forming ubiquitin suggesting multi-mono ubiquitination is occurring, in addition to any K27 chain formation.

FIGS. 5A-5D depict MS/MS validation of ubiquitin preference for autoubiquitination and PTEN substrate ubiquitination by WWP1. In vitro ubiquitination reactions were digested with Trypsin/LysC and labeled with TMT quantification reagents. Samples were analyzed and quantified by LC-MS/MS. Various non-K27 chains form during the reaction with wild type ubiquitin, and K27 chains are largely only formed using K27-only ubiquitin. Non-K27 chains are more numerous (greater S/N of TMT in the reaction). Specific ubiquitin sites on PTEN demonstrate greater occupancy (e.g., K237 and K342) when K27-only ubiquitin is used in the reaction vs wild type ubiquitin. Different site of PTEN have different ubiquitin kinetics; moreover, K342 rapidly reaches a high occupancy in 2 hours, where as another site on PTEN, K223, becomes ubiquitinated slower.

FIGS. 6A and 6B depict a lead allosteric series compound, AS-004-115, inhibiting WWP1 dependent ubiquitination of PTEN in a dose-dependent manner. In vitro ubiquitination reactions were carried out for 2 hours using the indicated doses of inhibitor. Increasing doses of inhibitor block PTEN substrate ubiquitination, which correlates with remaining free ubiquitin. The “0” concentration is DMSO only (0.5%, same as was used for compound doses). No E1 and No E3 negative controls are present (only free ubiquitin present). Analysis occurred by western blot using an anti-ubiquitin antibody. A fraction of these samples was digested with Trypsin/LysC and labeled with TMT quantification reagents prior to LC-MS/MS analysis. Mass spectrometry results confirm the inhibition of ubiquitin transfer to a lysine on PTEN and inhibition of K27 ubiquitin chain formation.

FIG. 7 depicts in vitro ubiquitination reactions that were carried out for 2 hours using the indicated doses of inhibitor. Increasing doses of inhibitor block PTEN substrate ubiquitination, which correlates with remaining free ubiquitin. The “0” concentration is DMSO only (0.5%, same as was used for compound doses). No E1 and No E3 negative controls are present (only free ubiquitin present). Analysis occurred by western blot using an anti-ubiquitin antibody. A fraction of these samples was digested with Trypsin/LysC and labeled with TMT quantification reagents prior to LC-MS/MS analysis. Mass spectrometry results confirm the inhibition of ubiquitin transfer to a lysine on PTEN and inhibition of K27 ubiquitin chain formation.

FIG. 8 depicts knockdown of WWP1 by siRNA in human breast cancer cell lines reducing phosphorylated AKT and phosphorylated Ribosomal Protein S6 levels, known oncogenic markers. Cells were treated with 10 nM pooled siRNA (IDT) and harvested 48 hours after. Cells were lysed in RIPA buffer containing protease and phosphatase inhibitors on ice. Lysates were analyzed by western blot using the indicated antibodies. siRNA successfully reduced WWP1 levels by several fold. This reduction in WWP1 protein levels strongly correlated with a reduction in phosphorylated AKT at serine 473 and a reduction in Ribosomal Protein S6 phosphorylation at serine 235/236. These data indicate a reduction in growth pathway signaling with the knockdown of WWP1.

FIGS. 9A and 9B depict lead I₃C series candidate COMPOUND 1 reducing phosphorylated AKT and phosphorylated Ribosomal Protein S6 levels in a dose-dependent manner. Hs578T cells (human triple negative breast cancer) were treated with the indicated concentration of COMPOUND 1 for 48 hours prior to harvesting. The “0” concentration is DMSO only (0.5%, same as was used for compound doses). Cells were lysed in RIPA buffer containing protease and phosphatase inhibitors on ice. Analysis occurred via western blot using the indicated antibodies. Quantification by densitometry of the bands from the western blot are presented for phosphorylated AKT/Total AKT and phosphorylated Ribosomal Protein S6/Total Ribosomal Protein S6. COMPOUND 1 reduced both AKT and S6 phosphorylation relative to their respective total protein levels.

FIGS. 10A and 10B depicts lead I₃C series candidate COMPOUND 1 increasing PD-L1 levels and the glycosylated/non-glycosylated PD-L1 ratio. Hs578T cells (human triple negative breast cancer) were treated with the indicated concentration of COMPOUND 1 for 48 hours prior to harvesting. The “0” concentration is DMSO only (0.5%, same as was used for compound doses). Cells were lysed in RIPA buffer containing protease and phosphatase inhibitors on ice. COMPOUND 1 treatment increased both PD-L1 levels and PD-L1 glycosylation (higher MW band). These results were observed in both the presence and absence of interferon gamma (IFN, 25 ng/mL in cell culture media during the drug treatment), a stimulator of PD-L1 expression. Analysis occurred via western blot using the indicated antibodies. Quantification by densitometry of the bands from the western blot are presented for the PD-L1/Vinculin (loading control) in the absence of interferon gamma, and glycosylated/non-glycosylated PD-L1 in the presence of interferon gamma.

FIG. 11 depicts replication of COMPOUND 1 treatment data in Hs578T and BT-20 TNBC cells, AKT and S6 phosphorylation effects. The data presented here are consistent with the previous replicate in Hs578T cells, and the observation is preserved in another TNBC cell line, BT-20. COMPOUND 1 reduces the phospho AKT/vinculin loading control and phospho-S6/total S6 ratios. Methods are the same as the previous replicate, using the indicated doses.

FIG. 12 depicts replication of COMPOUND 1 treatment data in Hs578T and BT-20 TNBC cells, PD-L1 effects. The data presented here are consistent with the previous replicate in Hs578T cells, and the observation is preserved in another TNBC cell line, BT-20. COMPOUND 1 increases PD-L1 levels and the glycosylated PD-L1/non-glycosylated PD-L1 ratio. Methods are the same as the previous replicate, using the indicated doses.

FIG. 13 depicts lead I₃C series candidate COMPOUND 6 reducing phosphorylated AKT and phosphorylated Ribosomal Protein S6 levels in a dose-dependent manner. Hs578T cells (human triple negative breast cancer) were treated with the indicated concentration of COMPOUND 6 for 48 hours prior to harvesting. The “0” concentration is DMSO only (0.5%, same as was used for compound doses). Cells were lysed in RIPA buffer containing protease and phosphatase inhibitors on ice. Analysis occurred via western blot using the indicated antibodies. COMPOUND 6 treatment reduced both AKT and S6 phosphorylation relative to the vinculin loading control and total S6 protein levels, respectively.

FIG. 14 depicts lead I₃C series candidate COMPOUND 6 reducing PD-L1 levels in a dose-dependent manner. Hs578T cells (human triple negative breast cancer) were treated with the indicated concentration of COMPOUND 6 for 48 hours before harvesting. The “0” concentration is DMSO only (0.5%, same as was used for compound doses). Cells were lysed in RIPA buffer containing protease and phosphatase inhibitors on ice. Analysis occurred via western blot using the indicated antibodies. PD-L1 protein levels were reduced relative to the vinculin loading control upon compound treatment. This effect is observed in the presence and absence of interferon gamma (IFN, 25 ng/mL in cell culture media during the drug treatment).

FIG. 15 depicts replication of COMPOUND 6 treatment data in Hs578T cells, AKT and S6 phosphorylation effects. The data presented here are consistent with the previous replicate in Hs578T cells. COMPOUND 6 reduces the phospho-AKT/vinculin loading control and phospho-S6/total S6 ratios. Methods are the same as the previous replicate, using the indicated doses.

FIG. 16 depicts replication of COMPOUND 6 treatment data, AKT and S6 phosphorylation effects using the BT-20 cell line. The data presented here are consistent with the previous replicate in Hs578T cells. COMPOUND 6 reduces the phospho-AKT/vinculin loading control and phospho-S6/total S6 ratios in BT-20 cells. Methods are the same as the previous replicate, using the indicated doses.

FIG. 17 depicts replication of COMPOUND 6 treatment data and PD-L1 effects using the BT-20 cell line. The data presented here are consistent with the previous replicate in Hs578T cells. COMPOUND 6 reduces the PD-L1/vinculin loading control ratio in BT-20 cells. Additionally, COMPOUND 6 probably reduces PD-L1 glycosylation. Methods are the same as the previous replicate, using the indicated doses.

FIG. 18 depicts Lead I₃C series candidate COMPOUND 7 reducing phosphorylated AKT and phosphorylated Ribosomal Protein S6 levels in a dose-dependent manner. Hs578T cells (human triple negative breast cancer) were treated with the indicated concentration of COMPOUND 7 for 48 hours before harvesting. The “0” concentration is DMSO only (0.5%, same as was used for compound doses). Cells were lysed in RIPA buffer containing protease and phosphatase inhibitors on ice. Analysis occurred via western blot using the indicated antibodies. COMPOUND 7 treatment reduced both AKT and S6 phosphorylation relative to the vinculin loading control and total S6 protein levels, respectively.

FIG. 19 depicts lead I₃C series candidate COMPOUND 7 reducing PD-L1 in a dose-dependent manner. Hs578T cells (human triple negative breast cancer) were treated with the indicated concentration of COMPOUND 7 for 48 hours prior to harvesting. The “0” concentration is DMSO only (0.5%, same as was used for compound doses). Cells were lysed in RIPA buffer containing protease and phosphatase inhibitors on ice. Analysis occurred via western blot using the indicated antibodies. PD-L1 protein levels were reduced relative to the vinculin loading control upon 319-02 treatment.

FIG. 20 depicts replication of COMPOUND 7 treatment data in Hs578T cells, S6 phosphorylation effect. The data presented here are consistent with the previous replicate in Hs578T cells. COMPOUND 7 reduces the phospho S6/vinculin loading control ratio. Methods are the same as the previous replicate, using the indicated doses.

FIG. 21 depicts replication of COMPOUND 7 treatment data in Hs578T cells, PD-L1 effect. The data presented here are consistent with the previous replicate in Hs578T cells. COMPOUND 7 reduces the PD-L1/vinculin loading control ratio. Methods are the same as the previous replicate, using the indicated doses.

FIG. 22 depicts lead I₃C series candidate COMPOUND 8 reducing phosphorylated Ribosomal Protein S6 levels in a dose-dependent manner. Hs578T cells (human triple negative breast cancer) were treated with the indicated concentration of COMPOUND 8 for 48 hours, harvesting after. The “0” concentration is DMSO only (0.5%, same as was used for compound doses). Cells were lysed in RIPA buffer containing protease and phosphatase inhibitors on ice. Analysis occurred via western blot using the indicated antibodies. COMPOUND 8 treatment reduced the levels of S6 phosphorylation relative to the vinculin loading control.

FIG. 23 depicts lead I₃C series candidate COMPOUND 8 reducing PD-L1 levels in a dose dependent manner. Hs578T cells (human triple negative breast cancer) were treated with the indicated concentration of COMPOUND 8 for 48 hours and then harvested. The “0” concentration is DMSO only (0.5%, same as was used for compound doses). Cells were lysed in RIPA buffer containing protease and phosphatase inhibitors on ice. Analysis occurred via western blot using the indicated antibodies. COMPOUND 8 treatment reduced PD-L1 levels relative to the vinculin loading control.

FIGS. 24A and 24B depict that knockdown of WWP1 by siRNA increases PD-L1 protein levels, PD-L1 glycosylation and cell surface expression in Hs578T cells. Cells were treated with 10 nM pooled siRNA (IDT) and harvested 48 hours after, splitting cells for flow cytometry and western blot. For western blot, cells were lysed in RIPA buffer containing protease and phosphatase inhibitors on ice. Lysates were analyzed by western blot using the indicated antibodies. PD-L1-APC (BD Biosciences) and a matched isotype control were used to visualize PD-L1 cell surface expression by flow cytometry (1:250 dilution). As shown by western blot, the knockdown of WWP1 yielded increased PD-L1 protein levels, including an increase in the glycosylated band. Similarly, WWP1 knockdown dramatically increased PD-L1 cell surface expression by flow cytometry (10% positive in control vs. isotype to 75% positive after knockdown). The mean fluorescence intensity of the positive population was also increased by about 10-fold, suggesting that not only were more cells expressing PD-L1, but the cells that did also contained more total PD-L1 on the cell surface.

FIG. 25 depicts that WWP1 knockdown by siRNA increases PD-L1 protein levels and glycosylation in multiple cancer cell models. Cells were treated with 10 nM pooled siRNA (IDT) and harvested 48 hours after. Cells were lysed in RIPA buffer containing protease inhibitors on ice. Lysates were analyzed by western blot using the indicated antibodies. TNBCs Hs578T and BT-20, the colorectal cancer cell line Du145, and the ER+ lung cancer line A549 all demonstrated increased PD-L1 protein levels, including the glycosylated form (and presumably surface expression), upon WWP1 knockdown. These data are suggested of a general phenomenon and a reproducible connection between WWP1 and PD-L1.

FIG. 26 depicts that interferon gamma dramatically boosts PDL1 expression in triple negative breast cancer cell lines, but not receptor positive breast cancer cells. Hs578T, BT-20 and MCF-7 cells were cultured for 48 hours in the presence or absence of interferon gamma (25 ng/mL in the cell culture media). Cells were lysed in RIPA buffer containing protease inhibitors on ice. Lysates were analyzed by western blot using the indicated antibodies. Relative to the vinculin loading control, interferon gamma upregulates PD-L1 protein levels within TNBCs shown to express PD-L1 at baseline. Conversely, interferon gamma induced PD-L1 expression was not readily observable in the ER+ cell line MCF-7, which does not appear to express PD-L1 protein at baseline.

FIG. 27 depicts that interferon gamma moderately reduces WWP1 protein levels in multiple cancer cell lines. Prior to harvesting, A549 and Hs578T cells were cultured for 48 hours in the presence or absence of interferon gamma (25 ng/mL in the cell culture media) or 10 nM pooled siRNA (IDT). Cells were lysed in RIPA buffer containing protease inhibitors on ice. Lysates were analyzed by western blot using the indicated antibodies. Interferon gamma reduced WWP1 levels in both cell lines compared to control, but not to the extent observed by WWP1 siRNA knockdown. These data may indicate a connection between interferon gamma, WWP1 and PD-L1.

FIG. 28 depicts that interferon gamma and WWP1 siRNA cooperatively increase PD-L1 expression in multiple cancer cell lines. Hs578T, BT-20 and A549 cells were cultured for 48 hours in the presence or absence of interferon gamma (25 ng/mL in the cell culture media), presence of absence of 10 nM pooled siRNA (IDT), or both. Cells were lysed in RIPA buffer containing protease inhibitors on ice. Lysates were analyzed by western blot using the indicated antibodies. Both interferon or siRNA alone reduced WWP1 expression (siRNA have a more dramatic effect on WWP1 levels) and increased PD-L1 protein expression. The combination of WWP1 siRNA and interferon further augmented PD-L1 protein levels beyond that of siRNA or interferon treatment alone, suggesting a cooperative activity of WWP1 and interferon on the PD-L1 pathway.

FIGS. 29A and 29B depict that WWP1 shRNA expressing Du145 cell lines demonstrate moderate knockdown of WWP1 mRNA and protein levels. All shRNA constructs were introduced through lentiviral infection under puromycin selection. Cells were cultured for 48 hours in the presence or absence of interferon gamma (25 ng/mL in the cell culture media) at which time a fraction of cells was split for protein and mRNA evaluation. For protein, cells were lysed in RIPA buffer containing protease inhibitors on ice. Total RNA was isolated using the RNeasy kit from Qiagen, prior to reverse transcription to obtain cDNA. Compared to empty vector control lines, WWP1 shRNA expressing cells has about a 60% KD of WWP1 mRNA by qPCR, which translated to roughly 3-5-fold protein level reduction by western. Interferon itself modestly reduced WWP1 mRNA levels (15% reduction).

FIG. 30 depicts that WWP1 shRNA expressing Du145 cell lines demonstrate increased PD-L1 expression. All shRNA constructs were introduced through lentiviral infection under puromycin selection. Cells were cultured for 48 hours in the presence or absence of interferon gamma (25 ng/mL in the cell culture media). Cells were lysed in RIPA buffer containing protease inhibitors on ice. Analysis occurred via western blot using the indicated antibodies. In the absence of interferon gamma, relative to the vinculin loading control, WWP1 knockdown correlated with increased PD-L1 protein levels. Interferon treatment also increased PD-L1 protein levels relative to the vinculin loading control, in control shRNA lines (No WWP1 knockdown). WWP1 shRNA expression, resulting in WWP1 knockdown, increased PD-L1 protein levels relative to the vinculin loading control, in both the presence and absence of interferon. These data corroborate siRNA data presented previously.

FIG. 31 depicts that the NEDD4 family HECT ligases are expressed at comparable levels in Hs578T cells. Steady state cells were harvested, and total RNA was purified by an RNeasy kit from Qiagen. After reverse transcription to generate cDNA, qPCR was used to quantify each ligase mRNA level. All ligases tested were expressed at similar levels, suggesting each may be an active constituent of the Hs578T cell model. ITCH was also found to be expressed at similar levels (data not shown).

FIG. 32 depicts that PD-L1 mRNA is most affected by WWP1 siRNA treatment in Hs578T cells. Cells were treated with 10 nM pooled siRNA for the indicated gene (IDT) and harvested 48 hours after. Total RNA was purified by an RNeasy kit from Qiagen. After reverse transcription to generate cDNA, qPCR was used to quantify mRNA levels of each ligase and PD-L1. All ligases demonstrated >70% KD (data not shown). WWP1 knockdown, to a greater degree than WWP2 (13-vs. 2-fold), dramatically upregulated PD-L1 mRNA. These data demonstrate specificity in the pathway among closely related enzymes.

FIGS. 33A and 33B depict that PD-L1 protein levels are most affected by WWP1 siRNA treatment in Hs578T cells. Cells were treated with 10 nM pooled siRNA for the indicated gene (IDT) and harvested 48 hours after (split for protein and RNA analysis). For protein, cells were lysed in RIPA buffer containing protease inhibitors on ice. Lysates were analyzed by western blot using the indicated antibodies. Total RNA was purified by an RNeasy kit from Qiagen. After reverse transcription to generate cDNA, qPCR was used to quantify mRNA levels of each ligase (results displayed below the blot). WWP1 protein levels were reduced significantly and specifically to the WWP1 siRNA condition, relative to the actin loading control, though western blots for WWP2 were not successful; however, WWP2 demonstrated ˜80% knockdown by qPCR. Mirroring the qPCR results, WWP1 siRNA treatment significantly boosted PD-L1 levels, whereas WWP2 siRNA treatment may only modestly affect PD-L1.

FIGS. 34A and 34B depicts that PD-L1 protein levels are most affected by WWP1 siRNA treatment in Hs578T cells, replicate adding NEDD4 siRNA treatment. These data replicate and expand upon the previous example to include another related enzyme, NEDD4. Cells were treated with 10 nM pooled siRNA for the indicated gene (IDT) and harvested 48 hours after (split for protein and RNA analysis). For protein, cells were lysed in RIPA buffer containing protease inhibitors on ice. Lysates were analyzed by western blot using the indicated antibodies. Total RNA was isolated using an RNeasy kit from Qiagen After reverse transcription to generate cDNA, qPCR was used to quantify mRNA levels of each ligase. All siRNA targets demonstrated a >70% knockdown by qPCR. Only the WWP1 siRNA treatment resulted in significant upregulation of PD-L1 protein levels.

FIG. 35 depicts that ITCH siRNA treatment does not affect WWP1 or PD-L1 protein levels. Cells were treated with 10 nM pooled siRNA for the indicated gene (IDT) and harvested after 48 hours. Cells were lysed in RIPA buffer containing protease inhibitors on ice. Lysates were analyzed by western blot using the indicated antibodies. Knockdown of WWP1 was specific to WWP1 siRNA treatment, which also correlated with PD-L1 protein upregulation, relative to the actin and vinculin loading controls, respectively. ITCH siRNA (at both low and high concentrations) neither affected WWP1 levels nor PD-L1 levels, respective to actin and vinculin loading control.

FIG. 36 depicts that WWP1 knockdown is specific to WWP1 siRNA treatment. Cells were treated with 10 nM pooled siRNA for the indicated gene (IDT) and harvested after 48 hours. Cells were lysed in RIPA buffer containing protease inhibitors on ice. Lysates were analyzed by western blot using the indicated antibodies. Knockdown of WWP1 was specific to WWP1 siRNA treatment, relative to the vinculin loading control. siRNA constructs for WWP2 and NEDD4 did not affect WWP1 protein levels relative to the vinculin loading control.

FIG. 37 depicts that ASO transfection methods are well optimized in Hs578T cells. RNAiMax Lipofectamine (Thermo) was used as the transfection reagent at the manufacturer's recommended concentration. “AKT1” samples were treated with an AKT1 ASO, previously demonstrated to efficiently knock down the AKT1 target gene. “NC1” and “NC2” are scrambled/non-targeting ASO controls expected to have no impact on the cell. “Parental” cells were treated with only lipofectamine. Cells were treated with either 10 or 20 nM ASO (or lipofectamine alone) and harvested after 48 hours. Total RNA was isolated using an RNeasy kit from Qiagen. After reverse transcription to generate cDNA, qPCR was used to quantify AKT1 mRNA levels. Values are relative to “Parental” replicate 1. AKT1 ASO conditions successfully knocked down the AKT1 mRNA (>80%) compared to either the parental or negative control ASOs, with the 20 nM condition demonstrating slightly more efficacy. The data are indicative of optimized transfection conditions for WWP1 ASO screening. Error bars are standard error of the mean.

FIG. 38 depicts that many WWP1 ASOs significantly knockdown WWP1 mRNA levels in Hs578T cells. RNAiMax Lipofectamine (Thermo) was used as the transfection reagent at the manufacturer's recommended concentration. “siRNA” samples were treated with control (scrambled) or pooled WWP1 siRNA at 10 nM. “AKT1” samples were treated with an AKT1 ASO, previously demonstrated to efficiently knock down the AKT1 target gene. “NC1” and “NC2” are scrambled/non-targeting ASO controls expected to have no impact on the cell. “Parental” cells were treated with only lipofectamine. ASOs with the prefix “ASO” indicate those targeting WWP1 (44 in total). Cells were treated with either 10 or 20 nM ASO (or 10 nM siRNA, or lipofectamine alone) and harvested after 48 hours. Total RNA was isolated using an RNeasy kit from Qiagen. After reverse transcription to generate cDNA, qPCR was used to quantify WWP1 mRNA levels. Values are relative to “Parental” replicate 1. Control samples did not significantly affect WWP1 mRNA levels (“Parental”, “NC” and “AKT1” samples, green, blue, and purple bars, respectively), whereas the WWP1 siRNA positive control efficiently knocked down WWP1 mRNA by >80% (orange bars). WWP1 targeting ASOs showed varied efficacy depending on their location within the WWP1 gene body (e.g., ASOs 5-8 vs. 9-12), and some dose dependence (blue 10 nM vs. red 20 nM, e.g., “ASO8”). Ct values for “ASO1” transfection at the 20 nM treatment condition were off by ˜10 cycles compared to the 10 nM condition, suggesting a sample preparation effort. Error bars are standard error of the mean.

FIG. 39 depicts that a replicate screen of WWP1 ASO efficacy in Hs578T cells by Thermo Life Technologies confirms many WWP1 ASOs significantly reduce mRNA levels. Under similar conditions, all ASOs were rescreening with a large level of agreement with the prior screen.

FIG. 40 depicts that a replicate screen of WWP1 ASO efficacy in VERO-E6 cells by Thermo Life Technologies confirms many WWP1 ASOs significantly reduce mRNA levels. ASOs were also screen in non-human primate cells (VERO E6, African green monkey) under similar conditions, with a large level of agreement with the prior screen.

FIG. 41 depicts that candidate ASOs from the screen demonstrate efficient knockdown of WWP1 protein levels. RNAiMax Lipofectamine (Thermo) was used as the transfection reagent at the manufacturer's recommended concentration. Hs578T cells were treated with either control siRNA, pooled WWP1 siRNA (IDT) or the listed ASO at 10 nM prior to harvest at the 48-hour time point. Cells were lysed in RIPA buffer containing protease inhibitors. Western blots were used to quantify protein levels, using antibodies against Vinculin and WWP1. Relative to the vinculin loading control, WWP1 siRNA and the ASOs that demonstrated efficacy in the previous qPCR screen efficiently reduced WWP1 protein levels, compared to the appropriate controls.

FIGS. 42A and 42B depicts that candidate ASOs from the screen demonstrate efficient knockdown of WWP1 protein levels, data replication. The experiment from the previous example was replicated, using 20 nM ASO and a fraction of cells was used for qPCR analysis of mRNA. There was a very good correlation between the qPCR results and the western blot for WWP1 knockdown on the mRNA and protein levels, respectively.

FIG. 43 depicts that I3C shows promise as a potential SARS-CoV-2 inhibitor. Vero cells were grown to 70-90% confluency. The compounds being tested were dissolved to 10 mM DMSO and used to treat cells from 150 nM up to 10 μM in 2-fold increments. The compounds were incubated with the cells for up to 1 hour and then challenged with SARS-CoV-2 at an MOI of up to 0.2. After 36 hours, the cells were fixed in formalin and then stained with SARS-CoV-2 antibody against the N protein and a fluorescently tagged secondary antibody. Cell nuclei were stained with Hoechst 33342 dye. The cells were imaged using a Cytation (Biotek) automated imaging system to visualize the blue fluorescent nuclei and the green fluorescent infected cells expressing virus N protein. Images were analyzed by CellProfiler software using a customized analysis pipeline to count the nuclei and the infected cells. Infection efficiency is expressed as a function of infected cells/cell nuclei counted. Wells where there were less than 50% cells remaining were flagged due to the treatment causing cell detachment, which may indicate toxicity. For such treatments, these measurements were not interpreted for infection inhibition due to potential inaccuracies of counting small numbers of cells. The assay was performed with 3 replicates per dose. Effects were compared by one way ANOVA with multiple comparison to the control group using Graphpad Prism software. Controls included were 0.5% DMSO treated cells (vehicle) or treatment with E-64d at 5 μM. All treatments except for Indole-3 carbinol were ineffective for the doses tested. Indole-3-carbinol (I3C) was effective at inhibiting >75% infection at 5 μM. At 10 μM, >50% cell loss was seen across each replicate indicating the potential for cytotoxicity.

### DETAILED DESCRIPTION

It is to be understood that both the foregoing general description and the following detailed description are exemplary and explanatory only and are not restrictive of the present disclosure, as claimed. Herein, the use of the singular includes the plural unless specifically stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. Where permitted, all patents, applications, published applications and other publications, gene accession numbers and associated sequence information obtainable through databases such as National Center for Biotechnology Information (NCBI) and other data referred to throughout in the disclosure are incorporated by reference in their entirety for any purpose. Any conflict between the teachings of these patents and publications and this specification shall be resolved in favor of the latter.

### Antisense Oligonucleotides

### Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art. Standard techniques may be used for chemical synthesis, and chemical analysis. Certain such techniques and procedures may be found for example in “Carbohydrate Modifications in Antisense Research” Edited by Sangvi and Cook, American Chemical Society, Washington D.C., 1994; “Remington's Pharmaceutical Sciences,” Mack Publishing Co., Easton, Pa., 21st edition, 2005; and “Antisense Drug Technology, Principles, Strategies, and Applications” Edited by Stanley T. Crooke, CRC Press, Boca Raton, Fla.; and Sambrook et al., “Molecular Cloning, A laboratory Manual” 4th ed. (2012) Cold Spring Harbor Laboratory Press, Cold Spring Harbor, N.Y., which are hereby incorporated by reference for any purpose. As appropriate, procedures involving the use of commercially available kits and reagents are generally carried out in accordance with manufacturer-defined protocols and conditions unless otherwise noted.

For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001.

Unless otherwise indicated, the following terms have the following meanings:

As used herein, the singular forms “a,” “an,” and “the” include the plural referents unless the context clearly indicates otherwise. The terms “include,” “such as,” and the like are intended to convey inclusion without limitation, unless otherwise specifically indicated.

As used herein, the term “comprising” also specifically includes embodiments “consisting of” and “consisting essentially of” the recited elements, unless specifically indicated otherwise.

The term “about” indicates and encompasses an indicated value and a range above and below that value. In certain embodiments, the term “about” indicates the designated value ±10%, ±5%, or ±1%. In certain embodiments, where applicable, the term “about” indicates the designated value(s) one standard deviation of that value(s).

As used herein, the term “antisense” refers to an oligomer having a sequence of nucleotide bases and a subunit-to-subunit backbone that allows the antisense oligomer to hybridize to a target sequence in a nucleic acid (typically RNA) by Watson-Crick base pairing, to form an nucleic acid:oligomer heteroduplex within the target sequence. The oligomer may have exact sequence complementarity to the target sequence or near complementarity. In some embodiments, such an an antisense oligomer can be designed to block or inhibit translation of mRNA or to inhibit natural pre-mRNA splice processing and may be said to be “directed to” or “targeted against” a target sequence with which it hybridizes.

As used herein, “target region” means a portion of a target nucleic acid to which an antisense oligonucleotide is designed to hybridize. The term, “target nucleic acid” means a naturally occurring, identified nucleic acid. In certain embodiments, target nucleic acids are endogenous cellular nucleic acids, including, but not limited to RNA transcripts, pre-RNA, mRNA, microRNA. In certain embodiments, target nucleic acids are viral nucleic acids. In certain embodiments, target nucleic acids are nucleic acids that an antisense oligonucleotide is designed to affect.

“Hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

The terms “complementary” and “complementarity” refer to oligonucleotides (i.e., a sequence of nucleotides) related by base-pairing rules

As used herein, the term “complementary” refers to the capacity of an oligomeric oligonucleotide to hybridize to another oligomeric oligonucleotide or nucleic acid through nucleobase complementarity. For example, the sequence “T-G-A (5′-3′),” is complementary to the sequence “T-C-A (5′-3′)”. In certain embodiments, an antisense oligonucleotide and its target are complementary to each other when a sufficient number of corresponding positions in each molecule are occupied by nucleobases that can bond with each other to allow stable association between the antisense oligonucleotide and the target. One skilled in the art recognizes that the inclusion of mismatches is possible without eliminating the ability of the oligomeric oligonucleotides to remain in association. Complementarity may be “partial,” in which only some of the nucleic acids' bases are matched according to base pairing rules. Or, there may be “complete” or “total” or “100%” complementarity between the nucleic acids. As used herein, “100% complementary” in reference to an oligonucleotide or portion thereof means that each nucleobase of the oligonucleotide or portion thereof is capable of pairing with a nucleobase of a complementary nucleic acid or contiguous portion thereof. Thus, 100% complementary region comprises no mismatches or unhybridized nucleobases in either strand.

As used herein, “percent complementarity” means the percentage of nucleobases of an oligomeric oligonucleotide that are complementary to an equal-length portion of a target nucleic acid. Percent complementarity is calculated by dividing the number of nucleobases of the oligomeric oligonucleotide that are complementary to nucleobases at corresponding positions in the target nucleic acid by the total length of the oligomeric oligonucleotide. For example, complementary oligomeric oligonucleotides or regions may be 80% complementary. In certain embodiments, complementary oligomeric oligonucleotides or regions are 90% complementary. In certain embodiments, complementary oligomeric oligonucleotides or regions are 95% complementary.

As used herein, “oligonucleotide” means a strand of linked nucleosides connected via internucleoside linkages, wherein each nucleoside and internucleoside linkage may be modified or unmodified. As used herein, “modified oligonucleotide” means an oligonucleotide comprising at least one modified nucleoside and/or at least one modified internucleoside linkage. In certain embodiments, an oligonucleotide comprises one or more unmodified ribonucleosides (RNA) and/or unmodified deoxyribonucleosides (DNA) and/or one or more modified nucleosides.

As used herein “oligonucleoside” means an oligonucleotide in which none of the internucleoside linkages contains a phosphorus atom. As used herein, oligonucleotides include oligonucleosides.

As used herein “internucleoside linkage” means a covalent linkage between adjacent nucleosides in an oligonucleotide.

As used herein, “modified internucleoside linkage” means any internucleoside linkage other than a naturally occurring internucleoside linkage. It refers to a substitution or any change from a naturally occurring internucleoside bond (i.e. a phosphodiester internucleoside bond).

As used herein, the term “nucleoside” means a oligonucleotide comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. Modified nucleosides include abasic nucleosides, which lack a nucleobase. The term “nucleoside” generally refers to compounds consisting of a sugar usually selected from the group of ribose, deoxyribose, pentose, arabinose, substituted arabinose (e.g. 2′-F-arabino (FANA)), or hexose, and a purine or pyrimidine base.

As used herein, “nucleobase” means a group of atoms that can be linked to a sugar moiety to create a nucleoside that is capable of incorporation into an oligonucleotide, and wherein the group of atoms is capable of bonding with a complementary naturally occurring nucleobase of another oligonucleotide or nucleic acid. Nucleobases may be naturally occurring or may be modified. As used herein the terms, “unmodified nucleobase” or “naturally occurring nucleobase” means the naturally occurring heterocyclic nucleobases of RNA or DNA: the purine bases adenine (A) and guanine (G), and the pyrimidine bases thymine (T), cytosine (C) (including 5-methyl C), and uracil (U).

As used herein, “modified nucleobase” means any nucleobase that is not a naturally occurring nucleobase.

As used herein, “sugar moiety” means a naturally occurring sugar moiety or a modified sugar moiety of a nucleoside. The term, “naturally occurring sugar moiety” means a ribofuranosyl as found in naturally occurring RNA or a deoxyribofuranosyl as found in naturally occurring DNA.

As used herein, “modified sugar moiety” means a substituted sugar moiety or a sugar surrogate. The term, “substituted sugar moiety” means a furanosyl that is not a naturally occurring sugar moiety. Substituted sugar moieties include but are not limited to furanosyls comprising substituents at the 2′-position, the 3′-position, the 5′-position and/or the 4′-position. Certain substituted sugar moieties are bicyclic sugar moieties. As used herein, “2′-substituted sugar moiety” means a furanosyl comprising a substituent at the 2′-position other than H or OH. Unless otherwise indicated, a 2′-substituted sugar moiety is not a bicyclic sugar moiety (i.e., the 2′-substituent of a 2′-substituted sugar moiety does not form a bridge to another atom of the furanosyl ring.

As used herein, “MOE” means —OCH2CH2OCH3.

As used herein the term “sugar surrogate” means a structure that does not comprise a furanosyl and that is capable of replacing the naturally occurring sugar moiety of a nucleoside, such that the resulting nucleoside sub-units are capable of linking together and/or linking to other nucleosides to form an oligomeric oligonucleotide which is capable of hybridizing to a complementary oligomeric oligonucleotide. Such structures include rings comprising a different number of atoms than furanosyl (e.g., 4, 6, or 7-membered rings); replacement of the oxygen of a furanosyl with a non-oxygen atom (e.g., carbon, sulfur, or nitrogen); or both a change in the number of atoms and a replacement of the oxygen. Such structures may also comprise substitutions corresponding to those described for substituted sugar moieties (e.g., 6-membered carbocyclic bicyclic sugar surrogates optionally comprising additional substituents). Sugar surrogates also include more complex sugar replacements (e.g., the non-ring systems of peptide nucleic acid). Sugar surrogates include without limitation morpholinos, cyclohexenyls and cyclohexitols.

As used herein, “bicyclic sugar moiety” means a modified sugar moiety comprising a 4 to 7 membered ring (including but not limited to a furanosyl) comprising a bridge connecting two atoms of the 4 to 7 membered ring to form a second ring, resulting in a bicyclic structure. In certain embodiments, the 4 to 7 membered ring is a sugar ring. In certain embodiments the 4 to 7 membered ring is a furanosyl. In certain such embodiments, the bridge connects the 2′-carbon and the 4′-carbon of the furanosyl.

As used herein, “locked nucleic acid nucleoside” or “LNA” means a nucleoside comprising a bicyclic sugar moiety comprising a 4′-CH₂—O-2′-bridge.

As used herein, “constrained ethyl nucleoside” or “cEt” means a nucleoside comprising a bicyclic sugar moiety comprising a 4′-CH(CH₃)—O-2′-bridge.

As used herein, “substituent” and “substituent group,” means an atom or group that replaces the atom or group of a named parent oligonucleotide. For example, a substituent of a modified nucleoside is any atom or group that differs from the atom or group found in a naturally occurring nucleoside (e.g., a modified 2′-substituent is any atom or group at the 2′-position of a nucleoside other than H or OH). Substituent groups can be protected or unprotected. In certain embodiments, oligonucleotides of the present disclosure have substituents at one or at more than one position of the parent oligonucleotide. Substituents may also be further substituted with other substituent groups and may be attached directly or via a linking group such as an alkyl or hydrocarbyl group to a parent oligonucleotide.

Likewise, as used herein, “substituent” in reference to a chemical functional group means an atom or group of atoms differs from the atom or a group of atoms normally present in the named functional group. In certain embodiments, a substituent replaces a hydrogen atom of the functional group (e.g., in certain embodiments, the substituent of a substituted methyl group is an atom or group other than hydrogen which replaces one of the hydrogen atoms of an unsubstituted methyl group).

As used herein, “expression” means the process by which a gene ultimately results in a protein. Expression includes, but is not limited to, transcription, post-transcriptional modification (e.g., splicing, polyadenlyation, addition of 5′-cap), and translation.

As used herein, “Contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “cell-targeting moiety” means a conjugate group or portion of a conjugate group that is capable of binding to a particular cell type or particular cell types.

As used herein, “gapmer” means an antisense oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.”

As used herein, “inhibiting the expression or activity” refers to a reduction or blockade of the expression or activity relative to the expression of activity in an untreated or control sample and does not necessarily indicate a total elimination of expression or activity.

As used herein, “effective amount” or “therapeutically effective amount” means the amount of active pharmaceutical agent sufficient to effectuate a desired physiological outcome in an individual in need of the agent. The effective amount can vary among individuals depending on the health and physical condition of the individual to be treated, the taxonomic group of the individuals to be treated, the formulation of the composition, assessment of the individual's medical condition, and other relevant factors.

As used herein, “pharmaceutically acceptable carrier or diluent” means any substance suitable for use in administering to an animal. Certain such carriers enable pharmaceutical compositions to be formulated as, for example, tablets, pills, dragees, capsules, liquids, gels, symps, slurries, suspension and lozenges for the oral ingestion by a subject. In certain embodiments, a pharmaceutically acceptable carrier or diluent is sterile water; sterile saline; or sterile buffer solution.

As used herein, “pharmaceutical composition” means a mixture of substances suitable for administering to a subject. For example, a pharmaceutical composition may comprise an antisense oligonucleotide and a sterile aqueous solution. In certain embodiments, a pharmaceutical composition shows activity in free uptake assay in certain cell lines.

Provided herein are methods, oligonucleotides, and compositions for reducing levels of at least one target protein in a cell, e.g., by reducing levels of the mRNA of the target protein or inhibiting translation of the mRNA. In some embodiments, the target protein can be the ubiquitin ligase polypeptide, including, but not limited to WWP1, WWP2, and NEDD4. Also provided herein are methods, oligonucleotides, and compositions for treating, ameliorating, delaying or reducing a symptom of a cancer.

The term “inhibitor” refers to a natural or synthetic compound that has a biological effect to inhibit or significantly reduce or down-regulate the expression of a gene and/or a protein or that has a biological effect to inhibit or significantly reduce the biological activity of a protein.

As used herein, “inhibiting” refers to a reduction or blockade of the activity or expression relative to the activity in an untreated or control sample and does not necessarily indicate a total elimination of expression or activity. Inhibition can cause an overall decrease 20% or more, 30% or more, 40% or more, 45% or more, more preferably 50% or more, 55% or more, 60% or more, 65% or more, 70% or more, 75% or more, 80% or more, 85% or more, 90% or more, or 95% or more or 100% in the activity compared to activity under the same conditions but without the presence of a compound described herein.

### Certain Embodiments

The present disclosure includes but is not limited to the following embodiments:

Certain embodiments provide antisense oligonucleotides targeted to a human WWP1 nucleic acid. In certain embodiments, the WWP1 nucleic acid has the sequence set forth in RefSeq or GENBANK Accession No NM_007013.4 (incorporated by reference).

Certain embodiments provide antisense oligonucleotides targeted to a human WWP2 nucleic acid. In certain embodiments, the WWP1 nucleic acid has the sequence set forth in RefSeq or GENBANK Accession No NM_007014.5 (incorporated by reference).

Certain embodiments provide antisense oligonucleotides targeted to a human NEDD4 nucleic acid. In certain embodiments, the NEDD4 nucleic acid has the sequence set forth in RefSeq or GENBANK Accession No NM_006154.4 (incorporated by reference).

Certain embodiments provide methods, oligonucleotides, and compositions for reducing levels of at least one target protein in a cell, e.g., by reducing levels of the mRNA of the target protein or inhibiting translation of the mRNA.

Certain embodiments provide a method of reducing a mRNA in a mammal including administering to the mammal a composition comprising a modified antisense oligonucleotide targeted to the mRNA.

### Antisense Oligonucleotides

An oligonucleotide can be “antisense” to a target nucleic acid, meaning that is capable of undergoing hybridization to a target nucleic acid through hydrogen bonding.

In certain embodiments, an antisense oligonucleotide has a nucleobase sequence that, when written in the 5′ to 3′ direction, comprises the reverse complement of the target segment of a target nucleic acid to which it is targeted. In certain such embodiments, an antisense oligonucleotide has a nucleobase sequence that, when written in the 5′ to 3′ direction, comprises the reverse complement of the target segment of a target nucleic acid to which it is targeted.

In certain embodiments, an antisense oligonucleotide targeted to a mRNA nucleic acid is 10 to 30 nucleotides in length. In other words, antisense oligonucleotides are from 10 to 30 linked nucleobases. In other embodiments, the antisense oligonucleotide comprises a modified oligonucleotide consisting of 8 to 80, 10-80, 12 to 50, 15 to 30, 18 to 24, 19 to 22, or 20 linked nucleobases. In certain such embodiments, the antisense oligonucleotide comprises a modified oligonucleotide consisting of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked nucleobases in length, or a range defined by any two of the above values.

It is possible to increase or decrease the length of an antisense oligonucleotide, such as an antisense oligonucleotide, and/or introduce mismatch bases without eliminating activity. For example, in Woolf et al. (Proc. Natl. Acad. Sci. USA 89:7305-7309, 1992), a series of antisense oligonucleotides 13-25 nucleobases in length were tested for their ability to induce cleavage of a target RNA in an oocyte injection model. Antisense oligonucleotides 25 nucleobases in length with 8 or 11 mismatch bases near the ends of the antisense oligonucleotides were able to direct specific cleavage of the target mRNA, albeit to a lesser extent than the antisense oligonucleotides that contained no mismatches. Similarly, target specific cleavage was achieved using 13 nucleobase antisense oligonucleotides, including those with 1 or 3 mismatches.

In certain embodiments, antisense oligonucleotides consist of linked nucleosides.

In certain embodiments, an antisense oligonucleotide is a modified oligonucleotide. In certain embodiments, methods disclosed herein comprise contacting a cell with an antisense oligonucleotide. In some embodiments, antisense oligonucleotides may comprise unmodified nucleosides. In certain embodiments, oligonucleotides comprise modified nucleosides.

In general, modified oligonucleotides comprise at least one modification relative to unmodified RNA or DNA.

In certain embodiments, the modification comprises a modified nucleoside. In certain embodiments, the modified nucleoside comprises a modified sugar moiety. In certain embodiments, the modified nucleoside comprises a modified nucleobase. In certain embodiments, modified nucleosides comprise a modified sugar moiety and a modified nucleobase. In certain embodiments, the modification comprises a modified internucleoside linkage. In certain embodiments, the modification comprises a modified sugar moiety and/or a modified internucleoside linkage.

In certain embodiments, modified oligonucleotides have one or more asymmetric center and thus give rise to enantiomers, diastereomers, and other stereoisomeric configurations that may be defined, in terms of absolute stereochemistry, as (R) or (S), as a or f such as for sugar anomers, or as (D) or (L) such as for amino acids etc. Included in the modified oligonucleotides provided herein are all such possible isomers, including their racemic and optically pure forms, unless specified otherwise. Likewise, all cis- and trans-isomers and tautomeric forms are also included.

The oligonucleotides described herein include variations in which one or more atoms are replaced with a non-radioactive isotope or radioactive isotope of the indicated element.

For example, nucleotides herein that comprise hydrogen atoms encompass all possible deuterium substitutions for each of the ¹H hydrogen atoms. Isotopic substitutions encompassed by the nucleotides herein include but are not limited to: ²H or ³H in place of ¹H. ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S.

In certain embodiments, non-radioactive isotopic substitutions may impart new properties on the nucleotide that are beneficial for use as a therapeutic or research tool. In certain embodiments, radioactive isotopic substitutions may make the nucleotide suitable for research or diagnostic purposes, such as an imaging assay.

### Certain Mechanisms

In certain embodiments, nucleotides described herein selectively affect one or more target nucleic acid. Such nucleotides comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in a significant undesired antisense activity.

In certain antisense activities, hybridization of a nucleotide described herein to a target nucleic acid results in recruitment of a protein that cleaves the target nucleic acid. For example, certain nucleotides described herein result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, nucleotides described herein are sufficiently “DNA-like” to elicit RNase H activity. Further, in certain embodiments, one or more non-DNA-like nucleoside in the gap of a gapmer is tolerated.

In certain antisense activities, nucleotides described herein or a portion of the nucleotide is loaded into an RNA-induced silencing complex (RISC), ultimately resulting in catalytic cleavage of the target nucleic acid. For example, certain nucleotides described herein result in cleavage of the target nucleic acid by Argonaute. Nucleotides that are loaded into RISC are RNAi nucleotides. RNAi nucleotides may be double-stranded (siRNA) or single-stranded (ss-RNA).

In certain embodiments, hybridization of nucleotides described herein to a target nucleic acid does not result in recruitment of a protein that cleaves that target nucleic acid.

In certain such embodiments, hybridization of the nucleotide to the target nucleic acid results in alteration of splicing of the target nucleic acid. In certain embodiments, hybridization of the nucleotide to a target nucleic acid results in inhibition of a binding interaction between the target nucleic acid and a protein or other nucleic acid. In certain such embodiments, hybridization of the nucleotide to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein, and/or a phenotypic change in a cell or animal.

### Target Nucleic Acids, Target Regions and Nucleotide Sequences

Antisense nucleotide sequences that hybridizes with a target mRNA include, without limitation, those described herein, including those listed in Table 5 and Table 6.

It is understood that the sequence set forth in each SEQ ID NO in the Examples contained herein is independent of any modification to a sugar moiety, an internucleotide linkage, or a nucleobase. As such, antisense nucleotides defined by a SEQ ID NO can comprise, independently, one or more modifications to a sugar moiety, an internucleotide linkage, or a nucleobase.

In certain embodiments, a target region is a structurally defined region of the target nucleic acid. For example, a target region can encompass a 3′ UTR, a 5′ UTR, an exon, an intron, an exon/intron junction, a coding region, a translation initiation region, translation termination region, or other defined nucleic acid region. The structurally defined regions for a mRNA can be obtained by accession number from sequence databases such as NCBI and such information is incorporated herein by reference. In certain embodiments, a target region can encompass the sequence from a 5′ target site of one target segment within the target region to a 3′ target site of another target segment within the target region.

Targeting includes determination of at least one target segment to which an antisense nucleotide hybridizes, such that a desired effect occurs. In certain embodiments, the desired effect is a reduction in mRNA target nucleic acid levels. In certain embodiments, the desired effect is reduction of levels of protein encoded by the target nucleic acid or a phenotypic change associated with the target nucleic acid.

A target region can contain one or more target segments. Multiple target segments within a target region can be overlapping. Alternatively, they can be non-overlapping. In certain embodiments, target segments within a target region are separated by no more than about 300 nucleotides. In certain embodiments, target segments within a target region are separated by a number of nucleotides that is, is about, is no more than, is no more than about, 250, 200, 150, 100, 90, 80, 70, 60, 50, 40, 30, 20, or 10 nucleotides on the target nucleic acid, or is a range defined by any two of the preceding values. In certain embodiments, target segments within a target region are separated by no more than, or no more than about, 5 nucleotides on the target nucleic acid. In certain embodiments, target segments are contiguous. Contemplated are target regions defined by a range having a starting nucleic acid that is any of the 5′ target sites or 3′ target sites listed herein.

Suitable target segments can be found within a 5′ UTR, a coding region, a 3′ UTR, an intron, an exon, or an exon/intron junction. Target segments containing a start codon or a stop codon are also suitable target segments. A suitable target segment can specifically exclude a certain structurally defined region such as the start codon or stop codon.

The determination of suitable target segments can include a comparison of the sequence of a target nucleic acid to other sequences throughout the genome. For example, the BLAST algorithm can be used to identify regions of similarity amongst different nucleic acids. This comparison can prevent the selection of antisense nucleotide sequences that can hybridize in a non-specific manner to sequences other than a selected target nucleic acid (i.e., non-target or off-target sequences).

There can be variation in activity (e.g., as defined by percent reduction of target nucleic acid levels) of the antisense nucleotides within an active target region. In certain embodiments, phenotypic changes, such as a treating, ameliorating, delaying or reducing a symptom of a disease or disorder associated with a nuclear-retained RNA, are indicative of inhibition of a mRNA.

In certain embodiments, an antisense oligonucleotide comprises a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: an mRNA and a pre-mRNA, including intronic, exonic and untranslated regions. In certain embodiments, the target RNA is an mRNA. In certain embodiments, the target nucleic acid is a pre-mRNA. In certain such embodiments, the target region is entirely within an intron. In certain embodiments, the target region spans an intron/exon junction. In certain embodiments, the target region is at least 50% within an intron.

### Hybridization

In some embodiments, hybridization occurs between an antisense oligonucleotide disclosed herein and a mRNA. In some embodiments, hybridization occurs between an antisense oligonucleotide disclosed herein and a WWP1 and/or WWP2 and/or NEDD4 nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Hybridization conditions are sequence-dependent and are determined by the nature and composition of the nucleic acid molecules to be hybridized.

Methods of determining whether a sequence is specifically hybridizable to a target nucleic acid are well known in the art. In certain embodiments, the oligonucleotides provided herein are specifically hybridizable with a WWP1 or WWP2 and/or NEDD4 nucleic acid.

Methods of determining whether a sequence is specifically hybridizable to a target nucleic acid are well known in the art (Sambrooke and Russell, Molecular Cloning: A Laboratory Manual, Yd Ed., 2001). In certain embodiments, the antisense nucleotides provided herein are specifically hybridizable with a mRNA.

### Complementarity

An oligonucleotide is said to be complementary to another nucleic acid when the nucleobase sequence of such oligonucleotide or one or more regions thereof matches the nucleobase sequence of another oligonucleotide or nucleic acid or one or more regions thereof when the two nucleobase sequences are aligned in opposing directions. Nucleobase matches or complementary nucleobases, as described

herein, are limited to the following pairs: adenine (A) and thymine (T), adenine (A) and uracil (U), cytosine (C) and guanine (G), and 5-methyl cytosine (mC) and guanine (G) unless otherwise specified. Complementary oligonucleotides and/or nucleic acids need not have nucleobases complementarity at each nucleoside and may include one or more nucleobase mismatches. An oligonucleotide is fully complementary or 100% complementary when such oligonucleotides have nucleobase matches at each nucleoside without any nucleobase mismatches.

Non-complementary nucleobases between an oligonucleotide and a WWP1 and/or WWP2 and/or NEDD4 nucleic acid may be tolerated provided that the oligonucleotide remains able to specifically hybridize to a target nucleic acid. Moreover, a oligonucleotide may hybridize over one or more segments of a WWP1, WWP2, NEDD4 nucleic acid such that intervening or adjacent segments are not involved in the hybridization event (e.g., a loop structure, mismatch or hairpin structure).

In certain embodiments, the oligonucleotides provided herein, or a specified portion thereof, are, are at least, or are up to 70%, 80%, 85%, 86%, 87%, 88%, 89%, 90%, 91%, 92%, 93%, 94%, 95%, 96%, 97%, 98%, 99%, or 100% complementary to a WWP1, WWP2, or NEDD4 nucleic acid, a target region, target segment, or specified portion thereof. In certain embodiments, the oligonucleotides provided herein, or a specified portion thereof, are 70% to 75%, 75% to 80%, 80% to 85%, 85% to 90%, 90% to 95%, 95% to 100%, or any number in between these ranges, complementary to a WWP1, WWP2, or NEDD4 nucleic acid, a target region, target segment, or specified portion thereof. Percent complementarity of an oligonucleotide with a target nucleic acid can be determined using routine methods.

For example, an oligonucleotide in which 18 of 20 nucleobases of the oligonucleotide are complementary to a target region, and would therefore specifically hybridize, would represent 90 percent complementarity. In this example, the remaining non-complementary nucleobases may be clustered or interspersed with complementary nucleobases and need not be contiguous to each other or to complementary nucleobases. As such, a oligonucleotide which is 18 nucleobases in length having four non-complementary nucleobases which are flanked by two regions of complete complementarity with the target nucleic acid would have 77.8% overall complementarity with the target nucleic acid. Percent complementarity of an oligonucleotide with a region of a target nucleic acid can be determined routinely using BLAST programs (basic local alignment search tools) and PowerBLAST programs known in the art (Altschul et al., J. Mol. Biol., 1990, 215,403 410; Zhang and Madden, Genome Res., 1997, 7, 649-656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, oligonucleotides described herein, or specified portions thereof, are fully complementary (i.e. 100% complementary) to a target nucleic acid, or specified portion thereof. For example, a oligonucleotide may be fully complementary to a WWP1, WWP2, or NEDD4 nucleic acid, or a target region, or a target segment or target sequence thereof. As used herein, “fully complementary” means each nucleobase of a oligonucleotide is complementary to the corresponding nucleobase of a target nucleic acid.

### Antisense Oligonucleotide Motifs

In certain embodiments, antisense oligonucleotides targeted to a mRNA nucleic acid have chemically modified subunits arranged in patterns, or motifs, to confer to the antisense oligonucleotides properties such as enhanced the inhibitory activity, increased binding affinity for a target nucleic acid, or resistance to degradation by in vivo nucleases.

Chimeric antisense oligonucleotides typically contain at least one region modified so as to confer increased resistance to nuclease degradation, increased cellular uptake, increased binding affinity for the target nucleic acid, and/or increased inhibitory activity. A second region of a chimeric antisense oligonucleotide can optionally serve as a substrate for the nuclear ribonuclease RNase H, which cleaves the RNA strand of an RNA:DNA duplex.

Antisense oligonucleotides having a gapmer motif are considered chimeric antisense oligonucleotides. In a gapmer an internal region having a plurality of nucleotides that supports RNaseH cleavage is positioned between external regions having a plurality of nucleotides that are chemically distinct from the nucleosides of the internal region. In the case of an antisense oligonucleotide having a gapmer motif, the gap segment generally serves as the substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer can in some embodiments include β-D-ribonucleosides, β-D-deoxyribonucleosides, 2-modified nucleosides (such 2-modified nucleosides can include 2′-MOE, and 2′-O—CH3, among others), and bicyclic sugar modified nucleosides (such bicyclic sugar modified nucleosides can include those having a 4′-(CH2)n-O-2′ bridge, where n=1 or n=2). Preferably, each distinct region comprises uniform sugar moieties. The wing-gap-wing motif is frequently described as “X-Y-Z”, where “X” represents the length of the 5′ wing region, “Y” represents the length of the gap region, and “Z” represents the length of the 3′ wing region. As used herein, a gapmer described as “X-Y-Z” has a configuration such that the gap segment is positioned immediately adjacent each of the 5′ wing segment and the 3′ wing segment. Thus, no intervening nucleotides exist between the 5′ wing segment and gap segment, or the gap segment and the 3′ wing segment. Any of the antisense oligonucleotide described herein can have a gapmer motif. In some embodiments, X and Z are the same, in other embodiments they are different. In a preferred embodiment, Y is between 8 and 15 nucleotides. X, Y or Z can be any of 1, 2, 3, 4, 5, 6, 7, 8, 9, 10, 11, 12, 13, 14, 15, 16, 17, 18, 19, 20, 25, 30 or more nucleotides. Thus, gapmers include, but are not limited to, for example 5-10-5, 4-8-4, 4-12-3, 4-12-4, 3-14-3, 2-13-5, 2-16-2, 1-18-1, 3-10-3, 2-10-2, 1-10-1, 2-8-2, 6-8-6, 5-8-5, 1-8-1, or 2-6-2.

In certain embodiments, the antisense oligonucleotide as a “wingmer” motif, having a wing-gap or gap-wing configuration, i.e. an X-Y or Y-Z configuration as described above for the gapmer configuration. Thus, wingmer configurations include, but are not limited to, for example 5-10, 8-4, 4-12, 12-4, 3-14, 16-2, 18-1, 10-3, 2-10, 1-10, 8-2, 2-13, or 5-13.

In certain embodiments, antisense oligonucleotides targeted to a mRNA nucleic acid possess a 5-10-5 gapmer motif. In certain embodiments, an antisense nucleotide targeted to a mRNA nucleic acid has a gap-widened motif.

### Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide. Modifications to antisense nucleotides encompass substitutions or changes to internucleoside linkages, sugar moieties, or nucleobases. Modified antisense nucleotides are often preferred over native forms because of desirable properties such as, for example, enhanced cellular uptake, enhanced affinity for nucleic acid target, increased stability in the presence of nucleases, or increased inhibitory activity.

Chemically modified nucleosides can also be employed to increase the binding affinity of a shortened or truncated antisense oligonucleotide for its target nucleic acid. Consequently, comparable results can often be obtained with shorter antisense nucleotides that have such chemically modified nucleosides.

### Modified Internucleoside Linkages

The naturally occurring internucleoside linkage of RNA and DNA is a 3′ to 5′ phosphodiester linkage. Antisense nucleotides having one or more modified, i.e. non-naturally occurring, internucleoside linkages are often selected over antisense nucleotides having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, antisense nucleotides targeted to a mRNA comprise one or more modified internucleoside linkages. In certain embodiments, the modified internucleoside linkages are phosphorothioate linkages. In certain embodiments, each internucleoside linkage of an antisense nucleotide is a phosphorothioate internucleoside linkage.

### Modified Nucleobases

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications can impart nuclease stability, binding affinity or some other beneficial biological property to antisense compounds. Modified nucleobases include synthetic and natural nucleobases such as, for example, 5-methylcytosine (5-me-C). Certain nucleobase substitutions, including 5-methylcytosine substitutions, are particularly useful for increasing the binding affinity of an antisense compound for a target nucleic acid. For example, 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278).

Additional unmodified nucleobases include 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

Heterocyclic base moieties can also include those in which the purine or pyrimidine base is replaced with other heterocycles, for example 7-deaza-adenine, 7-deazaguanosine, 2-aminopyridine and 2-pyridone. Nucleobases that are particularly useful for increasing the binding affinity of antisense compounds include 5-substituted pyrimidines, 6-azapyrimidines and N-2, N-6 and 0-6 substituted purines, including 2 aminopropyladenine, 5-propynyluracil and 5-propynylcytosine.

In certain embodiments, antisense nucleotides targeted to a mRNA comprise one or more modified nucleobases. In certain embodiments, gap-widened antisense oligonucleotides targeted to a mRNA comprise one or more modified nucleobases. In certain embodiments, the modified nucleobase is 5-methylcytosine. In certain embodiments, each cytosine is a 5-methylcytosine.

### Modified Sugar Moieties

Antisense oligonucleotides described in the present disclosure can optionally contain one or more nucleosides wherein the sugar group has been modified. Such sugar modified nucleosides may impart enhanced nuclease stability, increased binding affinity, or some other beneficial biological property to the antisense oligonucleotides. In certain embodiments, nucleosides comprise a chemically modified ribofuranose ring moieties. Examples of chemically modified ribofuranose rings include without limitation, addition of substitutent groups (including 5′ and 2′ substituent groups, bridging of non-geminal ring atoms to form bicyclic nucleic acids (BNA), replacement of the ribosyl ring oxygen atom with S, N(R), or C(R1)(R)2 (R═H, C1-C12 alkyl or a protecting group) and combinations thereof. Examples of chemically modified sugars include 2′-F-5′-methyl substituted nucleoside (see PCT International Application WO 2008/101157 Published on Aug. 21, 2008 for other disclosed 5′,2′-bis substituted nucleosides) or replacement of the ribosyl ring oxygen atom with S with further substitution at the 2′-position (see published U.S. Patent Application US2005-0130923, published on Jun. 16, 2005) or alternatively 5′-substitution of a BNA (see PCT International Application WO 2007/134181 Published on Nov. 22, 2007 wherein LNA is substituted with for example a 5′-methyl or a 5′-vinyl group). Examples of nucleosides having modified sugar moieties include without limitation nucleosides comprising 5′-vinyl, 5′-methyl (R or S), 4′-S, 2′-F, 2′-OCH3 and 2′-O(CH2)2OCH3 substituent groups. The substituent at the 2′ position can also be selected from allyl, amino, azido, thio, O-allyl, O-C1-C10 alkyl, OCF3, O(CH2)2SCH3, O(CH2)2-O—N(Rm)(Rn), and O-CH2-C(═O)—N(Rm)(Rn), where each Rm and Rn are, independently, H or substituted or unsubstituted C1-C10 alkyl.

In certain embodiments each nucleoside unit includes a heterocyclic base and a pentofuranosyl, trehalose, arabinose, 2′-deoxy-2′-substituted arabinose, 2-O-substituted arabinose, 2′-F-substituted arabinose or hexose sugar group.

As used herein, “bicyclic nucleosides” refer to modified nucleosides comprising a bicyclic sugar moiety. Examples of bicyclic nucleosides include without limitation nucleosides comprising a bridge between the 4′ and the 2′ ribosyl ring atoms. In certain embodiments, antisense oligonucleotides provided herein include one or more bicyclic nucleosides wherein the bridge comprises a 4′ to 2′ bicyclic nucleoside.

Examples of such 4′ to 2′ bicyclic nucleosides, include but are not limited to one of the formulae: 4′-(CH2)-0-2′ (LNA); 4′-(CH2)-S-2′; 4′-(CH2)2-O-2′ (ENA); 4′-CH (CH3)_O-2′ and 4′-CH(CH2OCH3)-O-2′ (and analogs thereof see U.S. Pat. No. 7,399,845, issued on Jul. 15, 2008); 4′ C(CH3)(CH3)-O-2′ (and analogs thereof see published International Application WO/2009/006478, published Jan. 8, 2009); 4′-CH2-N(O('113)-2′ (and analogs thereof see published International Application WO/2008/150729, published Dec. 11, 2008); 4′-CH2-O—N(CH3)-2′ (see published

U.S. Patent Application US2004-0171570, published Sep. 2, 2004); 4′-CH2-N(R)—O-2′, wherein R is H, C1-C2 alkyl, or a protecting group (see U.S. Pat. No. 7,427,672, issued on Sep. 23, 2008); 4′-CH2-C(H)(CH3)-2′ (see Chattopadhyaya, et al., J. Org. Chem., 2009, 74, 118-134); and 4′-CH2-C(═CH2)-2′ (and analogs thereof see published International Application WO 2008/154401, published on Dec. 8, 2008). See, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al, Bioorg. Med. Chem. Lett., 1998, 8, 2219-2222; Singh et al, J. Org. Chem., 1998, 63, 10035-10039; Srivastava et al, J. Am. Chem. Soc., 129(26) 8362-8379 (Jul. 4, 2007); U.S. Pat. Nos. 7,053,207; 6,268,490; 6,770,748; 6,794,499; 7,034,133; and 6,525,191; Elayadi et al, Curr. Opinion Invens. Drugs, 2001, 2, 558-561; Braasch et al, Chem. Biol., 2001, 8, 1-7; and Orum et al, Curr. Opinion Mol. Then, 2001, 3, 239-243; and U.S. Pat. No. 6,670,461; International applications WO 2004/106356; WO 94/14226; WO 2005/021570; U.S. Patent Publication Nos. US2004-0171570; US2007-0287831; US2008-0039618; U.S. Pat. No. 7,399,845; U.S. patent Ser. Nos. 12/129,154; 60/989,574; 61/026,995; 61/026,998; 61/056,564; 61/086,231; 61/097,787; 61/099,844; PCT International Applications Nos. PCT/US2008/064591; PCT/US2008/066154; PCT/US2008/068922; and Published PCT International Applications WO 2007/134181. Each of the foregoing bicyclic nucleosides can be prepared having one or more stereochemical sugar configurations including for example a-L-ribofuranose and β-D-ribofuranose (see PCT international application PCT/DK98/00393, published on Mar. 25, 1999 as WO 99/14226).

In certain embodiments, bicyclic sugar moieties of BNA nucleosides include, but are not limited to, compounds having at least one bridge between the 4′ and the 2′ position of the pentofuranosyl sugar moiety wherein such bridges independently comprises 1 or from 2 to 4 linked groups independently selected from —[C(Ra)(Rb)]n-, —C(Ra)═C(Rb)-, —C(Ra)═N—, —C(═NRa)—, —C(═O)—, —C(═S)—, —O—, —Si(Ra)2-, —S(═O)X—, and —N(Ra)—;

wherein:

x is 0, 1, or 2;

n is 1, 2, 3, or 4;

each Ra and Rb is, independently, H, a protecting group, hydroxyl, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, heterocycle radical, substituted heterocycle radical, heteroaryl, substituted heteroaryl, C5-C7 alicyclic radical, substituted C5-C7 alicyclic radical, halogen, OJ1, NJ1J2, SJ1, N3, COOJ1, acyl (C(═O)—H), substituted acyl, CN, sulfonyl (S(═O)2-J1). or sulfoxyl (S(═O)-J1); and each J and J2 is, independently, H, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, acyl (C(═O)—H), substituted acyl, a heterocycle radical, a substituted heterocycle radical, C1-C12 aminoalkyl, substituted Cx-C12 aminoalkyl or a protecting group.

In certain embodiments, the bridge of a bicyclic sugar moiety is, —[C(Ra)(Rb)]n-, —[C(Ra)(Rb)]n-O—, —C(RaRb)—N(R)—O— or —C(RaR4)-O—N(R)—. In certain embodiments, the bridge is 4′-CH2-2′, 4′-(CH2)2-2′, 4′-(CH2)3-2′, 4′-CH2-O-2′, 4′-(CH2)2-O-2′, 4′-CH2-O—N(R)-2′ and 4′-CH2-N(R)—O-2′- wherein each R is, independently, H, a protecting group or C1-C12 alkyl. In certain embodiments, bicyclic nucleosides are further defined by isomeric configuration. For example, a nucleoside comprising a 4′-2′ methylene-oxy bridge, may be in the α-L configuration or in the β-D configuration. Previously, α-L-methyleneoxy (4′-CH2-O-2′) BNA's have been incorporated into antisense oligonucleotides that showed antisense activity (Frieden et al., Nucleic Acids Research, 2003, 21, 6365-6372).

In certain embodiments, bicyclic nucleosides include, but are not limited to, α-L-Methyleneoxy (4′-CH2-O-2′) BNA, β-D-Methyleneoxy (4′-CH2-O-2′) BNA, Ethyleneoxy (4′-(CH2)2-O-2′) BNA, Aminooxy (4′-CH2-O—N(R)-2′) BNA, Oxyamino (4′-CH2-N(R)—O-2′) BNA, and Methyl (methyleneoxy) (4′-CH(CH3)-O-2′) BNA, methylene-thio (4′-CH2-S-2′) BNA, methylene-amino (4′-CH2-N(R)-2′) BNA, methyl carbocyclic (4′-CH2-CH(CH3)-2′) BNA, and propylene carbocyclic (4′-(CH2)3-2′) BNA.

As used herein, “4′-2′ bicyclic nucleoside” or “4′ to 2′ bicyclic nucleoside” refers to a bicyclic nucleoside comprising a furanose ring comprising a bridge connecting two carbon atoms of the furanose ring connects the 2′ carbon atom and the 4′ carbon atom of the sugar ring.

As used herein, “monocyclic nucleosides” refer to nucleosides comprising modified sugar moieties that are not bicyclic sugar moieties. In certain embodiments, the sugar moiety, or sugar moiety analogue, of a nucleoside may be modified or substituted at any position.

As used herein, “2-modified sugar” means a furanosyl sugar modified at the 2′ position. In certain embodiments, such modifications include substituents selected from: a halide, including, but not limited to substituted and unsubstituted alkoxy, substituted and unsubstituted thioalkyl, substituted and unsubstituted amino alkyl, substituted and unsubstituted alkyl, substituted and unsubstituted allyl, and substituted and unsubstituted alkynyl.

In certain embodiments, 2′ modifications are selected from substituents including, but not limited to: O[(CH2)nO]mCH3, O(CH2)nNH2, O(CH2)nCH3, O(CH2)nONH2, OCH2C(═O) N(H)CH3, and O(CH2)nON[(CH2)nCH3]2, where n and m are from 1 to about 10. Other 2′-substituent groups can also be selected from: C1-C12 alkyl, substituted alkyl, alkenyl, alkynyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving pharmacokinetic properties, or a group for improving the pharmacodynamic properties of an antisense compound, and other substituents having similar properties.

In certain embodiments, modified nucleosides comprise a 2′-MOE side chain (Baker et al., J. Biol. Chem., 1997, 272, 11944-12000). Such 2′-MOE substitution have been described as having improved binding affinity compared to unmodified nucleosides and to other modified nucleosides, such as 2′-O-methyl, O-propyl, and O-aminopropyl. Oligonucleotides having the 2′-MOE substituent also have been shown to be antisense inhibitors of gene expression with promising features for in vivo use (Martin, P., Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926).

As used herein, “2′-modified” or ‘2′-substituted” refers to a nucleoside comprising a sugar comprising a substituent at the 2′ position other than H or OH. 2-modified nucleosides, include, but are not limited to, bicyclic nucleosides wherein the bridge connecting two carbon atoms of the sugar ring connects the 2′ carbon and another carbon of the sugar ring; and nucleosides with non-bridging 2′substituents, such as allyl, amino, azido, thio, O-allyl, O-C1-C10 alkyl, —OCF3, O—(CH2)2-O—CH3, 2′-O(CH2)2SCH3, O—(CH2)2-O—N(Rm)(Rn), or O-CH2-C(═O)—N(Rm)(R), where each Rm and Rn is, independently, H or substituted or unsubstituted C1-C10 alkyl. 2-modified nucleosides may further comprise other modifications, for example at other positions of the sugar and/or at the nucleobase.

As used herein, “2-F” refers to a nucleoside comprising a sugar comprising a fluoro group at the 2′ position.

As used herein, “2′-OMe” or “2′-OCH3” or “2′-O-methyl” each refers to a nucleoside comprising a sugar comprising an —OCH3 group at the 2′ position of the sugar ring.

As used herein, “MOE” or “2′-MOE” or “2′-OCH2CH2OCH3” or “2′-O-methoxyethyl” each refers to a nucleoside comprising a sugar comprising a —OCH2CH2OCH3 group at the 2′ position of the sugar ring.

As used herein, “oligonucleotide” refers to a compound comprising a plurality of linked nucleosides. In certain embodiments, one or more of the plurality of nucleosides is modified. In certain embodiments, an oligonucleotide comprises one or more ribonucleosides (RNA) and/or deoxyribonucleosides (DNA).

Many other bicyclo and tricyclo sugar surrogate ring systems are also known in the art that can be used to modify nucleosides for incorporation into antisense compounds (see for example review article: Leumann, J. C, Bioorganic & Medicinal Chemistry, 2002, 10, 841-854). Such ring systems can undergo various additional substitutions to enhance activity.

Methods for the preparations of modified sugars are well known to those skilled in the art.

In nucleotides having modified sugar moieties, the nucleobase moieties (natural, modified or a combination thereof) are maintained for hybridization with an appropriate nucleic acid target.

In certain embodiments, antisense compounds comprise one or more nucleotides having modified sugar moieties. In certain embodiments, the modified sugar moiety is 2′-MOE.

In certain embodiments, the 2′-MOE modified nucleotides are arranged in a gapmer motif. In certain embodiments, the modified sugar moiety is a cEt. In certain embodiments, the cEt modified nucleotides are arranged throughout the wings of a gapmer motif.

Certain Conjugated Antisense Oligonucleotides

Disclosed herein are antisense oligonucleotides and optionally one or more conjugate groups and/or terminal groups. In certain embodiments, conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides.

In certain embodiments, conjugate groups are attached near the 5′-end of oligonucleotides.

### Certain Terminal Groups

In certain embodiments, antisense oligonucleotides comprise one or more terminal groups. In certain such embodiments, antisense oligonucleotides comprise a stabilized 5′-phosphate. Stabilized 5′-phosphates include, but are not limited to 5′-phosphanates, including, but not limited to 5′-vinyl phosphonates. In certain embodiments, terminal groups comprise one or more abasic nucleosides and/or inverted nucleosides. In certain embodiments, terminal groups comprise one or more 2′-linked nucleosides. In certain such embodiments, the 2′-linked nucleoside is an abasic nucleoside.

Examples of terminal groups include but are not limited to conjugate groups, capping groups, phosphate moieties, protecting groups, abasic nucleosides, modified or unmodified nucleosides, and two or more nucleosides that are independently modified or unmodified.

### Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-trifylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBOJ., 1991, 10, 1111-1118; Kabanov et al., FEBSLett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3 Hphosphonate (Manoharan et al., Tetrahedron Lett., 1995, 36, 3651-3654; Shea et al, Nucl. Acids Res., 1990, 18, 3777-3783), a polyamine or a polyethylene glycol chain (Manoharan et al. Nucleosides & Nucleotides, 1995, 14, 969-973), or adamantane acetic, a palmityl moiety (Mishra et al, Biochim. Biophys. Acta, 1995, 1264, 229-237), an octadecylamine or hexylamino-carbonyl-oxycholesterol moiety (Crooke et al, J. Pharmacol. Exp. Ther., 1996, i, 923-937), a tocopherol group (Nishina et al. Molecular Therapy Nucleic Acids, 2015, 4, e220; doi:10.1038/mtna.2014.72 and Nishina et al. Molecular Therapy, 2008, 16, 734-740), or a GalNAc cluster (e.g, WO2014/179620).

### Conjugate Moieties

Conjugate moieties include, without limitation, intercalators, reporter molecules, polyamines, polyamides, peptides, carbohydrates (e.g, GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

### Certain Cell-Targeting Conjugate Moieties

In certain embodiments, a conjugate group comprises a cell-targeting conjugate moiety.

In certain embodiments, conjugate groups comprise cell targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group. In certain embodiments, the cell-targeting moiety comprises a branching group comprising one or more groups selected from alkyl, amino, oxo, amide, disulfide, polyethylene glycol, ether, thioether and hydroxylamino groups. In certain embodiments, the branching group comprises a branched aliphatic group comprising groups selected from alkyl, amino, oxo, amide, disulfide, polyethylene glycol, ether, thioether and hydroxylamino groups. In certain such embodiments, the branched aliphatic group comprises groups selected from alkyl, amino, oxo, amide and ether groups. In certain such embodiments, the branched aliphatic group comprises groups selected from alkyl, amino and ether groups. In certain such embodiments, the branched aliphatic group comprises groups selected from alkyl and ether groups. In certain embodiments, the branching group comprises a mono or polycyclic ring system.

In certain embodiments, each tether of a cell-targeting moiety comprises one or more groups selected from alkyl, substituted alkyl, ether, thioether, disulfide, amino, oxo, amide, phosphodiester, and polyethylene glycol, in any aliphatic group comprising one or more groups selected from alkyl, ether, thioether, disulfide, amino, oxo, amide, and polyethylene glycol, in any combination. In certain embodiments, each tether is a linear aliphatic group comprising one or more groups selected from alkyl, phosphodiester, ether, amino, oxo, and amide, in any combination. In certain embodiments, each tether is a linear aliphatic group comprising one or more groups selected from alkyl, ether, amino, oxo, and amid, in any combination. In certain embodiments, each tether is a linear aliphatic group comprising one or more groups selected from alkyl, amino, and oxo, in any combination. In certain embodiments, each tether is a linear aliphatic group comprising one or more groups selected from alkyl and oxo, in any combination. In certain embodiments, each tether is a linear aliphatic group comprising one or more groups selected from alkyl and phosphodiester, in any combination.

In certain embodiments, each ligand of a cell-targeting moiety is a carbohydrate, carbohydrate derivative, modified carbohydrate, polysaccharide, modified polysaccharide, or polysaccharide derivative. In certain such embodiments, the conjugate group comprises a carbohydrate cluster (see, e.g., Maier et al., “Synthesis of Antisense Oligonucleotides Conjugated to a Multivalent Carbohydrate Cluster for Cellular Targeting,” Bioconjugate Chemistry, 2003, 14, 18-29, or Rensen et al., “Design and Synthesis of Novel N-Acetylgalactosamine—Terminated Glycolipids for Targeting of Lipoproteins to the Hepatic Asialoglycoprotein Receptor,” J. Med. Chem. 2004, 47, 5798-5808, which are incorporated herein by reference in their entirety).

### Conjugate Linkers

Conjugate moieties are attached to oligonucleotides through conjugate linkers. In certain compounds, a conjugate group is a single chemical bond (i.e. conjugate moiety is attached to an oligonucleotide via a conjugate linker through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide

groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group. In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups.

One of the functional groups is selected to bind to a particular site on a compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl. Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted CpC\hat{ } alkyl, substituted or unsubstituted C2-C10 alkenyl or substituted or unsubstituted C2-C10 alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, such linkernucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-Nbenzoylcytosine, 5-methylcytosine, 4-N-benzoyl-5-methylcytosine, adenine, 6-N-benzoyladenine, guanine and 2-Nisobutyrylguanine. It is typically desirable for linkernucleosides to be cleaved from the compound after it reaches a target tissue. Accordingly, linker-nucleosides are typically linked to one another and to the remainder of the compound through cleavable bonds. In certain embodiments, such cleavable bonds are phosphodiester bonds.

Herein, linker-nucleosides are not considered to be part of the oligonucleotide. Accordingly, in embodiments in which a compound comprises an oligonucleotide consisting of a specified number or range of linked nucleosides and/or a specified percent complementarity to a reference nucleic acid and the compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, a compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such a compound is more than 30. Alternatively, a compound may comprise a modified oligonucleotide consisting of 8-30 nucleosides and no conjugate group. The total number of contiguous linked nucleosides in such a compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside. In certain embodiments, it is desirable for a conjugate group to be cleaved from the oligonucleotide. For example, in certain circumstances compounds comprising a particular conjugate moiety are better taken up by a particular cell type, but once the compound has been taken up, it is desirable that the conjugate group be cleaved to release the unconjugated or parent oligonucleotide. Thus, certain conjugate may comprise one or more cleavable moieties, typically within the conjugate linker. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases. In certain embodiments, a cleavable bond is selected from among: an amide, an ester, an ether, one or both esters of a phosphodiester, a phosphate ester, a carbamate, or a disulfide.

In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, one or more linker-nucleosides are linked to one another and/or to the remainder of the compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

Representative publications that teach the preparation of certain of the above noted conjugate groups and compounds comprising conjugate groups, tethers, conjugate linkers, branching groups, ligands, cleavable moieties as well as other modifications include without limitation, U.S. Pat. Nos. 5,994,517, 6,300,319, 6,660,720, 6,906,182, 7,262, 60 177, 7,491,805, 8,106,022, 7,723,509, 9,127,276, US 2006/0148740, US 2011/0123520, WO 2013/033230 and WO 2012/037254, Biessen et al, J. Med. Chem. 1995, 38, 1846-1852, Lee et al, Bioorganic & Medicinal Chemistry 2011, 19, 2494-2500, Rensen et al, J. Biol. Chem. 2001, 65276, 37577-37584, Rensen et al, J. Med. Chem. 2004, 47, 5798-5808, Sliedregt et al, J. Med. Chem. 1999, 42, 609.

In certain embodiments compounds described herein comprise a conjugate group found in any of the following references: Lee, CarbohydrRes, 1978, 67, 509-514; Connolly et M. J Biol Chem, 1982, 257, 939-945; Pavia et at, Int J Pep Protein Res, 1983, 22, 539-548; Lee et al, Biochem, 1984, 23, 4255-4261; Lee et al. Glycoconjugate J, 1987, 4, 317-328; Toyokuni et al. Tetrahedron Lett, 1990, 31, 2673-2676; Biessen et al, J Med Chem, 1995, 38, 1538-1546; Valentijn et al. Tetrahedron, 1997, 53, 759-770; Kim et al. Tetrahedron Lett, 1997, 38, 3487-3490; Lee et al, Bioconjug Chem, 1997, 8, 762-765; Kato et al., Glycobiol, 2001, 11, 821-829; Rensen et al., J Biol Chem, 2001, 276, 37577-37584; Lee et al., Methods Enzymol, 2003, 362, 38-43; Westerlind et al., Glycoconj J, 2004, 21, 227-241; Lee et al., Bioorg Med Chem Lett, 2006, 16(19), 5132-5135; Maierhofer et al., Bioorg Med Chem, 2007, 15, 7661-7676; Khorev et al., Bioorg Med Chem, 2008, 16, 5216-5231; Lee et al, Bioorg Med Chem, 2011, 19, 2494-2500; Korilova et al, Analyt Biochem, 2012, 425, 43-46; Pujol et al, Angew Chemie Int Ed Engl, 2012, 51, 7445-7448; Biessen et al, J Med Chem, 1995, 38, 1846-1852; Sliedregt et al, J Med Chem, 1999, 42, 609-618; Rensen et al, J Med Chem, 2004, 47, 5798-5808; Rensen et al, Arterioscler Thromb Vase Biol, 2006, 26, 169-175; van Rossenbeig et al. Gene Ther, 2004, 11, 457-464; Sato et al, J Am Chem Soc, 2004, 126, 14013-14022; Lee et al, J Org Chem, 2012, 77, 7564-7571; Biessen et al, FASEB J, 2000, 14, 1784-1792; Rajur et al, Bioconjug Chem, 1997, 8, 935-940; Duff et al. Methods Enzymol, 2000, 313, 297-321; Maier et al, Bioconjug Chem, 2003, 14, 18-29; Jayaprakash et al, OrgLett, 2010, 12, 5410-5413; Manoharan, Antisense Nucleic Acid Drug Dev, 2002, 12, 103-128; Merwin et al, Bioconjug Chem, 1994, 5, 612-620; Tomiya et al, Bioorg Med Chem, 2013, 21, 5275-5281; International applications WO1998/013381; WO2011/038356; WO1997/046098; WO2008/098788; WO2004/101619; WO2012/037254; WO2011/120053; WO2011/100131; WO2011/163121; WO2012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. Nos. 4,751,219; 8,552,163; 6,908,903; 7,262,177; 5,994,517; 6,300,319; 8,106,022; 7,491,805; 7,491,805; 7,582,744; 8,137,695; 6,383,812; 6,525,031; 6,660,720; 7,723,509; 8,541,548; 8,344,125; 8,313,772; 8,349,308; 8,450,467; 8,501,930; 8,158,601; 7,262,177; 6,906,182; 6,620,916; 8,435,491; 8,404,862; 7,851,615; Published U.S. Patent Application Publications US2011/0097264; US2011/0097265; US2013/0004427; US2005/0164235; US2006/0148740; US2008/0281044; US2010/0240730; US2003/0119724; US2006/0183886; US2008/0206869; US2011/0269814; US2009/0286973; US2011/0207799; US2012/0136042; US2012/0165393; US2008/0281041; US2009/0203135; US2012/0035115; US2012/0095075; US2012/0101148; US2012/0128760; US2012/0157509; US2012/0230938; US2013/0109817; US2013/0121954; US2013/0178512; US2013/0236968; US2011/0123520; US2003/0077829; US2008/0108801; and US2009/0203132; each of which is incorporated by reference in its entirety.

### Linked Oligonucleotides

In some embodiments, the present disclosure provides antisense oligonucleotides comprising two or more single stranded antisense oligonucleotides linked at their 5′ ends, wherein the compounds have two or more accessible 3′ ends. The linkage at the 5′ ends of the component oligonucleotides is independent of the other oligonucleotide linkages and may be directly via 5′, 3′ or 2′ hydroxyl groups, or indirectly, via a non-nucleotide linker or a nucleoside, utilizing either the 2′ or 3′ hydroxyl positions of the nucleoside. Linkages may also utilize a functionalized sugar or nucleobase of a 5′ terminal nucleotide.

Oligonucleotides according to the present disclosure comprise two identical or different sequences linked at their 5′-5′ ends via a phosphodiester, phosphorothioate or non-nucleoside linker Such oligomeric duplexes comprise a first oligonucleotide having a region complementary to a target nucleic acid and a second oligonucleotide having a region complementary to the first oligonucleotide.

Some non-nucleotide linkers according to the present disclosure permit attachment of more than two oligonucleotide-based compounds described herein. For example, the small molecule linker glycerol has three hydroxyl groups to which such oligonucleotides may be covalently attached. Some oligonucleotide-based compounds described in the present disclosure, therefore, comprise two or more oligonucleotides linked to a nucleotide or a non-nucleotide linker. Such oligonucleotides according to the present disclosure are referred to as being “branched”. Oligonucleotide-based compounds according to the present disclosure may comprise at least two linked antisense oligonucleotides with two or more free 3′ ends.

In some embodiments, the present disclosure provides antisense oligonucleotides comprising two or more single stranded antisense oligonucleotides linked at their 3′ ends, wherein the compounds have two or more accessible 5′ ends. In certain embodiments, antisense oligonucleotides described herein comprise at least two oligonucleotides having a nucleobase sequence complementary to that of a target nucleic acid. In certain embodiments, an oligonucleotide is linked with a second oligonucleotide to form an oligomeric duplex. In certain embodiments, the 3′end of the first oligonucleotide is paired with 3′ or 5′ end of the second oligonucleotide. In certain embodiments, the 5′end of the first oligonucleotide is paired with 3′ or 5′ end of the second oligonucleotide.

In certain embodiments, the first oligonucleotide of an oligomeric duplex comprises or consists of (1) a modified or unmodified oligonucleotide and optionally a conjugate group and (2) a second modified or unmodified oligonucleotide and optionally a conjugate group. Either or both antisense oligonucleotide of an oligomeric duplex may comprise a conjugate group. The oligonucleotides of each oligonucleotide of an oligomeric duplex may include non-complementary overhanging nucleosides.

In certain such embodiments, the second oligonucleotide improves a property of the first oligonucleotide compared to the property in the absence of the second, shorter oligonucleotide.

In certain such embodiments, the second oligonucleotide improves a property of the first oligomeric compound compared to the property where the second oligonucleotide is of equal or greater length than the first oligonucleotide. In certain embodiments, the improved property is one or more of: distribution to a target tissue, uptake into a target cell, potency, and/or efficacy. In certain embodiments, the improved property is penetration of the blood brain barrier. In certain embodiments, the improved property is penetration of the blood brain barrier which allows systemic administration of a duplex in order to reduce a target nucleic acid in the CNS tissue. In certain embodiments, the target tissue is in the CNS. In certain embodiments, the target tissue is muscle tissue. In certain embodiments, the target tissue is other than liver (extra-hepatic). In certain embodiments, it is desirable to reduce target in more than one tissue. In certain such embodiments, it is desirable to reduce target in the liver and one or more other tissues. In certain embodiments, it is desirable to reduce target in more than one extra-hepatic tissue.

In certain embodiments, the first oligonucleotide of a duplex is a gapmer. In certain such embodiments, the wings of the gapmer comprise 2′-MOE modified nucleosides. In certain embodiments, the wings of the gapmer comprise cEt nucleosides. In certain embodiments the wings of the gapmer comprise LNA nucleosides. In certain embodiments, the wings of a gapmer comprise at least one 2′-MOE modified nucleoside and at least one bicyclic nucleoside. In certain such embodiments, each such bicyclic nucleoside is selected from among an LNA nucleoside and a cEt nucleoside. In certain embodiments, the gap constitutes 7-10 2′-deoxynucleosides.

In certain embodiments, the second oligonucleotide of the duplex has a motif consisting of cEt nucleosides and DNA nucleosides. For example, the second oligonucleotide may have an A-B-C motif, wherein A and C are RNA-like nucleosides and B is DNA-like nucleosides. In certain embodiments, A and C are selected from either cEt or LNA and B is one or more 2′-deoxynucleosides. In certain embodiments, the second oligonucleotide of the duplex has an A-B-C motif selected from 4-2-3, 2-4-2, and 3-2-4. In certain embodiments, the second oligonucleotide comprises at least one bicyclic nucleoside. In certain embodiments, the bicyclic nucleoside is selected from cEt or LNA. In certain embodiments, the second oligonucleotide of the duplex has one or more 2′-deoxynucleosides. In certain embodiments, the second oligonucleotide comprises at least one 2′-MOE nucleoside. In certain embodiments, the second oligonucleotide comprises 2′-MOE and 2′-deoxynucleosides. In certain embodiments, the second oligonucleotide comprises at least one bicyclic nucleoside. In certain embodiments, the second oligonucleotide comprises at least one cEt nucleoside. In certain embodiments, the second oligonucleotide comprises at least one LNA nucleoside. In certain embodiments, the second oligonucleotide comprises cEt and 2′-deoxynucleosides. In certain embodiments, the second oligonucleotide has sugar motif of alternating modification types (including no modification). In certain such embodiments, the sugar motif of the second oligonucleotide alternates between 2′-MOE nucleosides and 2′-deoxynucleosides. In certain such embodiments, the sugar motif of the second oligonucleotide alternates between cEt nucleosides and 2′-deoxynucleosides. In certain embodiments, the second oligonucleotide has a sugar motif similar to a gapmer (as described above) except that it may not elicit cleavage of a target nucleic acid. Such gapmer-like motifs have a central region and flanking wing regions. In certain such embodiments, the central region is comprised of 2′-deoxynucleosides and the wing regions are cEt modified nucleosides. In certain such embodiments, the central region is comprised of 2′-deoxynucleosides and the wing regions are LNA modified nucleosides. In certain embodiments, the central region is comprised of 2′-deoxynucleosides and the wing regions are 2′-MOE modified nucleosides. The internucleoside linkages of the second oligonucleotide may be modified or phosphodiester. In certain embodiments, the internucleoside linkages of the second oligonucleotide follow a gapmer-like motif—phosphorothioate wings and phosphdiester in the center. Such internucleoside linkage motif may or may not track the sugar motif.

In certain embodiments, at least one of the first and second oligonucleotides comprises a conjugate group (as described above). Typically, the second oligonucleotide comprises a conjugate group. The conjugate group may be attached at either the 3′- or 5′-end of the oligonucleotide. In certain embodiments, a conjugate group is attached to both ends.

### Uses of the Antisense Oligonucleotides

The present disclosure is further directed to the use of the antisense oligonucleotides described herein, or pharmaceutically acceptable salts and solvates thereof, as inhibitors of HECT domain E3 ligases.

In one embodiment, the antisense oligonucleotides described in the present disclosure are inhibitors of WWP1 and/or WWP2 and/or NEDD4. In one embodiment, the antisense oligonucleotides described in the present disclosure are inhibitors of WWP1 and WWP2 and NEDD4. In one embodiment, the antisense oligonucleotides described in the present disclosure are inhibitors of WWP1, preferably selective inhibitors of WWP1. In one embodiment, the antisense oligonucleotides described in the present disclosure are inhibitors selective of WWP1, with respect to other HECT domain E3 ligases. In other embodiment, the antisense oligonucleotides described in the present disclosure are inhibitors selective of WWP2, with respect to other HECT domain E3 ligases. In other embodiment, the antisense oligonucleotides described in the present disclosure are inhibitors selective of NEDD4, with respect to other HECT domain E3 ligases.

The present disclosure provides a method for inhibiting HECT domain E3 ligases in a patient, preferably a warm-blooded animal, and even more preferably a human, in need thereof, which comprises administering to said patient an effective amount of a compound described herein, or a pharmaceutically acceptable salt and solvate thereof.

The present disclosure is further directed to the use of the antisense oligonucleotides described herein as a medicament, i.e. for medical use. Thus, in one embodiment, the present disclosure provides the use of the antisense oligonucleotides described herein for the manufacturing of a medicament. Especially, the present disclosure provides the use of the antisense oligonucleotides described herein for the manufacturing of a medicament.

Especially, the present disclosure provides the antisense oligonucleotides described herein, for use in the treatment and/or prevention of proliferative disorders, including cancers. Thus, in one embodiment, the present disclosure provides the use of the antisense oligonucleotides described herein for the manufacture of a medicament for treating and/or preventing cancer. The present disclosure also provides a method of treatment of cancer, which comprises administering to a mammal species in need thereof a therapeutically effective amount of the antisense oligonucleotides described herein.

The present disclosure also provides for a method for delaying in patient the onset of cancer comprising the administration of a pharmaceutically effective amount of the antisense oligonucleotides described herein to a patient in need thereof.

Various cancers are known in the art. Cancers that can be treated using the methods described in the present disclosure include solid cancers and non-solid cancers, especially benign and malignant solid tumors and benign and malignant non-solid tumors. The cancer may be metastatic or non-metastatic. The cancer may be familial or sporadic.

In one embodiment, the cancer to be treated according to the present disclosure is a solid cancer. As used herein, the term “solid cancer” encompasses any cancer (also referred to as malignancy) that forms a discrete tumor mass, as opposed to cancers (or malignancies) that diffusely infiltrate a tissue without forming a mass.

Examples of solid tumors include, but are not limited to: biliary tract cancer, brain cancer (including glioblastomas and medulloblastomas), breast cancer, carcinoid, cervical cancer, choriocarcinoma, colon cancer, colorectal cancer, endometrial cancer, esophageal cancer, gastric cancer, glioma, head and neck cancer, intraepithelial neoplasms (including Bowen's disease and Paget's disease), liver cancer, lung cancer, neuroblastomas, oral cancer (including squamous cell carcinoma), ovarian cancer (including those arising from epithelial cells, stromal cells, germ cells and mesenchymal cells), pancreatic cancer, prostate cancer, rectal cancer, renal cancer (including adenocarcinoma and Wilms tumor), sarcomas (including leiomyosarcoma, rhabdomyosarcoma, liposarcoma, fibrosarcoma and osteosarcoma), skin cancer (including melanoma, Kaposi's sarcoma, basocellular cancer and squamous cell cancer), testicular cancer including germinal tumors (seminomas, and non-seminomas such as teratomas and choriocarcinomas), stromal tumors, germ cell tumors, thyroid cancer (including thyroid adenocarcinoma and medullary carcinoma) and urothelial cancer.

In another embodiment, the cancer to be treated according to the present disclosure is a non-solid cancer. Examples of non-solid tumors include but are not limited to hematological neoplasms. As used herein, a hematologic neoplasm is a term of art which includes lymphoid disorders, myeloid disorders, and AIDS associated leukemias.

Lymphoid disorders include but are not limited to acute lymphocytic leukemia and chronic lymphoproliferative disorders (e.g., lymphomas, myelomas, and chronic lymphoid leukemias). Lymphomas include, for example, Hodgkin's disease, non-Hodgkin's lymphoma lymphomas, and lymphocytic lymphomas). Chronic lymphoid leukemias include, for example, T cell chronic lymphoid leukemias and B cell chronic lymphoid leukemias.

In a specific embodiment, the cancer is selected from breast, carcinoid, cervical, colorectal, endometrial, glioma, head and neck, liver, lung, melanoma, ovarian, pancreatic, prostate, renal, gastric, thyroid and urothelial cancers.

In a specific embodiment, the cancer is breast cancer. In a specific embodiment, the cancer is carcinoid cancer. In a specific embodiment, the cancer is cervical cancer. In a specific embodiment, the cancer is colorectal cancer. In a specific embodiment, the cancer is endometrial cancer. In a specific embodiment, the cancer is glioma. In a specific embodiment, the cancer is head and neck cancer. In a specific embodiment, the cancer is liver cancer. In a specific embodiment, the cancer is lung cancer. In a specific embodiment, the cancer is melanoma. In a specific embodiment, the cancer is ovarian cancer. In a specific embodiment, the cancer is pancreatic cancer. In a specific embodiment, the cancer is prostate cancer. In a specific embodiment, the cancer is renal cancer. In a specific embodiment, the cancer is gastric cancer. In a specific embodiment, the cancer is thyroid cancer. In a specific embodiment, the cancer is urothelial cancer. In a specific embodiment, the cancer is prostate cancer.

In another specific embodiment, the cancer is selected from the group consisting of: leukemia and multiple myeloma.

Preferably, the patient is a warm-blooded animal, more preferably a human.

In one embodiment, the subject receiving the antisense oligonucleotides described in the present disclosure is treated with an additional therapeutic agent in combination with the antisense oligonucleotides described herein, or has received the additional therapeutic agent within about fourteen, twenty, or thirty days of administration of the antisense oligonucleotides described herein. In one embodiment, the additional therapeutic agent comprises radiotherapeutic agent, chemotherapeutic agent, anti-angiogenic agent, apoptosis-inducing agent, anti-tubulin drug, anti-cellular or cytotoxic agent, steroid, check point inhibitor, cytokine antagonist, cytokine expression inhibitor, chemokine antagonist, chemokine expression inhibitor, anti-inflammatory corticosteroid or NSAIDs, coagulant or antiviral agent.

In one embodiment, the subject has previously received at least one prior therapeutic treatment, and has progressed subsequent to the administration of the at least one prior therapeutic treatment and prior to administration of the antisense oligonucleotides described in the present disclosure. In one embodiment, the prior therapeutic treatment is selected from the group consisting of chemotherapy, immunotherapy, radiation therapy, stem cell transplant, hormone therapy, and surgery.

The present disclosure also provides the antisense oligonucleotides described herein, for use in the treatment of a viral infection. The antisense oligonucleotides described herein are effective for inhibiting viruses, preferably enveloped viruses, in vivo or ex vivo. In certain embodiments, the compounds described herein shows broad spectrum antiviral activity. In certain embodiments a method of inhibiting the growth, and/or the proliferation, and/or the infectivity, of an enveloped virus, is provided where the method comprises contacting the virus with an effective amount of antisense oligonucleotides as described herein. In certain embodiments the method comprises inhibiting the growth and/or proliferation of the virus. In certain embodiments the method comprises inhibiting the infectivity of the virus. In certain embodiments the method comprises reducing the titer of a viral infection in the mammal.

In certain embodiments the method comprises administering the antisense oligonucleotides described in the present disclosure to a mammal infected with the virus. In certain embodiments the method comprises administering the antisense oligonucleotides described in the present disclosure to a mammal at risk for infection by the virus. In certain embodiments the mammal is a non-human mammal. In certain embodiments the mammal is a human. In certain embodiments the virus comprises an enveloped virus.

In certain embodiments the virus is a member of a family selected from the group consisting of Herpesviridae, Poxviridae, Hepadnaviridae, Coronaviridae, Flaviviridae, Togaviridae, Retroviridae, Orthomyxoviridae, Arenaviridae, Bunyaviridae, Filoviridae, Paramyxoviridae, and Rhabdoviridae.

In certain embodiments the virus comprises a virus selected from the group consisting of SARS-CoV-2, SARS-CoV, MERS-CoV, Herpes simplex, type 1, Herpes simplex, type 2, Varicella-zoster virus, Epstein-Barr virus, Human cytomegalovirus, Human herpesvirus, type 8, Smallpox, Hepatitis B virus, Severe acute respiratory syndrome virus, Hepatitis C virus, yellow fever virus, dengue virus, West Nile virus, TBE virus Zika virus, Rubella virus, Human immunodeficiency virus (HIV), Influenza virus, Lassa virus, Crimean-5 Congo hemorrhagic fever virus, Hantaan virus, Ebola virus, Marburg virus, Measles virus, Mumps virus, Parainfluenza virus, Respiratory syncytial virus, Rabies virus, and Hepatitis D virus (HDV). In certain embodiments the virus is Zika virus or HIV-1 In certain embodiments administering the compound(s) in an amount sufficient to ameliorates one or more symptoms of a pathology caused by the virus and/or to slow or prevent infection of the mammal by the virus.

In one embodiment, the antisense oligonucleotides described in the present disclosure is administered prior to, concomitant with, or subsequent to administration of the additional therapeutic agent, such as an adenosine receptor antagonist.

### Compositions and Methods for Formulating Pharmaceutical Compositions

In certain embodiments, the oligonucleotide is an antisense oligonucleotide. Oligonucleotides described herein may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, or extent of disease.

Certain embodiments provide pharmaceutical compositions comprising one or more oligonucleotides or a salt thereof. In certain embodiments, the oligonucleotides comprise or consist of a modified oligonucleotide. In certain such embodiments, the pharmaceutical composition comprises a suitable pharmaceutically acceptable diluent or carrier. In certain embodiments, a pharmaceutical composition comprises a sterile saline solution and one or more oligonucleotide. In certain embodiments, such pharmaceutical composition consists of a sterile saline solution and one or more oligonucleotide. In certain embodiments, the sterile saline is pharmaceutical grade saline. In certain embodiments, a pharmaceutical composition comprises one or more oligonucleotide and sterile water. In certain embodiments, a pharmaceutical composition consists of one oligonucleotide and sterile water. In certain embodiments, the sterile water is pharmaceutical grade water. In certain embodiments, a pharmaceutical composition comprises one or more oligonucleotide and phosphate-buffered saline (PBS).

In certain embodiments, a pharmaceutical composition consists of one or more antisense oligonucleotide and sterile PBS. In certain embodiments, the sterile PBS is pharmaceutical grade PBS. Compositions and methods for the formulation of pharmaceutical compositions are dependent upon a number of criteria, including, but not limited to, route of administration, or extent of disease.

An oligonucleotide described herein targeted to WWP1 and/or WWP2 and/or NEDD4 nucleic acid can be utilized in pharmaceutical compositions by combining the oligonucleotide with a suitable pharmaceutically acceptable diluent or carrier. In certain embodiments, a pharmaceutically acceptable diluent is water, such as sterile water suitable for injection. Accordingly, in one embodiment, employed in the methods described herein is a pharmaceutical composition comprising a oligonucleotide targeted to WWP1 and/or WWP2 and/or NEDD4 nucleic acid and a pharmaceutically acceptable diluent. In certain embodiments, the pharmaceutically acceptable diluent is water. In certain embodiments, the oligonucleotide comprises or consists of a modified oligonucleotide provided herein.

Pharmaceutical compositions comprising oligonucleotides provided herein encompass any pharmaceutically acceptable salts, esters, or salts of such esters, or any other oligonucleotide which, upon administration to an animal, including a human, is capable of providing (directly or indirectly) the biologically active metabolite or residue thereof. In certain embodiments, the oligonucleotide comprises or consists of a modified oligonucleotide.

Accordingly, for example, the disclosure is also drawn to pharmaceutically acceptable salts of oligonucleotides, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of a oligonucleotide which are cleaved by endogenous nucleases within the body, to form the active oligonucleotide.

In certain embodiments, the oligonucleotides or compositions further comprise a pharmaceutically acceptable carrier or diluent.

In certain embodiments, pharmaceutical compositions comprise a delivery system. Examples of delivery systems include, but are not limited to, liposomes, nanoparticles e.g. gold nanoparticles, lipid nanoparticles, spherical nucleic acids, emulsions etc. Certain delivery systems are useful for preparing certain pharmaceutical compositions including those comprising hydrophobic compounds. In certain embodiments, certain organic solvents such as dimethylsulfoxide are used.

In certain embodiments, antisense oligonucleotide may be admixed with pharmaceutically acceptable active and/or inert substances for the preparation of pharmaceutical compositions or formulations.

Compositions and methods for the formulation of pharmaceutical compositions depend on a number of criteria, including, but not limited to, route of administration, extent of disease, or dose to be administered.

In certain embodiments, pharmaceutical compositions comprising an antisense oligonucleotide encompass any pharmaceutically acceptable salts of the oligomeric compound, esters of the oligomeric compound, or salts of such esters. In certain embodiments, pharmaceutical compositions comprising antisense oligonucleotide comprising one or more oligonucleotide, upon administration to a subject, including a human, are capable of providing (directly or indirectly) the biologically active metabolite or residue thereof.

Accordingly, for example, the disclosure is also drawn to pharmaceutically acceptable salts of oligomeric compounds, prodrugs, pharmaceutically acceptable salts of such prodrugs, and other bioequivalents. Suitable pharmaceutically acceptable salts include, but are not limited to, sodium and potassium salts. In certain embodiments, prodrugs comprise one or more conjugate group attached to an oligonucleotide, wherein the conjugate group is cleaved by endogenous nucleases within the body.

Lipid moieties have been used in nucleic acid therapies in a variety of methods. In certain such methods, the nucleic acid, such as an oligomeric compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue.

In certain embodiments, a lipid moiety is selected to increase distribution of a pharmaceutical agent to muscle tissue.

In certain embodiments, pharmaceutical compositions comprise one or more tissue-specific delivery molecules designed to deliver the one or more pharmaceutical agents of the present disclosure to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), etc.). In certain of such embodiments, a pharmaceutical composition comprises a carrier and is formulated in aqueous solution, such as water or physiologically compatible buffers such as Hanks's solution, Ringer's solution, or physiological saline buffer.

Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

The compositions of the present disclosure may also be admixed, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption.

Representative United States patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

### Small Molecule Modulators

### Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art. Standard techniques may be used for chemical synthesis, and chemical analysis. Certain such techniques and procedures may be found for example in “Carbohydrate Modifications in Antisense Research” Edited by Sangvi and Cook, American Chemical Society, Washington D.C., 1994; “Remington's Pharmaceutical Sciences,” Mack Publishing Co., Easton, Pa., 21st edition, 2005; and “Antisense Drug Technology, Principles, Strategies, and Applications” Edited by Stanley T. Crooke, CRC Press, Boca Raton, Fla.; and Sambrook et al., “Molecular Cloning, A laboratory Manual” 4th ed. (2012) Cold Spring Harbor Laboratory Press, Cold Spring Harbor, N.Y., which are hereby incorporated by reference for any purpose. As appropriate, procedures involving the use of commercially available kits and reagents are generally carried out in accordance with manufacturer-defined protocols and conditions unless otherwise noted.

For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001.

Unless otherwise indicated, the following terms have the following meanings:

As used herein, the singular forms “a,” “an,” and “the” include the plural referents unless the context clearly indicates otherwise. The terms “include,” “such as,” and the like are intended to convey inclusion without limitation, unless otherwise specifically indicated.

As used herein, the term “comprising” also specifically includes embodiments “consisting of” and “consisting essentially of” the recited elements, unless specifically indicated otherwise.

The term “about” indicates and encompasses an indicated value and a range above and below that value. In certain embodiments, the term “about” indicates the designated value ±10%, ±5%, or 1%. In certain embodiments, where applicable, the term “about” indicates the designated value(s) one standard deviation of that value(s).

The term “aldehyde” refers to a group —CHO.

The term “alkenyl” refers to unsaturated hydrocarbyl group, which may be linear or branched, comprising one or more carbon-carbon double bonds. Suitable alkenyl groups comprise between 2 and 6 carbon atoms, preferably between 2 and 4 carbon atoms, still more preferably between 2 and 3 carbon atoms. Examples of alkenyl groups are ethenyl, 2-propenyl, 2-butenyl, 3-butenyl, 2-pentenyl and its isomers, 2-hexenyl and its isomers, 2,4-pentadienyl and the like.

The term “alkenylcarbonyl” refers to a group —(C═O)-alkenyl wherein alkenyl is as herein defined.

The term “alkenylcarbonylamino” refers to a group —NH—(C═O)-alkenyl wherein alkenyl is as herein defined.

The term “alkoxy” refers to a group —O-alkyl wherein alkyl is as herein defined.

The term “alkyl” refers to an group (hydrocarbyl radical of formula CₙH₂ₙ₊₁ wherein n is a number greater than or equal to 1) or an alkyl group substituted by, for example, one to four substituents, such as, halo, trifluoromethyl, trifluoromethoxy, hydroxy, alkoxy, cycloalkoxy, heterocyclooxy, oxo, alkanoyl, aryloxy, alkanoyloxy, amino, alkylamino, arylamino, aralkylamino, cycloalkylamino, heterocycloamino, disubstituted amines in which the 2 amino substituents are selected from alkyl, aryl or aralkyl, alkanoylamino, aroylamino, aralkanoylamino, substituted alkanoylamino, substituted arylamino, substituted aralkanoylamino, thiol, alkylthio, arylthio, aralkylthio, cycloalkylthio, heterocyclothio, alkylthiono, arylthiono, aralkylthiono, alkylsulfonyl, arylsulfonyl, aralkylsulfonyl, sulfonamido (e.g. SO2NH2), substituted sulfonamido, nitro, cyano, carboxy, carbamyl (e.g. CONH2), substituted carbamyl (e.g. CONH alkyl, CONH aryl, CONH aralkyl or cases where there are two substituents on the nitrogen selected from alkyl, aryl or aralkyl), alkoxycarbonyl, aryl, substituted aryl, guanidino and heterocyclos, such as, indolyl, imidazolyl, furyl, thienyl, thiazolyl, pyrrolidyl, pyridyl, pyrimidyl and the like. Where noted above where the substituent is further substituted it will be with halogen, alkyl, alkoxy, aryl or aralkyl.

In some embodiments, an alkyl group is substituted by OH, OAlk, CF₃, NR₂.

Generally, alkyl groups of the present disclosure comprise from 1 to 8 carbon atoms, more preferably, alkyl groups of the present disclosure comprise from 1 to 6 carbon atoms. Alkyl groups may be linear or branched. Suitable alkyl groups include methyl, ethyl, propyl, butyl, pentyl, hexyl, heptyl and octyl.

The term “substituted alkyl” refers to an alkyl group substituted with, for example, one to four substituents, such as alkoxy, acyl, acylamino, acyloxy, amino, substituted amino, aminoacyl, aryl, substituted aryl, aryloxy, substituted aryloxy, cyano, halogen, hydroxyl, nitro, carboxyl, carboxyl esters, cycloalkyl, substituted cycloalkyl, heteroaryl, substituted heteroaryl, heterocyclyl, and substituted heterocyclyl.

The term “alkylaminoalkyl” refers to a group -alkyl-NH-alkyl wherein alkyl is as herein defined.

The term “alkylaminoalkylaminocarbonyl” refers to a group —(C═O)—NH-alkyl-NH-alkyl wherein alkyl is as herein defined.

The term “(alkylaminoalkyl)(alkyl)aminocarbonyl” refers to a group —(C═O)—NR¹R² wherein R¹ is an alkyl group and R² is a -alkyl-NH-alkyl group, wherein alkyl is as herein defined.

The term “alkylaminoalkylcarbonyl” refers to a group —(C═O)-alkyl-NH-alkyl wherein alkyl is as herein defined.

The term “alkylcarbonyl” refers to a group —(C═O)-alkyl wherein alkyl is as herein defined.

The term “alkylcarbonylamine” refers to a group —NH—(C═O)-alkyl wherein alkyl is as herein defined.

The term “alkylcarbonyloxyalkyl” refers to a group -alkyl-O—(C═O)-alkyl wherein alkyl is as herein defined.

The term “alkylheteroaryl” refers to any heteroaryl substituted by an alkyl group wherein alkyl is as herein defined.

The term “alkyloxyalkyl” refers to a group -alkyl-O-alkyl wherein alkyl is as herein defined.

The term “alkyloxycarbonyl” refers to a group —(C═O)—O-alkyl wherein alkyl is as herein defined.

The term “alkylsulfonyl” refers to a group —SO₂-alkyl wherein alkyl is as herein defined.

The term “alkylsulfonylaminoalkyl” refers to a group -alkyl-NH—SO₂-alkyl wherein alkyl is as herein defined.

The term “alkylsulfonealkyl” refers to a group -alkyl-SO₂-alkyl wherein alkyl is as herein defined.

The term “alkylsulfonimidoyl” refers to a group —S(═O)(═NH)-alkyl wherein alkyl is as herein defined.

The term “alkylsulfoxide” refers to a group —(S═O)-alkyl wherein alkyl is as herein defined.

The term “alkylsulfoxidealkyl” refers to a group -alkyl-SO-alkyl wherein alkyl is as herein defined.

The term “alkylene,” as used herein, refers to an alkyl group, as defined above, wherein one of the alkyl group's hydrogen atoms has been replaced with a bond. Alkylene group possess two points of attachment. Non-limiting examples of alkylene groups include —CH₂—, —CH₂CH₂—, —CH₂CH₂CH₂—, —CH₂CH₂CH₂CH₂—, —CH(CH₃)CH₂CH₂—, —CH(CH₃)— and CH₂CH(CH₃)CH₂—. In one embodiment, an alkylene group has from 1 to about 6 carbon atoms. In another embodiment, an alkylene group has from about 3 to about 5 carbon atoms. In another embodiment, an alkylene group is branched. In another embodiment, an alkylene group is linear. In one embodiment, an alkylene group is —CH₂—. In one embodiment, at least one hydrogen atom of an alkylene group is substituted by a substituent such as halo, trifluoromethyl, trifluoromethoxy, hydroxy, alkoxy, cycloalkoxy, heterocyclooxy, oxo, alkanoyl, aryloxy, alkanoyloxy, amino, alkylamino, arylamino, aralkylamino, cycloalkylamino, heterocycloamino, disubstituted amines in which the 2 amino substituents are selected from alkyl, aryl or aralkyl, alkanoylamino, aroylamino, aralkanoylamino, substituted alkanoylamino, substituted arylamino, substituted aralkanoylamino, thiol, alkylthio, arylthio, aralkylthio, cycloalkylthio, heterocyclothio, alkylthiono, arylthiono, aralkylthiono, alkylsulfonyl, arylsulfonyl, aralkylsulfonyl, sulfonamido (e.g. SO2NH2), substituted sulfonamido, nitro, cyano, carboxy, carbamyl (e.g. CONH2), substituted carbamyl (e.g. CONH alkyl, CONH aryl, CONH aralkyl or cases where there are two substituents on the nitrogen selected from alkyl, aryl or aralkyl), alkoxycarbonyl, aryl, substituted aryl, guanidino and heterocyclos, such as, indolyl, imidazolyl, furyl, thienyl, thiazolyl, pyrrolidyl, pyridyl, pyrimidyl and the like. Where noted above where the substituent is further substituted it will be with halogen, alkyl, alkoxy, aryl or aralkyl. In another embodiment, at least one hydrogen atom of an alkylene group is substituted by OH, OAlk, CF₃, NR₂.

The term “alkyne” refers to a class of monovalent unsaturated hydrocarbyl groups, wherein the unsaturation arises from the presence of one or more carbon-carbon triple bonds. Alkynyl groups typically, and preferably, have the same number of carbon atoms as described above in relation to alkyl groups. Non-limiting examples of alkynyl groups are ethynyl, 2-propynyl, 2-butynyl, 3-butynyl, 2-pentynyl and its isomers, 2-hexynyl and its isomers and the like.

The term “alkynealkyl” refers to a group -alkyl-alkyne wherein alkyl and alkyne are as herein defined.

The term “amino” refers to a group —NH₂.

The term “aminoalkyl” refers to a group -alkyl-NH₂ wherein alkyl is as herein defined.

The term “aminoalkylaminocarbonyl” refers to a group —(C═O)—NH-alkyl-NH₂ wherein alkyl is as herein defined.

The term “aminoalkylcarbonylamino” refers to a group —NH—(C═O)-alkyl-NH₂ wherein alkyl is as herein defined.

The term “aminocarbonyl” or “aminocarboxy” refers to a group —(C═O)—NH₂.

The term “(aminocarbonylalkyl)(alkyl)amino” refers to a group —NR¹R² wherein R¹ is an alkyl group and R² is a -alkyl-(C═O)—NH₂ group, wherein alkyl is as herein defined.

The term “aminocarbonylalkylamino” refers to a group —NH-alkyl-(C═O)—NH₂ wherein alkyl is as herein defined.

The term “aminosulfonyl” refers to a group —SO₂—NH₂.

The term “aryl” refers to a polyunsaturated, aromatic hydrocarbyl group having a single ring (i.e. phenyl) or multiple aromatic rings fused together (e.g. naphtyl), typically containing 5 to 12 atoms; preferably 5 to 10; more preferably the aryl is a 5- or 6-membered aryl. Non-limiting examples of aryl comprise phenyl, naphthalenyl.

The term “arylalkyl” refers to a group -alkyl-aryl wherein alkyl and aryl are as herein defined.

The term “aryloxyalkyl” refers to a group -alkyl-O-aryl wherein alkyl and aryl are as herein defined.

The term “carbonyl” refers to a group —(C═O)—.

The term “carbonylamino” refers to a group —NH—(C═O)—.

The term “cyano” refers to a group —CN.

The term “cyano” refers to a group -alkyl-CN.═ wherein alkyl is as herein defined.

The term “cyanopyridin” refers to a group consists of a pyridine ring with a nitrile group attached to it.

The term “cycloalkyl” refers to a cyclic alkyl group, that is to say, a monovalent, saturated, or unsaturated hydrocarbyl group having 1 or 2 cyclic structures. Cycloalkyl includes monocyclic or bicyclic hydrocarbyl groups. Cycloalkyl groups may comprise 3 or more carbon atoms in the ring and generally, according to the present disclosure comprise from 3 to 10, more preferably from 3 to 8 carbon atoms; still more preferably more preferably the cycloalkyl is a 5- or 6-membered cycloalkyl. Examples of cycloalkyl groups include but are not limited to cyclopropyl, cyclobutyl, cyclopentyl, cyclohexyl.

The term “cycloalkyloxy” refers to a group —O-cycloalkyl wherein cycloalkyl is as herein defined.

The term “dialkylamino” refers to a group —NR¹R² wherein R¹ and R² are both independently alkyl group as herein defined.

The term “dialkylaminoalkyl” refers to a group -alkyl-NR¹R² wherein R¹ and R² are both independently alkyl group, as herein defined.

The term “dialkylaminoalkylaminocarbonyl” refers to a group —(C═O)—NH-alkyl-NR¹R² wherein R¹ and R² are both alkyl group, as herein defined.

The term “dialkylaminoalkylcarbonyl” refers to a group —(C═O)-alkyl-NR¹R² wherein R¹ and R² are both alkyl group, as herein defined.

The term “dihydroxyalkyl” refers to a group alkyl is as herein defined substituted by two hydroxyl (—OH) groups.

The term “halo” or “halogen” refers to fluoro, chloro, bromo, or iodo.

The term “haloalkyl” refers to an alkyl group in which one or more hydrogen atom is replace by a halogen atom.

The term “haloalkyloxy” refers to a group —O-haloalkyl wherein alkyl is as herein defined.

The term “heteroaryl” refers to an aryl group as herein defined wherein at least one carbon atom is replaced with a heteroatom. In other words, it refers to 5 to 12 carbon-atom aromatic single rings or ring systems containing 2 rings which are fused together, typically containing 5 to 6 atoms; in which one or more carbon atoms is replaced by oxygen, nitrogen and/or sulfur atoms where the nitrogen and sulfur heteroatoms may optionally be oxidized and the nitrogen heteroatoms may optionally be quaternized. Non-limiting examples of such heteroaryl, include: pyrrolyl, furanyl, thiophenyl, pyrazolyl, imidazolyl, oxazolyl, isoxazolyl, thiazolyl, isothiazolyl, triazolyl, oxadiazolyl, thiadiazolyl, pyridinyl, pyridazinyl, pyrimidinyl and pyrazinyl.

The term “heteroarylalkyl” refers to a group -alkyl-heteroaryl wherein alkyl and heteroaryl are as herein defined.

The term “heterocyclyl” or “heterocycle” refers to non-aromatic, fully saturated or partially unsaturated cyclic groups (for example, 3 to 7 member monocyclic, 7 to 11 member bicyclic, or containing a total of 3 to 10 ring atoms) which have at least one heteroatom in at least one carbon atom-containing ring. Preferably the heterocyclyl is a 5- or 6-membered heterocyclyl. Each ring of the heterocyclic group containing a heteroatom may have 1, 2, 3 or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system, where valence allows. The rings of multi-ring heterocycles may be fused, bridged and/or joined through one or more spiro atoms. Non limiting exemplary heterocyclic groups include piperidinyl, piperazinyl, azetidinyl, azocanyl, diazepanyl, diazocanyl, morpholin-4-yl, oxazepanyl, pyrrolidinyl, thiomorpholin-4-yl, tetrahydrofuranyl, tetrahydropyranyl, aziridinyl, oxiranyl, thiiranyl, 2-imidazolinyl, pyrazolidinyl imidazolidinyl, isoxazolinyl, oxazolidinyl, isoxazolidinyl, thiazolidinyl, isothiazolidinyl, succinimidyl, 3H-indolyl, indolinyl, isoindolinyl, 2H-pyrrolyl, 1-pyrrolinyl, 2-pyrrolinyl, 3-pyrrolinyl, 4H-quinolizinyl, 2-oxopiperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, oxetanyl, thietanyl, 3-dioxolanyl, 1,4-dioxanyl, 2,5-dioximidazolidinyl, 2-oxopiperidinyl, 2-oxopyrrolodinyl, indolinyl, tetrahydrothiophenyl, tetrahydroquinolinyl, tetrahydroisoquinolinyl, 1-oxido-1-thiomorpholin-4-yl, 1-dioxido-1-thiomorpholin-4-yl, 1,3-dioxolanyl, 1,4-oxathianyl, 1,4-dithianyl, 1,3,5-trioxanyl, 1H-pyrrolizinyl, tetrahydro-1,1-dioxothiophenyl, N-formylpiperazinyl, dihydrotriazolopyrazine, dihydroimidazopyrazine, hexahydropyrrolopyrrole, hexahydropyrrolopyrazine.

The term “heterocyclylalkyl” refers to a group -alkyl-heterocyclyl wherein alkyl and heterocyclyl are as herein defined.

The term “heterocyclylalkylaminocarbonyl” refers to a group —(C═O)—NH-alkyl-heterocyclyl, wherein alkyl and heterocyclyl are as herein defined.

The term “(heterocyclyl)(alkyl)aminoalkyl” refers to a group -alkyl-NR¹R² wherein R¹ is an alkyl group and R² is a heterocyclyl group, wherein alkyl and heterocyclyl are as herein defined.

The term “heterocyclylalkyloxyalkyl” refers to a group -alkyl-O-alkyl-heterocyclyl wherein alkyl and heterocyclyl are as herein defined.

The term “heterocyclylcarbonyl” refers to a group —(C═O)-heterocyclyl wherein heterocyclyl is as herein defined.

The term “heterocyclyloxy” to a group —O-heterocyclyl wherein heterocyclyl is as herein defined.

The term “heterocyclylsulfonyl” refers to a group —SO₂-heterocyclyl wherein heterocyclyl is as herein defined.

The term “hydroxy” or “hydroxyl” refers to a group —OH.

The term “hydroxyalkyl” refers to a group -alkyl-OH wherein alkyl is as herein defined.

The term “hydroxyalkylaminoalkyl” refers to a group -alkyl-NH-alkyl-OH wherein alkyl is as herein defined.

The term “hydroxycarbonyl” refers to a group —C(═O)—OH wherein carbonyl is as herein defined. In other words, “hydroxycarbonyl” corresponds to a carboxylic acid group.

The term “oxo” refers to a ═O substituent.

The term “sulfonylamino” refers to a group —NH—SO₂.

The term “intermediate” or “intermediate compound” refers to a compound which is produced in the course of a chemical synthesis, which is not itself the final product, but is used in further reactions which produce the final product. There may be many different intermediate compounds between the starting material and end product in the course of a complex synthesis.

The term “about”, preceding a figure encompasses plus or minus 10%, or less, of the value of said figure. It is to be understood that the value to which the term “about” refers is itself also specifically, and preferably, disclosed.

The term “administration”, or a variant thereof (e.g. “administering”), means providing the active agent or active ingredient, alone or as part of a pharmaceutically acceptable composition, to the patient in whom/which the condition, symptom, or disease is to be treated or prevented.

As used herein, “IC50,” is intended to refer to the concentration of a substance (e.g., a compound or a drug) that is required for 50% inhibition of a biological process, or component of a process, including a protein, subunit, organelle, ribonucleoprotein, etc. In some embodiments, an IC50 can refer to the concentration of a substance that is required for 50% inhibition in vivo, as further defined elsewhere herein. In a further aspect, IC50 refers to the half maximal (50%) inhibitory concentration (IC) of a substance.

In a further aspect, the compound has an EC50 of less than about 500 μM, less than about 300 M less than about 100 μM, less than about 50 μM, less than about 10 μM, less than about 5 μM, or less than about 1 μM. In a further aspect, the compound has an IC50 of less than about 50 μM, less than about 30 μM, less than about 20 μM, less than about 1 μM, less than about 0.1 μM.

The term “inhibitor” refers to a natural or synthetic compound that has a biological effect to inhibit or significantly reduce or down-regulate the expression of a gene and/or a protein or that has a biological effect to inhibit or significantly reduce the biological activity of a protein.

As used herein, “inhibiting” refers to a reduction or blockade of the activity or expression relative to the activity in an untreated or control sample and does not necessarily indicate a total elimination of expression or activity. Inhibition can cause an overall decrease 20% or more, 30% or more, 40% or more, 45% or more, more preferably 50% or more, 55% or more, 60% or more, 65% or more, 70% or more, 75% or more, 80% or more, 85% or more, 90% or more, or 95% or more or 100% in the activity compared to activity under the same conditions but without the presence of a compound described in the present disclosure described herein.

As used herein, the term “modulate” refers to a change or an alteration (e.g. inhibition, reduction, increase, enhancement etc.) of any, or all, chemical and biological activities or properties of a cell or a biochemical entity in response to exposure to a compound described in the present disclosure.

In particular, “modulate” can refer to a change or an alteration in one or more biological or physiological mechanisms, effects, responses, functions, pathways, activities, signaling pathways or metabolisms involving HECT domain E3 ligases and their associated substrates. In preferred embodiments, modulation is reduction or inhibition of the activity of HECT domain E3 ligases by one or more compounds of the present disclosure.

The terms “therapeutically effective amount” or “effective amount” or “therapeutically effective dose” refer to the amount or dose of active ingredient that is aimed at, without causing significant negative or adverse side effects to the subject, (1) delaying or preventing the onset of a cancer in the subject; (2) reducing the severity or incidence of a cancer; (3) slowing down or stopping the progression, aggravation, or deterioration of one or more symptoms of a cancer affecting the subject; (4) bringing about ameliorations of the symptoms of a cancer affecting the subject; or (5) curing a cancer affecting the subject. A therapeutically effective amount may be administered prior to the onset of a cancer for a prophylactic or preventive action. Alternatively, or additionally, a therapeutically effective amount may be administered after initiation of a cancer for a therapeutic action.

The term “human” refers to a subject of both genders and at any stage of development (i.e. neonate, infant, juvenile, adolescent, adult).

The term “patient” refers to a mammal, more preferably a human, who/which is awaiting the receipt of, or is receiving medical care or is/will be the object of a medical procedure.

The expression “pharmaceutically acceptable” refers to the ingredients of a pharmaceutical composition are compatible with each other and not deleterious to the subject to which it is administered.

The expression “pharmaceutically acceptable carrier, diluent, excipient and/or adjuvant” refers to a substance that does not produce an adverse, allergic or other untoward reaction when administered to an animal, preferably a human. It includes any and all inactive substance such as for example solvents, cosolvents, antioxidants, surfactants, stabilizing agents, emulsifying agents, buffering agents, pH modifying agents, preserving agents (or preservating agents), antibacterial and antifungal agents, isotonifiers, granulating agents or binders, lubricants, disintegrants, glidants, diluents or fillers, adsorbents, dispersing agents, suspending agents, coating agents, bulking agents, release agents, absorption delaying agents, sweetening agents, flavoring agents and the like. For human administration, preparations should meet sterility, pyrogenicity, general safety and purity standards as required by regulatory offices, such as, e.g., FDA Office or EMA.

The terms “prevent”, “preventing” and “prevention”, as used herein, refer to a method of delaying or precluding the onset of a condition or disease and/or its attendant symptoms, barring a patient from acquiring a condition or disease, or reducing a patient's risk of acquiring a condition or disease.

The term, “pharmaceutical composition” refers to a mixture of substances suitable for administering to a subject. For example, a pharmaceutical composition may comprise an antisense oligonucleotide and a sterile aqueous solution. In certain embodiments, a pharmaceutical composition shows activity in free uptake assay in certain cell lines.

The term “small molecule” or “small molecular compound” generally refers to an organic molecule that is less than about 5 kilodaltons (Kd) in size. In some embodiments, the small molecule is less than about 4 Kd, 3 Kd, about 2 Kd, or about 1 Kd. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, small molecules are non-polymeric. Small molecules are not proteins, polypeptides, oligopeptides, peptides, polynucleotides, oligonucleotides, polysaccharides, glycoproteins, proteoglycans, etc. A derivative of a small molecule refers to a molecule that shares the same structural core as the original small molecule, but which can be prepared by a series of chemical reactions from the original small molecule.

The terms “treating” or “treatment” refer to therapeutic treatment; wherein the object is to prevent or slow down the targeted pathologic condition or disease. A subject or mammal is successfully “treated” for a disease or affection or condition if, after receiving the treatment according to the present disclosure, the subject or mammal shows observable and/or measurable reduction in or absence of one or more of the following: reduction of the number of cancer cells; and/or relief to some extent, for one or more of the symptoms associated with the specific disease or condition; reduced morbidity and mortality, and improvement in quality of life issues. The above parameters for assessing successful treatment and improvement in the disease are readily measurable by routine procedures familiar to a physician.

The term “subject” refers to a mammal, preferably a human. In one embodiment, the subject is diagnosed with a cancer. In one embodiment, the subject is a patient, preferably a human patient, who/which is awaiting the receipt of, or is receiving, medical care or was/is/will be the subject of a medical procedure or is monitored for the development or progression of a disease, such as a cancer. In one embodiment, the subject is a human patient who is treated and/or monitored for the development or progression of a cancer. In one embodiment, the subject is a male. In another embodiment, the subject is a female. In one embodiment, the subject is an adult. In another embodiment, the subject is a child.

### Certain Embodiments

The present disclosure includes but is not limited to the following embodiments:

Provided herein are compounds, methods, and compositions for inhibiting at least one target protein in a cell. In some embodiments, the target protein is ubiquitin ligase polypeptide, including, but not limited to WWP1, WWP2, and NEDD4. Also provided herein are compounds, methods and compositions for treating, ameliorating, delaying or reducing a symptom of a cancer and/or a viral infection.

Certain embodiments provide small molecule modulators (SMMs) targeted to a human WWP1 protein. In certain embodiments, the WWP1 has the sequence set forth in RefSeq or GENBANK Accession No NM_007013.4 (incorporated by reference).

Certain embodiments provide small molecule modulators (SMMs) targeted to a human WWP2 protein. In certain embodiments, the WWP2 has the sequence set forth in RefSeq or GENBANK Accession No NM_007014.5 (incorporated by reference).

Certain embodiments provide SMMs targeted to a human NEDD4 protein. In certain embodiments, the NEDD4 has the sequence set forth in RefSeq or GENBANK Accession No NG_051072.1 (incorporated by reference).

### Compounds—Small Molecule Modulators

The present disclosure thus provides small molecules, which may be useful as HECT domain E3 ligases. In one embodiment, the present disclosure thus provides compounds of formula I:

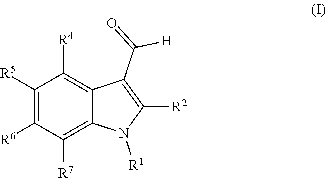

or a pharmaceutically acceptable salt or solvate thereof, wherein:
R¹ is selected from the group consisting of phenyl, naphthyl, phenylC₁-C₂alkyl, and naphthylC₁-C₂alkyl;
R² is H or C₁-C₃alkyl;
R⁴ is selected from the group consisting of H, halogen, and nitrile;
R⁵ is selected from the group consisting of H, halogen, and —CONHR⁸;
R⁶ is H or C₂-C₆alkynyl;
R⁷ is selected from the group consisting of H, C₁-C₃alkyl, and halogen; and
R⁸ is C₂-C₆alkynyl or 6-membered heteroaryl optionally substituted with halogen or nitrile;
wherein R⁵ and R⁶ are not both H.

In one embodiment, the present disclosure thus provides compounds of formula I or a pharmaceutically acceptable salt or solvate thereof, wherein:

R¹ is selected from the group consisting of C₆ to C₁₀ aryl, and C₆ to C₁₀ aryl C₁ to C₂ alkyl;

R² is selected from the group consisting of H and C₁ to C₃ alkyl;
R⁴ is selected from the group consisting of H, fluorine, and nitrile;
R⁵ is selected from the group consisting of H, fluorine, and CONHR⁸;
R⁶ is selected from the group consisting of H and C≡CH;
R⁷ is selected from the group consisting of H, C₁ to C₃ alkyl, and fluorine;
R⁸ is selected from the group consisting of propyn-3-yl and 2-cyanopyridin-4-yl.

In some embodiments, a compound of formula I is a compound of formula Ia:

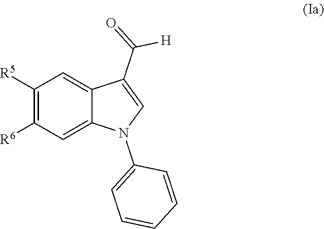

or a pharmaceutically acceptable salt or solvate thereof, wherein R⁵ and R⁶ are defined herein.

In another embodiment, the present disclosure also provides compounds of formula II:

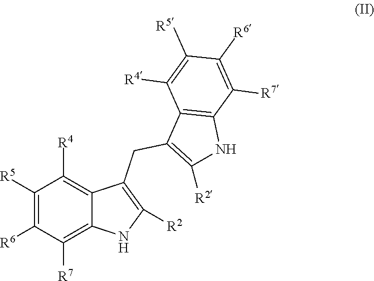

or a pharmaceutically acceptable salt or solvate thereof, wherein:
each of R² and R^{2′} are independently H or C₁-C₃alkyl;
each of R⁴ and R^{4′} are independently H or halogen;
each of R⁵ and R^{5′} are independently H or —CONR⁸R⁹;
each of R⁶ and R^{6′} are independently selected from the group consisting of H, —CO₂H, and —CONR⁸R⁹;
each of R⁷ and R^{7′} are independently selected from the group consisting of H, C₁-C₃alkyl, and halogen;
each R⁸ is independently C₂-C₆alkynyl or 6-membered heteroaryl optionally substituted with halogen or nitrile; and
each R⁹ is independently selected from H or C₁-C₃alkyl optionally substituted with —OH;
   wherein R⁵ and R⁶ are not both H.

In some embodiments, a compound of formula II is a compound of formula IIa:

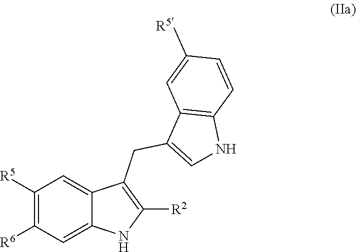

or a pharmaceutically acceptable salt or solvate thereof, wherein R², R⁵, R^{5′}, and R⁶ are defined herein.

In one embodiment, the present disclosure thus provides compounds of formula II or a pharmaceutically acceptable salt or solvate thereof, or a pharmaceutically acceptable salt or solvate thereof, wherein independently and individually:

R² and R^{2′} is selected from the group consisting of H and C₁ to C₃ alkyl;
R⁴ and R^{4′} are selected from the group consisting of H and fluorine;
R⁵ and R^{5′} are selected from the group consisting of H, fluorine, CO₂H, and CONR⁸R⁹;
R⁶ and R^{6′} are selected from the group consisting of H, CO₂H, and CONR⁸R⁹;
R⁷ and R^{7′} are selected from the group consisting of H, C₁ to C₃ alkyl, and fluorine;
R⁸ is selected from the group consisting of propyn-3-yl and 2-cyanopyridin-4-yl;
R⁹ is selected from the group consisting of H, methyl and 2-hydroxyethyl.

In some embodiments, R² is H or C₁-C₃alkyl. In some embodiments, R² is H. In some embodiments, R² is C₁-C₃alkyl. In some embodiments, R² is methyl.

In some embodiments, R^{2′} is H or C₁-C₃alkyl. In some embodiments, R^{2′} is H. In some embodiments, R^{2′} is C₁-C₃alkyl. In some embodiments, R^{2′} is methyl.

In some embodiments, R⁴ is selected from the group consisting of H, halogen, and nitrile. In some embodiments, R⁴ is selected from the group consisting of H, fluorine, and nitrile. In some embodiments, R⁴ is H or halogen. In some embodiments, R⁴ is H or fluorine. In some embodiments, R⁴ is H. In some embodiments, R⁴ is fluorine. In some embodiments, R⁴ is nitrile.

In some embodiments, R^{4′} is selected from the group consisting of H, halogen, and nitrile. In some embodiments, R^{4′} is selected from the group consisting of H, fluorine, and nitrile. In some embodiments, R^{4′} is H or halogen. In some embodiments, R^{4′} is H or fluorine. In some embodiments, R^{4′} is H. In some embodiments, R^{4′} is fluorine. In some embodiments, R^{4′} is nitrile.

In some embodiments, R⁵ selected from the group consisting of H, halogen, and —CONHR⁸. In some embodiments, R⁵ is selected from the group consisting of H, fluorine, and CONHR⁸. In some embodiments, R⁵ is selected from the group consisting of H, fluorine, CO₂H, and CONR⁸R⁹. In some embodiments, R⁵ is H or —CONHR⁸. In some embodiments, R⁵ is H. In some embodiments, R⁵ is H, and R⁶ is not H. In some embodiments, R⁵ is —CONHR⁸.

In some embodiments, R^{5′} selected from the group consisting of H, halogen, and —CONHR⁸. In some embodiments, R^{5′} is selected from the group consisting of H, fluorine, and CONHR⁸. In some embodiments, R^{5′} is selected from the group consisting of H, fluorine, CO₂H, and CONR⁸R⁹. In some embodiments, R^{5′} is H or —CONHR⁸. In some embodiments, R⁵ is H. In some embodiments, R⁵ is H, and R⁶ is not H. In some embodiments, R^{5′} is —CONHR⁸.

In some embodiments, R⁶ is H or C₂-C₆alkynyl. In some embodiments, R⁶ is selected from the group consisting of H, CO₂H, and CONR⁸R⁹. In some embodiments, R⁶ is H or —C≡CH. In some embodiments, R⁶ is H. In some embodiments, R⁶ is H and R⁵ is not H. In some embodiments, R⁶ is —C≡CH. In some embodiments, R⁶ is —CO₂H.

In some embodiments, R^{6′} is H or C₂-C₆alkynyl. In some embodiments, R^{6′} is selected from the group consisting of H, CO₂H, and CONR⁸R⁹. In some embodiments, R⁶ is H or —C≡CH. In some embodiments, R^{6′} is H. In some embodiments, R^{6′} is H and R⁵ is not H. In some embodiments, R^{6′} is —C≡CH. In some embodiments, R^{6′} is —CO₂H.

In some embodiments, R⁷ is selected from the group consisting of H, C₁-C₃alkyl, and halogen. In some embodiments, R⁷ is selected from the group consisting of H, C₁-C₃alkyl, and fluorine. In some embodiments, R⁷ is H. In some embodiments, R⁷ is C₁-C₃alkyl. In some embodiments, R⁷ is fluorine.

In some embodiments, R^{7′} is selected from the group consisting of H, C₁-C₃alkyl, and halogen. In some embodiments, R^{7′} is selected from the group consisting of H, C₁-C₃alkyl, and fluorine. In some embodiments, R^{7′} is H. In some embodiments, R^{7′} is C₁-C₃alkyl. In some embodiments, R^{7′} is fluorine.

In some embodiments, each R⁸ is independently C₂-C₆alkynyl or 6-membered heteroaryl optionally substituted with halogen or nitrile. In some embodiments, R⁸ is C₂-C₆alkynyl. In some embodiments, R⁸ is 6-membered heteroaryl optionally substituted with halogen or nitrile. In some embodiments, each R⁸ is independently selected from the group consisting of propyn-3-yl and 2-cyanopyridin-4-yl.

In some embodiments, each R⁹ is independently selected from H or C₁-C₃alkyl optionally substituted with —OH. In some embodiments each R⁹ is independently selected from H, methyl, and 2-hydroxyethyl.

Particularly preferred compound structures of formula I and II provided in the present disclosure are those listed in Table 1 hereafter.

TABLE 1

| Compound | Structure | Name |
|---|---|---|
| 1 | 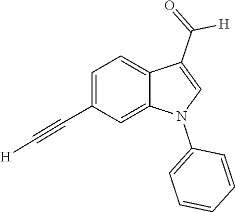 [SMILES: C#Cc1ccc2c(C=O)cn(-c3ccccc3)c2c1] | 6-ethynyl-1-phenyl-1H- indole-3-carbaldehyde |
| 2 | 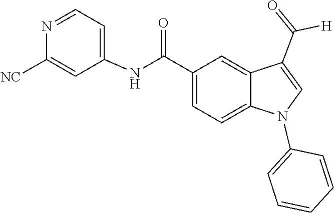 [SMILES: N#Cc1cc(NC(=O)c2ccc3c(c2)c(C=O)cn3-c2ccccc2)ccn1] | N-(2-cyanopyridin-4-yl)-3- formyl-1-phenyl-1H- indole-5-carboxamide |
| 3 | 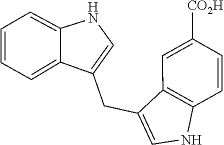 [SMILES: O=C(O)c1ccc2[nH]cc(Cc3c[nH]c4ccccc34)c2c1] | 3-((1H-indol-3-yl)methyl)- 1H-indole-5-carboxylic acid |
| 4 | 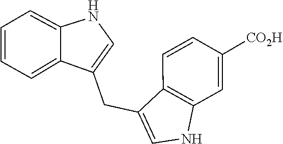 [SMILES: O=C(O)c1ccc2c(Cc3c[nH]c4ccccc34)c[nH]c2c1] | 3-((1H-indol-3-yl)methyl)- 1H-indole-6-carboxylic acid |
| 5 | 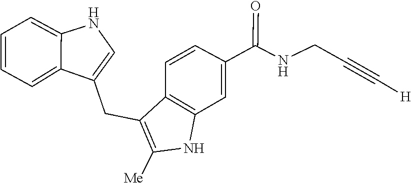 [SMILES: C#CCNC(=O)c1ccc2c(Cc3c[nH]c4ccccc34)c(C)[nH]c2c1] | 3-((1H-indol-3-yl)methyl)- 2-methyl-N-(prop-2-yn-1- yl)-1H-indole-6- carboxamide |
| 6 | 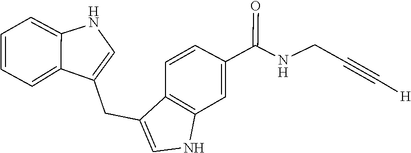 [SMILES: C#CCNC(=O)c1ccc2c(Cc3c[nH]c4ccccc34)c[nH]c2c1] | 3-((1H-indol-3-yl)methyl)- N-(prop-2-yn-1-yl)-1H- indole-6-carboxamide |
| 7 | 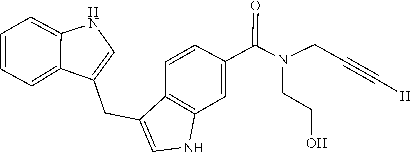 [SMILES: C#CCN(CCO)C(=O)c1ccc2c(Cc3c[nH]c4ccccc34)c[nH]c2c1] | 3-((1H-indol-3-yl)methyl)- N-(2-hydroxyethyl)-N- (prop-2-yn-1-yl)-1H- indole-6-carboxamide |
| 8 | 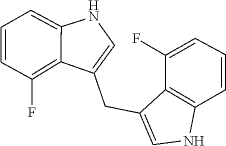 [SMILES: Fc1cccc2[nH]cc(Cc3c[nH]c4cccc(F)c34)c12] | bis(4-fluoro-1H-indol-3- yl)methane |
| 9 | 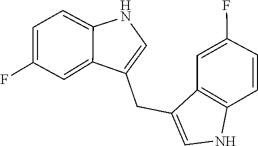 [SMILES: Fc1ccc2[nH]cc(Cc3c[nH]c4ccc(F)cc34)c2c1] | bis(5-fluoro-1H-indol-3- yl)methane |
| 10 | 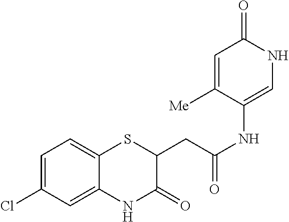 [SMILES: Cc1cc(=O)[nH]cc1NC(=O)CC1Sc2ccc(Cl)cc2NC1=O] | 2-(6-chloro-3-oxo-3,4- dihydro-2H- benzo[b][1,4]thiazin-2-yl)- N-(4-methyl-6-oxo-1,6- dihydropyridin-3- yl)acetamide |

or a pharmaceutically acceptable salt thereof.

The compounds of Table 1 were named using ChemBioDraw® Ultra version 12.0 (PerkinElmer).

In one embodiment, the present disclosure also relates to salts, solvates, enantiomers, isomers (including optical, geometric and tautomeric isomers), polymorphs, multi-component complexes, liquid crystals, prodrugs of compounds of formula I or II.

In one embodiment, the present disclosure relates to enantiomers and isomers (including optical, geometric and tautomeric isomers) of compounds of formula I and II. Indeed, the compounds of formula I or II may contain an asymmetric center and thus may exist as different stereoisomeric forms. Accordingly, the present disclosure includes all possible stereoisomers and includes not only racemic compounds but the individual enantiomers and their non-racemic mixtures as well. When a compound is desired as a single enantiomer, such may be obtained by stereospecific synthesis, by resolution of the final product or any convenient Intermediate compound, or by chiral chromatographic methods as each are known in the art. Resolution of the final product, an Intermediate compound, or a starting material may be performed by any suitable method known in the art.

In one embodiment, the present disclosure also relates to salts of compounds of formula I or II. Especially, the compounds described in the present disclosure may be in the form of pharmaceutically acceptable salts. Pharmaceutically acceptable salts of the compounds of formula I or II, ammonium salt, aspartate, benzoate, besylate, benzenesulfonate, bicarbonate/carbonate, bisulphate/sulphate, bitartrate, borate, calcium edetate, camsylate, citrate, clavulanate, cyclamate, dihydrochloride, edetate, edisylate, estolate, esylate, formate, fumarate, gluceptate, gluconate, glucuronate, glutamate, glycollylarsanilate, hexafluorophosphate, hexylresorcinate, hibenzate, hydrabamine, hydrochloride/chloride, hydrobromide/bromide, hydroiodide/iodide, hydroxynaphthoate, isethionate, isothionate, lactate, lactobionate, laurate, malate, maleate, malonate, mandelate, mesylate, methylbromide, N-methylglucamine, methylnitrate, methylsulphate, mucate, panoate, naphthylate, 2-napsylate, nicotinate, nitrate, oleate, orotate, oxalate, palmitate, pamoate, pantothenate, phosphate/hydrogen phosphate/dihydrogen phosphate, polygalacturonate, pyroglutamate, saccharate, salicylate, stearate, subacetate, succinate, tannate, tartrate, teoclate, tosylate, triethiodide, trifluoroacetate, valerate and xinofoate salts. Preferred pharmaceutically acceptable acid addition salts include hydrochloride/chloride, hydrobromide/bromide, bisulphate/sulphate, nitrate, citrate, tosylate, esylate and acetate. Suitable base salts are formed from bases which form non-toxic salts. Examples include the aluminum, ammonia, arginine, benzathine, N-benzylphenethyl-amine, calcium, chloroprocaine, choline, N,N′-dibenzylethylene-diamine, diethanolamine, diethylamine, 2-(diethylamino)ethanol, diolamine, ethanolamine, ethylenediamine, glycine, lithium, lysine, magnesium, meglumine, N-methyl-glutamine, morpholine, 4-(2-hydroxyethyl)morpholine, olamine, ornithine, piperazine, potassium, procaine, sodium, tetramethylammonium hydroxide, tris(hydroxymethyl)aminomethane, tromethamine and zinc salts. Hemisalts of acids and bases may also be formed, for example, hemisulphate and hemicalcium salts. When the compounds described in the present disclosure contain a hydrogen-donating heteroatom (e.g. NH), the present disclosure also covers salts and/or isomers formed by transfer of said hydrogen atom to a basic group or atom within the molecule.

Pharmaceutically acceptable salts of compounds of formula I or II may be prepared by reacting the compound of formula I or II with the desired base.

The reactions are typically carried out in solution. The salt may precipitate from solution and be collected by filtration or may be recovered by evaporation of the solvent. The degree of ionization in the salt may vary from completely ionized to almost non-ionized.

In addition, although generally, with respect to the salts of the compounds described in the present disclosure, pharmaceutically acceptable salts are preferred, it should be noted that the present disclosure in its broadest sense also included non-pharmaceutically acceptable salts, which may for example be used in the isolation and/or purification of the compounds described in the present disclosure. For example, salts formed with optically active acids or bases may be used to form diastereoisomeric salts that can facilitate the separation of optically active isomers of the compounds of formula I or II above.

In one embodiment, the present disclosure also relates to solvates of compounds of formula I or II. The compounds described in the present disclosure may be in the form of pharmaceutically acceptable solvates. Pharmaceutically acceptable solvates of the compounds of formula I or II contains stoichiometric or sub-stoichiometric amounts of one or more pharmaceutically acceptable solvent molecule such as ethanol or water. The term “hydrate” refers to when the said solvent is water.

In one embodiment, the present disclosure also relates to prodrugs of compounds of formula I or II. For example, in the case of an alcohol group being present, pharmaceutically acceptable esters can be employed, e.g. acetate, maleate, pivaloyloxymethyl, and the like, and those esters known in the art for modifying solubility or hydrolysis characteristics for use as sustained release or prodrug formulations.

### Process of Manufacturing

The compounds of formula I or II can be prepared by different ways with reactions known by one skilled in the art. See WO1998050357 and WO2002028832 for substituted diindolylmethanes.

The present disclosure also provides a process of manufacturing of compounds of formula I.

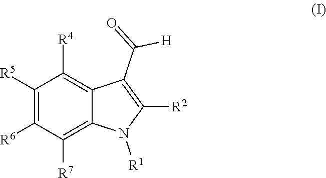

or a pharmaceutically acceptable salt or solvate thereof, wherein R¹, R², R⁴, R⁵, R⁶, and R⁷ are hereafter defined.

N-aryl 3-formylindoles of general formula (I) are prepared using a series of selective transformations to modify a suitably functionalized indole starting material. As shown in Scheme 1, a set of four reactions are used to elaborate the 3-formyl group, the N-aryl group and the groups at R⁵ and R⁶. As shown in Eq. 1 the Vilsmeier-Haack formylation of indoles using POCl₃-DMF is a general procedure for formylating an indole in its 3-position (Ferguson, Chem. Rev. 1949, 38, 230). As shown in Eq. 2 N-arylindoles are readily prepared through Cu(I)-promoted or Pd-promoted arylation of the indole nitrogen using aryl iodides or aryl boronic acids (Antilla, J. Amer. Chem. Soc. 2002, 121, 1168; Dilip, Tetrahedron Lett. 2014, 55, 931; Old, Org. Lett. 2000, 2, 1403; Wei, Tetrahedron 2008, 74, 19; Cost, J. Med. Chem. 2013, 56, 7431). As shown in Eq. 3 an alkyne group is attached to a brominated indole through a Sonagashira reaction (Sonogashira, Tetrahedron Lett. 1975, 16, 4467). As shown in Eq. 4 compounds of formula (I) that are substituted with amides at the 5-position are prepared through coupling a 5-carboxy indole with an appropriate primary or secondary amine using one of several widely known peptide coupling reagents (Sigma Aldrich).

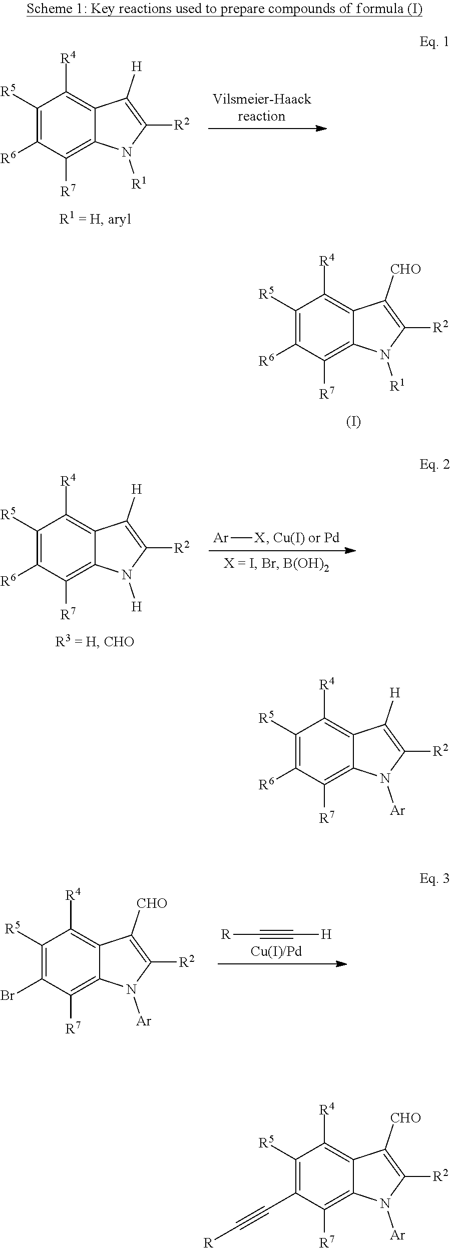

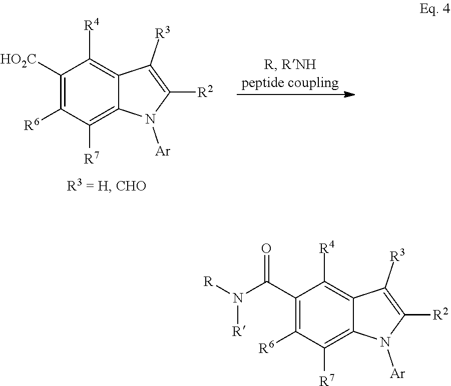

The present disclosure also provides a process of manufacturing of compounds of formula II:

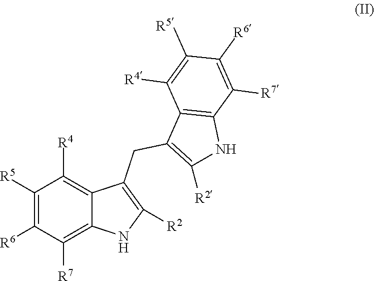

or a pharmaceutically acceptable salt or solvate thereof, wherein R², R⁴, R⁵, R⁶, R⁷, R^{2′}, R^{4′}, R^{5′}, R^{6′}, and R^{7′} are hereafter defined.

Scheme 2 shows key reactions used to prepare unsymmetrically or symmetrically substituted diindolylmethanes of formula (II). Unsymmetrically substituted compounds are prepared through a Cu(II)-promoted decarboxylative coupling of an indole-3-acetic acid with an indole that is unsubstituted in its 3-position (Pillaiyar, Advanced Synth. and Catalysis 2019, 361, 4286). Symmetrically substituted diindolylmethanes are prepared through the reaction of a 3-unsubstituted indole with aqueous formaldehyde (Pillaiyar, J. Med. Chem. 2017, 60, 3636). Amides of formula (II) are prepared through coupling a carboxy indole with an appropriate primary or secondary amine using one of several widely known peptide coupling reagents (Sigma Aldrich).

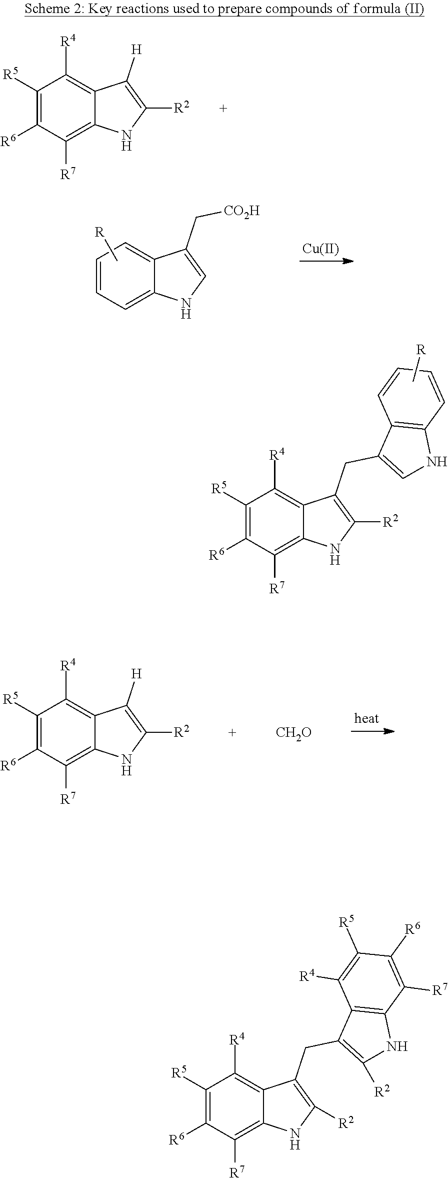

### Uses of the Compounds

The present disclosure is further directed to the use of the compounds described in the present disclosure, or pharmaceutically acceptable salts and solvates thereof, as modulators of HECT domain E3 ligases. Accordingly, in a particularly preferred embodiment, the present disclosure relates to the use of compounds of formula I or II in particular those of Table 1 above, or pharmaceutically acceptable salts and solvates thereof, as modulators of HECT domain E3 ligases.

In one embodiment, the compounds described in the present disclosure are modulators of WWP1 and/or WWP2 and/or NEDD4. In one embodiment, the compounds described in the present disclosure are modulators of WWP1 and WWP2 and NEDD4. In one embodiment, the compounds described in the present disclosure are modulators of WWP1, preferably selective modulators of WWP1. In one embodiment, the compounds described in the present disclosure are modulators selective of WWP1, with respect to other HECT domain E3 ligases. In other embodiment, the compounds described in the present disclosure are modulators selective of WWP2, with respect to other HECT domain E3 ligases. The compounds described in the present disclosure are modulators selective of NEDD4, with respect to other HECT domain E3 ligases.

The present disclosure provides a method for inhibiting HECT domain E3 ligases in a patient, preferably a warm-blooded animal, and even more preferably a human, in need thereof, which comprises administering to said patient an effective amount of a compound described herein, or a pharmaceutically acceptable salt and solvate thereof.

The present disclosure is further directed to the use of the compounds described herein as a medicament, i.e. for medical use. Thus, in one embodiment, the present disclosure provides the use of the compounds described herein for the manufacturing of a medicament. Especially, the present disclosure provides the use of the compounds described herein for the manufacturing of a medicament.

Especially, the present disclosure provides the compounds described herein, for use in the treatment and/or prevention of proliferative disorders, including cancers. Thus, in one embodiment, the present disclosure provides the use of the compounds described herein for the manufacture of a medicament for treating and/or preventing cancer. The present disclosure also provides a method of treatment of cancer, which comprises administering to a mammal species in need thereof a therapeutically effective amount of a compound described herein.

The present disclosure also provides for a method for delaying in patient the onset of cancer comprising the administration of a pharmaceutically effective amount of a compound described herein to a patient in need thereof.

Various cancers are known in the art. Cancers that can be treated using the methods described in the present disclosure include solid cancers and non-solid cancers, especially benign and malignant solid tumors and benign and malignant non-solid tumors. The cancer may be metastatic or non-metastatic. The cancer may be familial or sporadic.

In one embodiment, the cancer to be treated according to the present disclosure is a solid cancer. As used herein, the term “solid cancer” encompasses any cancer (also referred to as malignancy) that forms a discrete tumor mass, as opposed to cancers (or malignancies) that diffusely infiltrate a tissue without forming a mass.

Examples of solid tumors include, but are not limited to: biliary tract cancer, brain cancer (including glioblastomas and medulloblastomas), breast cancer, carcinoid, cervical cancer, choriocarcinoma, colon cancer, colorectal cancer, endometrial cancer, esophageal cancer, gastric cancer, glioma, head and neck cancer, intraepithelial neoplasms (including Bowen's disease and Paget's disease), liver cancer, lung cancer, neuroblastomas, oral cancer (including squamous cell carcinoma), ovarian cancer (including those arising from epithelial cells, stromal cells, germ cells and mesenchymal cells), pancreatic cancer, prostate cancer, rectal cancer, renal cancer (including adenocarcinoma and Wilms tumor), sarcomas (including leiomyosarcoma, rhabdomyosarcoma, liposarcoma, fibrosarcoma and osteosarcoma), skin cancer (including melanoma, Kaposi's sarcoma, basocellular cancer and squamous cell cancer), testicular cancer including germinal tumors (seminomas, and non-seminomas such as teratomas and choriocarcinomas), stromal tumors, germ cell tumors, thyroid cancer (including thyroid adenocarcinoma and medullary carcinoma) and urothelial cancer.

In another embodiment, the cancer to be treated according to the present disclosure is a non-solid cancer. Examples of non-solid tumors include but are not limited to hematological neoplasms. As used herein, a hematologic neoplasm is a term of art which includes lymphoid disorders, myeloid disorders, and AIDS associated leukemias.

Lymphoid disorders include but are not limited to acute lymphocytic leukemia and chronic lymphoproliferative disorders (e.g., lymphomas, myelomas, and chronic lymphoid leukemias). Lymphomas include, for example, Hodgkin's disease, non-Hodgkin's lymphoma lymphomas, and lymphocytic lymphomas). Chronic lymphoid leukemias include, for example, T cell chronic lymphoid leukemias and B cell chronic lymphoid leukemias.

In a specific embodiment, the cancer is selected from breast, carcinoid, cervical, colorectal, endometrial, glioma, head and neck, liver, lung, melanoma, ovarian, pancreatic, prostate, renal, gastric, thyroid and urothelial cancers.

In a specific embodiment, the cancer is breast cancer. In a specific embodiment, the cancer is carcinoid cancer. In a specific embodiment, the cancer is cervical cancer. In a specific embodiment, the cancer is colorectal cancer. In a specific embodiment, the cancer is endometrial cancer. In a specific embodiment, the cancer is glioma. In a specific embodiment, the cancer is head and neck cancer. In a specific embodiment, the cancer is liver cancer. In a specific embodiment, the cancer is lung cancer. In a specific embodiment, the cancer is melanoma. In a specific embodiment, the cancer is ovarian cancer. In a specific embodiment, the cancer is pancreatic cancer. In a specific embodiment, the cancer is prostate cancer. In a specific embodiment, the cancer is renal cancer. In a specific embodiment, the cancer is gastric cancer. In a specific embodiment, the cancer is thyroid cancer. In a specific embodiment, the cancer is urothelial cancer.

In another specific embodiment, the cancer is selected from the group consisting of: leukemia and multiple myeloma.

Preferably, the patient is a warm-blooded animal, more preferably a human.

In one embodiment, the subject receiving the compounds I and II described in the present disclosure is treated with an additional therapeutic agent in combination with the small molecule modulators described in the present disclosure, or has received the additional therapeutic agent within about fourteen, twenty, or thirty days of administration of the small molecule modulators described in the present disclosure. In one embodiment, the additional therapeutic agent comprises radiotherapeutic agent, chemotherapeutic agent, anti-angiogenic agent, apoptosis-inducing agent, anti-tubulin drug, anti-cellular or cytotoxic agent, steroid, check point inhibitor, cytokine antagonist, cytokine expression inhibitor, chemokine antagonist, chemokine expression inhibitor, anti-inflammatory corticosteroid or NSAIDs, coagulant or antiviral agent.

In one embodiment, the subject has previously received at least one prior therapeutic treatment, and has progressed subsequent to the administration of the at least one prior therapeutic treatment and prior to administration of the small molecule inhibitor described in the present disclosure. In one embodiment, the prior therapeutic treatment is selected from the group consisting of chemotherapy, immunotherapy, radiation therapy, stem cell transplant, hormone therapy, and surgery.

The present disclosure also provides the compounds described herein, for use in the treatment of a viral infection. The compounds described in the present disclosure are effective for inhibiting viruses, preferably enveloped viruses, in vivo or ex vivo. In certain embodiments, the compounds described in the present disclosure show broad spectrum antiviral activity. In certain embodiments a method of inhibiting the growth, and/or the proliferation, and/or the infectivity, of an enveloped virus, is provided where the method comprises contacting the virus with an effective amount of a compound formula I or II as described herein. In certain embodiments the method comprises inhibiting the growth and/or proliferation of the virus. In certain embodiments the method comprises inhibiting the infectivity of the virus. In certain embodiments the method comprises reducing the titer of a viral infection in the mammal.

In certain embodiments the method comprises administering the compounds described in the present disclosure to a mammal infected with the virus. In certain embodiments the method comprises administering the compound described in the present disclosure to a mammal at risk for infection by the virus. In certain embodiments the mammal is a non-human mammal. In certain embodiments the mammal is a human. In certain embodiments the virus comprises an enveloped virus.

In certain embodiments the virus is a member of a family selected from the group consisting of Herpesviridae, Poxviridae, Hepadnaviridae, Coronaviridae, Flaviviridae, Togaviridae, Retroviridae, Orthomyxoviridae, Arenaviridae, Bunyaviridae, Filoviridae, Paramyxoviridae, and Rhabdoviridae.

In certain embodiments the virus comprises a virus selected from the group consisting of SARS-CoV-2, SARS-CoV, MERS-CoV, Herpes simplex, type 1, Herpes simplex, type 2, Varicella-zoster virus, Epstein-Barr virus, Human cytomegalovirus, Human herpesvirus, type 8, Smallpox, Hepatitis B virus, Severe acute respiratory syndrome virus, Hepatitis C virus, yellow fever virus, dengue virus, West Nile virus, TBE virus Zika virus, Rubella virus, Human immunodeficiency virus (HIV), Influenza virus, Lassa virus, Crimean-5 Congo hemorrhagic fever virus, Hantaan virus, Ebola virus, Marburg virus, Measles virus, Mumps virus, Parainfluenza virus, Respiratory syncytial virus, Rabies virus, and Hepatitis D virus (HDV). In certain embodiments the virus is Zika virus or HIV-1 In certain embodiments administering the compound(s) in an amount sufficient to ameliorates one or more symptoms of a pathology caused by the virus and/or to slow or prevent infection of the mammal by the virus.

In one embodiment, the compounds described in the present disclosure are administered prior to, concomitant with, or subsequent to administration of the additional therapeutic agent, such as an adenosine receptor antagonist.

### Compositions and Methods for Formulating Pharmaceutical Compositions

The present disclosure also provides pharmaceutical compositions comprising a compound of formula I or II, or a pharmaceutically acceptable salt and solvate thereof, and at least one pharmaceutically acceptable carrier, diluent, excipient and/or adjuvant.

Another object of the present disclosure is a medicament comprising at least one compound described in the present disclosure, or a pharmaceutically acceptable salt and solvate thereof, as active ingredient.

Generally, for pharmaceutical use, the compounds described in the present disclosure may be formulated as a pharmaceutical preparation comprising at least one compound described in the present disclosure and at least one pharmaceutically acceptable carrier, diluent, excipient and/or adjuvant, and optionally one or more further pharmaceutically active compounds. Details regarding the presence of further pharmaceutically active compounds are provided hereafter.

By means of non-limiting examples, such a formulation may be in a form suitable for oral administration, for parenteral administration (such as by intravenous, intramuscular or subcutaneous injection or intravenous infusion), for topical administration (including ocular), for administration by inhalation, by a skin patch, by an implant, by a suppository, etc. Such suitable administration forms—which may be solid, semi-solid or liquid, depending on the manner of administration—as well as methods and carriers, diluents and excipients for use in the preparation thereof, will be clear to the skilled person; reference is made to the latest edition of Remington's Pharmaceutical Sciences.

Some preferred, but non-limiting examples of such preparations include tablets, pills, powders, lozenges, sachets, cachets, elixirs, suspensions, emulsions, solutions, syrups, aerosols, ointments, cremes, lotions, soft and hard gelatin capsules, suppositories, drops, sterile injectable solutions and sterile packaged powders (which are usually reconstituted prior to use) for administration as a bolus and/or for continuous administration, which may be formulated with carriers, excipients, and diluents that are suitable per se for such formulations, such as lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propylhydroxybenzoates, talc, magnesium stearate, edible oils, vegetable oils and mineral oils or suitable mixtures thereof. The formulations can optionally contain other substances that are commonly used in pharmaceutical formulations, such as lubricating agents, wetting agents, emulsifying and suspending agents, dispersing agents, desintegrants, bulking agents, fillers, preserving agents, sweetening agents, flavoring agents, flow regulators, release agents, etc. The compositions may also be formulated so as to provide rapid, sustained or delayed release of the active compound(s) contained therein.

The pharmaceutical preparations described in the present disclosure are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use.

Depending on the condition to be prevented or treated and the route of administration, the active compound described in the present disclosure may be administered as a single daily dose, divided over one or more daily doses, or essentially continuously, e.g. using a drip infusion.

Especially, the present disclosure provides the compounds of the described herein for use in the treatment and/or prevention of proliferative disorders, including cancers. Thus, in one embodiment, the present disclosure provides the use of the compounds described herein for the manufacture of a medicament for treating and/or preventing cancer. The present disclosure also provides a method of treatment of cancer, which comprises administering to a mammal species in need thereof a therapeutically effective amount of a compound described herein.

The present disclosure also provides for a method for delaying in patient the onset of cancer comprising the administration of a pharmaceutically effective amount of a compound described herein to a patient in need thereof.

### EXAMPLES

The present disclosure will be better understood with reference to the following examples. These examples are intended to representative of specific embodiments of the invention, and are not intended as limiting the scope of the invention. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1

### Antisense Nucleotide Selection

Appropriate nucleotide sequences for ASO selection from SEQ ID 45-SEQ ID 531 were identified using the enablement disclosed in Molecular Medicine Today, February 2000, vol. 6, pages 72-81. Specifically, regions near single stranded mRNA are preferential as are sequences without “gggg” nucleotide sequence 4mers or longer. The transcription region near the start codon at 499 to the stop codon at 3267 for WWP1 mRNA (NM_007013.4) is of focus for selecting oligonucleotide sequences for antisense oligonucleotides (ASO) complimentary binding and inhibition of WWP1 transcription. Most preferred sequences were chosen without known immune stimulatory motifs, secondary structures and off target homology. Oligomeric sequences in Table 5 and 6 and those in the examples are appropriate nucleotide sequences.

### Example 2

### Effect of Oligonucleotides Targeting Human WWP1

Oligomeric compounds comprising oligonucleotides complementary to human WWP1 mRNA were synthesized and are shown in the Table 2 below. These compounds were compared by Western Blot Analysis for WWP1 protein down regulation.

TABLE 2

| SEQ. | Cmpd | |
|---|---|---|
| ID NO | # | Sequence (5′→3′)^{a} |
| 532 | 502 | CCA TGT CCC AAA ATT CAG CT |
| 533 | 521 | GAC CTT GGT GAA GCA GTG GC |
| 534 | 527 | GTA TCA GAC CTT GGT GAA GC |
| 535 | 562 | CCT GTA ACT GCA ACC TTC CA |
| 536 | 579 | GGC ACT AGA AAC AGT TAC CT |
| 537 | 674 | GAA CTA CTG GAT TTT GCT GT |
| 538 | 706 | CAG TTA GCT GTT CAT CCC AT |
| 539 | 736 | ATT CCA ATG TAG TCT GTG GC |
| 540 | 751 | GAT GGC TCC AAA CTT GAA AT |
| 10 | 927 | CAC CAA TCC ATC AAG CAC AA |

### Example 3

### Effect of Modified Oligonucleotides Targeting Human WWP1 on Cell Proliferation

Oligomeric compounds comprising modified oligonucleotides at the 2′-sugar position, complementary to human WWP1 mRNA were synthesized and are shown in the table 3 below. These compounds were evaluated for knock-down of WWP1 and cancer cell proliferation.

TABLE 3

| SEQ. | Cmpd | |
|---|---|---|
| ID NO | # | Sequence (5′→3′)^{a} |
| 532 | 502 | CCA TGT CCC AAA ATT CAG CT |
| 533 | 521 | GAC CTT GGT GAA GCA GTG GC |
| 534 | 527 | GTA TCA GAC CTT GGT GAA GC |
| 535 | 562 | CCT GTA ACT GCA ACC TTC CA |
| 536 | 579 | GGC ACT AGA AAC AGT TAC CT |
| 537 | 674 | GAA CTA CTG GAT TTT GCT GT |
| 538 | 706 | CAG TTA GCT GTT CAT CCC AT |
| 539 | 736 | ATT CCA ATG TAG TCT GTG GC |
| 540 | 751 | GAT GGC TCC AAA CTT GAA AT |
| 10 | 927 | CAC CAA TCC ATC AAG CAC AA |

Compounds in bold have a 2′-OMe substituted sugar moiety.

### Example 4

### Effect of Modified Oligonucleotides Targeting Human WWP1 on PTEN Activation

Oligomeric compounds comprising modified oligonucleotides complementary to human WWP1 mRNA were synthesized and are shown in the table 4 below. These compounds were evaluated for knock-down of WWP1 protein production and subsequent PTEN activity enhancement.

TABLE 4

| SEQ. | | |
|---|---|---|
| ID | Cmpd | |
| NO | # | Sequence (5′→3′)^{a} |
| 532 | 502 | CCA TGT CCC AAA ATT CAG CT |
| 533 | 521 | GAC CTT GGT GAA GCA GTG GC |
| 534 | 527 | GTA TCA GAC CTT GGT GAA GC |
| 535 | 562 | CCT GTA ACT GCA ACC TTC CA |
| 536 | 579 | GGC ACT AGA AAC AGT TAC CT |
| 537 | 674 | GAA CTA CTG GAT TTT GCT GT |
| 538 | 706 | CAG TTA GCT GTT CAT CCC AT |
| 539 | 736 | ATT CCA ATG TAG TCT GTG GC |
| 540 | 751 | GAT GGC TCC AAA CTT GAA AT |
| 10 | 927 | CAC CAA TCC ATC AAG CAC AA |

Compounds in bold have a 2′-MOE substituted sugar moiety.

### Example 5

### Tolerability of Modified Oligonucleotides Complementary to Human WWP1 Assessment

Modified oligonucleotides described above are tested in Sprague Dawley rats to assess the tolerability of the oligonucleotides. Sprague Dawley rats each receive a single intrathecal (IT) dose of 3 mg of oligonucleotide. Each treatment group consists of 4 rats. A group of 4 rats receive PBS as a negative control for each experiment. At 3 hours post-injection, movement of 7 different parts of the body are evaluated for each rat. The 7 body parts are (1) the rat's tail; (2) the rat's posterior posture; (3) the rat's hind limbs; (4) the rat's hind paws; (5) the rat's forepaws; (6) the rat's anterior posture; (7) the rat's head. For each of the 7 different body parts, each rat is given a subscore of 0 if the body part was moving or 1 if the body part was not moving (the functional observational battery score or FOB. After all 7 criteria are evaluated, the scores are summed for each rat and averaged for each treatment group.

### Example 6

### SARS CoV2 Egression in VeroE6 Cells and Viral Neutralizing Assays

### 6.1 Measurement of Viral Egress Using Modulators Against SARS-Cov2

Most potent antisense oligonucleotides against WWP1 are selected and their viral egress against SARS-Cov2 and SARS-Cov1 are measured and compared. Antisense oligonucleotides demonstrated pan-activity to multiple relevant NEDD-4 family members which could demonstrate additional benefit as HECT ligase might work through hetero dimeric/trimeric structures.

VeroE6 cells used for this experiment is from African Green Monkey, expressing cell surface receptors for virus interaction and entry. The cells are analyzed in virus neutralization assays to analyze the efficacy of the small molecule modulators measuring viral proteins in cells and viral load in the supernatant.

### 6.2 Introducing Mutation in PPxY Motif of SARs-Cov2 to Establish Causal Linkage

Successful inhibitors of viral SARS-CoV2 are tested against SARS-CoV1 (in the same assay). Subsequently, the PPxY motif in the SARS-CoV2 envelop protein are mutated and viral egression is measured to establish a direct linkage between the WWP1 PPxY domain and increase in virulence.

### Example 7

### The Effect of WWP1 Inhibition on PD-L1 Expression

Although most cancer cells are highly antigenic, they utilize immune evasion strategies such as PD-L1 upregulation on the tumor cell. Interruption of the PD-L1/PD-1 checkpoint axis with mAbs has been established as a novel treatment paradigm and brought cures to many cancer patients. There are significant clinical opportunities to target the immune-suppressive effects of PD-L1 via a different and independent mechanism.

PD-L1 has been shown as target of WWP1. WWP1 ubiquitinates PD-L1 in catalytic dependent manner, forming polyubiquitin chains (C) (Lee Y R, Yehia L, Kishikawa T, et al. WWP1 Gain-of-Function Inactivation of PTEN in Cancer Predisposition. N Engl J Med. 2020; 382(22):2103-2116).

There is sufficient evidence that targeting either of these three mechanisms would drive additional clinical benefit in patients (independent of the current treatment forms). Patients with higher PD-L1 surface expression have a more severe progression. This clearly links PD-L1 cell surface expression to immune suppression and require no additional explanation.

The effect of WWP1 inhibition on PD-L1 expression is established via three independent methods.

(i) Western of total cellular and exosomal/soluble PD-L1
(ii) Flow cytometry to measure cell surface PD-L1
(iii) Immunoprecipitation to measuring of ubiquitination status of PD-L1

These methods (especially the flow assay) establish a rapid screening assay to enable antisense oligonucleotides discovery targeting WWP1.

### 7.1 Establishment of PD-L1 Expression on the Surface of TNBC Cancer Cells

The inhibition of WWP1 affects PD-L1 protein levels on cells surface via FACS analysis. Two cell lines are selected, HS578T (TNBC) and MCF-7 (ER+ luminal) with matching high/low PD-L1 expression. Both cell lines express WWP1 and other relevant biomarkers (PTEN and AKT). Normal tissue controls are HS578Bst from peripheral breast tissue of same donor, and MCF10A, a healthy control.
HS578T-PD-L1high (TNBC); HS578Bst (healthy tissue control)
MCF-7-PD-L1low (ER+); MCF-10A Control (healthy tissue control)

A direct measure of cell surface PD-L1 is performed using a conjugated anti-PD-L1 (e.g. Alexa Fluor 488).

### 7.2 Monitoring PD-L1 Degradation (in WWP1 Know-Down Cells)

Antisense oligonucleotides directed toward WWP1, WWP2, NEDD4 and ITCH are tested.

After establishing conditions for the 4 cell lines (by qPCR), WWP1, WWP2, NEDD4 and ITCH levels are monitored by Western. Westerns for PD-L1 and ubiquitin (analysis after immunoprecipitation) determine PD-L1 levels and modification in cells with ablated WWP1, WWP2, NEDD4 and ITCH. After confirming the effect of E3 ligase knockdown on PD-L1 levels, the mechanism is explored in more detail to understand where PD-L1 is acting. Similar assays including dual/triple target knockdown (WWP1+WWP2; WWP1+WWP2+NEDD4) are performed.

### 7.3 Measurement of PD-L1 Surface Expression (with HECT Ligase Knockdown)

Re-localization of PD-L1 to the cell membrane is crucial for its pathology in TNBC and other cancers. PD-L1 surface levels are monitored before and after WWP1 knockdown. Since the trafficking mechanism is unknown, it is possible that mono-ubiquitination may trigger PD-L1 translocation (trafficking to the membrane vs lysosome), whereas poly-ubiquitination may signal commitment to external presentation (either directly from the synthesis/Golgi or from recycling endosomes).

### 7.4 Measurement of WWP1-Mediated Inhibition in T-Cell Activation Assay

The influence of the PD-L1/PD1 axis on the activity of T cells is measured with commercially available assays. These T cell activation assay comprises (i) expression vectors containing a T-cell receptor (TCR) activator for the PD-L1 positive cancer cells and (ii) TCR/PD-1 reporter cells with NFAT:Luciferase reporter gene. Using antisense oligonucleotides, PD-L1 is downregulated in TNBC cell that results in T cell activation and increase luciferase in this luminescence assay.

### 7.5 Measure PD-L1 Exosomal Excretion and Soluble PD-L1

WWP1 and the level of PD-L1 containing exosome can affect T-cells activity in the tumor microenvironment after secretion (Tang et al., Front. Immunol, 2020). Direct exosomal PD-L1 secretion is changed while absolute levels of membrane PD-L1 remains stable (upon WWP1 knockdown). Exosomes are isolated from the media to quantify total soluble exosomal PD-L1 level, using simple ultrafiltration or ultracentrifugation (J Extracell Vesicles, 2012).

### Example 8

### Testing WWP1 Antisense Oligonucleotides to Evaluate Anti-Tumor Activity in GOF WWP1 Mutations

The goal of these experiments is to establish WWP1 mutations as a drug target for antisense oligonucleotide approaches. WWP1 is an oncogenic factor that has been shown be amplified or mutated in multiple cancer types. The mutation spectrum is interesting as most known cancer mutations are copy number gains and very small number are copy number losses/deletions.

### 8.1 Summary of Spectrum of Point Mutations and Evidence of Potential Function

There are several lines of evidence that suggest that WWP1 point mutation confer a gain-of-function phenotype and lead cancer transformation. WWP1 mutations have been found in all functional domains of the protein (catalytic HECT domain, C2 domain, WW domains).

WWP1 is involved in protein trafficking and protein degradation/stabilization. WWP1 maps on chromosome 8q21.3, a region involved in copy numbers gain in several cancers. WWP1 deficiency result in viable mice without major abnormality and resistant to MYC-driven prostate neoplasia. The mild phenotype of the WWP1 know out would point to a higher frequency of deletion. The mutation profile is intriguing and points to a potential role in cancer progression, but needs to be proven. The WW and C2 domain of WWP1 are regions where most mutations would disrupt their inhibitory function. We therefore interested in further characterizing mutation in these regions for gain-of-function.

### 8.2 Experimental Overview

A set of antisense oligonucleotides are designed to understand the effect of WWP1 knock-down in matched (WWP1GOF vs. wt) cancer cell lines. The following experiments is established to determined WWP1 hot-spot mutations as drug targets.
1. Analysis of Genomic Mutation Spectrum in cancers: Characterize mutation frequency across cancers and identify actionable WWP1 mutations linked to specific cancer.
2. Testing of WWP1 ASO in K740N GoF Mutation: Measure impact of WWP1 knock-down in WWP1K740N cell lines downstream signaling cascade.
3. Testing of WWP1 ASOs in other Hotspot GoF Mutations (i.e., in C2 domain): Use cell and organoid models to measure effect of WWP1 ablation on these GoF-mutant models.

### 8.3 Identification and Genetic Analysis of Most Frequent Somatic Mutations of WWP1 in Cancers

To gain understanding of the frequency and type of WWP1 mutations, appropriate genomics and bioinformatics tools (PolyPhen2, Mutation Taster, SIFT, homology modeling, MetaLR, and MetaSVM) are employed to mine the whole-exome sequencing datasets from different sources (TGCA database, GNOM, Broad Institute). These findings are compared with mutational pattern of other HECT E3 Ligase WWP2, ITCH, NEDD4, NEDDL, and E6AP. Mutational hotspots and regions of frequent mutation in WWP1 and across other HECT ligase family members are discovered.

### 8.4 Testing of ASO in K740N Gain-of-Function Mutation

DLD1 and HCT116 colon cancer cell lines harboring WWP1K740N gain-of-function mutation (GoF) are received. These cell lines are tested with antisense oligonucleotides. Published work by the Pandolfi (Lee Y R, Chen M, Lee J D, et al. Reactivation of PTEN tumor suppressor for cancer treatment through inhibition of a MYC-WWP1 inhibitory pathway. Science. 2019; 364(6441) suggest that genetic ablation or pharmacological inhibition of WWP1 can trigger PTEN reactivation and other tumor suppressive activity. The most active antisense oligonucleotides are tested in a battery of assays to analyze the effect of PTEN-PI3K-AKT/S6K axis and ultimately on the tumor growth using patient derived organoids.

Cell lines: Colon cancer cell lines DLD1 and HCT116 and their CRISPR GoF Knock-in counterparts

CRISPR knock-in WWP1K740N/+ DLD1, WWP1K740N/+ HCT116 and DLD1-WWP1 and HCT-WWP1 transfected with (HA)-PTEN cell lines. The CRISPR knock-in cells reproduce the mutant heterozygous genotype observed in human patients.

Prostate cancer cell lines with DU145, DU145-WWP1+/+, DU145-WWP1−/−, DU145-PTEN+/+ and DU145-PTEN−/− are received and tested. These paired cell lines are important tools to analyze the effect of different WWP1-level and PTEN downstream pathway in the same genetic background.

### 8.4.1 Effect of Antisense Oligonucleotides on PTEN-PI3K-AKT Pathway in GoF Cell Lines

Antisense Oligonucleotides (ASOs) blocking ubiquitination: The paired DLD1 and HCT 116 cell lines are treated with WWP1 ASOs at various condition. PTEN protein from the cell lysates (enriched by immunoprecipitation) is used for mass spectrometric peptide sequencing to analyze whether ASOs block the ubiquitination of K27. In addition, ubiquitination of PTEN is assessed using chain specific ubiquitin antibodies by Western blot in a quantitative manner.

### 8.4.2 Effects of WWP1 ASOs on PI3K-AKT-S6K Pathway:

ASOs are tested for their ability to reduce PTEN ubiquitination, increase PIP2 concentrations, and reduced phospho-AKT/S6 levels in the cells by ELISA or western blot. These assays are performed in WWP1K740N/+ and WWP1+/+ HCT116 cell lines. Post treatment with ASOs/siRNA, cell lysates are analyzed for the AKT pathway specific targets by Western blotting.

### 8.4.3 Effect of ASOs on Cell Growth and Tumorigenicity in GoF Cell Lines

Inhibition of cell proliferation: Cells are seeded (density of 3000 cells) and proliferation is measured using a proliferation assays. Cells are then treated with various doses of ASOs.

Soft agar colony formation assay: Assay is performed in plates testing various doses of ASOs.

Efficacy testing of lead ASOs in mouse human tumor-derived organoids: Organoids from primary colon, prostate, breast and pancreatic cancers are available from several biobanks. Recently, ASO-mediated knockdown of several genes in mammary tumor organoids has been demonstrated (BioProtoc. 2017 Aug. 20; 7(16): e2511). Effect of our ASOs on both prostate organoids and spheres derived from WT and Hi-Myc mice at 3 months of age is tested. These mice are available in the mouse hospital at BIDMC. The most efficacious ASOs are screened against a panel of patient derived organoids for colorectal, prostate, and breast (TNBC) cancers. A reduction in tumor growth in the presence of WWP1 ASO triggers testing in animal models. Positive model is analyzed for WWP1 status.

Xenotransplantation: For assaying tumor growth in the xenograft model, 7-week-old male FOXNnu nude mice housed in specific pathogen-free environments is injected s.c. with 1.0×106 DLD-1 derivatives harboring GoF mutant or 1.0×106 DLD-1 derivatives harboring GoF mutant and treated with lead ASO mixed with RPMI medium and Matrigel (vol/vol, 1:1).

### 8.5 Characterization of Hotspot WWP1 Mutations in Cancer Cell Lines

In several studies, mutations in the C2 and WW region have been attributed to gain of function through relieving the inhibitory effects of C2 and WW on the catalytic HECT activity. Our genomics analysis identify hotspot mutations in the C2 and WW domain. After the genomic analysis, the top two hotspot mutations that are representative for either WW or C2 regulatory domain are picked for further analysis.

Hotspot Mutation in WW-domain or C2 domain: Missense variant of R394C/H/S may alter WWP1 structure and substrate binding or catalytic activity.

Cell lines to test WW-domain GoF mutation: RT4, HT1376 and T24 (urinary bladder cancer (UBC) cell lines) are used to characterize the activities of WWP1R595C/H and WWP1R394C/H/S (or other newly defined hotspot mutations).

Assay to test hyperactivity of hotspot WWP1 mutations: WWP1 hyperactivity has been attributed to the relieving the inhibitory effects of C2-domain using immunoprecipitation and immunoblotting assays. Direct measurement of enzyme kinetics in GOF protein remains difficult due to complexity of assay (E1, E2, E3, Ubiquitin, and ATP). The activity of the newly identified WWP1 variants is analyzed by co-transfection of WWP1wt or WWP1 GOF variants along with HA-tagged potential substrates such as PTEN, TGFb, etc., followed by immunoprecipitation and immunoblotting.

Measuring Downstream effectors of GoF mutations: In UBC, the B-cell translocation gene 2 (BTG2) functions as a tumor suppressor gene and is induced by PTEN. The BTG2 expression is lower in cancer than in normal tissues. Moreover, the highly differentiated bladder cancer cells, RT4, expressed higher BTG2 than the less-differentiated bladder cancer cells, HT1376 and T24. Overexpression of BTG2 in T24 cells inhibited cell growth in vitro and in vivo. Overexpression of gain-of-function activity of WWP1 may cause decrease in BTG2 expression in RT-4 and PTEN-overexpressed T24 cells.

Establishment of CRISPR knock-in cells: To generate WWP1R595C/+ and WWP1R595H/+ mutant cells, Alt-R CRISPR-Cas9 System is performed in accordance with manufacturer's protocol. The knock-in cell lines are used to evaluate the ubiquitination of target proteins (PTEN, BTG2) by WB, expression levels of BTG2 mRNA by RT-PCR and WB, and growth inhibition using MITS reagents.

TABLE 5
Certain Antisense Oligonucleotide Sequences

| SEQ. | Target | |
|---|---|---|
| ID NO | Site | Sequence (5′→3′)^{a} |
| 1 | 502 | CCA UGT CCC AAA ATT CAG CU |
| 2 | 521 | GAC CUT GGT GAA GCA GUG GC |
| 3 | 527 | GUA UCA GAC CTT GGT GAA GC |
| 4 | 562 | CCU GUA ACT GCA ACC UUC CA |
| 5 | 579 | GGC ACT AGA AAC AGT UAC CU |
| 6 | 674 | GAA CUA CTG GAT TTT GCU GU |
| 7 | 706 | CAG UUA GCT GTT CAT CCC AU |
| 8 | 736 | AUU CCA ATG TAG TCT GUG GC |
| 9 | 751 | GAU GGC TCC AAA CTT GAA AU |
| 10 | 927 | CAC CAA TCC ATC AAG CAC AA |
| 11 | 1025 | CUU GCT GAA GGC TCT CCA UU |
| 12 | 1035 | GGC AGT TGT CCT TGC UGA AG |
| 13 | 1055 | GUG CCT TCA ACA GCC AAC CU |
| 14 | 1112 | GAG CAG CAT GAG TTT UGG AC |
| 15 | 1148 | GGA GAT GAA GGT GTG UUG UC |
| 16 | 1172 | UUG GGT CTG GCA GCA ACC UG |
| 17 | 1197 | GAG UGG TTT TGG AGC UGG UG |
| 18 | 1291 | CAG ACA CTA CTG GAG UAC CC |
| 19 | 1352 | UCU UGA ACT GGA GGA UCU UC |
| 20 | 1495 | CUG GCT GTC TTG ATT UGG CU |
| 21 | 1506 | CAU ACA CCC ATC TGG CUG UC |
| 22 | 1558 | CUG AUG GCA AGG TTT CUG UG |
| 23 | 1635 | UCU CUC CCA TGT GGT AGU UC |
| 24 | 1643 | GGU UGT GGT CTC TCC CAU GU |
| 25 | 1654 | CUG GAG GTA AAG GTT GUG GU |
| 26 | 1670 | ACU CUT CTT TCC CAA CCU GG |
| 27 | 1777 | GCU GAG ATT GCC ACT GUU CA |
| 28 | 1808 | AAC UGT TGC ATA GCT CCC UG |
| 29 | 1874 | GGC AAA GGT CCA TAA GGG UC |
| 30 | 1971 | GCC UUG AGT TCT TGG AUC UU |
| 31 | 2100 | UAC AGA TGA CTT CCC AUU GC |
| 32 | 2114 | GGA CCA CCT TTA GTT ACA GA |
| 33 | 2281 | GCC UCC TCA AGT CAT AGG GU |
| 34 | 2318 | UCA AGT CCT TCT TCT CCU CU |
| 35 | 2328 | GCC ACC ATA ATC AAG UCC UU |
| 36 | 2419 | GAC AAT AGT TGT TCT UGC CC |
| 37 | 2726 | CCU CCC AAC TTC AGG UCA UG |
| 38 | 2817 | GGU CUG TTC TTG TAC UCC UC |
| 39 | 2868 | CUG UAG CCA CTG AAG AGG AA |
| 40 | 2908 | UGC CAC ACA ACA TAA CCU CU |
| 41 | 2927 | GCC AAG TCA ACC TCC UGC AU |
| 42 | 2939 | CUC UGC CAA TCT GCC AAG UC |
| 43 | 3152 | GGU AAC CAA GTG TCT UUG CC |
| 44 | 3262 | CUU GUC CAA ATC CCT CUG UC |

^{a}All oligonucleotides have a phosphorothioate backbone.
Bold nucleotides indicate 2′-modified RNA nucleotides.
Plain text nucleotides indicate deoxyribonucleotides.
Cs may be with or without a 5-methyl group.
Target sites are selected from Human WWP1 mRNA accession NM_007013.4.
Target sites are referred to start nucleotide numbers.

TABLE 6
Tiled antisense sequences with
10-nucleotide overlap (WWP1 mRNA)

| SEQ ID | Target | |
|---|---|---|
| NO. | Site | Sequence (5′→3′) |
| 45 | 4861 | BACGCAGTCCTTTTTGAA |
| 46 | 4851 | CCTTTTTGAAGGCCAGTTTC |
| 47 | 4841 | GGCCAGTTTCCCTCTATCAC |
| 48 | 4831 | CCTCTATCACATAACCTGTC |
| 49 | 4821 | ATAACCTGTCACATACATTA |
| 50 | 4811 | ACATACATTAGAATTTTTTT |
| 51 | 4801 | GAATTTTTTTTTTAATTCTT |
| 52 | 4791 | TTTAATTCTTTAGGCTTGAA |
| 53 | 4781 | TAGGCTTGAAAATTACCAGA |
| 54 | 4771 | AATTACCAGAGTGTTCATAA |
| 55 | 4761 | GTGTTCATAATAAGGATTTG |
| 56 | 4751 | TAAGGATTTGGAAAAGGAGT |
| 57 | 4741 | GAAAAGGAGTCTATTCAACC |
| 58 | 4731 | CTATTCAACCAAGCAGATCT |
| 59 | 4721 | AAGCAGATCTCTGCCTTTCT |
| 60 | 4711 | CTGCCTTTCTTTCGTCTTTA |
| 61 | 4701 | TTCGTCTTTACTATGAATTC |
| 62 | 4691 | CTATGAATTCTGAATAAAAT |
| 63 | 4681 | TGAATAAAATGGAGAACACA |
| 64 | 4671 | GGAGAACACACTCTCAATAT |
| 65 | 4661 | CTCTCAATATGTAGAAAAAT |
| 66 | 4651 | GTAGAAAAATAAAATTTTGG |
| 67 | 4641 | AAAATTTTGGCAAAACAATA |
| 68 | 4631 | CAAAACAATAATTACTGTTT |
| 69 | 4621 | ATTACTGTTTTTCTGTATGT |
| 70 | 4611 | TTCTGTATGTACTTAGCAGT |
| 71 | 4601 | ACTTAGCAGTGAAATATTTA |
| 72 | 4591 | GAAATATTTATAGGCAACAG |
| 73 | 4581 | TAGGCAACAGATTTTAAGAA |
| 74 | 4571 | ATTTTAAGAAGTCAGTTCCA |
| 75 | 4561 | GTCAGTTCCATGGCTATTAA |
| 76 | 4551 | TGGCTATTAAAAAATGTTTA |
| 77 | 4541 | AAAATGTTTAAGTTAATGAA |
| 78 | 4531 | AGTTAATGAAATTTACAGTG |
| 79 | 4521 | ATTTACAGTGCATATTAAAA |
| 80 | 4511 | CATATTAAAAGCATGATACA |
| 81 | 4501 | GCATGATACAAATGGAAATT |
| 82 | 4491 | AATGGAAATTTAAAATTTTG |
| 83 | 4481 | TAAAATTTTGTATTAGGAAG |
| 84 | 4471 | TATTAGGAAGCCCAGTTTGC |
| 85 | 4461 | CCCAGTTTGCTAAAAGTAGC |
| 86 | 4451 | TAAAAGTAGCATTAATATCA |
| 87 | 4441 | ATTAATATCATTCACATCAG |
| 88 | 4431 | TTCACATCAGATTTTAATTT |
| 89 | 4421 | ATTTTAATTTGTTACACATT |
| 90 | 4411 | GTTACACATTTTTGTAGTTC |
| 91 | 4401 | TTTGTAGTTCAGCAAAGGCC |
| 92 | 4391 | AGCAAAGGCCCCTATGATGA |
| 93 | 4381 | CCTATGATGAAAAACAATTA |
| 94 | 4371 | AAAACAATTAAAAAGTGTTA |
| 95 | 4361 | AAAAGTGTTATGACAAAAAT |
| 96 | 4351 | TGACAAAAATAATTATCTGT |
| 97 | 4341 | AATTATCTGTGTAAAAAGGG |
| 98 | 4331 | GTAAAAAGGGGGAGACCCAA |
| 99 | 4321 | GGAGACCCAAAAGATTTTTC |
| 100 | 4311 | AAGATTTTTCATATGGTTAT |
| 101 | 4301 | ATATGGTTATTTAATTCAGT |
| 102 | 4291 | TTAATTCAGTTAAATAGAAC |
| 103 | 4281 | TAAATAGAACTGTTATGAAT |
| 104 | 4271 | TGTTATGAATTTGAGAGGAA |
| 105 | 4261 | TTGAGAGGAATGAAGATATT |
| 106 | 4251 | TGAAGATATTCAAACACTAA |
| 107 | 4241 | CAAACACTAACAGAAAATCA |
| 108 | 4231 | CAGAAAATCATTAGCCTTAT |
| 109 | 4221 | TTAGCCTTATATTAGAGCAT |
| 110 | 4211 | ATTAGAGCATTCAAATGCTT |
| 111 | 4201 | TCAAATGCTTATGTAACTCT |
| 112 | 4191 | ATGTAACTCTTCAATCCATC |
| 113 | 4181 | TCAATCCATCTGGCTTCATA |
| 114 | 4171 | TGGCTTCATAAAAACCAAAA |
| 115 | 4161 | AAAACCAAAATTTTGAACAC |
| 116 | 4151 | TTTTGAACACAAAAAAGTGG |
| 117 | 4141 | AAAAAAGTGGCAGAGTTTAC |
| 118 | 4131 | CAGAGTTTACATTACCTTTT |
| 119 | 4121 | ATTACCTTTTGTATGTTCCT |
| 120 | 4111 | GTATGTTCCTAAGTATGAAA |
| 121 | 4101 | AAGTATGAAATTGCATCAAT |
| 122 | 4091 | TTGCATCAATTTATTATGAA |
| 123 | 4081 | TTATTATGAATTAAATTTGA |
| 124 | 4071 | TTAAATTTGAAAAGCATCAC |
| 125 | 4061 | AAAGCATCACAGATTTAAAA |
| 126 | 4051 | AGATTTAAAAACAAAGTACA |
| 127 | 4041 | ACAAAGTACAAAAGAGCCTG |
| 128 | 4031 | AAAGAGCCTGATTCTTTTTA |
| 129 | 4021 | ATTCTTTTTAATTCCACAAA |
| 130 | 4011 | ATTCCACAAATACCTAGCAT |
| 131 | 4001 | TACCTAGCATCTCAAAGTAA |
| 132 | 3991 | CTCAAAGTAACATGTAAACA |
| 133 | 3981 | CATGTAAACAAACTTCTATG |
| 134 | 3971 | AACTTCTATGCTGCTCAATG |
| 135 | 3961 | CTGCTCAATGAATCCTTCCA |
| 136 | 3951 | AATCCTTCCAATTTCGATAA |
| 137 | 3941 | ATTTCGATAATAAACTAAAT |
| 138 | 3931 | TAAACTAAATAGTATTGGAT |
| 139 | 3921 | AGTATTGGATCTAGTATATG |
| 140 | 3911 | CTAGTATATGACTTTCATGT |
| 141 | 3901 | ACTTTCATGTGTAAGTTATG |
| 142 | 3891 | GTAAGTTATGGTTCTATCCA |
| 143 | 3881 | GTTCTATCCATTACTTTAAC |
| 144 | 3871 | TTACTTTAACAATATTACTG |
| 145 | 3861 | AATATTACTGATGTAACAGA |
| 146 | 3851 | ATGTAACAGAGAAAAATTTT |
| 147 | 3841 | GAAAAATTTTCAACTATTGT |
| 148 | 3831 | CAACTATTGTATTTATTTAA |
| 149 | 3821 | ATTTATTTAAAACAAACTGA |
| 150 | 3811 | AACAAACTGACAAGTTCAAG |
| 151 | 3801 | CAAGTTCAAGCACCTGTCTT |
| 152 | 3791 | CACCTGTCTTCAGAAAAGCC |
| 153 | 3781 | CAGAAAAGCCAGCAGCATTT |
| 154 | 3771 | AGCAGCATTTTTTTTTTTTT |
| 155 | 3761 | TTTTTTTTTTAACATACTCA |
| 156 | 3751 | AACATACTCAAAGTAAGATT |
| 157 | 3741 | AAGTAAGATTTGGCCTAAGC |
| 158 | 3731 | TGGCCTAAGCCCTTAATACC |
| 159 | 3721 | CCTTAATACCTTTCTGAACA |
| 160 | 3711 | TTTCTGAACAGCCATGCAAC |
| 161 | 3701 | GCCATGCAACTAAACACCCT |
| 162 | 3691 | TAAACACCCTCAGGAGATGT |
| 163 | 3681 | CAGGAGATGTTACATAAGGG |
| 164 | 3671 | TACATAAGGGAGAGAAGAAC |
| 165 | 3661 | AGAGAAGAACATGGAGCAAT |
| 166 | 3651 | ATGGAGCAATTTGCACTTTT |
| 167 | 3641 | TTGCACTTTTTCCCCTAGAT |
| 168 | 3631 | TCCCCTAGATAATATTAACA |
| 169 | 3621 | AATATTAACAAGGTAAAGCA |
| 170 | 3611 | AGGTAAAGCAAATCCAGATC |
| 171 | 3601 | AATCCAGATCTTTATGAATG |
| 172 | 3591 | TTTATGAATGAATGGCTGTC |
| 173 | 3581 | AATGGCTGTCATGTTTAATA |
| 174 | 3571 | ATGTTTAATACACTTGGAGC |
| 175 | 3561 | CACTTGGAGCTCTATAAAAC |
| 176 | 3551 | TCTATAAAACTAGAGCCACT |
| 177 | 3541 | TAGAGCCACTATCATATATG |
| 178 | 3531 | ATCATATATGTTTATATAGA |
| 179 | 3521 | TTTATATAGATATATATGTA |
| 180 | 3511 | TATATATGTATTTTTAATAA |
| 181 | 3501 | TTTTTAATAAAGTCTCTCAC |
| 182 | 3491 | AGTCTCTCACTTGCTTCAAA |
| 183 | 3481 | TTGCTTCAAAATTTAAGGAT |
| 184 | 3471 | ATTTAAGGATGATCTTTTTC |
| 185 | 3461 | GATCTTTTTCCTGGTCAGTC |
| 186 | 3451 | CTGGTCAGTCATTTCAAGGG |
| 187 | 3441 | ATTTCAAGGGAACAATAAAT |
| 188 | 3431 | AACAATAAATAAAGTAGAAA |
| 189 | 3421 | AAAGTAGAAAAGGATGAACT |
| 190 | 3411 | AGGATGAACTGTTTTGCATA |
| 191 | 3401 | GTTTTGCATATTTCTGTGGA |
| 192 | 3391 | TTTCTGTGGAAGAACGGAAA |
| 193 | 3381 | AGAACGGAAAACATACTTTG |
| 194 | 3371 | ACATACTTTGAGAGGTTCGG |
| 195 | 3361 | AGAGGTTCGGAAAATTCACT |
| 196 | 3351 | AAAATTCACTGTACAGAATG |
| 197 | 3341 | GTACAGAATGAACAGCTTAT |
| 198 | 3331 | AACAGCTTATATACACTATC |
| 199 | 3321 | ATACACTATCTGTGCAATTT |
| 200 | 3311 | TGTGCAATTTTTCTTGGCTG |
| 201 | 3301 | TTCTTGGCTGGGGTATTTAA |
| 202 | 3291 | GGGTATTTAAATGCAAGAGC |
| 203 | 3281 | ATGCAAGAGCTCCTCCAAAA |
| 204 | 3271 | TCCTCCAAAATAAGAAGCCA |
| 205 | 3261 | TAAGAAGCCACATTCATTCT |
| 206 | 3251 | CATTCATTCTTGTCCAAATC |
| 207 | 3241 | TGTCCAAATCCCTCTGTCTC |
| 208 | 3231 | CCTCTGTCTCTTCTATTGCA |
| 209 | 3221 | TTCTATTGCAAAAAGAAGTT |
| 210 | 3211 | AAAAGAAGTTTTTCCTTTAG |
| 211 | 3201 | TTTCCTTTAGTTGTTCATAA |
| 212 | 3191 | TTGTTCATAACTCTTATATG |
| 213 | 3181 | CTCTTATATGGTGGTAGATC |
| 214 | 3171 | GTGGTAGATCCAAGCGATTA |
| 215 | 3161 | CAAGCGATTAAAACATGTAT |
| 216 | 3151 | AAACATGTATGGCTTCTTGG |
| 217 | 3141 | GGCTTCTTGGTAACCAAGTG |
| 218 | 3131 | TAACCAAGTGTCTTTGCCAA |
| 219 | 3121 | TCTTTGCCAACTTTTTCAAT |
| 220 | 3111 | CTTTTTCAATGCAAAACTTT |
| 221 | 3101 | GCAAAACTTTTGAGGCCCAT |
| 222 | 3091 | TGAGGCCCATTACTTCCCAT |
| 223 | 3081 | TACTTCCCATGAGCTCAGCA |
| 224 | 3071 | GAGCTCAGCAAATCCTCCTA |
| 225 | 3061 | AATCCTCCTAGAGGTAAACG |
| 226 | 3051 | GAGGTAAACGGCAGGTTCCA |
| 227 | 3041 | GCAGGTTCCAGTGACGAACT |
| 228 | 3031 | GTGACGAACTGCAATAGTCG |
| 229 | 3021 | GCAATAGTCGCATTCTTACT |
| 230 | 3011 | CATTCTTACTTCATTGTCTG |
| 231 | 3001 | TCATTGTCTGTCTCTTTCAC |
| 232 | 2991 | TCTCTTTCACAAACTGCCAA |
| 233 | 2981 | AAACTGCCAAAACCAAATGA |
| 234 | 2971 | AACCAAATGATTTGCTTGCT |
| 235 | 2961 | TTTGCTTGCTGTTTCTTGTA |
| 236 | 2951 | GTTTCTTGTATAATGTCGAT |
| 237 | 2941 | TAATGTCGATAAACAGTATT |
| 238 | 2931 | AAACAGTATTTCTCTGCCAA |
| 239 | 2921 | TCTCTGCCAATCTGCCAAGT |
| 240 | 2911 | TCTGCCAAGTCAACCTCCTG |
| 241 | 2901 | CAACCTCCTGCATGCCACAC |
| 242 | 2891 | CATGCCACACAACATAACCT |
| 243 | 2881 | AACATAACCTCTAATTCTTT |
| 244 | 2871 | CTAATTCTTTTTCATCGAAG |
| 245 | 2861 | TTCATCGAAGTACTGTAGCC |
| 246 | 2851 | TACTGTAGCCACTGAAGAGG |
| 247 | 2841 | ACTGAAGAGGAACAACTTCA |
| 248 | 2831 | AACAACTTCATTAAAACCAT |
| 249 | 2821 | TTAAAACCATCAAGGAAAGC |
| 250 | 2811 | CAAGGAAAGCTTTGGTCTGT |
| 251 | 2801 | TTTGGTCTGTTCTTGTACTC |
| 252 | 2791 | TCTTGTACTCCTCGAGAAAA |
| 253 | 2781 | CTCGAGAAAAACGCCATTCT |
| 254 | 2771 | ACGCCATTCTGTCATTAAAC |
| 255 | 2761 | GTCATTAAACCAATATATTC |
| 256 | 2751 | CAATATATTCATCTTTGTTC |
| 257 | 2741 | ATCTTTGTTCTCCTCAGTCA |
| 258 | 2731 | TCCTCAGTCACCAGAATATT |
| 259 | 2721 | CCAGAATATTGGAACCTCCC |
| 260 | 2711 | GGAACCTCCCAACTTCAGGT |
| 261 | 2701 | AACTTCAGGTCATGTGAAGT |
| 262 | 2691 | CATGTGAAGTAACTTTTCCC |
| 263 | 2681 | AACTTTTCCCAAAATCTCCA |
| 264 | 2671 | AAAATCTCCATGTCAACAGA |
| 265 | 2661 | TGTCAACAGAAAAGTACATT |
| 266 | 2651 | AAAGTACATTTCTAAGCCAC |
| 267 | 2641 | TCTAAGCCACATTCTTCAAT |
| 268 | 2631 | ATTCTTCAATGTTGTTATCT |
| 269 | 2621 | GTTGTTATCTCTTATCCAGA |
| 270 | 2611 | CTTATCCAGATAAGGGAGTT |
| 271 | 2601 | TAAGGGAGTTATAAAATTCA |
| 272 | 2591 | ATAAAATTCAGTATCAATAG |
| 273 | 2581 | GTATCAATAGATTCCAAATC |
| 274 | 2571 | ATTCCAAATCCTTAATAGTA |
| 275 | 2561 | CTTAATAGTAAGTTTTTTAC |
| 276 | 2551 | AGTTTTTTACTTAACATACG |
| 277 | 2541 | TTAACATACGCTTGTAGAAT |
| 278 | 2531 | CTTGTAGAATGGTAAAGAGA |
| 279 | 2521 | GGTAAAGAGAAACCAGTATC |
| 280 | 2511 | AACCAGTATCGATAAACTTT |
| 281 | 2501 | GATAAACTTTCCATGAAATA |
| 282 | 2491 | CCATGAAATAGTGCCATGGC |
| 283 | 2481 | GTGCCATGGCAATAAAACGA |
| 284 | 2471 | AATAAAACGACCAATGAAAC |
| 285 | 2461 | CCAATGAAACAGAAGTATGA |
| 286 | 2451 | AGAAGTATGAAAGATGGTCT |
| 287 | 2441 | AAGATGGTCTGGATTAATGG |
| 288 | 2431 | GGATTAATGGTTGATGCTGG |
| 289 | 2421 | TTGATGCTGGATTTATCTGC |
| 290 | 2411 | ATTTATCTGCAGACAATAGT |
| 291 | 2401 | AGACAATAGTTGTTCTTGCC |
| 292 | 2391 | TGTTCTTGCCCGCATACTCA |
| 293 | 2381 | CGCATACTCAAATAAGCAAT |
| 294 | 2371 | AATAAGCAATACATTGGGTT |
| 295 | 2361 | ACATTGGGTTCAAAACTTCA |
| 296 | 2351 | CAAAACTTCATGTGAAAGCA |
| 297 | 2341 | TGTGAAAGCAAGAAAAACCA |
| 298 | 2331 | AGAAAAACCATTCTCTCGCT |
| 299 | 2321 | TTCTCTCGCTAGGCCACCAT |
| 300 | 2311 | AGGCCACCATAATCAAGTCC |
| 301 | 2301 | AATCAAGTCCTTCTTCTCCT |
| 302 | 2291 | TTCTTCTCCTCTAAATATTA |
| 303 | 2281 | CTAAATATTACATATAAGCG |
| 304 | 2271 | CATATAAGCGCCTCCTCAAG |
| 305 | 2261 | CCTCCTCAAGTCATAGGGTT |
| 306 | 2251 | TCATAGGGTTTTAATGCCAT |
| 307 | 2241 | TTAATGCCATAATCTGTTGG |
| 308 | 2231 | AATCTGTTGGAAGGAATCTT |
| 309 | 2221 | AAGGAATCTTCAAACAATGT |
| 310 | 2211 | CAAACAATGTCTGCCGGGAC |
| 311 | 2201 | CTGCCGGGACACATTGATCT |
| 312 | 2191 | ACATTGATCTTTACATGACT |
| 313 | 2181 | TTACATGACTAGGTAGTGCA |
| 314 | 2171 | AGGTAGTGCATTAGACTGGC |
| 315 | 2161 | TTAGACTGGCACAAATAACG |
| 316 | 2151 | ACAAATAACGGAAGTGAGCA |
| 317 | 2141 | GAAGTGAGCAAGCTTCCACC |
| 318 | 2131 | AGCTTCCACCTAAAGCCGCG |
| 319 | 2121 | TAAAGCCGCGTTCATAAGCA |
| 320 | 2111 | TTCATAAGCAATTTGTGGAC |
| 321 | 2101 | ATTTGTGGACCACCTTTAGT |
| 322 | 2091 | CACCTTTAGTTACAGATGAC |
| 323 | 2081 | TACAGATGACTTCCCATTGC |
| 324 | 2071 | TTCCCATTGCGAGGATCTTT |
| 325 | 2061 | GAGGATCTTTGAATGTTGTT |
| 326 | 2051 | GAATGTTGTTGTTCTTGTGT |
| 327 | 2041 | GTTCTTGTGTTATGATCAAC |
| 328 | 2031 | TATGATCAACAAAGTACCTT |
| 329 | 2021 | AAAGTACCTTACACCTTCAC |
| 330 | 2011 | ACACCTTCACGAGTATATCT |
| 331 | 2001 | GAGTATATCTAATTTCCCAG |
| 332 | 1991 | AATTTCCCAGCCTTCTGGCA |
| 333 | 1981 | CCTTCTGGCAGGGGTTCTTC |
| 334 | 1971 | GGGGTTCTTCATTCTGTAAG |
| 335 | 1961 | ATTCTGTAAGCCTTGAGTTC |
| 336 | 1951 | CCTTGAGTTCTTGGATCTTC |
| 337 | 1941 | TTGGATCTTCCCACTGGGTT |
| 338 | 1931 | CCACTGGGTTGTTTTTGTGT |
| 339 | 1921 | GTTTTTGTGTTATGATTCAC |
| 340 | 1911 | TATGATTCACAAAGTAAACC |
| 341 | 1901 | AAAGTAAACCCTGTCTGTTG |
| 342 | 1891 | CTGTCTGTTGAATCCACTCT |
| 343 | 1881 | AATCCACTCTTTTTTCCCAG |
| 344 | 1871 | TTTTTCCCAGCCTGGTGGCA |
| 345 | 1861 | CCTGGTGGCAAAGGTCCATA |
| 346 | 1851 | AAGGTCCATAAGGGTCATTT |
| 347 | 1841 | AGGGTCATTTTCTGCAGCTA |
| 348 | 1831 | TCTGCAGCTAACATTGAAGC |
| 349 | 1821 | ACATTGAAGCCGAATAGAGG |
| 350 | 1811 | CGAATAGAGGTATCGTTGGT |
| 351 | 1801 | TATCGTTGGTTAAACTGTTG |
| 352 | 1791 | TAAACTGTTGCATAGCTCCC |
| 353 | 1781 | CATAGCTCCCTGCAATTGGT |
| 354 | 1771 | TGCAATTGGTTCCGCTGAGA |
| 355 | 1761 | TCCGCTGAGATTGCCACTGT |
| 356 | 1751 | TTGCCACTGTTCAAAATTTC |
| 357 | 1741 | TCAAAATTTCGGACAGATTC |
| 358 | 1731 | GGACAGATTCCATGGTAGGC |
| 359 | 1721 | CATGGTAGGCCGCTGCCACG |
| 360 | 1711 | CGCTGCCACGTTGTTGTTCT |
| 361 | 1701 | TTGTTGTTCTGGTGTTATGA |
| 362 | 1691 | GGTGTTATGATCCACATAAT |
| 363 | 1681 | TCCACATAATAAACTCTTCT |
| 364 | 1671 | AAACTCTTCTACGATCATCA |
| 365 | 1661 | ACGATCATCAACTCTTCTTT |
| 366 | 1651 | ACTCTTCTTTCCCAACCTGG |
| 367 | 1641 | CCCAACCTGGAGGTAAAGGT |
| 368 | 1631 | AGGTAAAGGTTGTGGTCTCT |
| 369 | 1621 | TGTGGTCTCTCCCATGTGGT |
| 370 | 1611 | CCCATGTGGTAGTTCGAGTA |
| 371 | 1601 | AGTTCGAGTATTATGATCCA |
| 372 | 1591 | TTATGATCCACATAATAGGT |
| 373 | 1581 | CATAATAGGTTCTACCATGA |
| 374 | 1571 | TCTACCATGAGGATCTTTTC |
| 375 | 1561 | GGATCTTTTCTTTGTTCCCA |
| 376 | 1551 | TTTGTTCCCACCCTGATGGC |
| 377 | 1541 | CCCTGATGGCAAGGTTTCTG |
| 378 | 1531 | AAGGTTTCTGTGTTGGCATT |
| 379 | 1521 | TGTTGGCATTCCCAGACTGC |
| 380 | 1511 | CCCAGACTGCTGCCGTACAG |
| 381 | 1501 | TGCCGTACAGGATCCATACA |
| 382 | 1491 | GATCCATACACCCATCTGGC |
| 383 | 1481 | CCCATCTGGCTGTCTTGATT |
| 384 | 1471 | TGTCTTGATTTGGCTGCTTC |
| 385 | 1461 | TGGCTGCTTCAAAAGCAGAA |
| 386 | 1451 | AAAAGCAGAACTACTTCTAG |
| 387 | 1441 | CTACTTCTAGAATTAGAGGT |
| 388 | 1431 | AATTAGAGGTGTCAGGCTCT |
| 389 | 1421 | GTCAGGCTCTAATATACTTC |
| 390 | 1411 | AATATACTTCTAGCTTCAGA |
| 391 | 1401 | TAGCTTCAGATTCCAATTCT |
| 392 | 1391 | TTCCAATTCTGCACTGGTAG |
| 393 | 1381 | GCACTGGTAGAAGGAATACA |
| 394 | 1371 | AAGGAATACATTCATTGTTT |
| 395 | 1361 | TTCATTGTTTTCTGAGGAAG |
| 396 | 1351 | TCTGAGGAAGTCAGTATTTC |
| 397 | 1341 | TCAGTATTTCTTGAACTGGA |
| 398 | 1331 | TTGAACTGGAGGATCTTCAA |
| 399 | 1321 | GGATCTTCAACAGTAGTACT |
| 400 | 1311 | CAGTAGTACTAGTGCAATTT |
| 401 | 1301 | AGTGCAATTTGGAGACAAGG |
| 402 | 1291 | GGAGACAAGGCATTTTCTTC |
| 403 | 1281 | CATTTTCTTCAGACACTACT |
| 404 | 1271 | AGACACTACTGGAGTACCCG |
| 405 | 1261 | GGAGTACCCGTGACAGACGC |
| 406 | 1251 | TGACAGACGCATTATCAGTT |
| 407 | 1241 | ATTATCAGTTGGTGCAAATG |
| 408 | 1231 | GGTGCAAATGAGGATGATTC |
| 409 | 1221 | AGGATGATTCTCCATTAACA |
| 410 | 1211 | TCCATTAACAGTGTCATCGG |
| 411 | 1201 | GTGTCATCGGCAGGCTCAGA |
| 412 | 1191 | CAGGCTCAGATGCGAGTGGT |
| 413 | 1181 | TGCGAGTGGTTTTGGAGCTG |
| 414 | 1171 | TTTGGAGCTGGTGTATTTTT |
| 415 | 1161 | GTGTATTTTTGGGTCTGGCA |
| 416 | 1151 | GGGTCTGGCAGCAACCTGAG |
| 417 | 1141 | GCAACCTGAGACGGAGATGA |
| 418 | 1131 | ACGGAGATGAAGGTGTGTTG |
| 419 | 1121 | AGGTGTGTTGTCTCCATTAA |
| 420 | 1111 | TCTCCATTAACTACATACGA |
| 421 | 1101 | CTACATACGAGCAGCATGAG |
| 422 | 1091 | GCAGCATGAGTTTTGGACTA |
| 423 | 1081 | TTTTGGACTAGAGTGCTTGT |
| 424 | 1071 | GAGTGCTTGTAGGTACATGA |
| 425 | 1061 | AGGTACATGATTATCTATTC |
| 426 | 1051 | TTATCTATTCCATTCGTGCC |
| 427 | 1041 | CATTCGTGCCTTCAACAGCC |
| 428 | 1031 | TTCAACAGCCAACCTGGCAG |
| 429 | 1021 | AACCTGGCAGTTGTCCTTGC |
| 430 | 1011 | TTGTCCTTGCTGAAGGCTCT |
| 431 | 1001 | TGAAGGCTCTCCATTTTCAT |
| 432 | 991 | CCATTTTCATGTAAGGCATC |
| 433 | 981 | GTAAGGCATCACCATTTTCC |
| 434 | 971 | ACCATTTTCCTGTATTTCTA |
| 435 | 961 | TGTATTTCTATGGTTGGAGA |
| 436 | 951 | TGGTTGGAGATGAGCTGCAG |
| 437 | 941 | TGAGCTGCAGTTTGTTATAT |
| 438 | 931 | tttgttatattttcttgctc |
| 439 | 921 | TTTCTTGCTCAATCACCAAT |
| 440 | 911 | AATCACCAATCCATCAAGCA |
| 441 | 901 | CCATCAAGCACAACTGTCAA |
| 442 | 891 | CAACTGTCAATTCACCAGTT |
| 443 | 881 | TTCACCAGTTTGTGCTATGC |
| 444 | 871 | TGTGCTATGCCATTCTTGTT |
| 445 | 861 | CATTCTTGTTTTCCAAGGAA |
| 446 | 851 | TTCCAAGGAAAGTTTTAATT |
| 447 | 841 | AGTTTTAATTGTTCTTTCAC |
| 448 | 831 | GTTCTTTCACTCTTTCCAAT |
| 449 | 821 | TCTTTCCAATTTTCTATTGT |
| 450 | 811 | TTTCTATTGTGTATCAACAG |
| 451 | 801 | GTATCAACAGAGCTTGTTTC |
| 452 | 791 | AGCTTGTTTCAAATCTATCG |
| 453 | 781 | AAATCTATCGTTGCTTTTCC |
| 454 | 771 | TGCTTTTCCTAATAAAGCA |
| 455 | 761 | TAATAAAGCATCTGCTTTTA |
| 456 | 751 | TCTGCTTTTAAAGTGCGATG |
| 457 | 741 | AAGTGCGATGGCTCCAAACT |
| 458 | 731 | GCTCCAAACTTGAAATTCCA |
| 459 | 721 | TGAAATTCCAATGTAGTCTG |
| 460 | 711 | ATGTAGTCTGTGGCGTAACA |
| 461 | 701 | TGGCGTAACATTTACAGTTA |
| 462 | 691 | TTTACAGTTAGCTGTTCATC |
| 463 | 681 | GCTGTTCATCCCATTTTGGA |
| 464 | 671 | CCATTTTGGATTAGAAGAAC |
| 465 | 661 | TTAGAAGAACTACTGGATTT |
| 466 | 651 | TACTGGATTTTGCTGTTTTC |
| 467 | 641 | TGCTGTTTTCGTAATTTCTC |
| 468 | 631 | GTAATTTCTCCATCTACAAC |
| 469 | 621 | CATCTACAACTACTTCTGTA |
| 470 | 611 | TACTTCTGTATATATTGCTG |
| 471 | 601 | TATATTGCTGTTCCGAACCA |
| 472 | 591 | TTCCGAACCAGTTCTTTTTT |
| 473 | 581 | GTTCTTTTTTCTTTTAAGTT |
| 474 | 571 | CTTTTAAGTTTGGCACTAGA |
| 475 | 561 | TGGCACTAGAAACAGTTACC |
| 476 | 551 | AACAGTTACCTGTAACTGCA |
| 477 | 541 | TGTAACTGCAACCTTCCACT |
| 478 | 531 | ACCTTCCACTGTGGTTATTA |
| 479 | 521 | GTGGTTATTACTAGTATCAG |
| 480 | 511 | CTAGTATCAGACCTTGGTGA |
| 481 | 501 | ACCTTGGTGAAGCAGTGGCC |
| 482 | 491 | AGCAGTGGCCATGTCCCAAA |
| 483 | 481 | ATGTCCCAAAATTCAGCTAA |
| 484 | 471 | ATTCAGCTAAAACTATGTAT |
| 485 | 461 | AACTATGTATCAGGAACTGC |
| 486 | 451 | CAGGAACTGCTAGACACTGG |
| 487 | 441 | TAGACACTGGGGATACAAAG |
| 488 | 431 | GGATACAAAGATGAGTAAGA |
| 489 | 421 | ATGAGTAAGACACGATTCCC |
| 490 | 411 | CACGATTCCCTCTTTCAAGG |
| 491 | 401 | TCTTTCAAGGAGCTCATAGT |
| 492 | 391 | AGCTCATAGTCTGTGAGGAG |
| 493 | 381 | CTGTGAGGAGACAACCCCGG |
| 494 | 371 | ACAACCCCGGCGCGAAGCTG |
| 495 | 361 | CGCGAAGCTGTCGGCTCCCG |
| 496 | 351 | TCGGCTCCCGGCAGCCGGGC |
| 497 | 341 | GCAGCCGGGCGGCCACGGCA |
| 498 | 331 | GGCCACGGCAGCGGCACCTC |
| 499 | 321 | GCGGCACCTCCTCCTGCGGC |
| 500 | 311 | CTCCTGCGGCGGGCGCCCTC |
| 501 | 301 | GGGCGCCCTCACCCTTCCGC |
| 502 | 291 | ACCCTTCCGCGCCCAAGATC |
| 503 | 281 | GCCCAAGATCGCCATCTCGC |
| 504 | 271 | GCCATCTCGCAGCAGGCGCG |
| 505 | 261 | AGCAGGCGCGGCGGCAGCAG |
| 506 | 251 | GCGGCAGCAGCCACCCAGGC |
| 507 | 241 | CCACCCAGGCGTCGGCCCCG |
| 508 | 231 | GTCGGCCCCGGCAGCCCAGG |
| 509 | 221 | GCAGCCCAGGCCGCCAGCAG |
| 510 | 211 | CCGCCAGCAGCCACTCGGAA |
| 511 | 201 | CCACTCGGAACCCGCACGCG |
| 512 | 191 | CCCGCACGCGGAGCTCGCCG |
| 513 | 181 | GAGCTCGCCGACACCCGACC |
| 514 | 171 | ACACCCGACCCTCCCCCCAC |
| 515 | 161 | CTCCCCCCACCCCCACCCCG |
| 516 | 151 | CCCCACCCCGTCCACGCGCC |
| 517 | 141 | TCCACGCGCCACCCCCAGCC |
| 518 | 131 | ACCCCCAGCCGACCCCACAC |
| 519 | 121 | GACCCCACACCTCCCTTCCT |
| 520 | 111 | CTCCCTTCCTCGGGAGCGCG |
| 521 | 101 | CGGGAGCGCGCCGCGTGGCC |
| 522 | 91 | CCGCGTGGCCGCGTCGCCGG |
| 523 | 81 | GCGTCGCCGGCGCCGCGCCC |
| 524 | 71 | CGCCGCGCCCTAAGCGCGCG |
| 525 | 61 | TAAGCGCGCGCCTCCCCGCC |
| 526 | 51 | CCTCCCCGCCGGCCCCGGCA |
| 527 | 41 | GGCCCCGGCACCGCGCGCTA |
| 528 | 31 | CCGCGCGCTACGCCGCGGCC |
| 529 | 21 | CGCCGCGGCCGCCCAAAGCC |
| 530 | 11 | GCCCAAAGCCTCCAACTCCG |
| 531 | 1 | TCCAACTCCGGACCCGGAAT |

a: Target site corresponds to 5′-end nucleotide of mRNA.

### Example 9

### Selection of Small Molecule Modulators

The compounds of formula I and II of the present disclosure were designed through a combination of methods for small-molecule lead discovery including structure-based design using observed and predicted ligand docking sites (Review: Weber, et al. Frontiers in Physiology, 2019, 10; I3C: Lee, et al. Science, 2019, 364, 6441; NEDD4 covalent inhibitor: Kathman, et al. J. Amer. Chem. Soc. 2015, 137, 12442) as well as molecular modification of published modulators (NEDD4: Quirit, et al. Biochem. Pharmacol, 2017, 127, 13).

Preferred compound structures of formula I and II described in the present disclosure listed in Table 1 were chosen based on docking scores obtained by Schrödinger, LLC, New York, N.Y., USA. The compounds in Table 1 have docking scores exceeded a value of −8.0, which is considered as a good compound in terms of binding in the virtual screening experiment. The docking score criterion is described in details here Friesner, R. A.; Banks, J. L.; Murphy, R. B.; Halgren, T. A.; Klicic, J. J.; Mainz, D. T.; Repasky, M. P.; Knoll, E. H.; Shaw, D. E.; Shelley, M.; Perry, J. K.; Francis, P.; Shenkin, P. S., “Glide: A New Approach for Rapid, Accurate Docking and Scoring. 1. Method and Assessment of Docking Accuracy,” J. Med. Chem., 2004, 47, 1739-1749.)

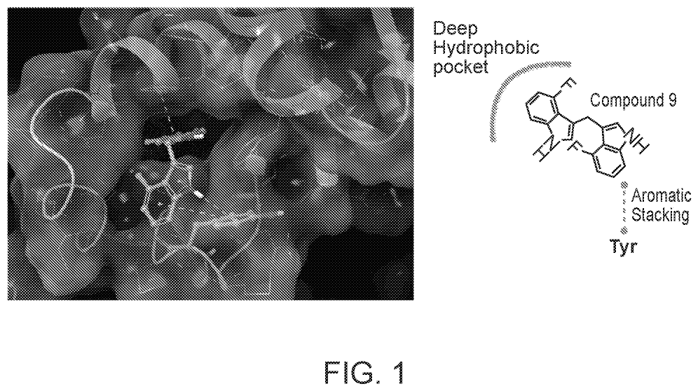

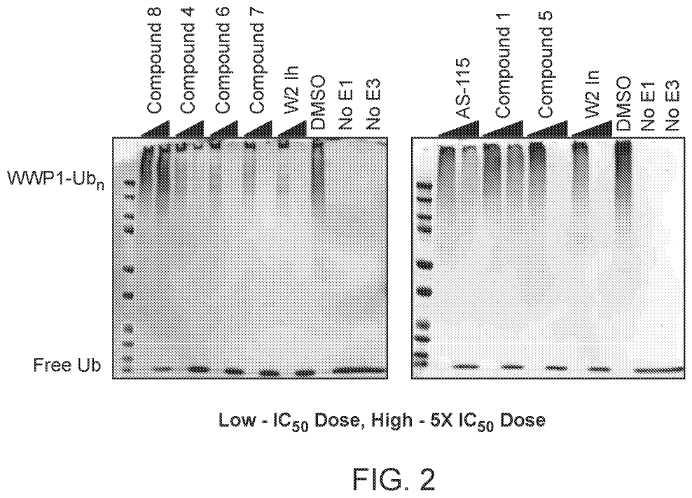

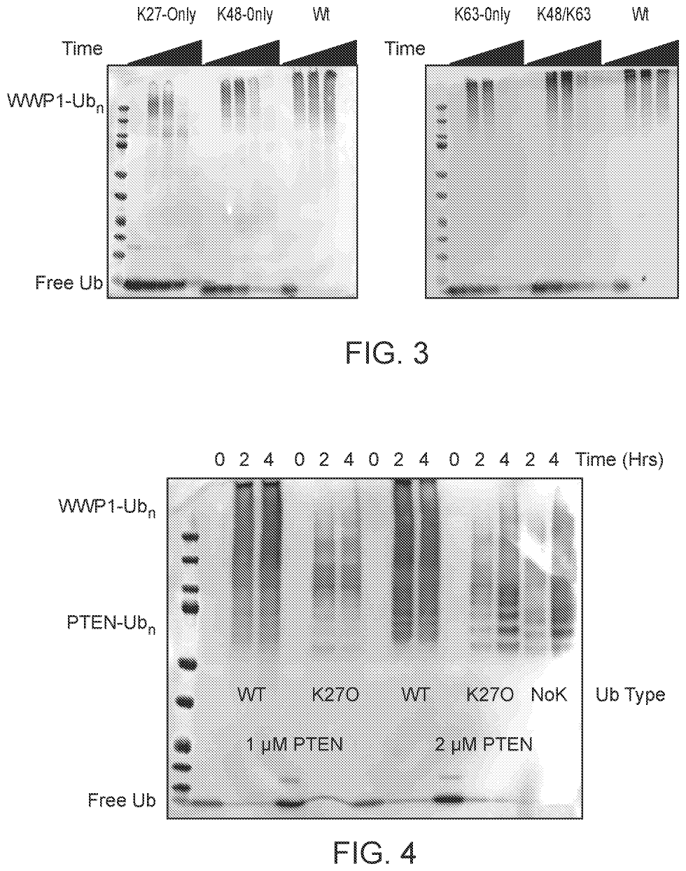

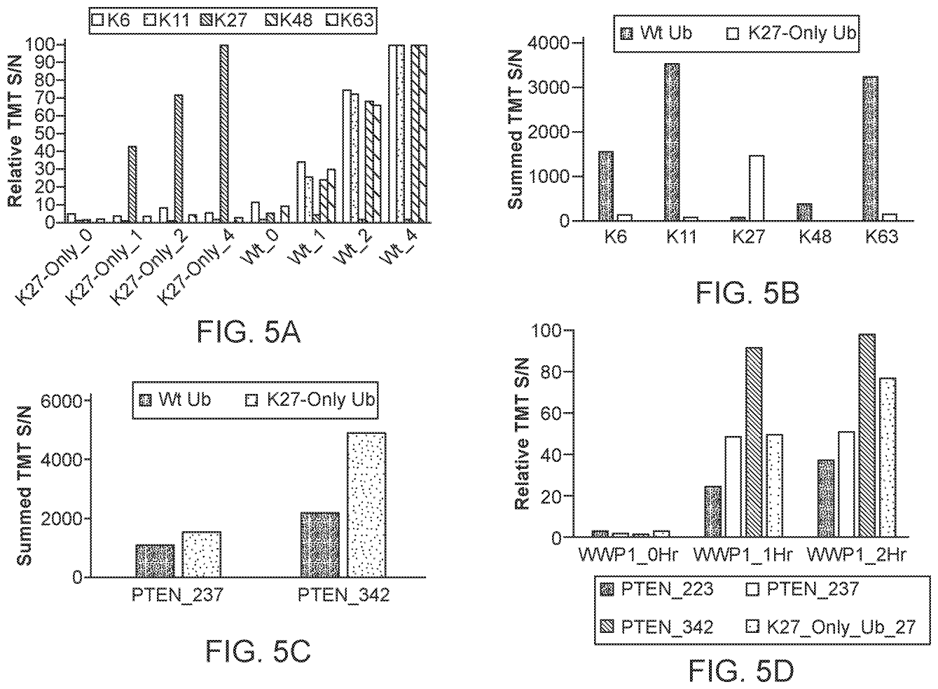

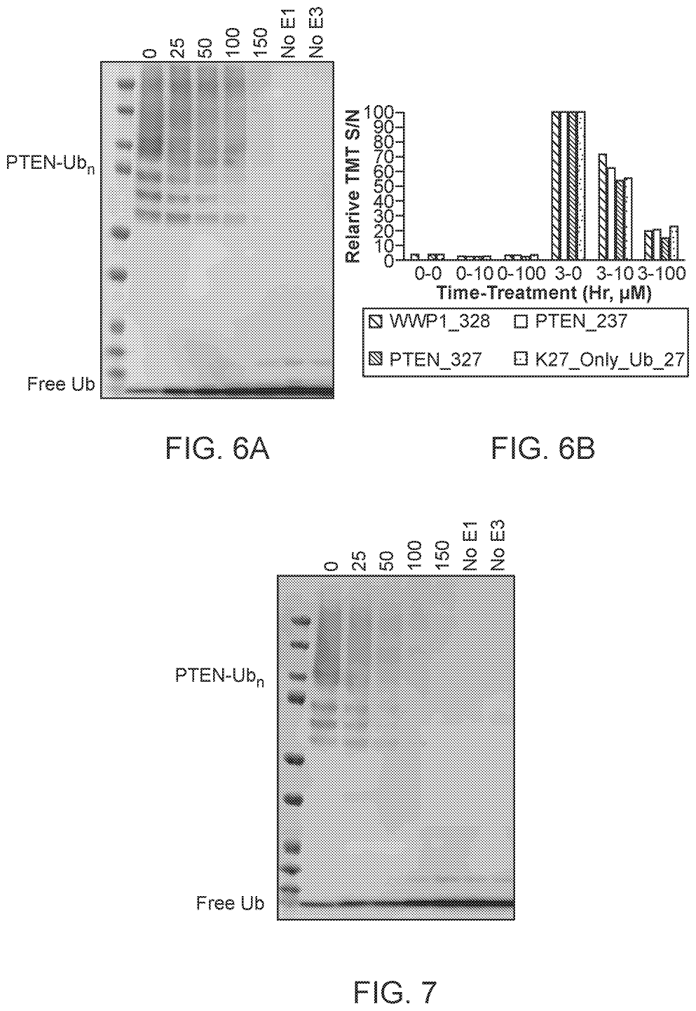

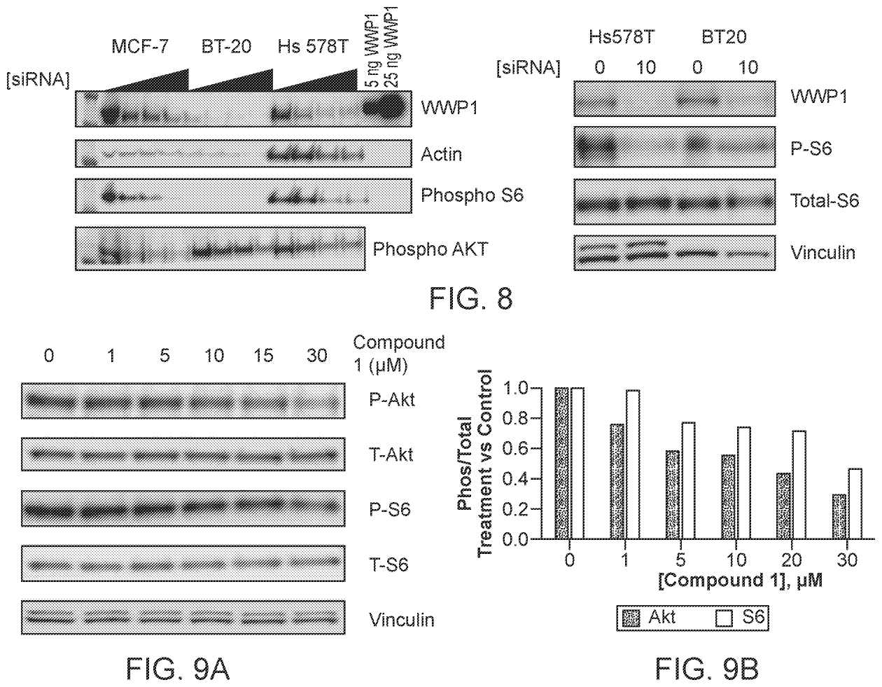

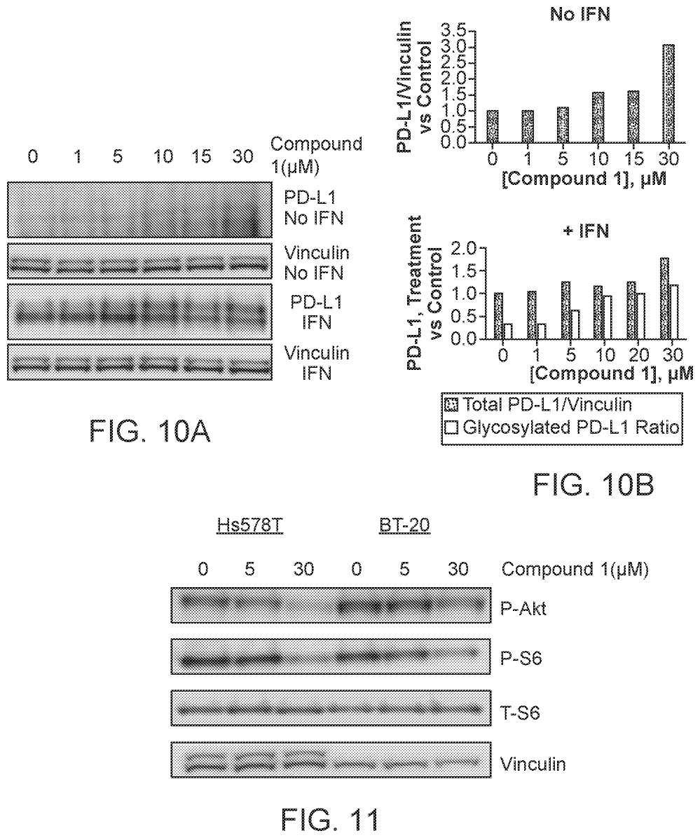

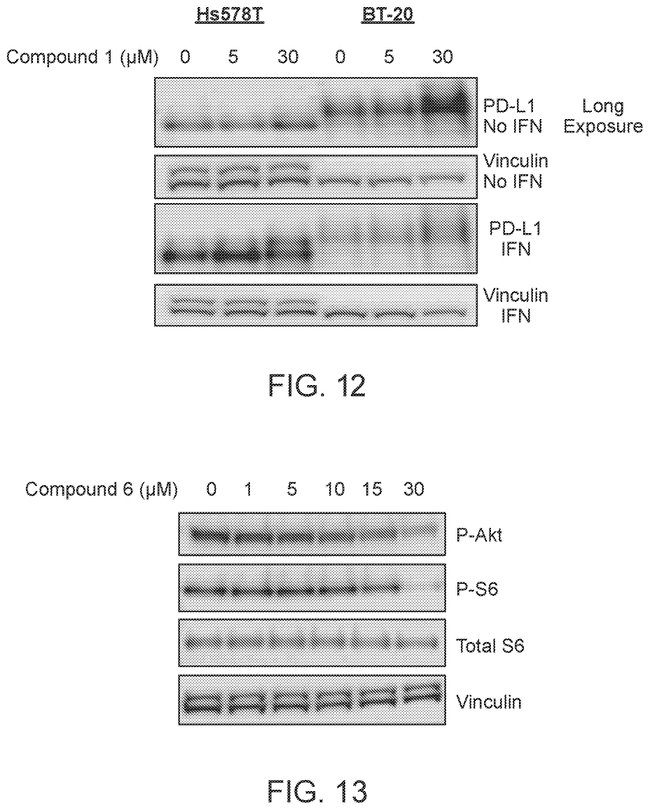

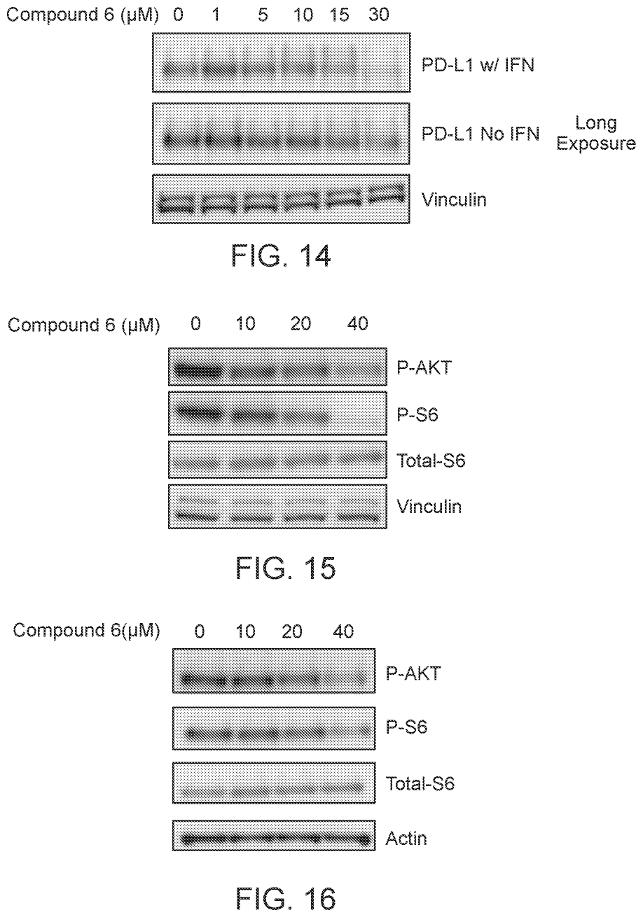

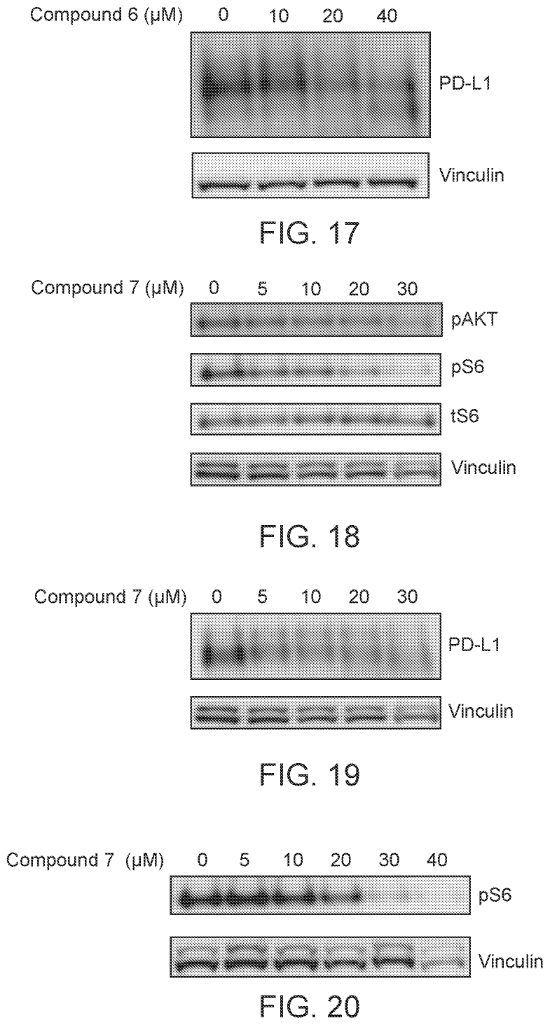

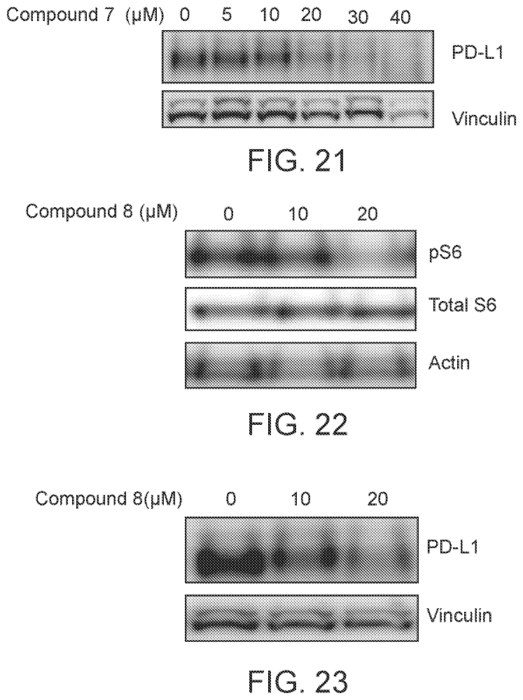

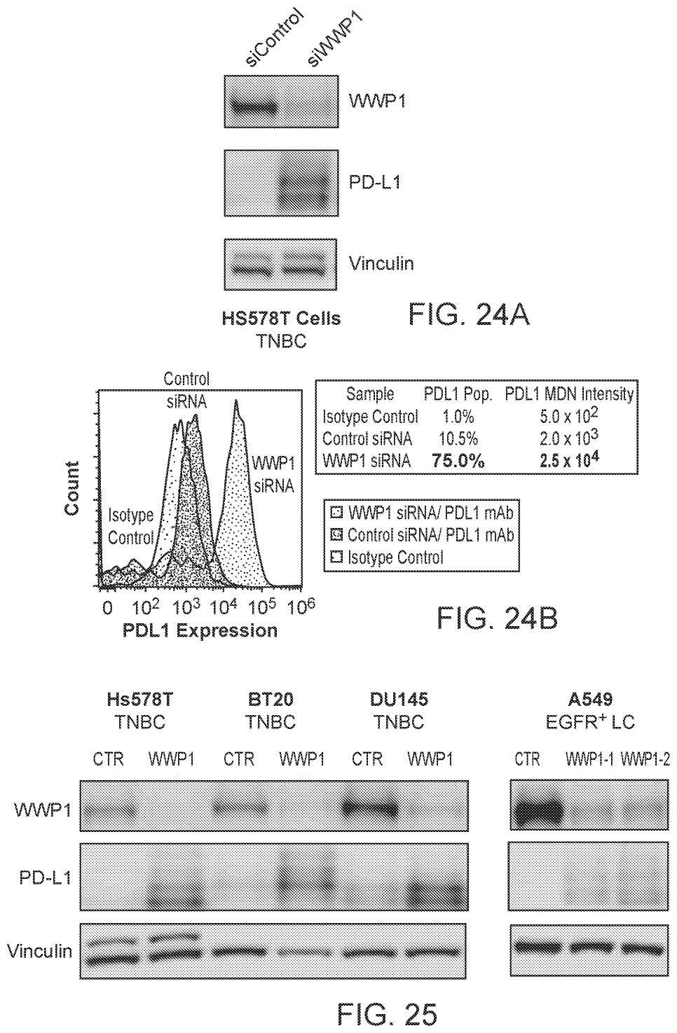

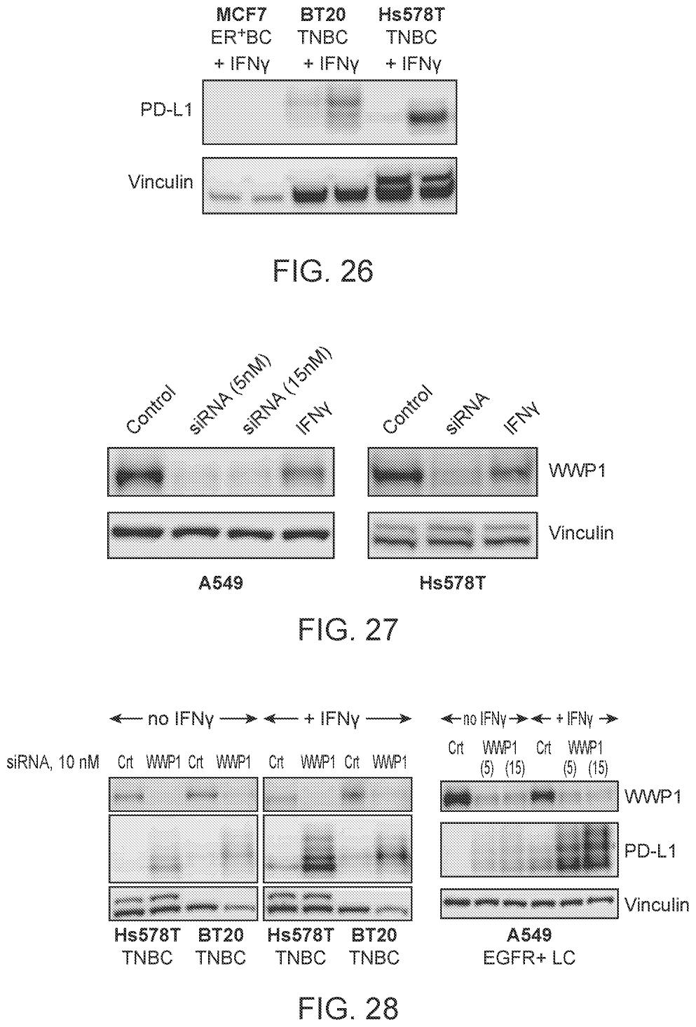

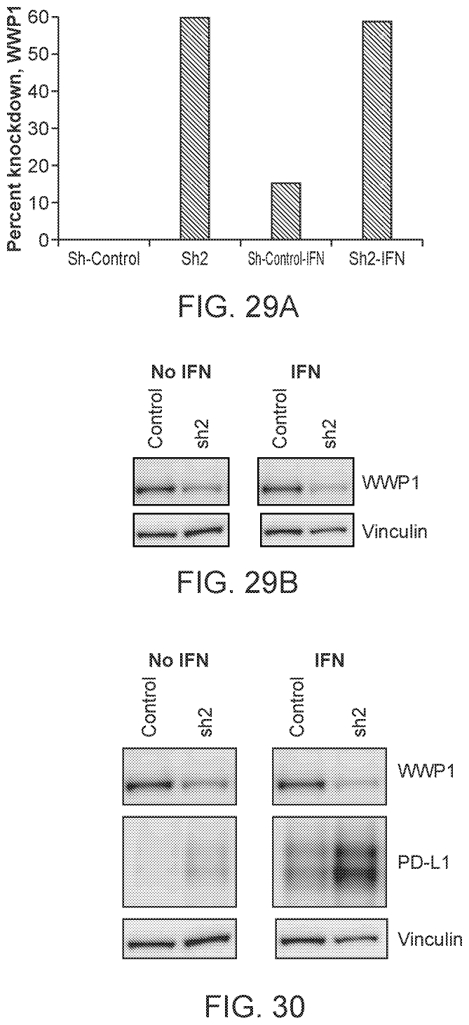

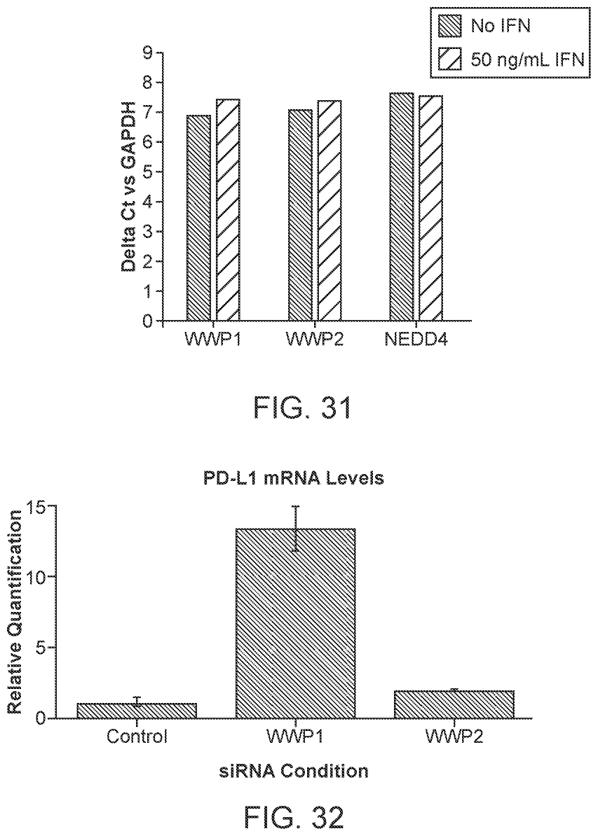

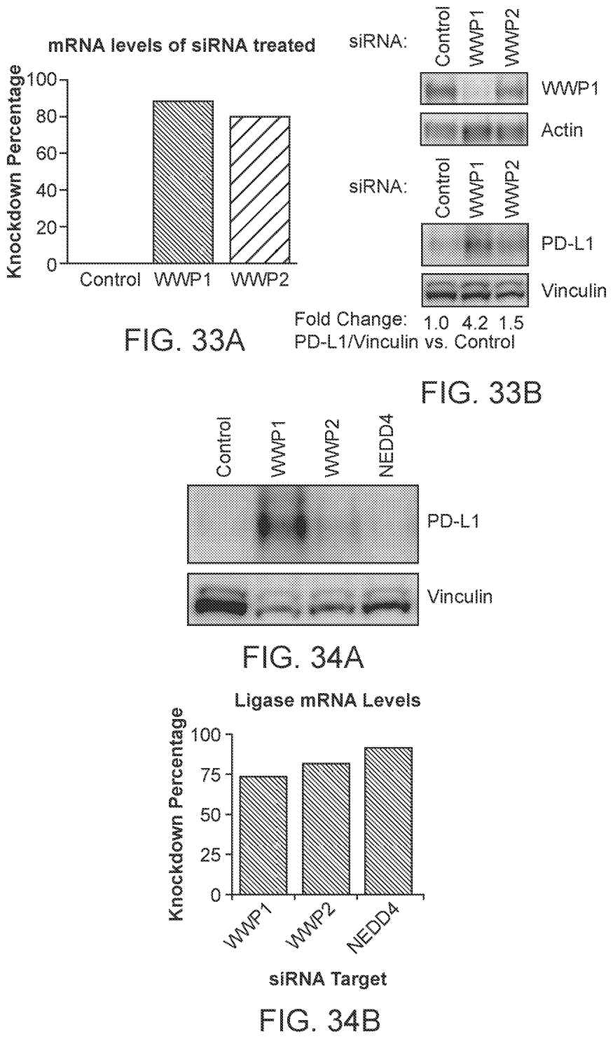

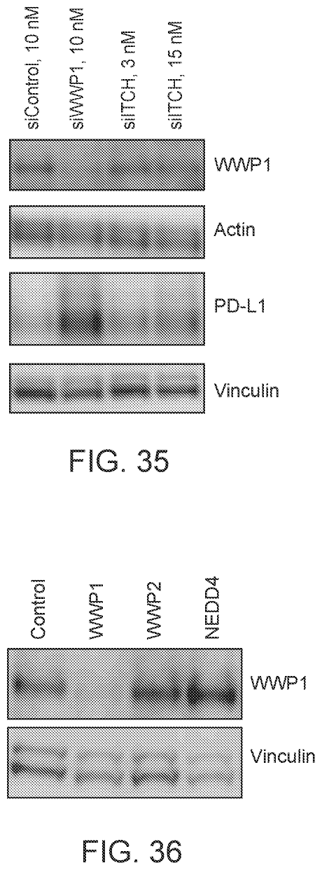

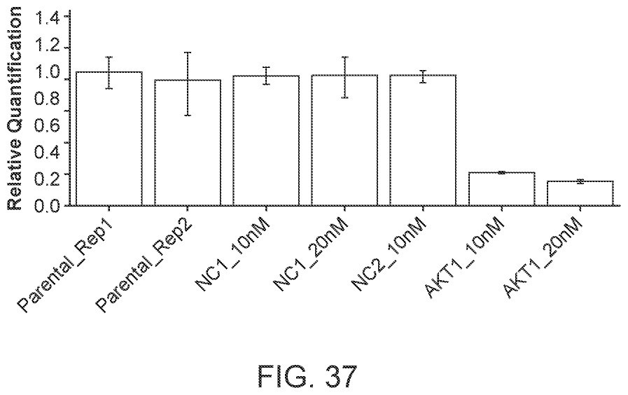

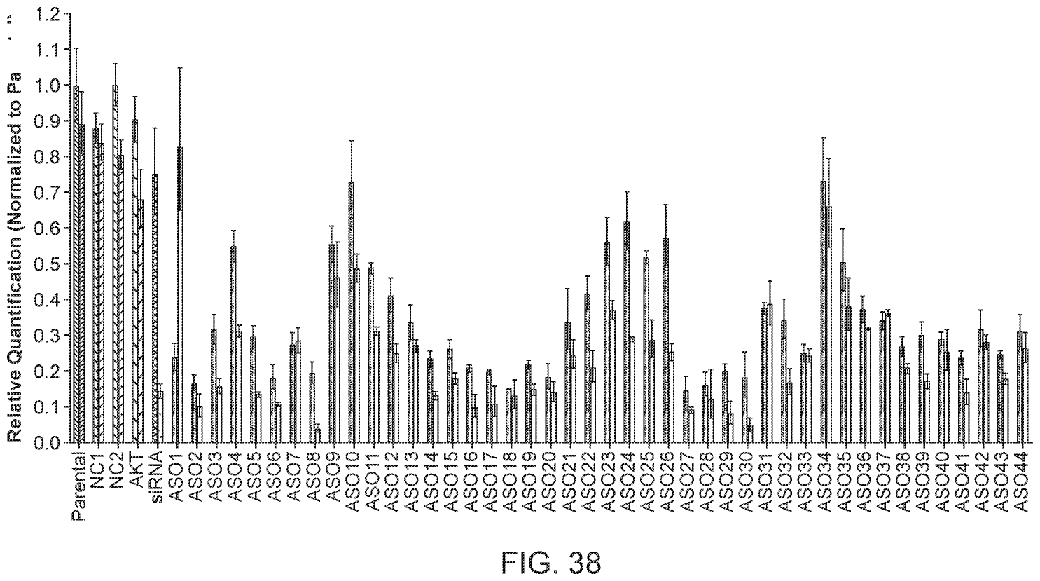

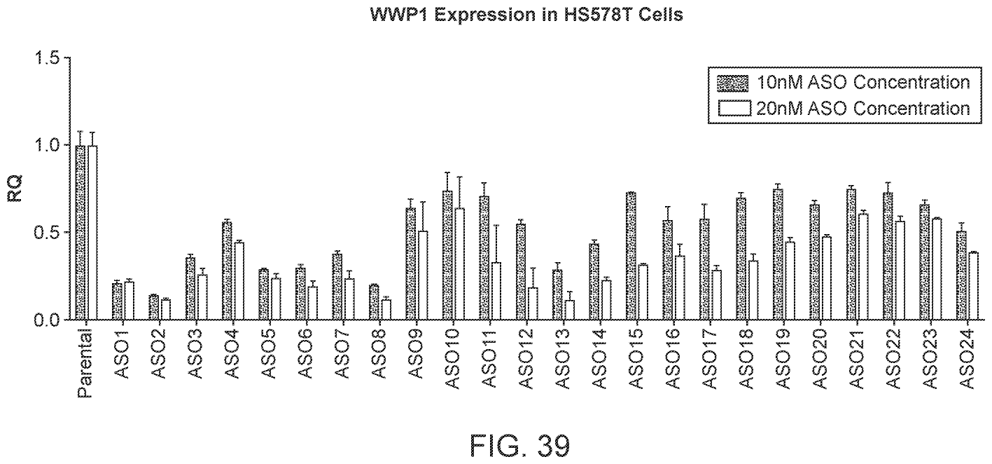

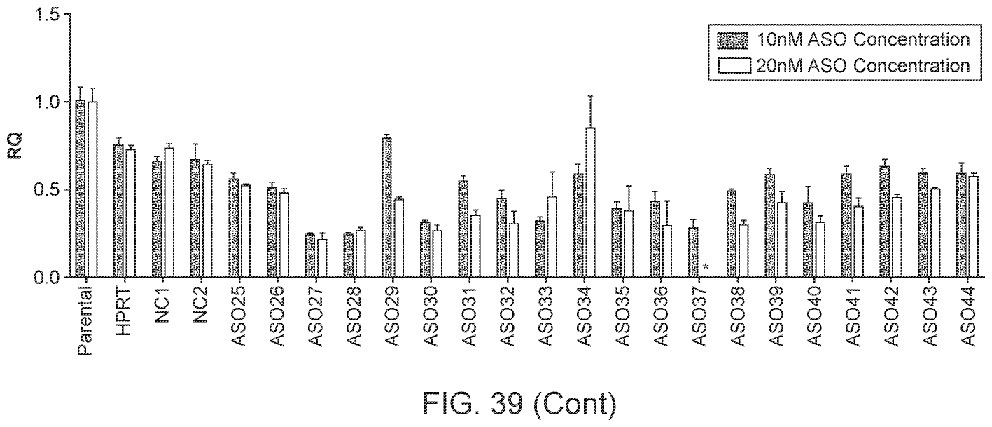

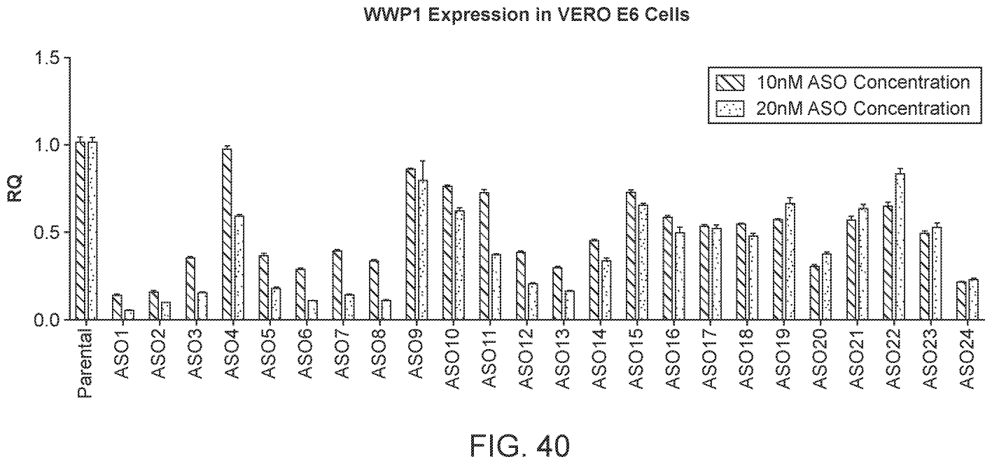

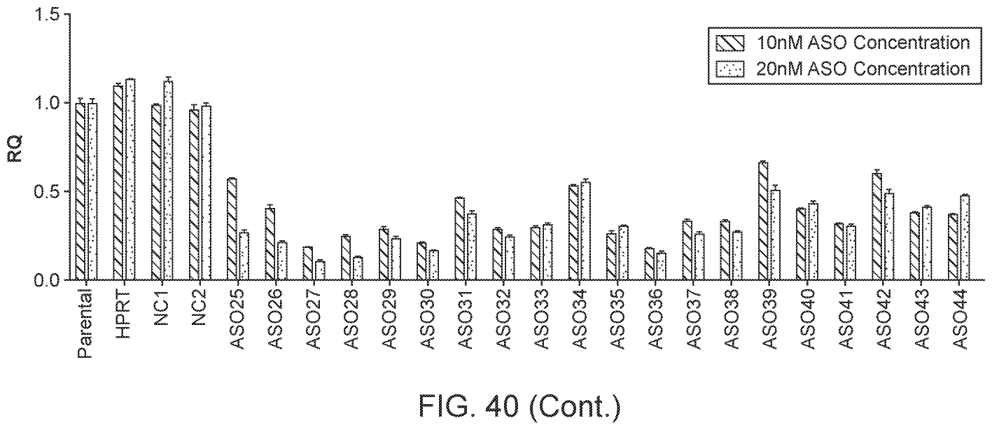

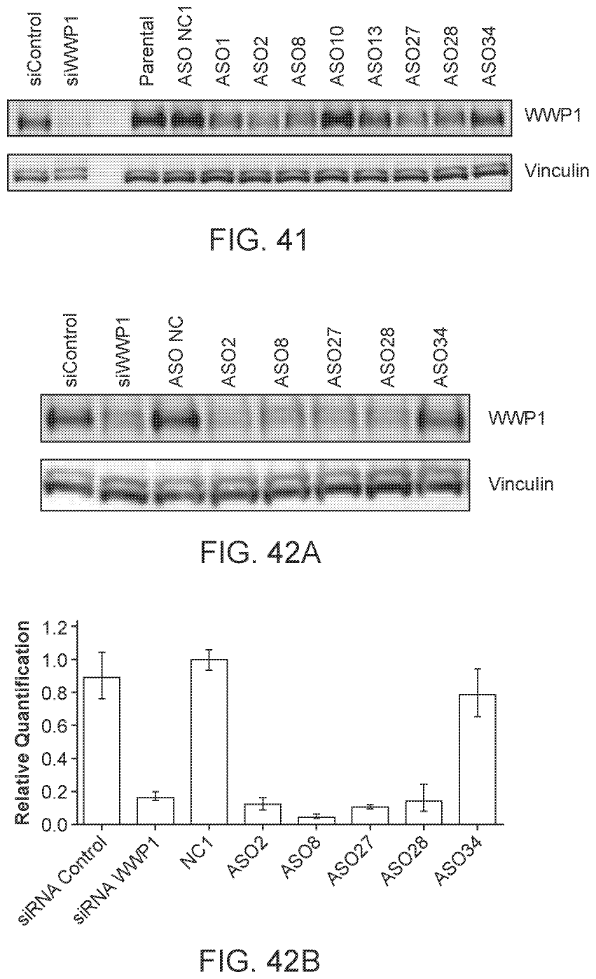

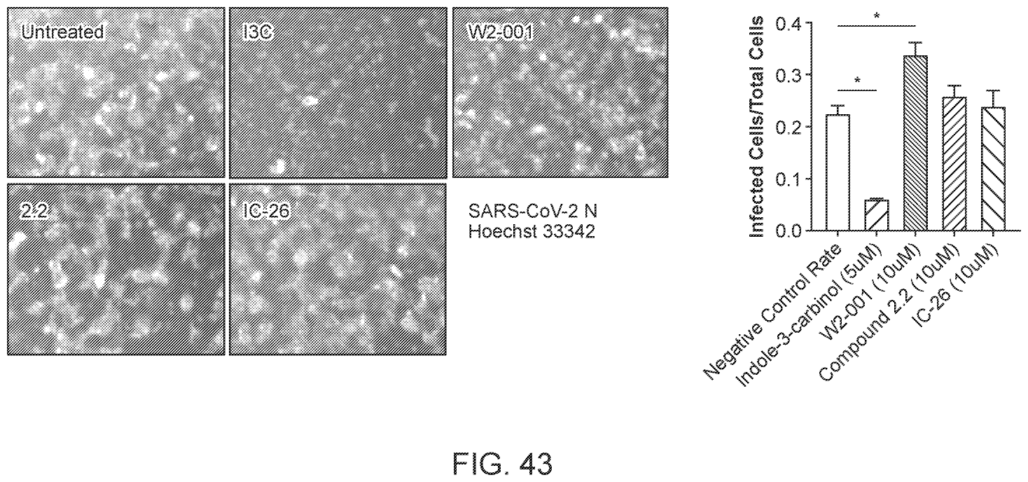

The molecular modelings also showed how the compounds bind specifically to HECT domain E3 ligases. FIG. 1 shows binding mode for compound 9 in the pocket of WWP1. The docked pose for the compound shows clear interactions between the molecule and protein residues including Try656 and Try628. These aromatic residues play a critical role in compound binding.

### Example 10

### Synthesis of Small Molecule Modulators

### 10.1 Synthesis of 6-ethynyl-1-phenyl-1H-indole-3-carbaldehyde (1)

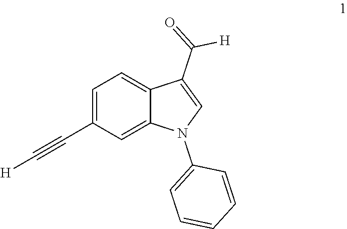 [SMILES: C#Cc1ccc2c(C=O)cn(-c3ccccc3)c2c1]

### Step-1

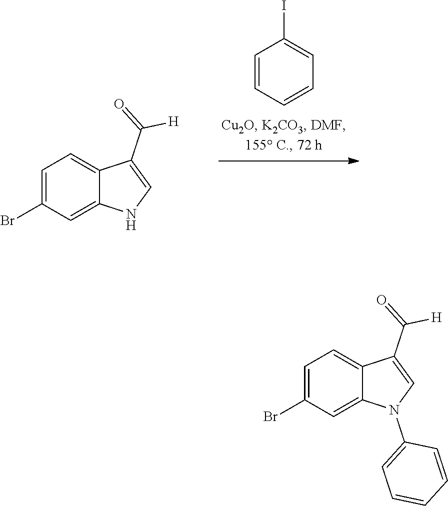 [SMILES: O=Cc1c[nH]c2cc(Br)ccc12 ; Ic1ccccc1 ; O=Cc1cn(-c2ccccc2)c2cc(Br)ccc12]

6-Bromo-1-phenyl-1H-indole-3-carbaldehyde: To a stirred solution of 6-bromo-1H-indole-3-carbaldehyde (2.0 g, 8.968 mmol) in DMF (30 mL) was added K₂CO₃ (2.46 g, 17.932 mmol), Cu₂O (384 mg, 2.69 mmol) and iodo benzene (1.9 mL, 17.937 mmol) at RT. The mixture was, heated at 155° C. for 72 h. TLC analysis showed the formation of required compound and ˜40% presence of the starting material. The reaction mixture was filtered through a celite bed, and was washed with EtOAc (500 mL). The combined organic layer was washed with cold water (2×250 mL), with brine solution (50 mL), was. dried over Na₂SO₄ and was concentrated in vacuo. The crude residue was purified by column chromatography (100-200 silica gel, 20% EtOAc-Hexane) to afford the title compound (1.1 g, 41%) as a pale brown solid. LCMS: m/z: 300, 302.09 [M, M+2]⁺

### Step-2

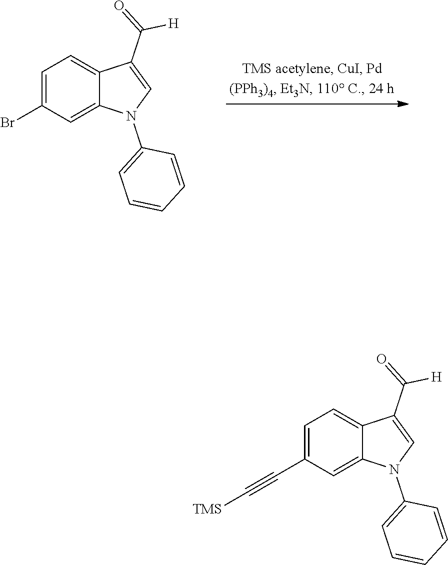 [SMILES: O=Cc1cn(-c2ccccc2)c2cc(Br)ccc12 ; C[Si](C)(C)C#Cc1ccc2c(C=O)cn(-c3ccccc3)c2c1]

1-Phenyl-6-((trimethylsilyl)ethynyl)-1H-indole-3-carbaldehyde: To a stirred solution of 6-bromo-1H-indole-3-carbaldehyde (1.1 g, 3.666 mmol) in Et₃N (20 mL) was added Pd (PPh₃)₄(423 mg, 0.3666 mmol) and CuI (50 mg, 0.2676 mmol) at RT. The reaction mixture was degassed with argon for 10 min, and TMS acetylene (0.75 mL, 5.49 mmol) was added at RT. The reaction mixture was heated at 110° C. for 24 h. Completion of the reaction was confirmed by TLC analysis. The reaction mixture was diluted with water (250 mL) and was extracted with EtOAc (3×250 mL). The combined extract was washed with brine (200 mL), was dried over Na₂SO₄ and was concentrated in vacuo. The crude residue was purified by column chromatography (100-200 silica gel, 10% EtOAc-Hexane) to afford compound the title compound (900 mg, 77%) as a yellow oil. R_{f}=0.45 (Mobile phase: 40% EtOAc-hexane).

### Step-3

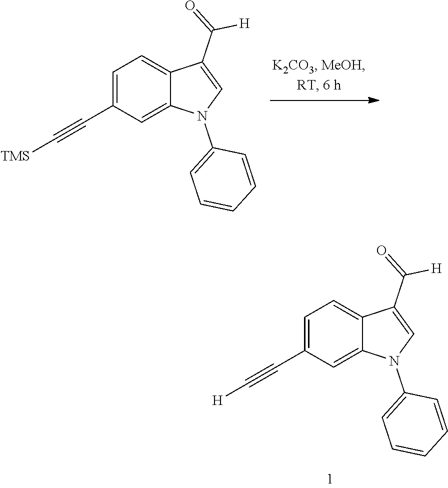 [SMILES: C[Si](C)(C)C#Cc1ccc2c(C=O)cn(-c3ccccc3)c2c1 ; C#Cc1ccc2c(C=O)cn(-c3ccccc3)c2c1]

6-Ethynyl-1-phenyl-1H-indole-3-carbaldehyde (1): To a stirred solution of 1-phenyl-6-((trimethylsilyl)ethynyl)-1H-indole-3-carbaldehyde (400 mg, 1.261 mmol) in MeOH (8 mL) was added K₂CO₃ (35 mg, 0.252 mmol) at RT. The reaction mixture was stirred at RT for 6 h. Completion of the reaction was confirmed by TLC analysis. The solvent was removed under reduced pressure to give a crude residue, which was diluted with water (100 mL) and was extracted with EtOAc (3×150 mL). The extract was washed with brine solution (50 mL), was dried over Na₂SO₄ and was concentrated in vacuo. The crude residue was purified by column chromatography (100-200 silica gel, 50% EtOAc-hexane) to afford 1 (220 mg, 71%) as a pale brown solid. ¹H NMR (400 MHz, CDCl₃): δ 10.10 (s, 1H), 8.32 (d, J=8.4 Hz, 1H), 7.96 (s, 1H), 7.62-7.57 (m, 3H), 7.53-7.26 (m, 4H), 3.07 (s, 1H). LCMS: m/z: 246.05 [M+H]⁺.

### 10.2 Synthesis of N-(2-cyanopyridin-4-yl)-3-formyl-1-phenyl-1H-indole-5-carboxamide (2)

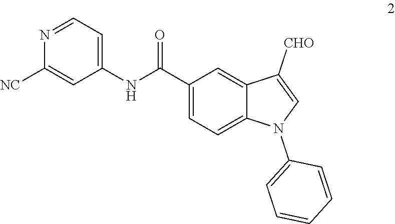 [SMILES: N#Cc1cc(NC(=O)c2ccc3c(c2)c(C=O)cn3-c2ccccc2)ccn1]

### Step-1

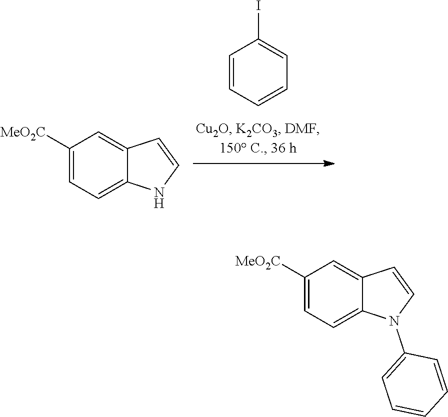 [SMILES: Ic1ccccc1 ; COC(=O)c1ccc2[nH]ccc2c1 ; COC(=O)c1ccc2c(ccn2-c2ccccc2)c1]

Methyl 1-phenyl-1H-indole-5-carboxylate: To a stirred solution of methyl 1H-indole-5-carboxylate (2.0 g, 11.428 mmol) in DMF (16 mL) was added K₂CO₃ (3.15 g, 22.857 mmol), Cu₂O (490 mg, 3.428 mmol) and Iodo benzene (1.9 mL, 17.142 mmol) at RT. The mixture was heated at 155° C. for 36 h. Completion of the reaction was confirmed by TLC analysis. The reaction mixture was filtered through a celite bed, and was washed the bed with EtOAc (500 mL). The combined organic filtrate was washed with cold water (2×250 mL) and with brine solution (50 mL) was dried over Na₂SO₄ and was concentrated in vacuo. The crude residue was purified by column chromatography (100-200 silica gel, 10% EtOAc-hexane) to afford the title compound (1.45 g, 51%) as a pale brown solid. LCMS: m/z: 252.25 [M+H]⁺.

### Step-2

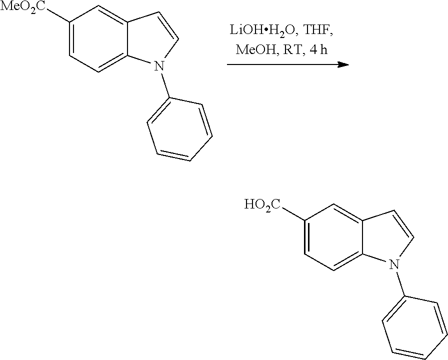 [SMILES: COC(=O)c1ccc2c(ccn2-c2ccccc2)c1 ; O=C(O)c1ccc2c(ccn2-c2ccccc2)c1]

1-Phenyl-1H-indole-5-carboxylic acid: To a stirred of solution of methyl 1-phenyl-1H-indole-5-carboxylate (500 mg, 1.989 mmol) in THF:MeOH:H₂O (1:1:1) (15 mL) was added LiOH·H₂O (250 mg, 5.969 mmol) at 0° C., The mixture was stirred at RT for 4 h. Completion the reaction was confirmed by TLC analysis. Volatiles were removed in vacuo to get a crude residue, which was acidified with 1N HCl (pH=2 to 3) and was extracted with EtOAc (3×100 mL). The extract was dried over Na₂SO₄ and was concentrated in vacuo to afford the title compound (450 mg, 95%) as an off white solid. LCMS: m/z: 238.22 [M+H]⁺.

### Step-3

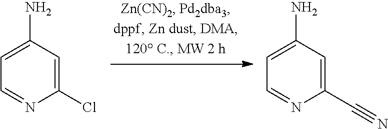 [SMILES: Nc1ccnc(Cl)c1 ; N#Cc1cc(N)ccn1]

4-Aminopicolinonitrile: To a stirred of solution of 2-chloropyridin-4-amine (500 mg, 3.889 mmol) in DMA (6 mL) was added Pd₂dba₃ (284 mg, 0.311 mmol), dppf (344 mg, 0.622 mmol), Zn(CN)₂ (1.27 g, 10.889 mmol), Zn powder (122 mg, 1.866 mmol) at RT. The mixture was irradiated in MW at 130° C. for 2 h. Completion the reaction was confirmed by TLC analysis The reaction mixture was diluted with EtOAc (300 mL) and the organic layer was washed with cold water (2×250 mL) and brine solution (50 mL). The organic layer was dried over Na₂SO₄ and was concentrated in vacuo. The crude residue was purified by column chromatography (100-200 silica gel, 50% EtOAc-hexane) to afford the title compound (160 mg, 21%) as a pale brown solid. LCMS: m/z: 120.14 [M+H]⁺.

### Step-4

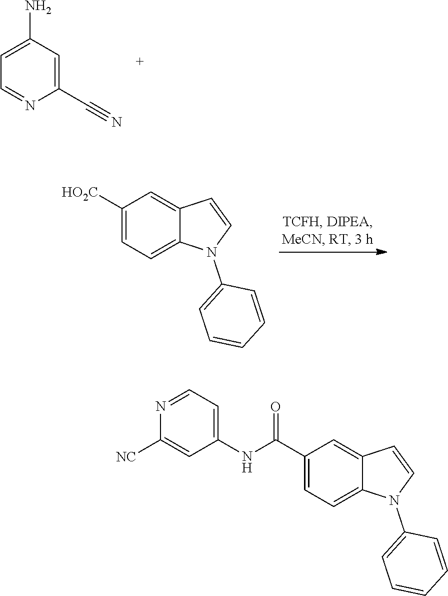 [SMILES: N#Cc1cc(N)ccn1 ; O=C(O)c1ccc2c(ccn2-c2ccccc2)c1 ; N#Cc1cc(NC(=O)c2ccc3c(ccn3-c3ccccc3)c2)ccn1]

N-(2-cyanopyridin-4-yl)-1-phenyl-1H-indole-5-carboxamide: To a stirred solution of 1-phenyl-1H-indole-5-carboxylic acid (100 mg, 0.421 mmol) and 4-aminopicolinonitrile (55 mg, 0.46 mmol) in MeCN (2 mL), N,N,N′N′-tetramethylchloroformamidium hexafluorophosphate (TCFH) (141 mg, 0.505 mmol) and DIPEA (0.3 mL, 1.68 mmol) were added at RT. The mixture was stirred at RT for 4 h. Completion the reaction was confirmed by TLC analysis. The reaction mixture was diluted with EtOAc (300 mL) and the combined organic layer was washed with cold water (2×250 mL) and brine solution (50 mL). The organic layer was dried over Na₂SO₄ and was concentrated in vacuo. The crude residue was purified by column chromatography (100-200 silica gel, 20% EtOAc-hexane) to afford the title compound (130 mg, 84%) as a pale brown solid. LCMS: m/z: 339.22 [M+H]⁺.

### Step-5

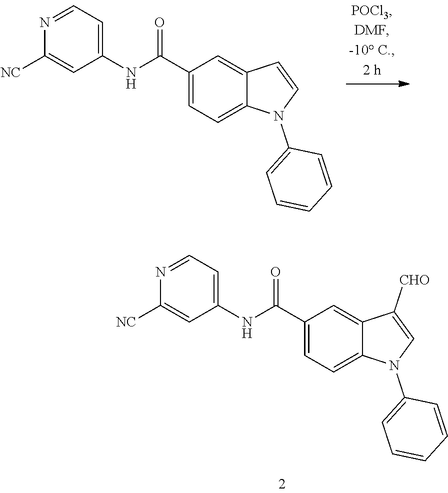 [SMILES: N#Cc1cc(NC(=O)c2ccc3c(ccn3-c3ccccc3)c2)ccn1 ; N#Cc1cc(NC(=O)c2ccc3c(c2)c(C=O)cn3-c2ccccc2)ccn1]

N-(2-cyanopyridin-4-yl)-3-formyl-1-phenyl-1H-indole-5-carboxamide (2): To a solution of N-(2-cyanopyridin-4-yl)-1-phenyl-1H-indole-5-carboxamide (120 mg, 0.355 mmol) in DMF (0.2 mL) was added a solution of POCl₃ (33 μL) in DMF (0.2 mL) at 0° C. The reaction mixture was stirred at 10° C. for 1 h at which time TLC analysis showed complete reaction. The reaction mixture was poured into ice water, was basified with 0.5 M NaOH solution (pH=12) and was extracted with EtOAc (3×40 mL). The organic layer was dried over Na₂SO₄ and was concentrated in vacuo. The crude residue was purified by C-18 RP CombiFlash (RediSep Rf Gold C18 column, eluted with 60% acetonitrile in water) to afford compound 2 (12 mg, 9%) as an off white solid. ¹H NMR (300 MHz, DMSO-d₆): δ 11.13 (s, 1H), 10.11 (s, 1H), 8.91 (s, 1H), 8.79 (s, 1H), 8.70-8.65 (m, 1H), 8.39 (br s, 1H), 8.12-8.10 (m, 1H), 8.00-7.97 (m, 1H), 7.75-7.60 (m, 5H), 7.60-7.55 (m, 1H).

### 10.3 Syntheses of 3-((1H-indol-3-yl)methyl)-1H-indole-5-carboxylic acid (3) and 3-((1H-indol-3-yl)methyl)-1H-indole-6-carboxylic acid (4)

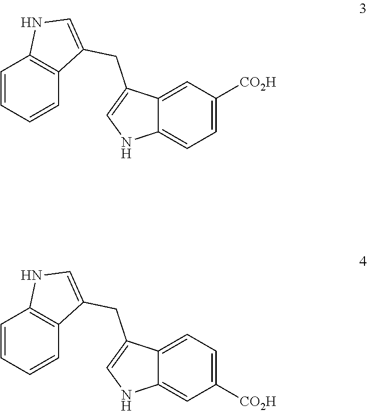 [SMILES: O=C(O)c1ccc2[nH]cc(Cc3c[nH]c4ccccc34)c2c1 ; O=C(O)c1ccc2c(Cc3c[nH]c4ccccc34)c[nH]c2c1]

### Step-1

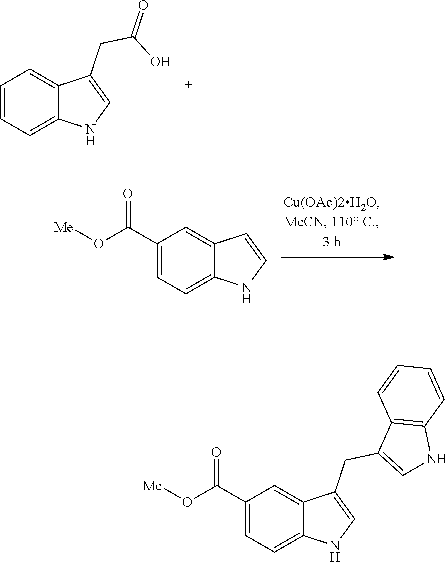 [SMILES: O=C(O)Cc1c[nH]c2ccccc12 ; COC(=O)c1ccc2[nH]ccc2c1 ; COC(=O)c1ccc2[nH]cc(Cc3c[nH]c4ccccc34)c2c1]

Methyl 3-((1H-indol-3-yl)methyl)-1H-indole-5-carboxylate: To a stirred of solution of 2-(1H-indol-3-yl)acetic acid (1.0 g, 5.708 mmol) and methyl 1H-indole-5-carboxylate (2 g, 11.41 mmol) in MeCN (10 mL) was added Cu(OAc)₂·H₂O (1.14 g, 5.708 mmol) at RT. The mixture was, heated at 80° C. for 3 h. Completion the reaction was confirmed by TLC analysis. The reaction mixture was filtered through celite bed, and was washed with EtOAc (250 mL). The combined organic filtrate was washed with brine solution (50 mL), was dried over Na₂SO₄ and was concentrated in vacuo. The crude residue was purified by column chromatography (100-200 silica gel, 15% EtOAc-hexane) to afford the title compound (700 mg, 40%) as a Pale brown solid. R_{f}=0.5 (Mobile phase: 40% EtOAc-hexane).

### Step-2

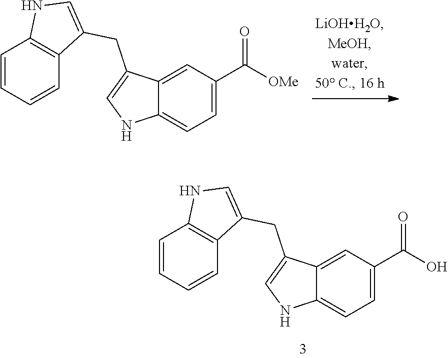 [SMILES: COC(=O)c1ccc2[nH]cc(Cc3c[nH]c4ccccc34)c2c1 ; O=C(O)c1ccc2[nH]cc(Cc3c[nH]c4ccccc34)c2c1]

3-((1H-indol-3-yl)methyl)-1H-indole-5-carboxylic acid (3): A stirred of solution of methyl 3-((1H-indol-3-yl)methyl)-1H-indole-5-carboxylate (700 mg, 2.302 mmol) and LiOH·H₂O (580 mg, 13.815 mmol) in THF:MeOH:H₂O (1:1:1) (15 mL) was stirred at 50° C. for 16 h. Completion the reaction was confirmed by TLC analysis. The mixture was concentrated in vacuo to get crude residue, which was acidified with 1N HCl (pH=2 to 3) and was, extracted with EtOAc (3×100 mL). The extract was dried over Na₂SO₄ and was concentrated in vacuo to afford compound 3 (810 mg, 85%) as a pale brown solid: MS: m/z=289.14 [M−1]⁺; ¹H NMR (400 MHz, DMSO-d₆): δ 12.33 (s, 1H), 11.13 (d, J=1.2 Hz, 1H), 10.76 (s, 1H), 8.19 (t, J=0.7 Hz, 1H), 7.67 (dd, J_{A}=9.6 Hz, J_{B}=4.5 Hz, 1H), 7.51 (d, J=8 Hz, 1H), 7.37 (d, J=8 Hz, 1H), 7.32 (d, J=8.4 Hz, 1H), 7.26 (d, J=2 Hz, 1H), 7.11 (d, J=2.4 Hz, 1H), 7.05-7.01 (m, 1H), 6.93-6.89 (m, 1H), 4.18 (s, 2H).

3-((1H-indol-3-yl)methyl)-1H-indole-6-carboxylic acid (4): This compound was prepared in a manner similar to that used for compound 3 by substituting methyl 1H-indole-6-carboxylate for methyl 1H-indole-5-carboxylate in Step-1: MS: m/z=289.14 [M−1]⁺; ¹H NMR (400 MHz, DMSO-d₆): δ 12.39 (s, 1H), 11.12 (s, 1H), 10.74 (s, 1H), 7.97 (s, 1H), 7.59-7.49 (m, 3H), 7.37 (d, J=2.4 Hz, 1H), 7.31 (d, J=8.4 Hz, 1H), 710 (d, J=0.8 Hz, 1H), 7.046-7.006 (m, 1H), 6.931-6.891 (m, 1H), 3.306 (s, 2H).

### 10.4 Syntheses of 3-((1H-indol-3-yl)methyl)-2-methyl-N-(prop-2-yn-1-yl)-1H-indole-6-carboxamide (5) and 3-((1H-indol-3-yl)methyl)-N-(prop-2-yn-1-yl)-1H-indole-6-carboxamide (6)

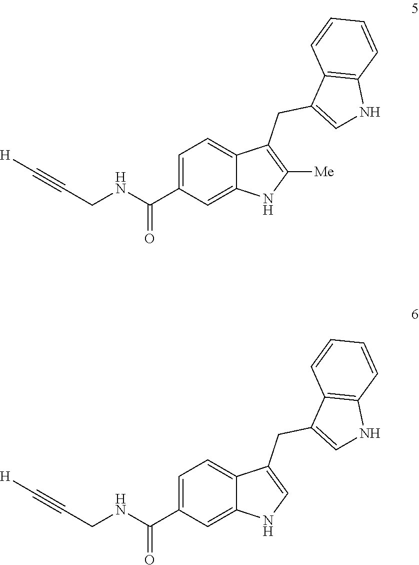 [SMILES: C#CCNC(=O)c1ccc2c(Cc3c[nH]c4ccccc34)c(C)[nH]c2c1 ; C#CCNC(=O)c1ccc2c(Cc3c[nH]c4ccccc34)c[nH]c2c1]

### Step-1

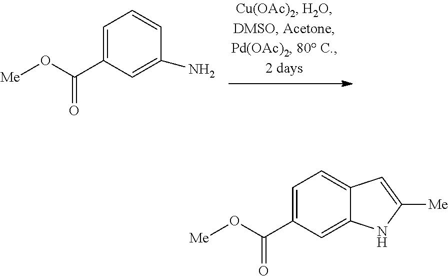 [SMILES: COC(=O)c1cccc(N)c1 ; COC(=O)c1ccc2cc(C)[nH]c2c1]

Methyl 2-methyl-1H-indole-6-carboxylate: To a stirred solution of methyl 3-aminobenzoate (2.0 g, 13.24 mmol) in DMSO (10 mL) was added Cu(OAc)₂·H₂O (7.9 g, 39.72 mmol) at RT. The mixture was, degassed with Ar for 20 min, then Acetone (20 mL), Pd(OAc)2 (220 mg, 1.324 mmol) were added at RT. The mixture was, heated at 80° C. for 48 h. Completion of the reaction was confirmed by TLC analysis. The reaction mixture was filtered through celite bed and was washed with EtOAc (250 mL). The combined organic layer was washed with cold water (2×250 mL) and brine solution (50 mL), was dried over Na₂SO₄ and was concentrated in vacuo. The crude residue was purified by column chromatography (100-200 silica gel, 25% EtOAc-hexane) to afford the title compound (1.4 g, 56%) as a pale brown solid.

### Step-2

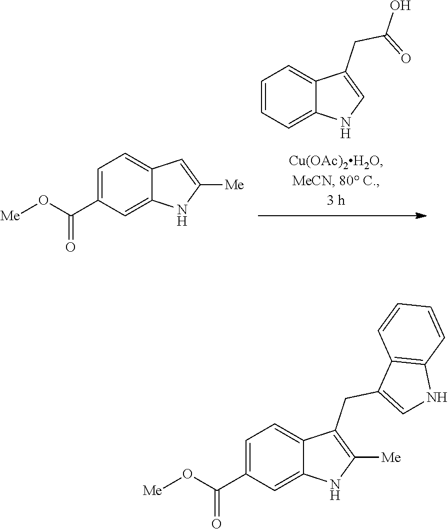 [SMILES: O=C(O)Cc1c[nH]c2ccccc12 ; COC(=O)c1ccc2cc(C)[nH]c2c1 ; COC(=O)c1ccc2c(Cc3c[nH]c4ccccc34)c(C)[nH]c2c1]

Methyl 3-((1H-indol-3-yl)methyl)-2-methyl-1H-indole-6-carboxylate: To a stirred solution of methyl 2-methyl-1H-indole-6-carboxylate (3.0 g, 17.14 mmol), and 2-(1H-indol-3-yl)acetic acid (6.5 g, 34.28 mmol) in MeCN (50 mL) was added Cu(OAc)₂·H₂O (3.4 g, 17.14 mmol) at RT. The mixture was heated at 80° C. for 3 h. Completion the reaction was confirmed by TLC analysis. The reaction mixture was filtered through celite bed, and was washed with EtOAc (250 mL). The combined organic layer was washed with brine solution (50 mL), was separated, was dried over Na₂SO₄ and was concentrated in vacuo. The crude residue was purified by column chromatography (100-200 silica gel, 30% EtOAc-hexane) to afford the title compound (2.6 g, 47%) as a pale brown solid.

### Step-3

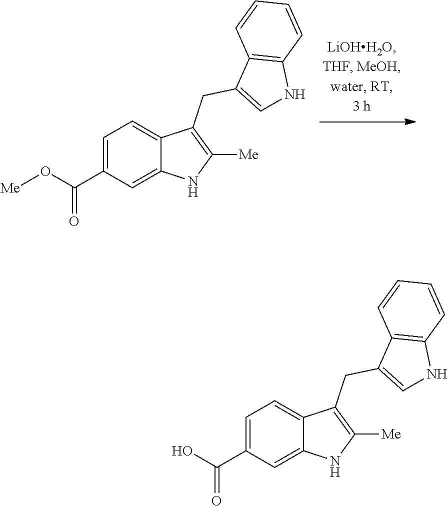 [SMILES: COC(=O)c1ccc2c(Cc3c[nH]c4ccccc34)c(C)[nH]c2c1 ; Cc1[nH]c2cc(C(=O)O)ccc2c1Cc1c[nH]c2ccccc12]

3-((1H-indol-3-yl)methyl)-2-methyl-1H-indole-6-carboxylic acid: To a stirred of solution of methyl 3-((1H-indol-3-yl)methyl)-2-methyl-1H-indole-6-carboxylate (1.0 g, 3.144 mmol) in THF:MeOH:H₂O (1:1:1) (20 mL) was added LiOH·H₂O (396 mg, 9.433 mmol) at 0° C. The mixture was stirred at RT for 3 h. Completion the reaction was confirmed by TLC analysis. Volatiles were removed in vacuo to get crude residue, the obtained crude residue, which was acidified with 1N HCl (pH=2 to 3). The mixture was extracted with EtOAc (3×100 mL), was dried over Na₂SO₄ and was concentrated in vacuo to afford the title compound (810 mg, 85%) as a pale brown solid.

### Step-4

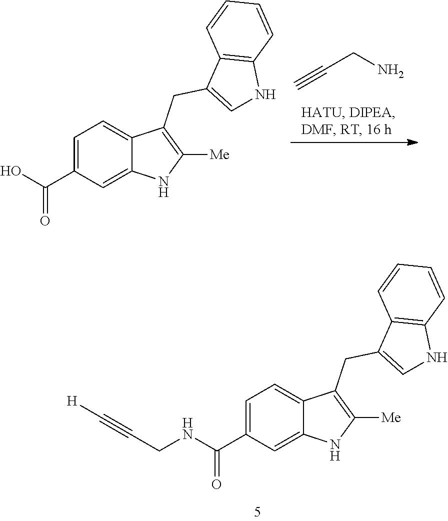

3-((1H-indol-3-yl)methyl)-2-methyl-N-(prop-2-yn-1-yl)-1H-indole-6-carboxamide (5): To a stirred solution of 3-((1H-indol-3-yl)methyl)-2-methyl-1H-indole-6-carboxylic acid (600 mg, 1.973 mmol) in DMF (6.0 mL) was added propargyl amine (0.15 mL, 2.368 mmol), HATU (1.1 g, 2.960 mmol), DIPEA (0.7 mL, 3.947 mmol) at RT. The mixture was stirred at RT for 16 h. Completion the reaction was confirmed by TLC analysis. The reaction mixture was diluted with cold water (200 mL), and was extracted with EtOAc (3×150 mL). The extract was dried over Na₂SO₄, was concentrated in vacuo to give a crude compound, which was purified by column chromatography (100-200 mesh silica gel, 60% EtOAc-hexane) as eluent to afford compound 5 (400 mg, 59%) as light brown solid: MS: m/z=342.34 [M+H]+; ¹H NMR (300 MHz, DMSO-d₆): δ 11.03 (s, 1H), 10.68 (s, 1H), 8.67 (t, J=5.4 Hz, 1H), 7.8 (s, 1H), 7.46 (d, J=8.1 Hz, 1H), 7.42 (s, 2H), 7.29 (d, J=8.1 Hz, 1H), 7.04-6.98 (m, 2H), 6.9 (t, J=7.5 Hz, 1H)), 4.07-4.03 (m, 4H), 3.06 (s, 1H), 2.42 (s, 3H).

3-((1H-indol-3-yl)methyl)-N-(prop-2-yn-1-yl)-1H-indole-6-carboxamide (6). This compound was prepared by substituting 3-((1H-indol-3-yl)methyl)-1H-indole-6-carboxylic acid (4) for 3-((1H-indol-3-yl)methyl)-2-methyl-1H-indole-6-carboxylic acid in step-4: MS 328.18 [M+H]⁺; ¹H NMR (300 MHz, DMSO-d₆): δ 11.1 (s, 1H), 10.75 (s, 1H) 8.76 (t, J=5.4 Hz, 1H), 7.9 (s, 1H), 7.57-7.45 (m, 3H), 7.32 (m, 2H), 7.15 (s, 1H), 7.14-6.89 (m, 2H), 4.14 (s, 2H), 4.04 (m, 2H), 3.09 (t, J=2.3 Hz, 1H).

### 10.5 Synthesis of 3-((1H-indol-3-yl)methyl)-N-(2-hydroxyethyl)-N-(prop-2-yn-1-yl)-1H-indole-6-carboxamide (7)

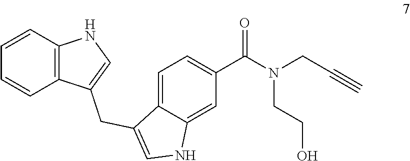 [SMILES: C#CCN(CCO)C(=O)c1ccc2c(Cc3c[nH]c4ccccc34)c[nH]c2c1]

### Step-1

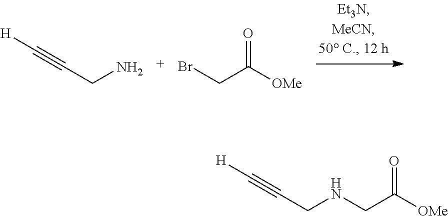

Methyl 2-(prop-2-yn-1-ylamino)acetate: To a stirred solution of propargylamine (5 g, 90.9 mmol) in MeCN (50 mL) was added Et₃N (19.1 mL, 136.36 mmol) at 0° C. followed by methyl bromoacetate (13 mL, 136.36 mmol). The mixture was heated at 50° C. for 12 h. Completion the reaction was confirmed by TLC analysis. The reaction mixture was diluted with water (500 mL) and was extracted with EtOAc (3×250 mL). The organic layer was dried over Na₂SO₄ and was concentrated in vacuo to get crude compound, which was purified by column chromatography (100-200 mesh silica gel, 20% EtOAc-hexane) as eluent to afford the title compound (6 g, 52%) as yellow oil. R_{f}=0.4 (Mobile phase: 40% EtOAc-Hexane).

### Step-2

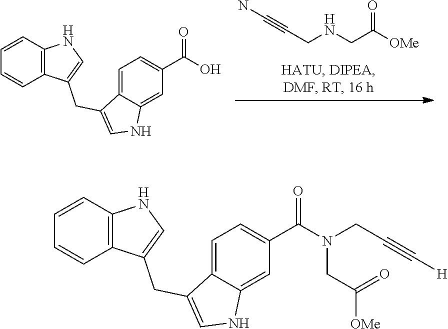 [SMILES: O=C(O)c1ccc2c(Cc3c[nH]c4ccccc34)c[nH]c2c1 ; COC(=O)CNCC#CN ; C#CCN(CC(=O)OC)C(=O)c1ccc2c(Cc3c[nH]c4ccccc34)c[nH]c2c1]

Methyl 2-(3-((1H-indol-3-yl)methyl)-N-(prop-2-yn-1-yl)-1H-indole-6-carboxamido)acetate: To a stirred solution of 3-((1H-indol-3-yl)methyl)-1H-indole-6-carboxylic acid (4) (700 mg, 2.41 mmol) in DMF (7.0 mL) was added methyl 2-(prop-2-yn-1-ylamino)acetate (360 mg, 2.89 mmol), HATU (1.37 g, 3.62 mmol), and DIPEA (1.1 mL, 6.03 mmol) at RT. The mixture was, stirred at RT for 16 h. Completion the reaction was confirmed by TLC analysis. The reaction mixture was diluted with cold water (200 mL) and was extracted with EtOAc (3×150 mL). The organic extract was dried over Na₂SO₄ and was concentrated in vacuo to get crude compound, which was purified by column chromatography (100-200 mesh silica gel, 50% EtOAc-hexane) as eluent to afford the title compound (260 mg, 31%) as light brown solid.

### Step-3

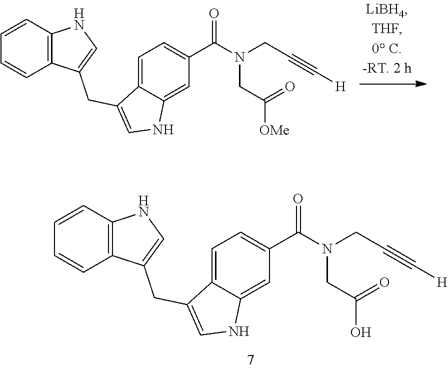 [SMILES: C#CCN(CC(=O)OC)C(=O)c1ccc2c(Cc3c[nH]c4ccccc34)c[nH]c2c1 ; C#CCN(CC(=O)O)C(=O)c1ccc2c(Cc3c[nH]c4ccccc34)c[nH]c2c1]

3-((1H-indol-3-yl)methyl)-N-(2-hydroxyethyl)-N-(prop-2-yn-1-yl)-1H-indole-6-carboxamide (7): To a stirred solution of methyl 2-(3-((1H-indol-3-yl)methyl)-N-(prop-2-yn-1-yl)-1H-indole-6-carboxamido)acetate (120 mg, 0.300 mmol) in THF (6 mL) was added LiBH4 (20 mg, 0.900 mmol) at 0° C. The mixture was, stirred at RT for 2 h. The reaction was quenched with sat.aq.NH₄Cl (50 mL) and extracted with EtOAc (3×50 mL). The extract was dried over Na₂SO₄ and was concentrated in vacuo to get crude compound, which was purified by column chromatography (100-200 mesh silica gel, 70% EtOAc-hexane) as eluent to afford compound 7 (40 mg, 36%) as light brown solid: MS: m/z=372.17 [M+H]⁺; ¹H NMR (300 MHz, DMSO-d₆): δ 10.98 (s, 1H), 10.74 (s, 1H), 7.57 (d, J=8.4 Hz, 1H)), 7.53 (d, J=8 Hz, 1H), 7.42 (s, 1H), 7.31 (d, J=8 Hz, 1H), 7.27 (s, 1H), 7.16 (s, 1H), 7.05-7.01 (m, 2H), 6.94-6.9 (m, 1H), 4.7 (s, 1H), 4.24 (s, 2H), 4. 14 (s, 2H), 3.58 (bs, 2H), 3.48 bs, 2H), 3.3 (s, 1H).

### Example 11

### WWP1 MALDI-MS Ubiquitin Depletion Assay

Modulators designed in these ways were tested for HECT domain E3 ligase activity in a MALDI TOF assay of auto-ubiquitination (De Cesare et al, Cell Chem. Biol, 2018, 25, 1117)

### Materials

UBE1 (E-305 Boston Biochem), UBE2D1 (E2-616 Boston Biochem), WWP1 (Ubiquigent #63-0034-025/30033/D), ubiquitin variants (Boston Biochem; ubiquitin: U-100H; WT-His6-Ub: U-530; K27R-His6-Ub:UM-HK27R; K27O-His6-Ub: UM-HK27O), alpha-cyano-4-hydroxycinnamic acid (CHCA), ATP, trifluoroacetic acid (Millipore Sigma, St. Louis, Mo.), 384-well round-bottom polypropylene microplates (781280, Greiner, Stonehouse, UK), 384-well Anchorchip MALDI targets and Peptide Calibration Standard II (Bruker Daltonics, Billerica, Mass.).

Protocol: The WWP1 ubiquitin depletion assay consists of recombinant UBE1 (5 nM), UBE2D1 conjugating enzyme (60 nM) and WWP1 ligase (50 nM) in a 10 mM HEPES pH 8.0 containing 0.1 mg/mL BSA, 10 mM MgCl2, 1 mM ATP and 1500 nM ubiquitin. Screening was performed by dispensing 3.5 μL of an enzyme solution containing UBE1 (10 nM), UBE2D1 (120 nM), WWP1 (100 nM), ubiquitin (3000 uM) to a 384-well round bottom polypropylene plates followed by incubation at 37° C. for 30 min. Reactions were initiated by the addition of 3.5 μL of an ATP solution (2000 nM) then incubated at 37° C. for 90 minutes before being quenched by the addition of 5 μL of a 50 mM EDTA solution containing 750 nM of a ubiquitin labelled with a histidine expression tag (m/z=9460.0 Da).

For detection of ubiquitin and the ubiquitin-6His internal standards, 50 nL of quenched reaction was dispensed onto a 384-well Anchorchip MALDI target plate (Bruker) pre-coated with a uniform layer of CHCA crystals immediately followed by 200 nL of a 3 mg/mL CHCA matrix solution (6 mg of CHCA dissolved in 0.7 mL of 100% ethanol and 1.3 mL of 0.31% aqueous TFA) using a TTP Labtech Mosquito liquid handler. Spectra for all reactions were acquired on a Bruker AutoFlex II in positive linear TOF mode from 1000 laser shots with ion suppression below 4000 Da. Spectra were analyzed for response using a centroid peak-fitting algorithm to quantify ubiquitin (m/z=8564.0 Da) and ubiquitin-6His (m/z=9460.0 Da) followed taking the ratio of intensities of ubiquitin and the ubiquitin-6His internal standard.

For compound screening, 140 nL of diluted compound in 100% DMSO was added to the WWP1 enzyme solution and pre-incubated for 30 minutes prior to reaction initiation. IC₅₀ values for selected compounds are shown in Table 6.

TABLE 6
IC₅₀ values for auto-ubiquitination of WWP1

| Compound # | IC₅₀ (μM) |
|---|---|
| 1 | 5 |
| 2 | 26 |
| 3 | ~300 |
| 4 | 29 |
| 5 | 45 |
| 6 | 32 |
| 7 | 15 |
| 8 | 49 |
| 9 | 66 |

### Example 12

### Functional Assays—Cellular Proliferation Assays

DU-145 cells were trypsinized, counted via Trypan blue method and resuspended in DMEM complete medium (10% FBS with Pen-strep antibiotics) as described in Lieu et al, Mol Cel Endorinol, 87:19-28, 1992. Cells were seeded at a density of 100 cells per well in 1001 volume of 96-well cell culture plate and were incubated overnight at 37° C. in 5% CO₂ incubator. The next day, cells were given fresh media and the test compounds were added in different concentrations. After 72 hours of incubation, MTS reagent (procured from Promega) was added to the cells and they were incubated for an additional 2 hours. Finally, the plates read in the absorbance mode at 490 nm using an ELISA plate reader (Perkin Elmer Victor X3 plate reader). The % growth values were calculated as [(T−T₀)/(C−T₀)]×100, where T₀ is the OD of cells at the time of compound addition, C is the vehicle control, T is the OD of the test concentrations at 72 hours. Percentage inhibition values were calculated using 100−([(T−T₀)/(C−T₀)]×100). IC₅₀ values were calculated in graph pad prism software using dose response curve nonlinear regression method. IC50 values for selected compounds are shown in Table 7.

TABLE 7
IC₅₀ values for selected compounds in DU-145 cells

| Compound # | IC₅₀ (μM) |
|---|---|
| 1 | 13.51 |
| 4 | 0.08 |
| 5 | 5.48 |
| 6 | 8.08 |
| 7 | 5.01 |
| 8 | 12 |

### Example 13

### SARS CoV2 Egression in VeroE6 Cells and Viral Neutralizing Assays

### 13.1 Measurement of Viral Egress Using Modulators Against SARS-Cov2

Most potent small molecule modulators (SMMs) against WWP1 (e.g. Compound 1 and Compound 9 from Table 1) are selected and their viral egress against SARS-Cov2 and SARS-Cov1 are measured and compared.

Small molecule modulators demonstrated pan-activity to multiple relevant NEDD-4 family members which demonstrate additional benefit as HECT ligase work through hetero dimeric/trimeric/multimeric structures.

VeroE6 cells used for this experiment is from African Green Monkey, expressing cell surface receptors for virus interaction and entry. The cells are analyzed in virus neutralization assays to analyze the efficacy of the small molecule modulators measuring viral proteins in cells and viral load in the supernatant.

### 13.2 Introducing Mutation in PPxY Motif of SARs-Cov2 to Establish Causal Linkage

Successful modulators of viral SARS-CoV2 are tested against SARS-CoV1 (in the same assay). PPxY motif in the SARS-CoV2 envelop protein are mutated and viral egression is measured to establish a direct linkage between the WWP1 PPxY domain and increase in virulence.

### Example 14

### The Effect of WWP1 Inhibition on PD-L1 Expression

Most cancer cells utilize immune evasion strategies such as PD-L1 upregulation on the tumor cell. PD-L1 has been shown as target of WWP1. WWP1 ubiquitinates PD-L1 in catalytic dependent manner, forming polyubiquitin chains (C) (Lee Y R, Yehia L, Kishikawa T, et al. WWP1 Gain-of-Function Inactivation of PTEN in Cancer Predisposition. N Engl J Med. 2020; 382(22):2103-2116).

The effect of WWP1 inhibition on PD-L1 expression is established via three independent methods:

(i) Western of total cellular and exosomal/soluble PD-L1
(ii) Flow cytometry to measure cell surface PD-L1
(iii) Immunoprecipitation to measuring of ubiquitination status of PD-L1

These methods (especially the flow assay) establish a rapid screening assay to enable the small molecule discovery targeting WWP1.

### 14.1 Establishment of PD-L1 Expression on the Surface of TNBC Cancer Cells

The inhibition of WWP1 is found to be effective on PD-L1 protein levels on cells surface via FACS analysis. Two cell lines are selected, HS578T (TNBC) and MCF-7 (ER+ luminal) with matching high/low PD-L1 expression. Both cell lines express WWP1 and other relevant biomarkers (PTEN and AKT). Normal tissue controls are HS578Bst from peripheral breast tissue of same donor, and MCF10A, a healthy control.
HS578T-PD-L1high (TNBC); HS578Bst (healthy tissue control)
MCF-7-PD-L1low (ER+); MCF-10A Control (healthy tissue control)

A direct measure of cell surface PD-L1 is performed using a conjugated anti-PD-L1 (e.g. Alexa Fluor 488).

### 14.2 Monitoring PD-L1 Degradation (in WWP1 Know-Down Cells)

Small molecule modulators directed toward WWP1, WWP2, NEDD4 and ITCH are tested. After establishing conditions for the 4 cell lines (by qPCR), WWP1, WWP2, NEDD4 and ITCH levels are monitored by Western. Westerns for PD-L1 and ubiquitin (analysis after immunoprecipitation) determine PD-L1 levels and modification in cells with ablated WWP1, WWP2, NEDD4 and ITCH. After confirming the effect of E3 ligase knockdown on PD-L1 levels, the mechanism is explored in more detail to understand where PD-L1 is acting. Similar assays including dual/triple target knockdown (WWP1+WWP2; WWP1+WWP2+NEDD4) are performed.

### 14.3 Measurement of PD-L1 Surface Expression (with HECT Ligase Knockdown)

Re-localization of PD-L1 to the cell membrane is crucial for its pathology in TNBC and other cancers. PD-L1 surface levels are monitored before and after WWP1 knockdown.

### 14.4 Measurement of WWP1-Mediated Inhibition in T-Cell Activation Assay

The influence of the PD-L1/PD1 axis on the activity of T cells is measured with commercially available assays. These T cell activation assay comprises (i) expression vectors containing a T-cell receptor (TCR) activator for the PD-L1 positive cancer cells and (ii) TCR/PD-1 reporter cells with NFAT:Luciferase reporter gene. Using small molecule modulators, PD-L1 is downregulated in TNBC cell that results in T cell activation and increase luciferase in this luminescence assay.

### 14.5 Measure PD-L1 Exosomal Excretion and Soluble PD-L1

WWP1 and the level of PD-L1 containing exosome can affect T-cells activity in the tumor microenvironment after secretion (Tang et al., Front. Immunol, 2020). Direct exosomal PD-L1 secretion is changed while absolute levels of membrane PD-L1 remains stable (upon WWP1 knockdown). Exosomes are isolated from the media to quantify total soluble exosomal PD-L1 level, using simple ultrafiltration or ultracentrifugation (J Extracell Vesicles, 2012).

### Example 15

### Testing WWP1 Small Molecule Modulators to Evaluate Anti-Tumor Activity in GOF WWP1 Mutations

The goal of these experiments is to establish WWP1 mutations as a drug target for small molecule approaches.

### 15.1 Summary of Spectrum of Point Mutations and Evidence of Potential Function

There are several lines of evidence that suggest that WWP1 point mutation confer a gain-of-function phenotype and lead cancer transformation. WWP1 mutations have been found in all functional domains of the protein (catalytic HECT domain, C2 domain, WW domains).

WWP1 is involved in protein trafficking and protein degradation/stabilization. WWP1 maps on chromosome 8q21.3, a region involved in copy numbers gain in several cancers. WWP1 deficiency result in viable mice without major abnormality and resistant to MYC-driven prostate neoplasia. The mild phenotype of the WWP1 know out would point to a higher frequency of deletion. The mutation profile is intriguing and points to a potential role in cancer progression, but needs to be proven. The WW and C2 domain of WWP1 are regions where most mutations would disrupt their inhibitory function. Therefore, we characterizes mutations in these regions for gain-of-function.

### 15.2 Experimental Overview

A set of small molecule modulators (SSMs) are designed to understand the effect of WWP1 knock-down in matched (WWP1GOF vs. wt) cancer cell lines. The following experiments is established to determined WWP1 hot-spot mutations as drug targets.
1. Analysis of Genomic Mutation Spectrum in cancers: Characterize mutation frequency across cancers and identify actionable WWP1 mutations linked to specific cancer.
2. Testing of WWP1 SSMs in K740N GoF Mutation: Measure impact of WWP1 knock-down in WWP1K740N cell lines downstream signaling cascade.
3. Testing of WWP1 SSMs in other Hotspot GoF Mutations (i.e., in C2 domain): Use cell and organoid models to measure effect of WWP1 ablation on these GoF-mutant models.

### 15.3 Identification and Genetic Analysis of Most Frequent Somatic Mutations of WWP1 in Cancers

To gain understanding of the frequency and type of WWP1 mutations, appropriate genomics and bioinformatics tools (PolyPhen2, Mutation Taster, SIFT, homology modeling, MetaLR, and MetaSVM) are employed to mine the whole-exome sequencing datasets from different sources (TGCA database, GNOM, Broad Institute). These findings are compared with mutational pattern of other HECT E3 Ligase WWP2, ITCH, NEDD4, NEDDL, and E6AP. Mutational hotspots and regions of frequent mutation in WWP1 and across other HECT ligase family members are discovered. The goal of this set of experiments it to understand if cancers have WWP1 hotspot mutations or clusters of mutations that could be targeted with small molecule discovery approaches.

### 15.4 Testing of ASO in K740N Gain-of-Function Mutation

DLD1 and HCT116 colon cancer cell lines harboring WWP1K740N gain-of-function mutation (GoF) are received. These cell lines are tested with small molecule modulators. Published work by Pandolfi lab (Lee Y R, Chen M, Lee J D, et al. Reactivation of PTEN tumor suppressor for cancer treatment through inhibition of a MYC-WWP1 inhibitory pathway. Science. 2019; 364(6441) suggest that genetic ablation or pharmacological inhibition of WWP1 can trigger PTEN reactivation and other tumor suppressive activity. The most active small molecule modulators are tested in a battery of assays to analyze the effect of PTEN-PI3K-AKT/S6K axis and ultimately on the tumor growth using patient derived organoids.

Cell Lines: Colon Cancer Cell Lines DLD1 and HCT 116 and their CRISPR GoF Knock-In Counterparts

CRISPR knock-in WWP1K740N/+ DLD1, WWP1K740N/+ HCT116 and DLD1-WWP1 and HCT-WWP1 transfected with (HA)-PTEN cell lines. The CRISPR knock-in cells reproduce the mutant heterozygous genotype observed in human patients.

Prostate cancer cell lines with DU145, DU145-WWP1+/+, DU145-WWP1−/−, DU145-PTEN+/+ and DU145-PTEN−/− are received and tested. These paired cell lines are important tools to analyze the effect of different WWP1-level and PTEN downstream pathway in the same genetic background.

### 15.4.1 Effect of Small Molecule Modulators (SMMs) on PTEN-PI3K-AKT Pathway in GoF Cell Lines

SMMs blocking ubiquitination: The paired DLD1 and HCT 116 cell lines are treated with WWP1 SMMs at various condition. PTEN protein from the cell lysates (enriched by immunoprecipitation) is used for mass spectrometric peptide sequencing to analyze whether SMMs block the ubiquitination of K27. In addition, ubiquitination of PTEN is assessed using chain specific ubiquitin antibodies by Western blot in a quantitative manner.

### 15.4.2 Effects of WWP1 SMMs on PI3K-AKT-S6K Pathway

SMMs are tested for their ability to reduce PTEN ubiquitination, increase PIP2 concentrations, and reduced phospho-AKT/S6 levels in the cells by ELISA or western blot. These assays are performed in WWP1K740N/+ and WWP1+/+ HCT116 cell lines. Post treatment with SMMs, cell lysates are analyzed for the AKT pathway specific targets by Western blotting.

### 15.4.3 Effect of ASOs on Cell Growth and Tumorigenicity in GoF Cell Lines

Inhibition of cell proliferation: Cells are seeded (density of 3000 cells) and proliferation is measured using a proliferation assays. Cells are then treated with various doses of SMMs.

Soft agar colony formation assay: Assay is performed in plates testing various doses of SMMs.

Efficacy testing of lead SMMs in mouse human tumor-derived organoids: Organoids from primary colon, prostate, breast and pancreatic cancers are available from several biobanks. Recently, SSMs-mediated knockdown of several genes in mammary tumor organoids has been demonstrated (BioProtoc. 2017 Aug. 20; 7(16): e2511). Effect of our SSMs on both prostate organoids and spheres derived from WT and Hi-Myc mice at 3 months of age is tested. These mice are available in the mouse hospital at BIDMC. The most efficacious SSMs are screened against a panel of patient derived organoids for colorectal, prostate, and breast (TNBC) cancers. A reduction in tumor growth in the presence of WWP1 SSM triggers testing in animal models. Positive model is analyzed for WWP1 status.

Xenotransplantation: For assaying tumor growth in the xenograft model, 7-week-old male FOXNnu nude mice housed in specific pathogen-free environments is injected s.c. with 1.0×10⁶ DLD-1 derivatives harboring GoF mutant or 1.0×10⁶ DLD-1 derivatives harboring GoF mutant and treated with lead SSMs mixed with RPMI medium and Matrigel (vol/vol, 1:1).

### 15.5 Characterization of Hotspot WWP1 Mutations in Cancer Cell Lines

There is very limited understanding of the function of most WWP1 mutations, besides the two gain of function WWP1 mutations. There are other high frequency WWP1 mutations such as R595C/H (section between W4 and HECT domain) and R394C/H/S (W2 domain) with unknown function. These mutations might be interesting to characterize. In several studies, mutations in the C2 and WW region have been attributed to gain of function through relieving the inhibitory effects of C2 and WW on the catalytic HECT activity. Our genomics analysis identify hotspot mutations in the C2 and WW domain. After the genomic analysis, the top two hotspot mutations that are representative for either WW or C2 regulatory domain are picked for further analysis.

Hotspot Mutation in WW-domain or C2 domain: Missense variant of R394C/H/S may alter WWP1 structure and substrate binding or catalytic activity.

Cell lines to test WW-domain GoF mutation: RT4, HT1376 and T24 (urinary bladder cancer (UBC) cell lines) are used to characterize the activities of WWP1R595C/H and WWP1R394C/H/S (or other newly defined hotspot mutations).

Assay to test hyperactivity of hotspot WWP1 mutations: WWP1 hyperactivity has been attributed to the relieving the inhibitory effects of C2-domain using immunoprecipitation and immunoblotting assays. Direct measurement of enzyme kinetics in GOF protein remains difficult due to complexity of assay (E1, E2, E3, Ubiquitin, and ATP). The activity of the newly identified WWP1 variants is analyzed by co-transfection of WWP1wt or WWP1 GOF variants along with HA-tagged potential substrates such as PTEN, TGFb, etc., followed by immunoprecipitation and immunoblotting.

Measuring Downstream effectors of GoF mutations: In UBC, the B-cell translocation gene 2 (BTG2) functions as a tumor suppressor gene and is induced by PTEN. The BTG2 expression is lower in cancer than in normal tissues. Moreover, the highly differentiated bladder cancer cells, RT4, expressed higher BTG2 than the less-differentiated bladder cancer cells, HT1376 and T24. Overexpression of BTG2 in T24 cells inhibited cell growth in vitro and in vivo. Overexpression of gain-of-function activity of WWP1 may cause decrease in BTG2 expression in RT-4 and PTEN-overexpressed T24 cells.

Establishment of CRISPR knock-in cells: To generate WWP1R595C/+ and WWP1R595H/+ mutant cells, Alt-R CRISPR-Cas9 System is performed in accordance with manufacturer's protocol. The knock-in cell lines are used to evaluate the ubiquitination of target proteins (PTEN, BTG2) by WB, expression levels of BTG2 mRNA by RT-PCR and WB, and growth inhibition using MTS reagents.

### 16. Lead Small Molecule Candidates Inhibit WWP1 Autoubiquitination

WWP1 will auto-ubiquitinate in the presence of E1 enzyme, E2 enzyme, ATP, ubiquitin, and a buffer system. On a SDS PAGE gel, this reaction is visualized by the disappearance of free ubiquitin and the formation of a ubiquitin smear at a high molecular weight. In the presence of low (IC50) and high (5×IC50) concentrations of inhibitor, to varying degrees, lead candidates from the small molecule program inhibited WWP1 auto-ubiquitination compared to a DMSO only control. The reduction of a ubiquitin smear and maintained presence of free ubiquitin indicate inhibition of the reaction. “W2 In” is literature published compound known to inhibit NEDD4 family members (notably WWP2), used as a positive control. No E1 and No E3 samples are the in vitro ubiquitination reactions performed in the absence of E1 and E3 enzymes, respectively, a useful negative control. Reaction time was 4 hours. Analysis is via western blot using and anti-ubiquitin antibody.

### 17. Ubiquitin Chain Forming Ability Dictates WWP1 Autoubiquitination Kinetics

WWP1 rapidly auto-ubiquitinates in the presence of Wt ubiquitin, and to a lesser degree but still completely, in the presence of K63-only and K48-only ubiquitin. WWP1 largely does not auto-ubiquitinate in the presence of K27-only ubiquitin. Reaction was sampled hourly over a 4-hour time course. Analysis is via western blot using an anti-ubiquitin antibody.

### 18. The WWP1 Substrate PTEN is Preferentially Ubiquitinated Using K27-Only (K27O) or Non-Chain Forming Ubiquitin (NoK)

During an in vitro ubiquitination reaction containing the substrate PTEN and wild type ubiquitin, WWP1 will preferentially auto-ubiquitinate. Presumably WWP1 forms K63 and K48 chains in this reaction; however, in the presence of K27 only ubiquitin, PTEN substrate ubiquitin is preferred. PTEN is readily ubiquitinated with non-chain forming ubiquitin suggesting multi-mono ubiquitination is occurring, in addition to any K27 chain formation. 2 μM PTEN resulted in the best signal for the assay. Analysis is via western blot using an anti-ubiquitin antibody.

### 19. MS/MS Validation of Ubiquitin Preference for Autoubiquitination and PTEN Substrate Ubiquitination by WWP1

In vitro ubiquitination reactions were digested with Trypsin/LysC and labeled with TMT quantification reagents. Samples were analyzed and quantified by LC-MS/MS. Various non-K27 chains form during the reaction with wild type ubiquitin, and K27 chains are largely only formed using K27-only ubiquitin. Non-K27 chains are more numerous (greater S/N of TMT in the reaction). Specific ubiquitin sites on PTEN demonstrate greater occupancy (e.g., K237 and K342) when K27-only ubiquitin is used in the reaction vs wild type ubiquitin. Different sites of PTEN have different ubiquitin kinetics; moreover, K342 rapidly reaches a high occupancy in 2 hours, whereas another site on PTEN, K223, becomes ubiquitinated slower.

### 20. A Lead Allosteric Series Compound, AS-004-115, Inhibits WWP1 Dependent Ubiquitination of PTEN, in a Dose Dependent Manner

In vitro ubiquitination reactions were carried out for 2 hours using the indicated doses of inhibitor. Increasing doses of inhibitor block PTEN substrate ubiquitination, which correlates with remaining free ubiquitin. The “0” concentration is DMSO only (0.5%, same as was used for compound doses). No E1 and No E3 negative controls are present (only free ubiquitin present). Analysis occurred by western blot using an anti-ubiquitin antibody. A fraction of these samples was digested with Trypsin/LysC and labeled with TMT quantification reagents prior to LC-MS/MS analysis. Mass spectrometry results confirm the inhibition of ubiquitin transfer to a lysine on PTEN and inhibition of K27 ubiquitin chain formation.

### 21. A Lead I₃C Series Compound, COMPOUND 7, Inhibits WWP1-Dependent Ubiquitination of PTEN, in a Dose-Dependent Manner

In vitro ubiquitination reactions were carried out for 2 hours using the indicated doses of inhibitor. Increasing doses of inhibitor block PTEN substrate ubiquitination, which correlates with remaining free ubiquitin. The “0” concentration is DMSO only (0.5%, same as was used for compound doses). No E1 and No E3 negative controls are present (only free ubiquitin present). Analysis occurred by western blot using an anti-ubiquitin antibody.

### 22. Knockdown of WWP1 by siRNA in Human Breast Cancer Cell Lines Reduces Phosphorylated AKT and Phosphorylated Ribosomal Protein S6 Levels, Known Oncogenic Markers

Cells were treated with 10 nM pooled siRNA (IDT) and harvested 48 hours after. Cells were lysed in RIPA buffer containing protease and phosphatase inhibitors on ice. Lysates were analyzed by western blot using the indicated antibodies. siRNA successfully reduced WWP1 levels by several fold. This reduction in WWP1 protein levels strongly correlated with a reduction in phosphorylated AKT at serine 473 and a reduction in Ribosomal Protein S6 phosphorylation at serine 235/236. These data indicate a reduction in growth pathway signaling with the knockdown of WWP1.

### 23. Lead I₃C Series Candidate COMPOUND 1 Reduces Phosphorylated AKT and Phosphorylated Ribosomal Protein S6 Levels in a Dose-Dependent Manner

Hs578T cells (human triple negative breast cancer) were treated with the indicated concentration of COMPOUND 1 for 48 hours prior to harvesting. The “0” concentration is DMSO only (0.5%, same as was used for compound doses). Cells were lysed in RIPA buffer containing protease and phosphatase inhibitors on ice. Analysis occurred via western blot using the indicated antibodies. Quantification by densitometry of the bands from the western blot are presented for phosphorylated AKT/Total AKT and phosphorylated Ribosomal Protein S6/Total Ribosomal Protein S6. COMPOUND 1 reduced both AKT and S6 phosphorylation relative to their respective total protein levels.

### 24. Lead I₃C Series Candidate COMPOUND 1 Increases PD-L1 Levels and the Glycosylated/Non-Glycosylated PD-L1 Ratio

Hs578T cells (human triple negative breast cancer) were treated with the indicated concentration of COMPOUND 1 for 48 hours prior to harvesting. The “0” concentration is DMSO only (0.5%, same as was used for compound doses). Cells were lysed in RIPA buffer containing protease and phosphatase inhibitors on ice. COMPOUND 1 treatment increased both PD-L1 levels and PD-L1 glycosylation (higher MW band). These results were observed in both the presence and absence of interferon gamma (IFN, 25 ng/mL in cell culture media during the drug treatment), a stimulator of PD-L1 expression. Analysis occurred via western blot using the indicated antibodies. Quantification by densitometry of the bands from the western blot are presented for PD-L1/Vinculin (loading control) in the absence of interferon gamma, and glycosylated/non-glycosylated PD-L1 in the presence of interferon gamma.

### 25. Replication of COMPOUND 1 Treatment Data in Hs578T and BT-20 TNBC Cells, AKT and S6 Phosphorylation Effects

The data presented here are consistent with the previous replicate in Hs578T cells, and the observation is preserved in another TNBC cell line, BT-20. COMPOUND 1 reduces the phospho-AKT/vinculin loading control and phospho-S6/total S6 ratios. Methods are the same as the previous replicate, using the indicated doses.

### 26. Replication of COMPOUND 1 Treatment Data in Hs578T and BT-20 TNBC Cells, PD-L1 Effects

The data presented here are consistent with the previous replicate in Hs578T cells, and the observation is preserved in another TNBC cell line, BT-20. COMPOUND 1 increases PD-L1 levels and the glycosylated PD-L1/non-glycosylated PD-L1 ratio. Methods are the same as the previous replicate, using the indicated doses.

### 27. Lead I₃C Series Candidate COMPOUND 6 Reduces Phosphorylated AKT and Phosphorylated Ribosomal Protein S6 Levels in a Dose-Dependent Manner

Hs578T cells (human triple negative breast cancer) were treated with the indicated concentration of COMPOUND 6 for 48 hours prior to harvesting. The “0” concentration is DMSO only (0.5%, same as was used for compound doses). Cells were lysed in RIPA buffer containing protease and phosphatase inhibitors on ice. Analysis occurred via western blot using the indicated antibodies. COMPOUND 6 treatment reduced both AKT and S6 phosphorylation relative to the vinculin loading control and total S6 protein levels, respectively.

### 28. Lead I₃C Series Candidate COMPOUND 6 Reduces PD-L1 Levels in a Dose-Dependent Manner

Hs578T cells (human triple negative breast cancer) were treated with the indicated concentration of COMPOUND 6 for 48 hours before harvesting. The “0” concentration is DMSO only (0.5%, same as was used for compound doses). Cells were lysed in RIPA buffer containing protease and phosphatase inhibitors on ice. Analysis occurred via western blot using the indicated antibodies. PD-L1 protein levels were reduced relative to the vinculin loading control upon compound treatment. This effect is observed in the presence and absence of interferon gamma (IFN, 25 ng/mL in cell culture media during the drug treatment).

### 29. Replication of COMPOUND 6 Treatment Data in Hs578T Cells, AKT and S6 Phosphorylation Effects

The data presented here are consistent with the previous replicate in Hs578T cells. COMPOUND 6 reduces the phospho-AKT/vinculin loading control and phospho-S6/total S6 ratios. Methods are the same as the previous replicate, using the indicated doses.

### 30. Replication of COMPOUND 6 Treatment Data, AKT and S6 Phosphorylation Effects, Using the BT-20 Cell Line

The data presented here are consistent with the previous replicate in Hs578T cells. COMPOUND 6 reduces the phospho-AKT/vinculin loading control and phospho-S6/total S6 ratios in BT-20 cells. Methods are the same as the previous replicate, using the indicated doses.

### 31. Replication of COMPOUND 6 Treatment Data, PD-L1 Effect, Using the BT-20 Cell Line

The data presented here are consistent with the previous replicate in Hs578T cells. COMPOUND 6 reduces the PD-L1/vinculin loading control ratio in BT-20 cells. Additionally, COMPOUND 6 probably reduces PD-L1 glycosylation. Methods are the same as the previous replicate, using the indicated doses.

### 32. Lead I₃C Series Candidate COMPOUND 7 Reduces Phosphorylated AKT and Phosphorylated Ribosomal Protein S6 Levels in a Dose-Dependent Manner

Hs578T cells (human triple negative breast cancer) were treated with the indicated concentration of COMPOUND 7 for 48 hours before harvesting. The “0” concentration is DMSO only (0.5%, same as was used for compound doses). Cells were lysed in RIPA buffer containing protease and phosphatase inhibitors on ice. Analysis occurred via western blot using the indicated antibodies. COMPOUND 7 treatment reduced both AKT and S6 phosphorylation relative to the vinculin loading control and total S6 protein levels, respectively.

### 33. Lead I₃C Series Candidate COMPOUND 7 Reduces PD-L1 in a Dose-Dependent Manner

Hs578T cells (human triple negative breast cancer) were treated with the indicated concentration of COMPOUND 7 for 48 hours prior to harvesting. The “0” concentration is DMSO only (0.5%, same as was used for compound doses). Cells were lysed in RIPA buffer containing protease and phosphatase inhibitors on ice. Analysis occurred via western blot using the indicated antibodies. PD-L1 protein levels were reduced relative to the vinculin loading control upon 319-02 treatment.

### 34. Replication of COMPOUND 7 Treatment Data in Hs578T Cells, S6 Phosphorylation Effect

The data presented here are consistent with the previous replicate in Hs578T cells. COMPOUND 7 reduces the phospho-S6/vinculin loading control ratio. Methods are the same as the previous replicate, using the indicated doses.

### 35. Replication of COMPOUND 7 Treatment Data in Hs578T Cells, PD-L1 Effect

The data presented here are consistent with the previous replicate in Hs578T cells. COMPOUND 7 reduces the PD-L1/vinculin loading control ratio. Methods are the same as the previous replicate, using the indicated doses.

### 36. Lead I₃C Series Candidate COMPOUND 8 Reduces Phosphorylated Ribosomal Protein S6 Levels in a Dose-Dependent Manner

Hs578T cells (human triple negative breast cancer) were treated with the indicated concentration of COMPOUND 8 for 48 hours and then harvested. The “0” concentration is DMSO only (0.5%, same as was used for compound doses). Cells were lysed in RIPA buffer containing protease and phosphatase inhibitors on ice. Analysis occurred via western blot using the indicated antibodies. COMPOUND 8 treatment reduced the levels of S6 phosphorylation relative to the vinculin loading control.

### 37. Lead I₃C Series Candidate COMPOUND 8 Reduces PD-L1 Levels in a Dose-Dependent Manner

Hs578T cells (human triple negative breast cancer) were treated with the indicated concentration of COMPOUND 8 for 48 hours and then harvested. The “0” concentration is DMSO only (0.5%, same as was used for compound doses). Cells were lysed in RIPA buffer containing protease and phosphatase inhibitors on ice. Analysis occurred via western blot using the indicated antibodies. COMPOUND 8 treatment reduced PD-L1 levels relative to the vinculin loading control.

### 38. Knockdown of WWP1 by siRNA Increases PD-L1 Protein Levels, PD-L1 Glycosylation and Cell Surface Expression in Hs578T Cells

Cells were treated with 10 nM pooled siRNA (IDT) and harvested 48 hours after, splitting cells for flow cytometry and western blot. For western blot, cells were lysed in RIPA buffer containing protease and phosphatase inhibitors on ice. Lysates were analyzed by western blot using the indicated antibodies. PD-L1-APC (BD Biosciences) and a matched isotype control were used to visualize PD-L1 cell surface expression by flow cytometry (1:250 dilution). As shown by western blot, the knockdown of WWP1 yielded increased PD-L1 protein levels, including an increase in the glycosylated band. Similarly, WWP1 knockdown dramatically increased PD-L1 cell surface expression by flow cytometry (10% positive in control vs. isotype to 75% positive after knockdown). The mean fluorescence intensity of the positive population was also increased by about 10-fold, suggesting that not only were more cells expressing PD-L1, but the cells that did also contained more total PD-L1 on the cell surface.

### 39. WWP1 Knockdown by siRNA Increases PD-L1 Protein Levels and Glycosylation in Multiple Cancer Cell Models

Cells were treated with 10 nM pooled siRNA (IDT) and harvested 48 hours after. Cells were lysed in RIPA buffer containing protease inhibitors on ice. Lysates were analyzed by western blot using the indicated antibodies. TNBCs Hs578T and BT-20, the colorectal cancer cell line Du145, and the ER+ lung cancer line A549 all demonstrated increased PD-L1 protein levels, including the glycosylated form (and presumably surface expression), upon WWP1 knockdown. These data are suggestive of a general phenomenon and a reproducible connection between WWP1 and PD-L1.

### 40. Interferon Gamma Dramatically Boosts PDL1 Expression in Triple Negative Breast Cancer Cell Lines, but not Receptor Positive Breast Cancer Cells

Hs578T, BT-20 and MCF-7 cells were cultured for 48 hours in the presence or absence of interferon gamma (25 ng/mL in the cell culture media). Cells were lysed in RIPA buffer containing protease inhibitors on ice. Lysates were analyzed by western blot using the indicated antibodies. Relative to the vinculin loading control, interferon gamma upregulates PD-L1 protein levels within TNBCs shown to express PD-L1 at baseline. Conversely, interferon gamma induced PD-L1 expression was not readily observable in the ER+ cell line MCF-7, which does not appear to express PD-L1 protein at baseline.

### 41. Interferon Gamma Moderately Reduces WWP1 Protein Levels in Multiple Cancer Cell Lines

Prior to harvesting, A549 and Hs578T cells were cultured for 48 hours in the presence or absence of interferon gamma (25 ng/mL in the cell culture media) or 10 nM pooled siRNA (IDT). Cells were lysed in RIPA buffer containing protease inhibitors on ice. Lysates were analyzed by western blot using the indicated antibodies. Interferon gamma reduced WWP1 levels in both cell lines compared to control, but not to the extent observed by WWP1 siRNA knockdown. These data may indicate a connection between interferon gamma, WWP1 and PD-L1.

### 42. Interferon Gamma and WWP1 siRNA Cooperatively Increase PD-L1 Expression in Multiple Cancer Cell Lines

Hs578T, BT-20 and A549 cells were cultured for 48 hours in the presence or absence of interferon gamma (25 ng/mL in the cell culture media), presence of absence of 10 nM pooled siRNA (IDT), or both. Cells were lysed in RIPA buffer containing protease inhibitors on ice. Lysates were analyzed by western blot using the indicated antibodies. Both interferon or siRNA alone reduced WWP1 expression (siRNA have a more dramatic effect on WWP1 levels) and increased PD-L1 protein expression. The combination of WWP1 siRNA and interferon further augmented PD-L1 protein levels beyond that of siRNA or interferon treatment alone, suggesting a cooperative activity of WWP1 and interferon on the PD-L1 pathway.

### 43. WWP1 shRNA Expressing Du145 Cell Lines Demonstrate Moderate Knockdown of WWP1 mRNA and Protein Levels

All shRNA constructs were introduced through lentiviral infection under puromycin selection. Cells were cultured for 48 hours in the presence or absence of interferon gamma (25 ng/mL in the cell culture media) at which time a fraction of cells was split for protein and mRNA evaluation. For protein, cells were lysed in RIPA buffer containing protease inhibitors on ice. Total RNA was isolated using the RNeasy kit from Qiagen, prior to reverse transcription to obtain cDNA. Compared to empty vector control lines, WWP1 shRNA expressing cells has about a 60% KD of WWP1 mRNA by qPCR, which translated to roughly 3-5-fold protein level reduction by western. Interferon itself modestly reduced WWP1 mRNA levels (15% reduction).

### 44. WWP1 shRNA Expressing Du145 Cell Lines Demonstrate Increased PD-L1 Expression

All shRNA constructs were introduced through lentiviral infection under puromycin selection. Cells were cultured for 48 hours in the presence or absence of interferon gamma (25 ng/mL in the cell culture media). Cells were lysed in RIPA buffer containing protease inhibitors on ice. Analysis occurred via western blot using the indicated antibodies. In the absence of interferon gamma, relative to the vinculin loading control, WWP1 knockdown correlated with increased PD-L1 protein levels. Interferon treatment also increased PD-L1 protein levels relative to the vinculin loading control, in control shRNA lines (No WWP1 knockdown). WWP1 shRNA expression, resulting in WWP1 knockdown, increased PD-L1 protein levels relative to the vinculin loading control, in both the presence and absence of interferon. These data corroborate siRNA data presented previously.

### 45. The NEDD4 Family HECT Ligases are Expressed at Comparable Levels in Hs578T Cells

Steady state cells were harvested, and total RNA was purified by an RNeasy kit from Qiagen. After reverse transcription to generate cDNA, qPCR was used to quantify each ligase mRNA level. All ligases tested were expressed at similar levels, suggesting each may be an active constituent of the Hs578T cell model. ITCH was also found to be expressed at similar levels (data not shown).

### 46. PD-L1 mRNA is Most Affected by WWP1 siRNA Treatment in Hs578T Cells

Cells were treated with 10 nM pooled siRNA for the indicated gene (IDT) and harvested 48 hours after. Total RNA was purified by an RNeasy kit from Qiagen. After reverse transcription to generate cDNA, qPCR was used to quantify mRNA levels of each ligase and PD-L1. All ligases demonstrated >70% KD (data not shown). WWP1 knockdown, to a greater degree than WWP2 (13-vs. 2-fold), dramatically upregulated PD-L1 mRNA. These data demonstrate specificity in the pathway among closely related enzymes.

### 47. PD-L1 Protein Levels are Most Affected by WWP1 siRNA Treatment in Hs578T Cells

Cells were treated with 10 nM pooled siRNA for the indicated gene (IDT) and harvested 48 hours after (split for protein and RNA analysis). For protein, cells were lysed in RIPA buffer containing protease inhibitors on ice. Lysates were analyzed by western blot using the indicated antibodies. Total RNA was purified by an RNeasy kit from Qiagen. After reverse transcription to generate cDNA, qPCR was used to quantify mRNA levels of each ligase (results displayed below the blot). WWP1 protein levels were reduced significantly and specifically to the WWP1 siRNA condition, relative to the actin loading control, though western blots for WWP2 were not successful; however, WWP2 demonstrated ˜80% knockdown by qPCR. Mirroring the qPCR results, WWP1 siRNA treatment significantly boosted PD-L1 levels, whereas WWP2 siRNA treatment may only modestly affect PD-L1.

### 48. PD-L1 Protein Levels are Most Affected by WWP1 siRNA Treatment in Hs578T Cells, Replicate Adding NEDD4 siRNA Treatment

These data replicate and expand upon the previous example to include another related enzyme, NEDD4. Cells were treated with 10 nM pooled siRNA for the indicated gene (IDT) and harvested 48 hours after (split for protein and RNA analysis). For protein, cells were lysed in RIPA buffer containing protease inhibitors on ice. Lysates were analyzed by western blot using the indicated antibodies. Total RNA was isolated using an RNeasy kit from Qiagen. After reverse transcription to generate cDNA, qPCR was used to quantify mRNA levels of each ligase. All siRNA targets demonstrated a >70% knockdown by qPCR. Only the WWP1 siRNA treatment resulted in significant upregulation of PD-L1 protein levels.

### 49. ITCH siRNA Treatment does not Affect WWP1 or PD-L1 Protein Levels

Cells were treated with 10 nM pooled siRNA for the indicated gene (IDT) and harvested after 48 hours. Cells were lysed in RIPA buffer containing protease inhibitors on ice. Lysates were analyzed by western blot using the indicated antibodies. Knockdown of WWP1 was specific to WWP1 siRNA treatment, which also correlated with PD-L1 protein upregulation, relative to the actin and vinculin loading controls, respectively. ITCH siRNA (at both low and high concentrations) neither affected WWP1 levels nor PD-L1 levels, respective to actin and vinculin loading controls.

### 50. WWP1 Knockdown is Specific to WWP1 siRNA Treatment

Cells were treated with 10 nM pooled siRNA for the indicated gene (IDT) and harvested after 48 hours. Cells were lysed in RIPA buffer containing protease inhibitors on ice. Lysates were analyzed by western blot using the indicated antibodies. Knockdown of WWP1 was specific to WWP1 siRNA treatment, relative to the vinculin loading control. siRNA constructs for WWP2 and NEDD4 did not affect WWP1 protein levels relative to the vinculin loading control.

### 51. ASO Transfection Methods are Well Optimized in Hs578T Cells

RNAiMax Lipofectamine (Thermo) was used as the transfection reagent at the manufacturer's recommended concentration. “AKT1” samples were treated with an AKT1 ASO, previously demonstrated to efficiently knock down the AKT1 target gene. “NC1” and “NC2” are scrambled/non-targeting ASO controls expected to have no impact on the cell. “Parental” cells were treated with only lipofectamine. Cells were treated with either 10 or 20 nM ASO (or lipofectamine alone) and harvested after 48 hours. Total RNA was isolated using an RNeasy kit from Qiagen. After reverse transcription to generate cDNA, qPCR was used to quantify AKT1 mRNA levels. Values are relative to “Parental” replicate 1. AKT1 ASO conditions successfully knocked down the AKT1 mRNA (>80%) compared to either the parental or negative control ASOs, with the 20 nM condition demonstrating slightly more efficacy. The data are indicative of optimized transfection conditions for WWP1 ASO screening. Error bars are standard error of the mean.

### 52. Many WWP1 ASOs Significantly Knockdown WWP1 mRNA Levels in Hs578T Cells

RNAiMax Lipofectamine (Thermo) was used as the transfection reagent at the manufacturer's recommended concentration. “siRNA” samples were treated with control (scrambled) or pooled WWP1 siRNA at 10 nM. “AKT1” samples were treated with an AKT1 ASO, previously demonstrated to efficiently knock down the AKT1 target gene. “NC1” and “NC2” are scrambled/non-targeting ASO controls expected to have no impact on the cell. “Parental” cells were treated with only lipofectamine. ASOs with the prefix “ASO” indicate those targeting WWP1 (44 in total). Cells were treated with either 10 or 20 nM ASO (or 10 nM siRNA, or lipofectamine alone) and harvested after 48 hours. Total RNA was isolated using an RNeasy kit from Qiagen. After reverse transcription to generate cDNA, qPCR was used to quantify WWP1 mRNA levels. Values are relative to “Parental” replicate 1. Control samples did not significantly affect WWP1 mRNA levels (“Parental”, “NC” and “AKT1” samples, green, blue, and purple bars, respectively), whereas the WWP1 siRNA positive control efficiently knocked down WWP1 mRNA by >80% (orange bars). WWP1 targeting ASOs showed varied efficacy depending on their location within the WWP1 gene body (e.g., ASOs 5-8 vs. 9-12), and some dose dependence (blue 10 nM vs. red 20 nM, e.g., “ASO8”). Ct values for “ASO1” transfection at the 20 nM treatment condition were off by ˜10 cycles compared to the 10 nM condition, suggesting a sample preparation effort. Error bars are standard error of the mean.

### 53. Replicate Screen of WWP1 ASO Efficacy in Hs578T Cells by Thermo Life Technologies (CRO) Confirm Many WWP1 ASOs Significantly Reduce mRNA Levels

Under similar conditions, all ASOs were rescreening with a large level of agreement with the prior screen.

### 54. Replicate Screen of WWP1 ASO Efficacy in VERO-E6 Cells by Thermo Life Technologies (CRO) Confirm Many WWP1 ASOs Significantly Reduce mRNA Levels

ASOs were also screened in non-human primate cells (VERO E6, African green monkey) under similar conditions, with a large level of agreement with the prior screen.

### 55. Candidate ASOs from the Screen Demonstrate Efficient Knockdown of WWP1 Protein Levels

RNAiMax Lipofectamine (Thermo) was used as the transfection reagent at the manufacturer's recommended concentration. Hs578T cells were treated with either control siRNA, pooled WWP1 siRNA (IDT) or the listed ASO at 10 nM prior to harvest at the 48-hour time point. Cells were lysed in RIPA buffer containing protease inhibitors. Western blots were used to quantify protein levels, using antibodies against Vinculin and WWP1. Relative to the vinculin loading control, WWP1 siRNA and the ASOs that demonstrated efficacy in the previous qPCR screen efficiently reduced WWP1 protein levels, compared to the appropriate controls.

### 56. Candidate ASOs from the Screen Demonstrate Efficient Knockdown of WWP1 Protein Levels, Data Replication

The experiment from the previous example was replicated, using 20 nM ASO and a fraction of cells was used for qPCR analysis of mRNA. There was a very good correlation between the qPCR results and the western blot for WWP1 knockdown on the mRNA and protein levels, respectively.

### 57. I3C Shows Promise as a Potential SARS-CoV-2 Inhibitor

Vero cells were grown to 70-90% confluency. The compounds being tested were dissolved to 10 mM DMSO and used to treat cells from 150 nM up to 10 μM in 2-fold increments. The compounds were incubated with the cells for up to 1 hour and then challenged with SARS-CoV-2 at an MOI of up to 0.2. After 36 hours, the cells were fixed in formalin and then stained with SARS-CoV-2 antibody against the N protein and a fluorescently tagged secondary antibody. Cell nuclei were stained with Hoechst 33342 dye. The cells were imaged using a Cytation (Biotek) automated imaging system to visualize the blue fluorescent nuclei and the green fluorescent infected cells expressing virus N protein. Images were analyzed by CellProfiler software using a customized analysis pipeline to count the nuclei and the infected cells. Infection efficiency is expressed as a function of infected cells/cell nuclei counted. Wells where there were less than 50% cells remaining were flagged due to the treatment causing cell detachment, which may indicate toxicity. For such treatments, these measurements were not interpreted for infection inhibition due to potential inaccuracies of counting small numbers of cells. The assay was performed with 3 replicates per dose. Effects were compared by one way ANOVA with multiple comparison to the control group using Graphpad Prism software. Controls included were 0.5% DMSO treated cells (vehicle) or treatment with E-64d at 5 μM. All treatments except for Indole-3 carbinol were ineffective for the doses tested. Indole-3-carbinol (I3C) was effective at inhibiting >75% infection at 5 μM. At 10 μM, >50% cell loss was seen across each replicate indicating the potential for cytotoxicity. At 2.5 μM there was little impact on infection. The small window of activity made establishing a dose response curve difficult and so an EC50 was not calculated. I3C shows promise as a potential SARS-CoV-2 inhibitor; however, the small window of activity to where cells were lost suggests a potentially small selectivity index. Its use as a potential scaffold could be explored by making chemical derivatives.

### Additional Sequence

Exemplary WWP1 sequence:
(SEQ ID NO: 541)
AGCTGAATTTTGGGACATGGCCACTGCTTCACCAAG
GTCTGATACTAGTAATAACCACAGTGGAAGGTTGC
AGTTACAGGTAACTGTTTCTAGTGCCAAACTTAAA
AGAAAAAAGAACTGGTTCGGAACAGCAATATATAC
AGAAGTAGTTGTAGATGGAGAAATTACGAAAACAG
CAAAATCCAGTAGTTCTTCTAATCCAAAATGGGAT
GAACAGCTAACTGTAAATGTTACGCCACAGACTAC
ATTGGAATTTCAAGTTTGGAGCCATCGCACTTTAA
AAGCAGATGCTTTATTAGGAAAAGCAACGATAGAT
TTGAAACAAGCTCTGTTGATACACAATAGAAAATT
GGAAAGAGTGAAAGAACAATTAAAACTTTCCTTGG
AAAACAAGAATGGCATAGCACAAACTGGTGAATTG
ACAGTTGTGCTTGATGGATTGGTGATTGAGCAAGA
AAATATAACAAACTGCAGCTCATCTCCAACCATAG
AAATACAGGAAAATGGTGATGCCTTACATGAAAAT
GGAGAGCCTTCAGCAAGGACAACTGCCAGGTTGGC
TGTTGAAGGCACGAATGGAATAGATAATCATGTAC
CTACAAGCACTCTAGTCCAAAACTCATGCTGCTCG
TATGTAGTTAATGGAGACAACACACCTTCATCTCC
GTCTCAGGTTGCTGCCAGACCCAAAAATACACCAG
CTCCAAAACCACTCGCATCTGAGCCTGCCGATGAC
ACTGTTAATGGAGAATCATCCTCATTTGCACCAAC
TGATAATGCGTCTGTCACGGGTACTCCAGTAGTGT
CTGAAGAAAATGCCTTGTCTCCAAATTGCACTAGT
ACTACTGTTGAAGATCCTCCAGTTCAAGAAATACT
GACTTCCTCAGAAAACAATGAATGTATTCCTTCTA
CCAGTGCAGAATTGGAATCTGAAGCTAGAAGTATA
TTAGAGCCTGACACCTCTAATTCTAGAAGTAGTTC
TGCTTTTGAAGCAGCCAAATCAAGACAGCCAGATG
GGTGTATGGATCCTGTACGGCAGCAGTCTGGGAAT
GCCAACACAGAAACCTTGCCATCAGGGTGGGAACA
AAGAAAAGATCCTCATGGTAGAACCTATTATGTGG
ATCATAATACTCGAACTACCACATGGGAGAGACCA
CAACCTTTACCTCCAGGTTGGGAAAGAAGAGTTGA
TGATCGTAGAAGAGTTTATTATGTGGATCATAACA
CCAGAACAACAACGTGGCAGCGGCCTACCATGGAA
TCTGTCCGAAATTTTGAACAGTGGCAATCTCAGCG
GAACCAATTGCAGGGAGCTATGCAACAGTTTAACC
AACGATACCTCTATTCGGCTTCAATGTTAGCTGCA
GAAAATGACCCTTATGGACCTTTGCCACCAGGCTG
GGAAAAAAGAGTGGATTCAACAGACAGGGTTTACT
TTGTGAATCATAACACAAAAACAACCCAGTGGGAA
GATCCAAGAACTCAAGGCTTACAGAATGAAGAACC
CCTGCCAGAAGGCTGGGAAATTAGATATACTCGTG
AAGGTGTAAGGTACTTTGTTGATCATAACACAAGA
ACAACAACATTCAAAGATCCTCGCAATGGGAAGTC
ATCTGTAACTAAAGGTGGTCCACAAATTGCTTATG
AACGCGGCTTTAGGTGGAAGCTTGCTCACTTCCGT
TATTTGTGCCAGTCTAATGCACTACCTAGTCATGT
AAAGATCAATGTGTCCCGGCAGACATTGTTTGAAG
ATTCCTTCCAACAGATTATGGCATTAAAACCCTAT
GACTTGAGGAGGCGCTTATATGTAATATTTAGAGG
AGAAGAAGGACTTGATTATGGTGGCCTAGCGAGAG
AATGGTTTTTCTTGCTTTCACATGAAGTTTTGAAC
CCAATGTATTGCTTATTTGAGTATGCGGGCAAGAA
CAACTATTGTCTGCAGATAAATCCAGCATCAACCA
TTAATCCAGACCATCTTTCATACTTCTGTTTCATT
GGTCGTTTTATTGCCATGGCACTATTTCATGGAAA
GTTTATCGATACTGGTTTCTCTTTACCATTCTACA
AGCGTATGTTAAGTAAAAAACTTACTATTAAGGAT
TTGGAATCTATTGATACTGAATTTTATAACTCCCT
TATCTGGATAAGAGATAACAACATTGAAGAATGTG
GCTTAGAAATGTACTTTTCTGTTGACATGGAGATT
TTGGGAAAAGTTACTTCACATGACCTGAAGTTGGG
AGGTTCCAATATTCTGGTGACTGAGGAGAACAAAG
ATGAATATATTGGTTTAATGACAGAATGGCGTTTT
TCTCGAGGAGTACAAGAACAGACCAAAGCTTTCCT
TGATGGTTTTAATGAAGTTGTTCCTCTTCAGTGGC
TACAGTACTTCGATGAAAAAGAATTAGAGGTTATG
TTGTGTGGCATGCAGGAGGTTGACTTGGCAGATTG
GCAGAGAAATACTGTTTATCGACATTATACAAGAA
ACAGCAAGCAAATCATTTGGTTTTGGCAGTTTGTG
AAAGAGACAGACAATGAAGTAAGAATGCGACTATT
GCAGTTCGTCACTGGAACCTGCCGTTTACCTCTAG
GAGGATTTGCTGAGCTCATGGGAAGTAATGGGCCT
CAAAAGTTTTGCATTGAAAAAGTTGGCAAAGACAC
TTGGTTACCAAGAAGCCATACATGTTTTAATCGCT
TGGATCTACCACCATATAAGAGTTATGAACAACTA
AAGGAAAAACTTCTTTTTGCAATAGAAGAGACAGA
GGGATTTGGACAAGAATGA

### Other Embodiments

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in this application, in applications claiming priority from this application, or in related applications. Such claims, whether directed to a different invention or to the same invention, and whether broader, narrower, equal, or different in scope in comparison to the original claims, also are regarded as included within the subject matter of the inventions of the present disclosure.

What is claimed is:

1. A compound of formula I: 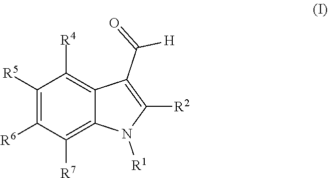
or a pharmaceutically acceptable salt or solvate thereof, wherein:
R¹ is selected from the group consisting of phenyl, naphthyl, phenylC₁-C₂alkyl, and naphthylC₁-C₂alkyl;
R² is H or C₁-C₃alkyl;
R⁴ is selected from the group consisting of H, halogen, and nitrile;
R⁵ is selected from the group consisting of H, halogen, and —CONHR⁸;
R⁶ is H or C₂-C₆alkynyl;
R⁷ is selected from the group consisting of H, C₁-C₃alkyl, and halogen; and
R⁸ is C₂-C₆alkynyl or 6-membered heteroaryl optionally substituted with halogen or nitrile;
wherein R⁵ and R⁶ are not both H.

2. The compound of claim 1, wherein the compound is of formula Ia: 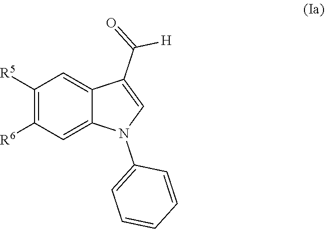
or a pharmaceutically acceptable salt or solvate thereof.

3. The compound of any of claims 1-2, wherein R⁵ is H or —CONHR⁸.

4. The compound of any of claims 1-3, wherein R⁵ is —CONHR⁸.

5. The compound of any of claims 1-4, wherein R⁸ is 6-membered heteroaryl optionally substituted with halogen or nitrile

6. The compound of any of claims 1-5, wherein R⁸ is 2-cyanopyridin-4-yl.

7. The compound of any of claims 1-6, wherein R⁶ is H or —C≡CH.

8. The compound of any of claims 1-7, wherein R⁶ is —C≡CH.

9. A compound of formula II: 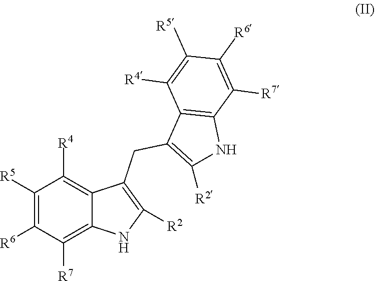
or a pharmaceutically acceptable salt or solvate thereof, wherein:
each of R² and R^{2′} are independently H or C₁-C₃alkyl;
each of R⁴ and R^{4′} are independently H or halogen;
each of R⁵ and R^{5′} are independently H or —CONR⁸R⁹;
each of R⁶ and R^{6′} are independently selected from the group consisting of H, —CO₂H, and —CONR⁸R⁹;
each of R⁷ and R^{7′} are independently selected from the group consisting of H, C₁-C₃alkyl, and halogen;
each R⁸ is independently C₂-C₆alkynyl or 6-membered heteroaryl optionally substituted with halogen or nitrile; and
each R⁹ is independently selected from H or C₁-C₃alkyl optionally substituted with —OH;
wherein R⁵ and R⁶ are not both H.

10. The compound of claim 9, wherein the compound is of formula IIa: 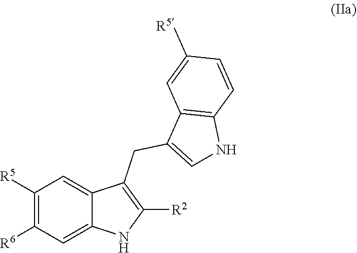
or a pharmaceutically acceptable salt or solvate thereof.

11. The compound of any of claim 9 or 10, wherein R² is H or methyl.

12. The compound of any of claims 9-11, wherein R^{5′} is H.

13. The compound of any of claims 9-12, wherein R⁶ is —CO₂H or —CONR⁸R⁹.

14. The compound of claim 13, wherein R⁶ is —CO₂H.

15. The compound of claim 13, wherein R⁶ is —CONR⁸R⁹.

16. The compound of any of claims 9-15, wherein R⁸ is propyn-3-yl or 2-cyanopyridin-4-yl.

17. The compound of any of claims 9-16, wherein R⁹ is selected from the group consisting of H, methyl, and 2-hydroxyethyl.

18. The compound of any of claims 9-17, wherein R⁸ is propyn-3-yl, and wherein R⁹ is H or 2-hydroxyethyl.

19. A compound selected from the group consisting of: 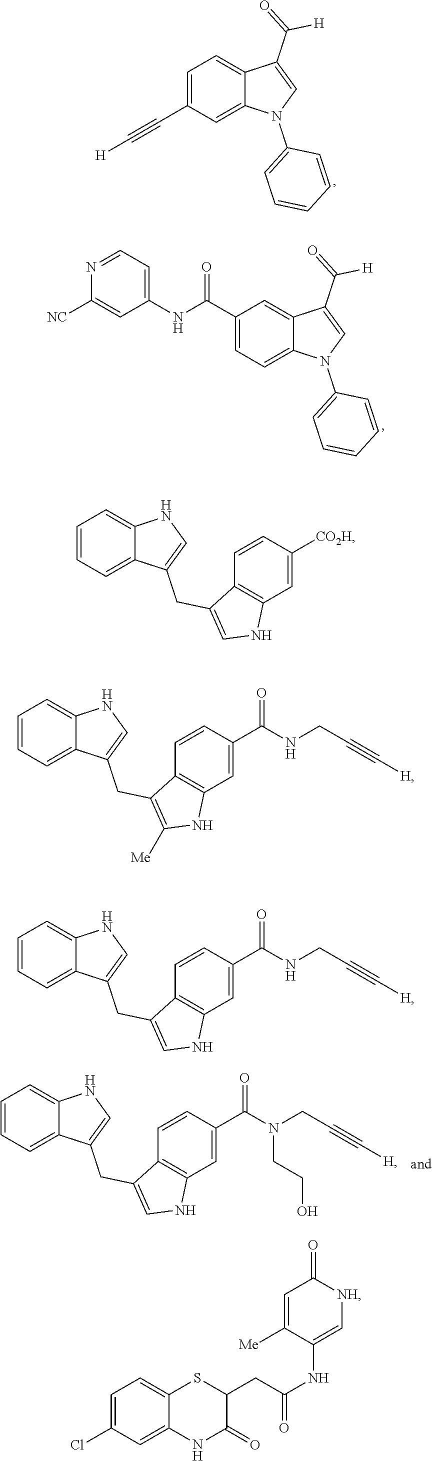
or a pharmaceutically acceptable thereof.

20. A pharmaceutical composition comprising at least one compound according to any one of claims 1-19 and at least one pharmaceutically acceptable excipient.

21. A method of modulating the activity of at least one HECT domain E3 ligase in a mammalian cell, comprising: contacting the mammalian cell with the compound of any of claims 1-19 or the pharmaceutical composition of claim 20.

22. The method according to 21, wherein the modulation is inhibition.

23. The method according to either of claim 21 or 22, wherein the HECT domain E3 ligase is selected from WWP1, WWP2, and NEDD4.

24. A method of inhibiting HECT domain E3 ligase in a patient in need thereof, comprising: administering to said patient a therapeutically effective amount of the compound of any of claims 1-19 or the pharmaceutical composition of claim 20.

25. A method of inhibiting the growth, and/or the proliferation, and/or the infectivity, of an enveloped virus, said method comprising: contacting said virus with a therapeutically effective amount of the compound of any of claims 1-19 or the pharmaceutical composition of claim 20.

26. The method of claim 25, wherein said method comprises administering said compound to a mammal infected with said virus or at risk of infection with said virus.

27. The method of claim 26, wherein the mammal is a mammal infected with said virus.

28. The method according to either of claim 26 or 27, wherein the mammal is a non-human mammal.

29. The method according to either of claim 26 or 27, wherein the mammal is a human.

30. The method according to any one of claims 25-29, wherein the virus is a member of a family selected from the group consisting of Herpesviridae, Poxviridae, Hepadnaviridae, Coronaviridae, Flaviviridae, Togaviridae, Retroviridae, Orthomyxoviridae, Arenaviridae, Bunyaviridae, Filoviridae, Paramyxoviridae, and Rhabdoviridae.

31. The method according to any one of claims 25-29, wherein the virus is selected from the group consisting of Herpes simplex, type 1, Herpes simplex, type 2, Varicella-zoster virus, Epstein-Barr virus, Human cytomegalovirus, Human herpesvirus, Smallpox, Hepatitis B virus, Severe acute respiratory syndrome virus, Hepatitis C virus, yellow fever virus, dengue virus, West Nile virus, TBE virus, Zika virus, Rubella virus, Human immunodeficiency virus (HIV), Influenza virus, Lassa virus, Crimean-Congo, hemorrhagic fever virus, Hantaan virus, Ebola virus, Marburg virus, Measles virus, Mumps virus, Parainfluenza virus, Respiratory syncytial virus, Rabies virus, and Hepatitis D virus (HDV).

32. The method according to any one of claims 25-29, wherein the virus is selected from SARS-CoV, MERS-CoV, 229E, NL63, OC43, and HKU1.

33. The method according to any one of claims 25-29, wherein the virus is SARS-CoV-2.

34. The method according to any one of claims 25-29, wherein the method ameliorates one of more symptoms of a pathology caused by said virus and/or slows or prevents infection of said mammal by said virus.

35. An antisense oligonucleotide comprising a nucleobase sequence complementary to at least 12 contiguous nucleotides in SEQ ID NO: 541 selected from the group consisting of: 1-45, 61-97, 173-192, 205-224, 235-272, 426-445, 524-573, 611-630, 647-715, 790-809, 851-870, 994-1024, 1057-1076, 1134-1188, 1276-1295, 1307-1326, 1373-1392, 1470-1489, 1599-1632, 1780-1799, 1817-1846, 1918-1937, 2225-2244, 2316-2335, 2367-2386, 2407-2457, 2651-2670, and 2761-2780.

36. The antisense oligonucleotide of claim 35, wherein the nucleobase sequence is (i) 12 nucleosides, (ii) 12-30 nucleosides or (iii) 15-30 linked nucleosides.

37. An antisense oligonucleotide comprising at least 12 linked nucleosides having a nucleobase sequence of any one of SEQ ID NOS: 1-44 and 532-540, or a variant sequence at least about 90% identical thereto.

38. The antisense oligonucleotide of claim 37, wherein the length of the antisense oligonucleotide is (i) 12 nucleosides, (ii) 12-30 nucleosides or (iii) 15-30 linked nucleosides.

39. The antisense oligonucleotide of any one of claims 35-38, wherein the oligonucleotide comprises a chemical modification.

40. The antisense oligonucleotide of any one of claims 37-38, wherein the antisense oligonucleotide is capable of modulating translation, expression or activity of WWP1 in a mammalian cell.

41. The antisense oligonucleotide of claim 40, wherein the modulation is inhibition or down-regulation.

42. The antisense oligonucleotide of any one of claims 35-40, wherein the nucleobases are independently selected from adenine, thymine, cytosine, guanine, uracil and 5-methylcytosine.

43. The antisense oligonucleotide of any one of claims 35-42, wherein the antisense oligonucleotide is capable of inhibiting or down-regulating translation, expression or activity of WWP1 in a mammalian cell.

44. The antisense oligonucleotide of any one of claims 35-43, wherein the antisense oligonucleotide is a modified oligonucleotide.

45. The antisense oligonucleotide of claim 44, wherein the modified oligonucleotide is a gapmer.

46. The antisense oligonucleotide of claim 44 or 45, wherein the modified oligonucleotide comprises at least one modified internucleoside linkage and/or at least one modified sugar moiety and/or at least one modified nucleobase.

47. The antisense oligonucleotide of claim 46, wherein the at least one modified internucleoside linkage is a phosphorothioate internucleoside linkage, the at least one modified sugar is a bicyclic sugar or 2′-O-methyl or -methyoxyethyl, and the at least one modified nucleobase is a 5 methylcytosine.

48. The antisense oligonucleotide of claim 46 or 47, wherein the modified internucleoside linkage comprises a phosphorothioate internucleoside linkage.

49. The antisense oligonucleotide of any one of claims 46-48, wherein the modified oligonucleotide is a chimeric oligonucleotide.

50. The antisense oligonucleotide of any one of claims 46-49, wherein the modified sugar moiety comprises a 2′-substituted sugar moiety, wherein the 2′-substituent is selected from among: 2′-OMe, 2′-F, and 2′-MOE.

51. The antisense oligonucleotide of any one of claims 46-50, wherein the 2′-modification is 2′-OR1, wherein R1 is optionally substituted C1-C6 aliphatic.

52. The antisense oligonucleotide of any one of claims 46-51, wherein the 2′-modification is 2′-OCH2CH2OMe.

53. The antisense oligonucleotide of any one of claims 46-52, wherein the modified sugar moiety comprises a bicyclic sugar moiety.

54. The antisense oligonucleotide of claim 53, wherein the bicyclic sugar moiety comprises LNA or cEt.

55. A pharmaceutical composition comprising at least one antisense oligonucleotide of any one of claims 35-54 and a pharmaceutically acceptable carrier, in an amount effective to inhibit or down-regulate translation, expression or activity of WWP1 in a mammalian cell.

56. The pharmaceutical composition according to claim 55, further comprising one or more vaccines, antigens, antibodies, cytotoxic agents, chemotherapeutic agents, kinase inhibitors, allergens, antibiotics, agonist, antagonist, antisense oligonucleotides, ribozymes, RNAi molecules, siRNA molecules, miRNA molecules, aptamers, proteins, gene therapy vectors, DNA vaccines, adjuvants, co-stimulatory molecules or combinations thereof.

57. A pharmaceutical composition according to claim 55 or 56, comprising: two or more antisense oligonucleotides of any one of claims 35-54 targeting different regions of WWP1.

58. A pharmaceutical composition comprising at least two antisense oligonucleotides comprising 15 to 30 linked nucleosides having a nucleobase sequence or chemical modification thereof comprising a complementary region comprising at least 12 contiguous nucleobases to a target region of equal length of a WWP1 and/or WWP2 and/or NEDD4.

59. The pharmaceutical composition according to claim 58, wherein the antisense oligonucleotides are chemically conjugated or co-formulated.

60. The pharmaceutical composition according to claim 58 or 59, wherein the target region of each oligonucleotide is different.

61. A method of treating autoimmune disease in a patient in need thereof, comprising: administering to said patient a therapeutically effective amount of the antisense oligonucleotide of any one of claims 35-54 or the pharmaceutical composition of any one of claims 55-60.

62. The method of claim 61, wherein the autoimmune disease is selected from the group consisting of: psoriasis, rheumatoid arthritis, systemic lupus erythematosus, ulcerative colitis, Crohn's disease, transplant rejection, immune disorder associated with graft transplantation rejection, benign lymphocytic angiitis, lupus erythematosus, Hashimoto's thyroiditis, primary myxedema, Graves's disease, pernicious anemia, autoimmune atrophic gastritis, Addison's disease, insulin dependent diabetes mellitis, Good pasture's syndrome, myasthenia gravis, pemphigus, sympathetic ophthalmia, autoimmune uveitis, autoimmune hemolytic anemia, idiopathic thrombocytopenia, primary biliary cirrhosis, chronic hepatitis, ulcerates colitis, Sjogren's syndrome, Wegener's sarcoidosis, antiphospholipid syndrome, inflammatory myopathy, polyarteritis, rheumatic disease, polymyositis, scleroderma, mixed connective tissue disease, inflammatory rheumatism, degenerative rheumatism, extra-articular rheumatism, collagen disease, chronic polyarthritis, psoriasis arthropathica, ankylosing spondylitis, juvenile rheumatoid arthritis, periarthritis humeroscapularis, panarteriitis nodosa, progressive systemic scleroderma, arthritis urica, dermatomyositis, muscular rheumatism, myositis, myogelosis, and chondrocalcinosis, thyroiditis, allergic oedema, granulomas, Alzheimer's disease, Parkinson's disease, multiple sclerosis, or amyotrophic lateral sclerosis (ALS).

63. A method of upregulating PD-L1, comprising: administering to a patient in need thereof a therapeutically effective amount of the antisense oligonucleotide of any one of claims 35-54 or the pharmaceutical composition of any one of claims 55-60.

64. A method of treating cancer in a patient in need thereof, comprising: administering to said patient a therapeutically effective amount of the compound of any of claims 1-19, the pharmaceutical composition of claim 20, the antisense oligonucleotide of any one of claims 35-54, or the pharmaceutical composition of any one of claims 55-60.

65. A method for modulating or inhibiting the activity of WWP1 and/or WWP2 and/or NEDD4, comprising: contacting a cell with the compound of any one of claims 1-19, the pharmaceutical composition of claim 20, the antisense oligonucleotide of any one of claims 35-54, or the pharmaceutical composition of any one of claims 55-60.

66. A method of enhancing apoptosis in a cell expressing WWP1 and/or WWP2 and/or NEDD4, comprising: contacting the cell with the compound of any of claims 1-19, the pharmaceutical composition of claim 20, the antisense oligonucleotide of any one of claims 35-54, or the pharmaceutical composition of any one of claims 55-60.

67. A method of inhibiting the growth of a cancer cell expressing WWP1 and/or WWP2 and/or NEDD4, comprising: contacting the cell with the compound of any one of claims 1-19, the pharmaceutical composition of claim 20, the antisense oligonucleotide of any one of claims 35-54, or the pharmaceutical composition of any one of claims 55-60.

68. A method for the treatment of a disease, disorder, or condition associated with WWP1 and/or WWP2 and/or NEDD4 in a mammal in need thereof, the method comprising: administering the compound of any one of claims 1-19, the pharmaceutical composition of claim 20, the antisense oligonucleotide of any one of claims 35-54, or the pharmaceutical composition of any one of claims 55-60.

69. A method for treating a cancer in a human, comprising: administering a therapeutically effective amount of the compound of any one of claims 1-19, the pharmaceutical composition of claim 20, the antisense oligonucleotide of any one of claims 35-54, or the pharmaceutical composition of any one of claims 55-60.

70. A method of inhibiting the growth of a human prostate cancer cell, comprising: contacting the cell with the compound of any of claims 1-19, the pharmaceutical composition of claim 20, the antisense oligonucleotide of any one of claims 35-54, or the pharmaceutical composition of any one of claims 55-60.

71. The method according to any one of claims 24, 26-34 and 61-70, wherein the route of administration of the compound or pharmaceutical composition is selected from, intrathecal, parenteral, intramuscular, subcutaneous, intraperitoneal, intravenous, mucosal delivery, oral, sublingual, transdermal, topical, inhalation, intranasal, aerosol, intraocular, intratracheal, intrarectal, vaginal, gene gun, dermal patch, eye drop or mouthwash.

72. The method of any of claims 24, 26-34 and 61-71, further comprising administration of one or more vaccines, antigens, antibodies, cytotoxic agents, chemotherapeutic agents, kinase inhibitors, allergens, antibiotics, agonist, antagonist, antisense oligonucleotides, ribozymes, RNAi molecules, siRNA molecules, miRNA molecules, aptamers, proteins, gene therapy vectors, DNA vaccines, adjuvants, co-stimulatory molecules or combinations thereof.

73. The method of any one of claims 24, 26-34 and 61-72, wherein the administration is daily.

74. A method of modulating the activity of at least one HECT domain E3 ligase in a mammalian cell, comprising: contacting a compound of formula (II) with the mammalian cell, wherein the compound of formula (II) is: 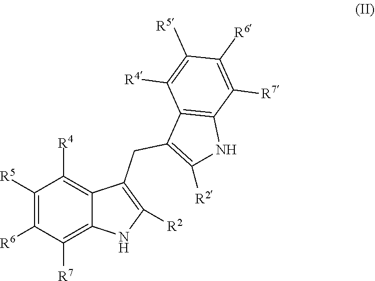
or a pharmaceutically acceptable salt or solvate thereof:
each of R² and R^{2′} are independently H or C₁-C₃ alkyl;
each of R⁴ and R^{4′} are independently H or halogen;
each of R⁵ and R^{5′} are independently H or CONR⁸R⁹;
each of R⁶ and R^{6′} are independently selected from the group consisting of H, CO₂H, and CONR⁸R⁹;
each of R⁷ and R^{7′} are independently selected from the group consisting of H, C₁-C₃ alkyl, and halogen;
each R⁸ is independently C₂-C₆alkynyl or 6-membered heteroaryl optionally substituted with halogen or nitrile; and
each R⁹ is independently selected from H or C₁-C₃alkyl optionally substituted with —OH;
wherein R⁵ and R⁶ are not both H.

75. The method according to 74, wherein the modulation is inhibition.

76. The method according to either of claim 74 or 75, wherein the HECT domain E3 ligase is selected from WWP1, WWP2, and NEDD4.

77. A method of inhibiting HECT domain E3 ligase in a patient in need thereof, comprising: administering to said patient a therapeutically effective amount of a compound of formula (II), wherein the compound of formula (II) is: 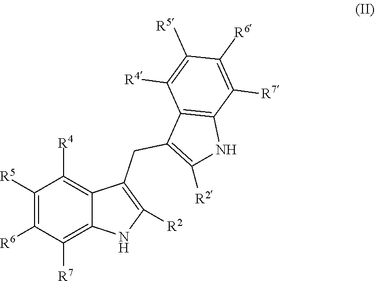
or a pharmaceutically acceptable salt or solvate thereof:
each of R² and R^{2′} are independently H or C₁-C₃ alkyl;
each of R⁴ and R^{4′} are independently H or halogen;
each of R⁵ and R^{5′} are independently H or CONR⁸R⁹;
each of R⁶ and R^{6′} are independently selected from the group consisting of H, CO₂H, and CONR⁸R⁹;
each of R⁷ and R^{7′} are independently selected from the group consisting of H, C₁-C₃ alkyl, and halogen;
each R⁸ is independently C₂-C₆alkynyl or 6-membered heteroaryl optionally substituted with halogen or nitrile; and
each R⁹ is independently selected from H or C₁-C₃alkyl optionally substituted with —OH;
wherein R⁵ and R⁶ are not both H.

78. A method of inhibiting the growth, and/or the proliferation, and/or the infectivity, of an enveloped virus, said method comprising contacting said virus with a therapeutically effective amount of a compound of formula (II), wherein the compound of formula (II) is: 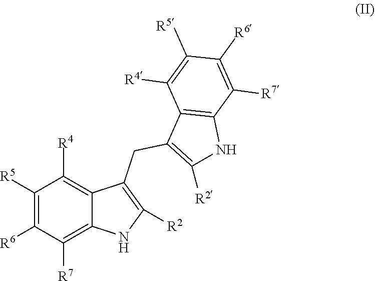
or a pharmaceutically acceptable salt or solvate thereof:
each of R² and R^{2′} are independently H or C₁-C₃ alkyl;
each of R⁴ and R^{4′} are independently H or halogen;
each of R⁵ and R^{5′} are independently H or CONR⁸R⁹;
each of R⁶ and R^{6′} are independently selected from the group consisting of H, CO₂H, and CONR⁸R⁹;
each of R⁷ and R^{7′} are independently selected from the group consisting of H, C₁-C₃ alkyl, and halogen;
each R⁸ is independently C₂-C₆alkynyl or 6-membered heteroaryl optionally substituted with halogen or nitrile; and
each R⁹ is independently selected from H or C₁-C₃alkyl optionally substituted with —OH;
wherein R⁵ and R⁶ are not both H.

79. The method of claim 78, wherein said method comprises administering said compound to a mammal infected with said virus or at risk of infection with said virus.

80. The method of claim 79, wherein the mammal is a mammal infected with said virus.

81. The method according to either of claim 79 or 80, wherein the mammal is a non-human mammal.

82. The method according to either of claim 79 or 80, wherein the mammal is a human.

83. The method according to any one of claims 79-82, wherein the virus is a member of a family selected from the group consisting of Herpesviridae, Poxviridae, Hepadnaviridae, Coronaviridae, Flaviviridae, Togaviridae, Retroviridae, Orthomyxoviridae, Arenaviridae, Bunyaviridae, Filoviridae, Paramyxoviridae, and Rhabdoviridae.

84. The method according to any one of claims 79-82, wherein the virus is selected from the group consisting of Herpes simplex, type 1, Herpes simplex, type 2, Varicella-zoster virus, Epstein-Barr virus, Human cytomegalovirus, Human herpesvirus, Smallpox, Hepatitis B virus, Severe acute respiratory syndrome virus, Hepatitis C virus, yellow fever virus, dengue virus, West Nile virus, TBE virus, Zika virus, Rubella virus, Human immunodeficiency virus (HIV), Influenza virus, Lassa virus, Crimean-Congo, hemorrhagic fever virus, Hantaan virus, Ebola virus, Marburg virus, Measles virus, Mumps virus, Parainfluenza virus, Respiratory syncytial virus, Rabies virus, and Hepatitis D virus (HDV).

85. The method according to any one of claims 79-82, wherein the virus is selected from SARS-CoV, MERS-CoV, 229E, NL63, OC43, and HKU1.

86. The method according to any one of claims 79-82, wherein the virus is SARS-CoV-2.

87. The method according to any one of claims 79-82, wherein the method ameliorates one of more symptoms of a pathology caused by said virus and/or slows or prevents infection of said mammal by said virus.

88. A method of treating cancer in a patient in need thereof, comprising: administering to said patient a therapeutically effective amount of a compound of formula (II), wherein the compound of formula (II) is: 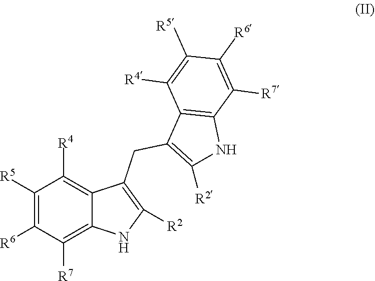
or a pharmaceutically acceptable salt or solvate thereof:
each of R² and R^{2′} are independently H or C₁-C₃ alkyl;
each of R⁴ and R^{4′} are independently H or halogen;
each of R⁵ and R^{5′} are independently H or CONR⁸R⁹;
each of R⁶ and R^{6′} are independently selected from the group consisting of H, CO₂H, and CONR⁸R⁹;
each of R⁷ and R^{7′} are independently selected from the group consisting of H, C₁-C₃ alkyl, and halogen;
each R⁸ is independently C₂-C₆alkynyl or 6-membered heteroaryl optionally substituted with halogen or nitrile; and
each R⁹ is independently selected from H or C₁-C₃alkyl optionally substituted with —OH;
wherein R⁵ and R⁶ are not both H.

89. A method for modulating or inhibiting the activity of WWP1 and/or WWP2 and/or NEDD4, comprising: contacting a cell with a compound of formula (II), wherein the compound of formula (II) is: 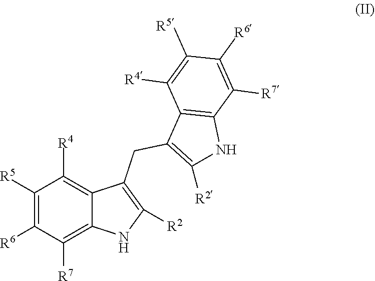
or a pharmaceutically acceptable salt or solvate thereof:
each of R² and R^{2′} are independently H or C₁-C₃ alkyl;
each of R⁴ and R^{4′} are independently H or halogen;
each of R⁵ and R^{5′} are independently H or CONR⁸R⁹;
each of R⁶ and R^{6′} are independently selected from the group consisting of H, CO₂H, and CONR⁸R⁹;
each of R⁷ and R^{7′} are independently selected from the group consisting of H, C₁-C₃ alkyl, and halogen;
each R⁸ is independently C₂-C₆alkynyl or 6-membered heteroaryl optionally substituted with halogen or nitrile; and
each R⁹ is independently selected from H or C₁-C₃alkyl optionally substituted with —OH;
wherein R⁵ and R⁶ are not both H.

90. A method of enhancing apoptosis in a cell expressing WWP1 and/or WWP2 and/or NEDD4, comprising: contacting the cell with a compound of formula (II), wherein the compound of formula (II) is: 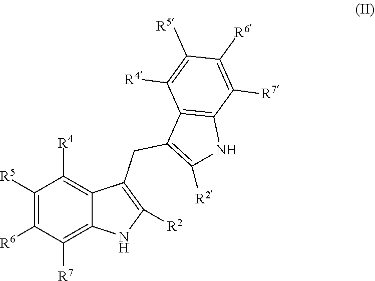
or a pharmaceutically acceptable salt or solvate thereof:
each of R² and R^{2′} are independently H or C₁-C₃ alkyl;
each of R⁴ and R^{4′} are independently H or halogen;
each of R⁵ and R^{5′} are independently H or CONR⁸R⁹;
each of R⁶ and R^{6′} are independently selected from the group consisting of H, CO₂H, and CONR⁸R⁹;
each of R⁷ and R^{7′} are independently selected from the group consisting of H, C₁-C₃ alkyl, and halogen;
each R⁸ is independently C₂-C₆alkynyl or 6-membered heteroaryl optionally substituted with halogen or nitrile; and
each R⁹ is independently selected from H or C₁-C₃alkyl optionally substituted with —OH;
wherein R⁵ and R⁶ are not both H.

91. A method of inhibiting the growth of a cancer cell expressing WWP1 and/or WWP2 and/or NEDD4, comprising: contacting the cell with a compound of formula (II), wherein the compound of formula (II) is: 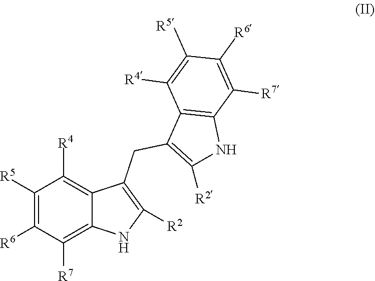
or a pharmaceutically acceptable salt or solvate thereof:
each of R² and R^{2′} are independently H or C₁-C₃ alkyl;
each of R⁴ and R^{4′} are independently H or halogen;
each of R⁵ and R^{5′} are independently H or CONR⁸R⁹;
each of R⁶ and R^{6′} are independently selected from the group consisting of H, CO₂H, and CONR⁸R⁹;
each of R⁷ and R^{7′} are independently selected from the group consisting of H, C₁-C₃ alkyl, and halogen;
each R⁸ is independently C₂-C₆alkynyl or 6-membered heteroaryl optionally substituted with halogen or nitrile; and
each R⁹ is independently selected from H or C₁-C₃alkyl optionally substituted with —OH;
wherein R⁵ and R⁶ are not both H.

92. A method for the treatment of a disease, disorder, or condition associated with WWP1 and/or WWP2 and/or NEDD4 in a mammal in need thereof, the method comprising: administering a compound of formula (II), wherein the compound of formula (II) is: 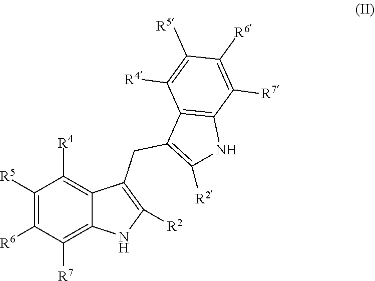
or a pharmaceutically acceptable salt or solvate thereof:
each of R² and R^{2′} are independently H or C₁-C₃ alkyl;
each of R⁴ and R^{4′} are independently H or halogen;
each of R⁵ and R^{5′} are independently H or CONR⁸R⁹;
each of R⁶ and R^{6′} are independently selected from the group consisting of H, CO₂H, and CONR⁸R⁹;
each of R⁷ and R^{7′} are independently selected from the group consisting of H, C₁-C₃ alkyl, and halogen;
each R⁸ is independently C₂-C₆alkynyl or 6-membered heteroaryl optionally substituted with halogen or nitrile; and
each R⁹ is independently selected from H or C₁-C₃alkyl optionally substituted with —OH;
wherein R⁵ and R⁶ are not both H.

93. A method for treating a cancer in a human, comprising: administering a therapeutically effective amount of a compound of formula (II), wherein the compound of formula (II) is: 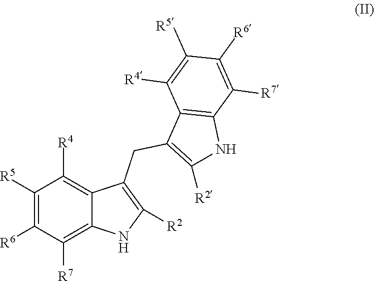
or a pharmaceutically acceptable salt or solvate thereof:
each of R² and R^{2′} are independently H or C₁-C₃ alkyl;
each of R⁴ and R^{4′} are independently H or halogen;
each of R⁵ and R^{5′} are independently H or CONR⁸R⁹;
each of R⁶ and R^{6′} are independently selected from the group consisting of H, CO₂H, and CONR⁸R⁹;
each of R⁷ and R^{7′} are independently selected from the group consisting of H, C₁-C₃ alkyl, and halogen;
each R⁸ is independently C₂-C₆alkynyl or 6-membered heteroaryl optionally substituted with halogen or nitrile; and
each R⁹ is independently selected from H or C₁-C₃alkyl optionally substituted with —OH;
wherein R⁵ and R⁶ are not both H.

94. A method of inhibiting the growth of a human prostate cancer cell, comprising: contacting the cell with a compound of formula (II), wherein the compound of formula (II) is: 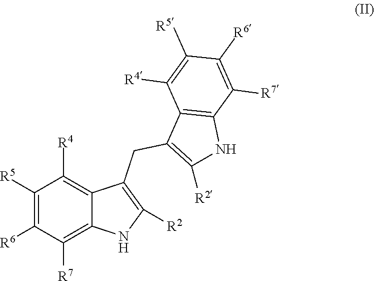
or a pharmaceutically acceptable salt or solvate thereof:
each of R² and R^{2′} are independently H or C₁-C₃ alkyl;
each of R⁴ and R^{4′} are independently H or halogen;
each of R⁵ and R^{5′} are independently H or CONR⁸R⁹;
each of R⁶ and R^{6′} are independently selected from the group consisting of H, CO₂H, and CONR⁸R⁹;
each of R⁷ and R^{7′} are independently selected from the group consisting of H, C₁-C₃ alkyl, and halogen;
each R⁸ is independently C₂-C₆alkynyl or 6-membered heteroaryl optionally substituted with halogen or nitrile; and
each R⁹ is independently selected from H or C₁-C₃alkyl optionally substituted with —OH;
wherein R⁵ and R⁶ are not both H.

95. The method according to any one of claims 74-94, wherein the route of administration of the compound or pharmaceutical composition is selected from, intrathecal, parenteral, intramuscular, subcutaneous, intraperitoneal, intravenous, mucosal delivery, oral, sublingual, transdermal, topical, inhalation, intranasal, aerosol, intraocular, intratracheal, intrarectal, vaginal, gene gun, dermal patch, eye drop or mouthwash.

96. The method of any of claims 74-95, further comprising administration of one or more vaccines, antigens, antibodies, cytotoxic agents, chemotherapeutic agents, kinase inhibitors, allergens, antibiotics, agonist, antagonist, antisense oligonucleotides, ribozymes, RNAi molecules, siRNA molecules, miRNA molecules, aptamers, proteins, gene therapy vectors, DNA vaccines, adjuvants, co-stimulatory molecules or combinations thereof.


SEQUENCE LISTING (541 sequences)

SEQ ID NO: 1 - DNA, 20 bp, Artificial Sequence
  Description of Artificial Sequence Synthetic oligonucleotide
  Description of Combined DNA/RNA Molecule Synthetic oligonucleotide
ccaugtcccaaaattcagcu

SEQ ID NO: 2 - DNA, 20 bp, Artificial Sequence
  Description of Artificial Sequence Synthetic oligonucleotide
  Description of Combined DNA/RNA Molecule Synthetic oligonucleotide
gaccutggtgaagcaguggc

SEQ ID NO: 3 - DNA, 20 bp, Artificial Sequence
  Description of Artificial Sequence Synthetic oligonucleotide
  Description of Combined DNA/RNA Molecule Synthetic oligonucleotide
guaucagaccttggtgaagc

SEQ ID NO: 4 - DNA, 20 bp, Artificial Sequence
  Description of Artificial Sequence Synthetic oligonucleotide
  Description of Combined DNA/RNA Molecule Synthetic oligonucleotide
ccuguaactgcaaccuucca

SEQ ID NO: 5 - DNA, 20 bp, Artificial Sequence
  Description of Artificial Sequence Synthetic oligonucleotide
  Description of Combined DNA/RNA Molecule Synthetic oligonucleotide
ggcactagaaacagtuaccu

SEQ ID NO: 6 - DNA, 20 bp, Artificial Sequence
  Description of Artificial Sequence Synthetic oligonucleotide
  Description of Combined DNA/RNA Molecule Synthetic oligonucleotide
gaacuactggattttgcugu

SEQ ID NO: 7 - DNA, 20 bp, Artificial Sequence
  Description of Artificial Sequence Synthetic oligonucleotide
  Description of Combined DNA/RNA Molecule Synthetic oligonucleotide
caguuagctgttcatcccau

SEQ ID NO: 8 - DNA, 20 bp, Artificial Sequence
  Description of Artificial Sequence Synthetic oligonucleotide
  Description of Combined DNA/RNA Molecule Synthetic oligonucleotide
auuccaatgtagtctguggc

SEQ ID NO: 9 - DNA, 20 bp, Artificial Sequence
  Description of Artificial Sequence Synthetic oligonucleotide
  Description of Combined DNA/RNA Molecule Synthetic oligonucleotide
gauggctccaaacttgaaau

SEQ ID NO: 10 - DNA, 20 bp, Artificial Sequence
  Description of Artificial Sequence Synthetic oligonucleotide
caccaatccatcaagcacaa

SEQ ID NO: 11 - DNA, 20 bp, Artificial Sequence
  Description of Artificial Sequence Synthetic oligonucleotide
  Description of Combined DNA/RNA Molecule Synthetic oligonucleotide
cuugctgaaggctctccauu

SEQ ID NO: 12 - DNA, 20 bp, Artificial Sequence
  Description of Artificial Sequence Synthetic oligonucleotide
  Description of Combined DNA/RNA Molecule Synthetic oligonucleotide
ggcagttgtccttgcugaag

SEQ ID NO: 13 - DNA, 20 bp, Artificial Sequence
  Description of Artificial Sequence Synthetic oligonucleotide
  Description of Combined DNA/RNA Molecule Synthetic oligonucleotide
gugccttcaacagccaaccu

SEQ ID NO: 14 - DNA, 20 bp, Artificial Sequence
  Description of Artificial Sequence Synthetic oligonucleotide
  Description of Combined DNA/RNA Molecule Synthetic oligonucleotide
gagcagcatgagtttuggac

SEQ ID NO: 15 - DNA, 20 bp, Artificial Sequence
  Description of Artificial Sequence Synthetic oligonucleotide
  Description of Combined DNA/RNA Molecule Synthetic oligonucleotide
ggagatgaaggtgtguuguc

SEQ ID NO: 16 - DNA, 20 bp, Artificial Sequence
  Description of Artificial Sequence Synthetic oligonucleotide
  Description of Combined DNA/RNA Molecule Synthetic oligonucleotide
uugggtctggcagcaaccug

SEQ ID NO: 17 - DNA, 20 bp, Artificial Sequence
  Description of Artificial Sequence Synthetic oligonucleotide
  Description of Combined DNA/RNA Molecule Synthetic oligonucleotide
gaguggttttggagcuggug

SEQ ID NO: 18 - DNA, 20 bp, Artificial Sequence
  Description of Artificial Sequence Synthetic oligonucleotide
  Description of Combined DNA/RNA Molecule Synthetic oligonucleotide
cagacactactggaguaccc

SEQ ID NO: 19 - DNA, 20 bp, Artificial Sequence
  Description of Artificial Sequence Synthetic oligonucleotide
  Description of Combined DNA/RNA Molecule Synthetic oligonucleotide
ucuugaactggaggaucuuc

SEQ ID NO: 20 - DNA, 20 bp, Artificial Sequence
  Description of Artificial Sequence Synthetic oligonucleotide
  Description of Combined DNA/RNA Molecule Synthetic oligonucleotide
cuggctgtcttgattuggcu

SEQ ID NO: 21 - DNA, 20 bp, Artificial Sequence
  Description of Artificial Sequence Synthetic oligonucleotide
  Description of Combined DNA/RNA Molecule Synthetic oligonucleotide
cauacacccatctggcuguc

SEQ ID NO: 22 - DNA, 20 bp, Artificial Sequence
  Description of Artificial Sequence Synthetic oligonucleotide
  Description of Combined DNA/RNA Molecule Synthetic oligonucleotide
cugauggcaaggtttcugug

SEQ ID NO: 23 - DNA, 20 bp, Artificial Sequence
  Description of Artificial Sequence Synthetic oligonucleotide
  Description of Combined DNA/RNA Molecule Synthetic oligonucleotide
ucucucccatgtggtaguuc

SEQ ID NO: 24 - DNA, 20 bp, Artificial Sequence
  Description of Artificial Sequence Synthetic oligonucleotide
  Description of Combined DNA/RNA Molecule Synthetic oligonucleotide
gguugtggtctctcccaugu

SEQ ID NO: 25 - DNA, 20 bp, Artificial Sequence
  Description of Artificial Sequence Synthetic oligonucleotide
  Description of Combined DNA/RNA Molecule Synthetic oligonucleotide
cuggaggtaaaggttguggu

SEQ ID NO: 26 - DNA, 20 bp, Artificial Sequence
  Description of Artificial Sequence Synthetic oligonucleotide
  Description of Combined DNA/RNA Molecule Synthetic oligonucleotide
acucutctttcccaaccugg

SEQ ID NO: 27 - DNA, 20 bp, Artificial Sequence
  Description of Artificial Sequence Synthetic oligonucleotide
  Description of Combined DNA/RNA Molecule Synthetic oligonucleotide
gcugagattgccactguuca

SEQ ID NO: 28 - DNA, 20 bp, Artificial Sequence
  Description of Artificial Sequence Synthetic oligonucleotide
  Description of Combined DNA/RNA Molecule Synthetic oligonucleotide
aacugttgcatagctcccug

SEQ ID NO: 29 - DNA, 20 bp, Artificial Sequence
  Description of Artificial Sequence Synthetic oligonucleotide
  Description of Combined DNA/RNA Molecule Synthetic oligonucleotide
ggcaaaggtccataaggguc

SEQ ID NO: 30 - DNA, 20 bp, Artificial Sequence
  Description of Artificial Sequence Synthetic oligonucleotide
  Description of Combined DNA/RNA Molecule Synthetic oligonucleotide
gccuugagttcttggaucuu

SEQ ID NO: 31 - DNA, 20 bp, Artificial Sequence
  Description of Artificial Sequence Synthetic oligonucleotide
  Description of Combined DNA/RNA Molecule Synthetic oligonucleotide
uacagatgacttcccauugc

SEQ ID NO: 32 - DNA, 20 bp, Artificial Sequence
  Description of Artificial Sequence Synthetic oligonucleotide
  Description of Combined DNA/RNA Molecule Synthetic oligonucleotide
ggaccacctttagttacaga

SEQ ID NO: 33 - DNA, 20 bp, Artificial Sequence
  Description of Artificial Sequence Synthetic oligonucleotide
  Description of Combined DNA/RNA Molecule Synthetic oligonucleotide
gccucctcaagtcatagggu

SEQ ID NO: 34 - DNA, 20 bp, Artificial Sequence
  Description of Artificial Sequence Synthetic oligonucleotide
  Description of Combined DNA/RNA Molecule Synthetic oligonucleotide
ucaagtccttcttctccucu

SEQ ID NO: 35 - DNA, 20 bp, Artificial Sequence
  Description of Artificial Sequence Synthetic oligonucleotide
  Description of Combined DNA/RNA Molecule Synthetic oligonucleotide
gccaccataatcaaguccuu

SEQ ID NO: 36 - DNA, 20 bp, Artificial Sequence
  Description of Artificial Sequence Synthetic oligonucleotide
  Description of Combined DNA/RNA Molecule Synthetic oligonucleotide
gacaatagttgttctugccc

SEQ ID NO: 37 - DNA, 20 bp, Artificial Sequence
  Description of Artificial Sequence Synthetic oligonucleotide
  Description of Combined DNA/RNA Molecule Synthetic oligonucleotide
ccucccaacttcaggucaug

SEQ ID NO: 38 - DNA, 20 bp, Artificial Sequence
  Description of Artificial Sequence Synthetic oligonucleotide
  Description of Combined DNA/RNA Molecule Synthetic oligonucleotide
ggucugttcttgtacuccuc

SEQ ID NO: 39 - DNA, 20 bp, Artificial Sequence
  Description of Artificial Sequence Synthetic oligonucleotide
  Description of Combined DNA/RNA Molecule Synthetic oligonucleotide
cuguagccactgaagaggaa

SEQ ID NO: 40 - DNA, 20 bp, Artificial Sequence
  Description of Artificial Sequence Synthetic oligonucleotide
  Description of Combined DNA/RNA Molecule Synthetic oligonucleotide
ugccacacaacataaccucu

SEQ ID NO: 41 - DNA, 20 bp, Artificial Sequence
  Description of Artificial Sequence Synthetic oligonucleotide
  Description of Combined DNA/RNA Molecule Synthetic oligonucleotide
gccaagtcaacctccugcau

SEQ ID NO: 42 - DNA, 20 bp, Artificial Sequence
  Description of Artificial Sequence Synthetic oligonucleotide
  Description of Combined DNA/RNA Molecule Synthetic oligonucleotide
cucugccaatctgccaaguc

SEQ ID NO: 43 - DNA, 20 bp, Artificial Sequence
  Description of Artificial Sequence Synthetic oligonucleotide
  Description of Combined DNA/RNA Molecule Synthetic oligonucleotide
gguaaccaagtgtctuugcc

SEQ ID NO: 44 - DNA, 20 bp, Artificial Sequence
  Description of Artificial Sequence Synthetic oligonucleotide
  Description of Combined DNA/RNA Molecule Synthetic oligonucleotide
cuuguccaaatccctcuguc

SEQ ID NO: 45 - DNA, 18 bp, Artificial Sequence
  Description of Artificial Sequence Synthetic oligonucleotide
bacgcagtcctttttgaa

SEQ ID NO: 46 - DNA, 20 bp, Artificial Sequence
  Description of Artificial Sequence Synthetic oligonucleotide
cctttttgaaggccagtttc

SEQ ID NO: 47 - DNA, 20 bp, Artificial Sequence
  Description of Artificial Sequence Synthetic oligonucleotide
ggccagtttccctctatcac

SEQ ID NO: 48 - DNA, 20 bp, Artificial Sequence
  Description of Artificial Sequence Synthetic oligonucleotide
cctctatcacataacctgtc

SEQ ID NO: 49 - DNA, 20 bp, Artificial Sequence
  Description of Artificial Sequence Synthetic oligonucleotide
ataacctgtcacatacatta

SEQ ID NO: 50 - DNA, 20 bp, Artificial Sequence
  Description of Artificial Sequence Synthetic oligonucleotide
acatacattagaattttttt

SEQ ID NO: 51 - DNA, 20 bp, Artificial Sequence
  Description of Artificial Sequence Synthetic oligonucleotide
gaattttttttttaattctt

SEQ ID NO: 52 - DNA, 20 bp, Artificial Sequence
  Description of Artificial Sequence Synthetic oligonucleotide
tttaattctttaggcttgaa

SEQ ID NO: 53 - DNA, 20 bp, Artificial Sequence
  Description of Artificial Sequence Synthetic oligonucleotide
taggcttgaaaattaccaga

SEQ ID NO: 54 - DNA, 20 bp, Artificial Sequence
  Description of Artificial Sequence Synthetic oligonucleotide
aattaccagagtgttcataa

SEQ ID NO: 55 - DNA, 20 bp, Artificial Sequence
  Description of Artificial Sequence Synthetic oligonucleotide
gtgttcataataaggatttg

SEQ ID NO: 56 - DNA, 20 bp, Artificial Sequence
  Description of Artificial Sequence Synthetic oligonucleotide
taaggatttggaaaaggagt

SEQ ID NO: 57 - DNA, 20 bp, Artificial Sequence
  Description of Artificial Sequence Synthetic oligonucleotide
gaaaaggagtctattcaacc

SEQ ID NO: 58 - DNA, 20 bp, Artificial Sequence
  Description of Artificial Sequence Synthetic oligonucleotide
ctattcaaccaagcagatct

SEQ ID NO: 59 - DNA, 20 bp, Artificial Sequence
  Description of Artificial Sequence Synthetic oligonucleotide
aagcagatctctgcctttct

SEQ ID NO: 60 - DNA, 20 bp, Artificial Sequence
  Description of Artificial Sequence Synthetic oligonucleotide
ctgcctttctttcgtcttta

SEQ ID NO: 61 - DNA, 20 bp, Artificial Sequence
  Description of Artificial Sequence Synthetic oligonucleotide
ttcgtctttactatgaattc

SEQ ID NO: 62 - DNA, 20 bp, Artificial Sequence
  Description of Artificial Sequence Synthetic oligonucleotide
ctatgaattctgaataaaat

SEQ ID NO: 63 - DNA, 20 bp, Artificial Sequence
  Description of Artificial Sequence Synthetic oligonucleotide
tgaataaaatggagaacaca

SEQ ID NO: 64 - DNA, 20 bp, Artificial Sequence
  Description of Artificial Sequence Synthetic oligonucleotide
ggagaacacactctcaatat

SEQ ID NO: 65 - DNA, 20 bp, Artificial Sequence
  Description of Artificial Sequence Synthetic oligonucleotide
ctctcaatatgtagaaaaat

SEQ ID NO: 66 - DNA, 20 bp, Artificial Sequence
  Description of Artificial Sequence Synthetic oligonucleotide
gtagaaaaataaaattttgg

SEQ ID NO: 67 - DNA, 20 bp, Artificial Sequence
  Description of Artificial Sequence Synthetic oligonucleotide
aaaattttggcaaaacaata

SEQ ID NO: 68 - DNA, 20 bp, Artificial Sequence
  Description of Artificial Sequence Synthetic oligonucleotide
caaaacaataattactgttt

SEQ ID NO: 69 - DNA, 20 bp, Artificial Sequence
  Description of Artificial Sequence Synthetic oligonucleotide
attactgtttttctgtatgt

SEQ ID NO: 70 - DNA, 20 bp, Artificial Sequence
  Description of Artificial Sequence Synthetic oligonucleotide
ttctgtatgtacttagcagt

SEQ ID NO: 71 - DNA, 20 bp, Artificial Sequence
  Description of Artificial Sequence Synthetic oligonucleotide
acttagcagtgaaatattta

SEQ ID NO: 72 - DNA, 20 bp, Artificial Sequence
  Description of Artificial Sequence Synthetic oligonucleotide
gaaatatttataggcaacag

SEQ ID NO: 73 - DNA, 20 bp, Artificial Sequence
  Description of Artificial Sequence Synthetic oligonucleotide
taggcaacagattttaagaa

SEQ ID NO: 74 - DNA, 20 bp, Artificial Sequence
  Description of Artificial Sequence Synthetic oligonucleotide
attttaagaagtcagttcca

SEQ ID NO: 75 - DNA, 20 bp, Artificial Sequence
  Description of Artificial Sequence Synthetic oligonucleotide
gtcagttccatggctattaa

SEQ ID NO: 76 - DNA, 20 bp, Artificial Sequence
  Description of Artificial Sequence Synthetic oligonucleotide
tggctattaaaaaatgttta

SEQ ID NO: 77 - DNA, 20 bp, Artificial Sequence
  Description of Artificial Sequence Synthetic oligonucleotide
aaaatgtttaagttaatgaa

SEQ ID NO: 78 - DNA, 20 bp, Artificial Sequence
  Description of Artificial Sequence Synthetic oligonucleotide
agttaatgaaatttacagtg

SEQ ID NO: 79 - DNA, 20 bp, Artificial Sequence
  Description of Artificial Sequence Synthetic oligonucleotide
atttacagtgcatattaaaa

SEQ ID NO: 80 - DNA, 20 bp, Artificial Sequence
  Description of Artificial Sequence Synthetic oligonucleotide
catattaaaagcatgataca

SEQ ID NO: 81 - DNA, 20 bp, Artificial Sequence
  Description of Artificial Sequence Synthetic oligonucleotide
gcatgatacaaatggaaatt

SEQ ID NO: 82 - DNA, 20 bp, Artificial Sequence
  Description of Artificial Sequence Synthetic oligonucleotide
aatggaaatttaaaattttg

SEQ ID NO: 83 - DNA, 20 bp, Artificial Sequence
  Description of Artificial Sequence Synthetic oligonucleotide
taaaattttgtattaggaag

SEQ ID NO: 84 - DNA, 20 bp, Artificial Sequence
  Description of Artificial Sequence Synthetic oligonucleotide
tattaggaagcccagtttgc

SEQ ID NO: 85 - DNA, 20 bp, Artificial Sequence
  Description of Artificial Sequence Synthetic oligonucleotide
cccagtttgctaaaagtagc

SEQ ID NO: 86 - DNA, 20 bp, Artificial Sequence
  Description of Artificial Sequence Synthetic oligonucleotide
taaaagtagcattaatatca

SEQ ID NO: 87 - DNA, 20 bp, Artificial Sequence
  Description of Artificial Sequence Synthetic oligonucleotide
attaatatcattcacatcag

SEQ ID NO: 88 - DNA, 20 bp, Artificial Sequence
  Description of Artificial Sequence Synthetic oligonucleotide
ttcacatcagattttaattt

SEQ ID NO: 89 - DNA, 20 bp, Artificial Sequence
  Description of Artificial Sequence Synthetic oligonucleotide
attttaatttgttacacatt

SEQ ID NO: 90 - DNA, 20 bp, Artificial Sequence
  Description of Artificial Sequence Synthetic oligonucleotide
gttacacatttttgtagttc

SEQ ID NO: 91 - DNA, 20 bp, Artificial Sequence
  Description of Artificial Sequence Synthetic oligonucleotide
tttgtagttcagcaaaggcc

SEQ ID NO: 92 - DNA, 20 bp, Artificial Sequence
  Description of Artificial Sequence Synthetic oligonucleotide
agcaaaggcccctatgatga

SEQ ID NO: 93 - DNA, 20 bp, Artificial Sequence
  Description of Artificial Sequence Synthetic oligonucleotide
cctatgatgaaaaacaatta

SEQ ID NO: 94 - DNA, 20 bp, Artificial Sequence
  Description of Artificial Sequence Synthetic oligonucleotide
aaaacaattaaaaagtgtta

SEQ ID NO: 95 - DNA, 20 bp, Artificial Sequence
  Description of Artificial Sequence Synthetic oligonucleotide
aaaagtgttatgacaaaaat

SEQ ID NO: 96 - DNA, 20 bp, Artificial Sequence
  Description of Artificial Sequence Synthetic oligonucleotide
tgacaaaaataattatctgt

SEQ ID NO: 97 - DNA, 20 bp, Artificial Sequence
  Description of Artificial Sequence Synthetic oligonucleotide
aattatctgtgtaaaaaggg

SEQ ID NO: 98 - DNA, 20 bp, Artificial Sequence
  Description of Artificial Sequence Synthetic oligonucleotide
gtaaaaagggggagacccaa

SEQ ID NO: 99 - DNA, 20 bp, Artificial Sequence
  Description of Artificial Sequence Synthetic oligonucleotide
ggagacccaaaagatttttc

SEQ ID NO: 100 - DNA, 20 bp, Artificial Sequence
  Description of Artificial Sequence Synthetic oligonucleotide
aagatttttcatatggttat

SEQ ID NO: 101 - DNA, 20 bp, Artificial Sequence
  Description of Artificial Sequence Synthetic oligonucleotide
atatggttatttaattcagt

SEQ ID NO: 102 - DNA, 20 bp, Artificial Sequence
  Description of Artificial Sequence Synthetic oligonucleotide
ttaattcagttaaatagaac

SEQ ID NO: 103 - DNA, 20 bp, Artificial Sequence
  Description of Artificial Sequence Synthetic oligonucleotide
taaatagaactgttatgaat

SEQ ID NO: 104 - DNA, 20 bp, Artificial Sequence
  Description of Artificial Sequence Synthetic oligonucleotide
tgttatgaatttgagaggaa

SEQ ID NO: 105 - DNA, 20 bp, Artificial Sequence
  Description of Artificial Sequence Synthetic oligonucleotide
ttgagaggaatgaagatatt

SEQ ID NO: 106 - DNA, 20 bp, Artificial Sequence
  Description of Artificial Sequence Synthetic oligonucleotide
tgaagatattcaaacactaa

SEQ ID NO: 107 - DNA, 20 bp, Artificial Sequence
  Description of Artificial Sequence Synthetic oligonucleotide
caaacactaacagaaaatca

SEQ ID NO: 108 - DNA, 20 bp, Artificial Sequence
  Description of Artificial Sequence Synthetic oligonucleotide
cagaaaatcattagccttat

SEQ ID NO: 109 - DNA, 20 bp, Artificial Sequence
  Description of Artificial Sequence Synthetic oligonucleotide
ttagccttatattagagcat

SEQ ID NO: 110 - DNA, 20 bp, Artificial Sequence
  Description of Artificial Sequence Synthetic oligonucleotide
attagagcattcaaatgctt

SEQ ID NO: 111 - DNA, 20 bp, Artificial Sequence
  Description of Artificial Sequence Synthetic oligonucleotide
tcaaatgcttatgtaactct

SEQ ID NO: 112 - DNA, 20 bp, Artificial Sequence
  Description of Artificial Sequence Synthetic oligonucleotide
atgtaactcttcaatccatc

SEQ ID NO: 113 - DNA, 20 bp, Artificial Sequence
  Description of Artificial Sequence Synthetic oligonucleotide
tcaatccatctggcttcata

SEQ ID NO: 114 - DNA, 20 bp, Artificial Sequence
  Description of Artificial Sequence Synthetic oligonucleotide
tggcttcataaaaaccaaaa

SEQ ID NO: 115 - DNA, 20 bp, Artificial Sequence
  Description of Artificial Sequence Synthetic oligonucleotide
aaaaccaaaattttgaacac

SEQ ID NO: 116 - DNA, 20 bp, Artificial Sequence
  Description of Artificial Sequence Synthetic oligonucleotide
ttttgaacacaaaaaagtgg

SEQ ID NO: 117 - DNA, 20 bp, Artificial Sequence
  Description of Artificial Sequence Synthetic oligonucleotide
aaaaaagtggcagagtttac

SEQ ID NO: 118 - DNA, 20 bp, Artificial Sequence
  Description of Artificial Sequence Synthetic oligonucleotide
cagagtttacattacctttt

SEQ ID NO: 119 - DNA, 20 bp, Artificial Sequence
  Description of Artificial Sequence Synthetic oligonucleotide
attaccttttgtatgttcct

SEQ ID NO: 120 - DNA, 20 bp, Artificial Sequence
  Description of Artificial Sequence Synthetic oligonucleotide
gtatgttcctaagtatgaaa

SEQ ID NO: 121 - DNA, 20 bp, Artificial Sequence
  Description of Artificial Sequence Synthetic oligonucleotide
aagtatgaaattgcatcaat

SEQ ID NO: 122 - DNA, 20 bp, Artificial Sequence
  Description of Artificial Sequence Synthetic oligonucleotide
ttgcatcaatttattatgaa

SEQ ID NO: 123 - DNA, 20 bp, Artificial Sequence
  Description of Artificial Sequence Synthetic oligonucleotide
ttattatgaattaaatttga

SEQ ID NO: 124 - DNA, 20 bp, Artificial Sequence
  Description of Artificial Sequence Synthetic oligonucleotide
ttaaatttgaaaagcatcac

SEQ ID NO: 125 - DNA, 20 bp, Artificial Sequence
  Description of Artificial Sequence Synthetic oligonucleotide
aaagcatcacagatttaaaa

SEQ ID NO: 126 - DNA, 20 bp, Artificial Sequence
  Description of Artificial Sequence Synthetic oligonucleotide
agatttaaaaacaaagtaca

SEQ ID NO: 127 - DNA, 20 bp, Artificial Sequence
  Description of Artificial Sequence Synthetic oligonucleotide
acaaagtacaaaagagcctg

SEQ ID NO: 128 - DNA, 20 bp, Artificial Sequence
  Description of Artificial Sequence Synthetic oligonucleotide
aaagagcctgattcttttta

SEQ ID NO: 129 - DNA, 20 bp, Artificial Sequence
  Description of Artificial Sequence Synthetic oligonucleotide
attctttttaattccacaaa

SEQ ID NO: 130 - DNA, 20 bp, Artificial Sequence
  Description of Artificial Sequence Synthetic oligonucleotide
attccacaaatacctagcat

SEQ ID NO: 131 - DNA, 20 bp, Artificial Sequence
  Description of Artificial Sequence Synthetic oligonucleotide
tacctagcatctcaaagtaa

SEQ ID NO: 132 - DNA, 20 bp, Artificial Sequence
  Description of Artificial Sequence Synthetic oligonucleotide
ctcaaagtaacatgtaaaca

SEQ ID NO: 133 - DNA, 20 bp, Artificial Sequence
  Description of Artificial Sequence Synthetic oligonucleotide
catgtaaacaaacttctatg

SEQ ID NO: 134 - DNA, 20 bp, Artificial Sequence
  Description of Artificial Sequence Synthetic oligonucleotide
aacttctatgctgctcaatg

SEQ ID NO: 135 - DNA, 20 bp, Artificial Sequence
  Description of Artificial Sequence Synthetic oligonucleotide
ctgctcaatgaatccttcca

SEQ ID NO: 136 - DNA, 20 bp, Artificial Sequence
  Description of Artificial Sequence Synthetic oligonucleotide
aatccttccaatttcgataa

SEQ ID NO: 137 - DNA, 20 bp, Artificial Sequence
  Description of Artificial Sequence Synthetic oligonucleotide
atttcgataataaactaaat

SEQ ID NO: 138 - DNA, 20 bp, Artificial Sequence
  Description of Artificial Sequence Synthetic oligonucleotide
taaactaaatagtattggat

SEQ ID NO: 139 - DNA, 20 bp, Artificial Sequence
  Description of Artificial Sequence Synthetic oligonucleotide
agtattggatctagtatatg

SEQ ID NO: 140 - DNA, 20 bp, Artificial Sequence
  Description of Artificial Sequence Synthetic oligonucleotide
ctagtatatgactttcatgt

SEQ ID NO: 141 - DNA, 20 bp, Artificial Sequence
  Description of Artificial Sequence Synthetic oligonucleotide
actttcatgtgtaagttatg

SEQ ID NO: 142 - DNA, 20 bp, Artificial Sequence
  Description of Artificial Sequence Synthetic oligonucleotide
gtaagttatggttctatcca

SEQ ID NO: 143 - DNA, 20 bp, Artificial Sequence
  Description of Artificial Sequence Synthetic oligonucleotide
gttctatccattactttaac

SEQ ID NO: 144 - DNA, 20 bp, Artificial Sequence
  Description of Artificial Sequence Synthetic oligonucleotide
ttactttaacaatattactg

SEQ ID NO: 145 - DNA, 20 bp, Artificial Sequence
  Description of Artificial Sequence Synthetic oligonucleotide
aatattactgatgtaacaga

SEQ ID NO: 146 - DNA, 20 bp, Artificial Sequence
  Description of Artificial Sequence Synthetic oligonucleotide
atgtaacagagaaaaatttt

SEQ ID NO: 147 - DNA, 20 bp, Artificial Sequence
  Description of Artificial Sequence Synthetic oligonucleotide
gaaaaattttcaactattgt

SEQ ID NO: 148 - DNA, 20 bp, Artificial Sequence
  Description of Artificial Sequence Synthetic oligonucleotide
caactattgtatttatttaa

SEQ ID NO: 149 - DNA, 20 bp, Artificial Sequence
  Description of Artificial Sequence Synthetic oligonucleotide
atttatttaaaacaaactga

SEQ ID NO: 150 - DNA, 20 bp, Artificial Sequence
  Description of Artificial Sequence Synthetic oligonucleotide
aacaaactgacaagttcaag

SEQ ID NO: 151 - DNA, 20 bp, Artificial Sequence
  Description of Artificial Sequence Synthetic oligonucleotide
caagttcaagcacctgtctt

SEQ ID NO: 152 - DNA, 20 bp, Artificial Sequence
  Description of Artificial Sequence Synthetic oligonucleotide
cacctgtcttcagaaaagcc

SEQ ID NO: 153 - DNA, 20 bp, Artificial Sequence
  Description of Artificial Sequence Synthetic oligonucleotide
cagaaaagccagcagcattt

SEQ ID NO: 154 - DNA, 20 bp, Artificial Sequence
  Description of Artificial Sequence Synthetic oligonucleotide
agcagcattttttttttttt

SEQ ID NO: 155 - DNA, 20 bp, Artificial Sequence
  Description of Artificial Sequence Synthetic oligonucleotide
ttttttttttaacatactca

SEQ ID NO: 156 - DNA, 20 bp, Artificial Sequence
  Description of Artificial Sequence Synthetic oligonucleotide
aacatactcaaagtaagatt

SEQ ID NO: 157 - DNA, 20 bp, Artificial Sequence
  Description of Artificial Sequence Synthetic oligonucleotide
aagtaagatttggcctaagc

SEQ ID NO: 158 - DNA, 20 bp, Artificial Sequence
  Description of Artificial Sequence Synthetic oligonucleotide
tggcctaagcccttaatacc

SEQ ID NO: 159 - DNA, 20 bp, Artificial Sequence
  Description of Artificial Sequence Synthetic oligonucleotide
ccttaatacctttctgaaca

SEQ ID NO: 160 - DNA, 20 bp, Artificial Sequence
  Description of Artificial Sequence Synthetic oligonucleotide
tttctgaacagccatgcaac

SEQ ID NO: 161 - DNA, 20 bp, Artificial Sequence
  Description of Artificial Sequence Synthetic oligonucleotide
gccatgcaactaaacaccct

SEQ ID NO: 162 - DNA, 20 bp, Artificial Sequence
  Description of Artificial Sequence Synthetic oligonucleotide
taaacaccctcaggagatgt

SEQ ID NO: 163 - DNA, 20 bp, Artificial Sequence
  Description of Artificial Sequence Synthetic oligonucleotide
caggagatgttacataaggg

SEQ ID NO: 164 - DNA, 20 bp, Artificial Sequence
  Description of Artificial Sequence Synthetic oligonucleotide
tacataagggagagaagaac

SEQ ID NO: 165 - DNA, 20 bp, Artificial Sequence
  Description of Artificial Sequence Synthetic oligonucleotide
agagaagaacatggagcaat

SEQ ID NO: 166 - DNA, 20 bp, Artificial Sequence
  Description of Artificial Sequence Synthetic oligonucleotide
atggagcaatttgcactttt

SEQ ID NO: 167 - DNA, 20 bp, Artificial Sequence
  Description of Artificial Sequence Synthetic oligonucleotide
ttgcactttttcccctagat

SEQ ID NO: 168 - DNA, 20 bp, Artificial Sequence
  Description of Artificial Sequence Synthetic oligonucleotide
tcccctagataatattaaca

SEQ ID NO: 169 - DNA, 20 bp, Artificial Sequence
  Description of Artificial Sequence Synthetic oligonucleotide
aatattaacaaggtaaagca

SEQ ID NO: 170 - DNA, 20 bp, Artificial Sequence
  Description of Artificial Sequence Synthetic oligonucleotide
aggtaaagcaaatccagatc

SEQ ID NO: 171 - DNA, 20 bp, Artificial Sequence
  Description of Artificial Sequence Synthetic oligonucleotide
aatccagatctttatgaatg

SEQ ID NO: 172 - DNA, 20 bp, Artificial Sequence
  Description of Artificial Sequence Synthetic oligonucleotide
tttatgaatgaatggctgtc

SEQ ID NO: 173 - DNA, 20 bp, Artificial Sequence
  Description of Artificial Sequence Synthetic oligonucleotide
aatggctgtcatgtttaata

SEQ ID NO: 174 - DNA, 20 bp, Artificial Sequence
  Description of Artificial Sequence Synthetic oligonucleotide
atgtttaatacacttggagc

SEQ ID NO: 175 - DNA, 20 bp, Artificial Sequence
  Description of Artificial Sequence Synthetic oligonucleotide
cacttggagctctataaaac

SEQ ID NO: 176 - DNA, 20 bp, Artificial Sequence
  Description of Artificial Sequence Synthetic oligonucleotide
tctataaaactagagccact

SEQ ID NO: 177 - DNA, 20 bp, Artificial Sequence
  Description of Artificial Sequence Synthetic oligonucleotide
tagagccactatcatatatg

SEQ ID NO: 178 - DNA, 20 bp, Artificial Sequence
  Description of Artificial Sequence Synthetic oligonucleotide
atcatatatgtttatataga

SEQ ID NO: 179 - DNA, 20 bp, Artificial Sequence
  Description of Artificial Sequence Synthetic oligonucleotide
tttatatagatatatatgta

SEQ ID NO: 180 - DNA, 20 bp, Artificial Sequence
  Description of Artificial Sequence Synthetic oligonucleotide
tatatatgtatttttaataa

SEQ ID NO: 181 - DNA, 20 bp, Artificial Sequence
  Description of Artificial Sequence Synthetic oligonucleotide
tttttaataaagtctctcac

SEQ ID NO: 182 - DNA, 20 bp, Artificial Sequence
  Description of Artificial Sequence Synthetic oligonucleotide
agtctctcacttgcttcaaa

SEQ ID NO: 183 - DNA, 20 bp, Artificial Sequence
  Description of Artificial Sequence Synthetic oligonucleotide
ttgcttcaaaatttaaggat

SEQ ID NO: 184 - DNA, 20 bp, Artificial Sequence
  Description of Artificial Sequence Synthetic oligonucleotide
atttaaggatgatctttttc

SEQ ID NO: 185 - DNA, 20 bp, Artificial Sequence
  Description of Artificial Sequence Synthetic oligonucleotide
gatctttttcctggtcagtc

SEQ ID NO: 186 - DNA, 20 bp, Artificial Sequence
  Description of Artificial Sequence Synthetic oligonucleotide
ctggtcagtcatttcaaggg

SEQ ID NO: 187 - DNA, 20 bp, Artificial Sequence
  Description of Artificial Sequence Synthetic oligonucleotide
atttcaagggaacaataaat

SEQ ID NO: 188 - DNA, 20 bp, Artificial Sequence
  Description of Artificial Sequence Synthetic oligonucleotide
aacaataaataaagtagaaa

SEQ ID NO: 189 - DNA, 20 bp, Artificial Sequence
  Description of Artificial Sequence Synthetic oligonucleotide
aaagtagaaaaggatgaact

SEQ ID NO: 190 - DNA, 20 bp, Artificial Sequence
  Description of Artificial Sequence Synthetic oligonucleotide
aggatgaactgttttgcata

SEQ ID NO: 191 - DNA, 20 bp, Artificial Sequence
  Description of Artificial Sequence Synthetic oligonucleotide
gttttgcatatttctgtgga

SEQ ID NO: 192 - DNA, 20 bp, Artificial Sequence
  Description of Artificial Sequence Synthetic oligonucleotide
tttctgtggaagaacggaaa

SEQ ID NO: 193 - DNA, 20 bp, Artificial Sequence
  Description of Artificial Sequence Synthetic oligonucleotide
agaacggaaaacatactttg

SEQ ID NO: 194 - DNA, 20 bp, Artificial Sequence
  Description of Artificial Sequence Synthetic oligonucleotide
acatactttgagaggttcgg

SEQ ID NO: 195 - DNA, 20 bp, Artificial Sequence
  Description of Artificial Sequence Synthetic oligonucleotide
agaggttcggaaaattcact

SEQ ID NO: 196 - DNA, 20 bp, Artificial Sequence
  Description of Artificial Sequence Synthetic oligonucleotide
aaaattcactgtacagaatg

SEQ ID NO: 197 - DNA, 20 bp, Artificial Sequence
  Description of Artificial Sequence Synthetic oligonucleotide
gtacagaatgaacagcttat

SEQ ID NO: 198 - DNA, 20 bp, Artificial Sequence
  Description of Artificial Sequence Synthetic oligonucleotide
aacagcttatatacactatc

SEQ ID NO: 199 - DNA, 20 bp, Artificial Sequence
  Description of Artificial Sequence Synthetic oligonucleotide
atacactatctgtgcaattt

SEQ ID NO: 200 - DNA, 20 bp, Artificial Sequence
  Description of Artificial Sequence Synthetic oligonucleotide
tgtgcaatttttcttggctg

SEQ ID NO: 201 - DNA, 20 bp, Artificial Sequence
  Description of Artificial Sequence Synthetic oligonucleotide
ttcttggctggggtatttaa

SEQ ID NO: 202 - DNA, 20 bp, Artificial Sequence
  Description of Artificial Sequence Synthetic oligonucleotide
gggtatttaaatgcaagagc

SEQ ID NO: 203 - DNA, 20 bp, Artificial Sequence
  Description of Artificial Sequence Synthetic oligonucleotide
atgcaagagctcctccaaaa

SEQ ID NO: 204 - DNA, 20 bp, Artificial Sequence
  Description of Artificial Sequence Synthetic oligonucleotide
tcctccaaaataagaagcca

SEQ ID NO: 205 - DNA, 20 bp, Artificial Sequence
  Description of Artificial Sequence Synthetic oligonucleotide
taagaagccacattcattct

SEQ ID NO: 206 - DNA, 20 bp, Artificial Sequence
  Description of Artificial Sequence Synthetic oligonucleotide
cattcattcttgtccaaatc

SEQ ID NO: 207 - DNA, 20 bp, Artificial Sequence
  Description of Artificial Sequence Synthetic oligonucleotide
tgtccaaatccctctgtctc

SEQ ID NO: 208 - DNA, 20 bp, Artificial Sequence
  Description of Artificial Sequence Synthetic oligonucleotide
cctctgtctcttctattgca

SEQ ID NO: 209 - DNA, 20 bp, Artificial Sequence
  Description of Artificial Sequence Synthetic oligonucleotide
ttctattgcaaaaagaagtt

SEQ ID NO: 210 - DNA, 20 bp, Artificial Sequence
  Description of Artificial Sequence Synthetic oligonucleotide
aaaagaagtttttcctttag

SEQ ID NO: 211 - DNA, 20 bp, Artificial Sequence
  Description of Artificial Sequence Synthetic oligonucleotide
tttcctttagttgttcataa

SEQ ID NO: 212 - DNA, 20 bp, Artificial Sequence
  Description of Artificial Sequence Synthetic oligonucleotide
ttgttcataactcttatatg

SEQ ID NO: 213 - DNA, 20 bp, Artificial Sequence
  Description of Artificial Sequence Synthetic oligonucleotide
ctcttatatggtggtagatc

SEQ ID NO: 214 - DNA, 20 bp, Artificial Sequence
  Description of Artificial Sequence Synthetic oligonucleotide
gtggtagatccaagcgatta

SEQ ID NO: 215 - DNA, 20 bp, Artificial Sequence
  Description of Artificial Sequence Synthetic oligonucleotide
caagcgattaaaacatgtat

SEQ ID NO: 216 - DNA, 20 bp, Artificial Sequence
  Description of Artificial Sequence Synthetic oligonucleotide
aaacatgtatggcttcttgg

SEQ ID NO: 217 - DNA, 20 bp, Artificial Sequence
  Description of Artificial Sequence Synthetic oligonucleotide
ggcttcttggtaaccaagtg

SEQ ID NO: 218 - DNA, 20 bp, Artificial Sequence
  Description of Artificial Sequence Synthetic oligonucleotide
taaccaagtgtctttgccaa

SEQ ID NO: 219 - DNA, 20 bp, Artificial Sequence
  Description of Artificial Sequence Synthetic oligonucleotide
tctttgccaactttttcaat

SEQ ID NO: 220 - DNA, 20 bp, Artificial Sequence
  Description of Artificial Sequence Synthetic oligonucleotide
ctttttcaatgcaaaacttt

SEQ ID NO: 221 - DNA, 20 bp, Artificial Sequence
  Description of Artificial Sequence Synthetic oligonucleotide
gcaaaacttttgaggcccat

SEQ ID NO: 222 - DNA, 20 bp, Artificial Sequence
  Description of Artificial Sequence Synthetic oligonucleotide
tgaggcccattacttcccat

SEQ ID NO: 223 - DNA, 20 bp, Artificial Sequence
  Description of Artificial Sequence Synthetic oligonucleotide
tacttcccatgagctcagca

SEQ ID NO: 224 - DNA, 20 bp, Artificial Sequence
  Description of Artificial Sequence Synthetic oligonucleotide
gagctcagcaaatcctccta

SEQ ID NO: 225 - DNA, 20 bp, Artificial Sequence
  Description of Artificial Sequence Synthetic oligonucleotide
aatcctcctagaggtaaacg

SEQ ID NO: 226 - DNA, 20 bp, Artificial Sequence
  Description of Artificial Sequence Synthetic oligonucleotide
gaggtaaacggcaggttcca

SEQ ID NO: 227 - DNA, 20 bp, Artificial Sequence
  Description of Artificial Sequence Synthetic oligonucleotide
gcaggttccagtgacgaact

SEQ ID NO: 228 - DNA, 20 bp, Artificial Sequence
  Description of Artificial Sequence Synthetic oligonucleotide
gtgacgaactgcaatagtcg

SEQ ID NO: 229 - DNA, 20 bp, Artificial Sequence
  Description of Artificial Sequence Synthetic oligonucleotide
gcaatagtcgcattcttact

SEQ ID NO: 230 - DNA, 20 bp, Artificial Sequence
  Description of Artificial Sequence Synthetic oligonucleotide
cattcttacttcattgtctg

SEQ ID NO: 231 - DNA, 20 bp, Artificial Sequence
  Description of Artificial Sequence Synthetic oligonucleotide
tcattgtctgtctctttcac

SEQ ID NO: 232 - DNA, 20 bp, Artificial Sequence
  Description of Artificial Sequence Synthetic oligonucleotide
tctctttcacaaactgccaa

SEQ ID NO: 233 - DNA, 20 bp, Artificial Sequence
  Description of Artificial Sequence Synthetic oligonucleotide
aaactgccaaaaccaaatga

SEQ ID NO: 234 - DNA, 20 bp, Artificial Sequence
  Description of Artificial Sequence Synthetic oligonucleotide
aaccaaatgatttgcttgct

SEQ ID NO: 235 - DNA, 20 bp, Artificial Sequence
  Description of Artificial Sequence Synthetic oligonucleotide
tttgcttgctgtttcttgta

SEQ ID NO: 236 - DNA, 20 bp, Artificial Sequence
  Description of Artificial Sequence Synthetic oligonucleotide
gtttcttgtataatgtcgat

SEQ ID NO: 237 - DNA, 20 bp, Artificial Sequence
  Description of Artificial Sequence Synthetic oligonucleotide
taatgtcgataaacagtatt

SEQ ID NO: 238 - DNA, 20 bp, Artificial Sequence
  Description of Artificial Sequence Synthetic oligonucleotide
aaacagtatttctctgccaa

SEQ ID NO: 239 - DNA, 20 bp, Artificial Sequence
  Description of Artificial Sequence Synthetic oligonucleotide
tctctgccaatctgccaagt

SEQ ID NO: 240 - DNA, 20 bp, Artificial Sequence
  Description of Artificial Sequence Synthetic oligonucleotide
tctgccaagtcaacctcctg

SEQ ID NO: 241 - DNA, 20 bp, Artificial Sequence
  Description of Artificial Sequence Synthetic oligonucleotide
caacctcctgcatgccacac

SEQ ID NO: 242 - DNA, 20 bp, Artificial Sequence
  Description of Artificial Sequence Synthetic oligonucleotide
catgccacacaacataacct

SEQ ID NO: 243 - DNA, 20 bp, Artificial Sequence
  Description of Artificial Sequence Synthetic oligonucleotide
aacataacctctaattcttt

SEQ ID NO: 244 - DNA, 20 bp, Artificial Sequence
  Description of Artificial Sequence Synthetic oligonucleotide
ctaattctttttcatcgaag

SEQ ID NO: 245 - DNA, 20 bp, Artificial Sequence
  Description of Artificial Sequence Synthetic oligonucleotide
ttcatcgaagtactgtagcc

SEQ ID NO: 246 - DNA, 20 bp, Artificial Sequence
  Description of Artificial Sequence Synthetic oligonucleotide
tactgtagccactgaagagg

SEQ ID NO: 247 - DNA, 20 bp, Artificial Sequence
  Description of Artificial Sequence Synthetic oligonucleotide
actgaagaggaacaacttca

SEQ ID NO: 248 - DNA, 20 bp, Artificial Sequence
  Description of Artificial Sequence Synthetic oligonucleotide
aacaacttcattaaaaccat

SEQ ID NO: 249 - DNA, 20 bp, Artificial Sequence
  Description of Artificial Sequence Synthetic oligonucleotide
ttaaaaccatcaaggaaagc

SEQ ID NO: 250 - DNA, 20 bp, Artificial Sequence
  Description of Artificial Sequence Synthetic oligonucleotide
caaggaaagctttggtctgt

SEQ ID NO: 251 - DNA, 20 bp, Artificial Sequence
  Description of Artificial Sequence Synthetic oligonucleotide
tttggtctgttcttgtactc

SEQ ID NO: 252 - DNA, 20 bp, Artificial Sequence
  Description of Artificial Sequence Synthetic oligonucleotide
tcttgtactcctcgagaaaa

SEQ ID NO: 253 - DNA, 20 bp, Artificial Sequence
  Description of Artificial Sequence Synthetic oligonucleotide
ctcgagaaaaacgccattct

SEQ ID NO: 254 - DNA, 20 bp, Artificial Sequence
  Description of Artificial Sequence Synthetic oligonucleotide
acgccattctgtcattaaac

SEQ ID NO: 255 - DNA, 20 bp, Artificial Sequence
  Description of Artificial Sequence Synthetic oligonucleotide
gtcattaaaccaatatattc

SEQ ID NO: 256 - DNA, 20 bp, Artificial Sequence
  Description of Artificial Sequence Synthetic oligonucleotide
caatatattcatctttgttc

SEQ ID NO: 257 - DNA, 20 bp, Artificial Sequence
  Description of Artificial Sequence Synthetic oligonucleotide
atctttgttctcctcagtca

SEQ ID NO: 258 - DNA, 20 bp, Artificial Sequence
  Description of Artificial Sequence Synthetic oligonucleotide
tcctcagtcaccagaatatt

SEQ ID NO: 259 - DNA, 20 bp, Artificial Sequence
  Description of Artificial Sequence Synthetic oligonucleotide
ccagaatattggaacctccc

SEQ ID NO: 260 - DNA, 20 bp, Artificial Sequence
  Description of Artificial Sequence Synthetic oligonucleotide
ggaacctcccaacttcaggt

SEQ ID NO: 261 - DNA, 20 bp, Artificial Sequence
  Description of Artificial Sequence Synthetic oligonucleotide
aacttcaggtcatgtgaagt

SEQ ID NO: 262 - DNA, 20 bp, Artificial Sequence
  Description of Artificial Sequence Synthetic oligonucleotide
catgtgaagtaacttttccc

SEQ ID NO: 263 - DNA, 20 bp, Artificial Sequence
  Description of Artificial Sequence Synthetic oligonucleotide
aacttttcccaaaatctcca

SEQ ID NO: 264 - DNA, 20 bp, Artificial Sequence
  Description of Artificial Sequence Synthetic oligonucleotide
aaaatctccatgtcaacaga

SEQ ID NO: 265 - DNA, 20 bp, Artificial Sequence
  Description of Artificial Sequence Synthetic oligonucleotide
tgtcaacagaaaagtacatt

SEQ ID NO: 266 - DNA, 20 bp, Artificial Sequence
  Description of Artificial Sequence Synthetic oligonucleotide
aaagtacatttctaagccac

SEQ ID NO: 267 - DNA, 20 bp, Artificial Sequence
  Description of Artificial Sequence Synthetic oligonucleotide
tctaagccacattcttcaat

SEQ ID NO: 268 - DNA, 20 bp, Artificial Sequence
  Description of Artificial Sequence Synthetic oligonucleotide
attcttcaatgttgttatct

SEQ ID NO: 269 - DNA, 20 bp, Artificial Sequence
  Description of Artificial Sequence Synthetic oligonucleotide
gttgttatctcttatccaga

SEQ ID NO: 270 - DNA, 20 bp, Artificial Sequence
  Description of Artificial Sequence Synthetic oligonucleotide
cttatccagataagggagtt

SEQ ID NO: 271 - DNA, 20 bp, Artificial Sequence
  Description of Artificial Sequence Synthetic oligonucleotide
taagggagttataaaattca

SEQ ID NO: 272 - DNA, 20 bp, Artificial Sequence
  Description of Artificial Sequence Synthetic oligonucleotide
ataaaattcagtatcaatag

SEQ ID NO: 273 - DNA, 20 bp, Artificial Sequence
  Description of Artificial Sequence Synthetic oligonucleotide
gtatcaatagattccaaatc

SEQ ID NO: 274 - DNA, 20 bp, Artificial Sequence
  Description of Artificial Sequence Synthetic oligonucleotide
attccaaatccttaatagta

SEQ ID NO: 275 - DNA, 20 bp, Artificial Sequence
  Description of Artificial Sequence Synthetic oligonucleotide
cttaatagtaagttttttac

SEQ ID NO: 276 - DNA, 20 bp, Artificial Sequence
  Description of Artificial Sequence Synthetic oligonucleotide
agttttttacttaacatacg

SEQ ID NO: 277 - DNA, 20 bp, Artificial Sequence
  Description of Artificial Sequence Synthetic oligonucleotide
ttaacatacgcttgtagaat

SEQ ID NO: 278 - DNA, 20 bp, Artificial Sequence
  Description of Artificial Sequence Synthetic oligonucleotide
cttgtagaatggtaaagaga

SEQ ID NO: 279 - DNA, 20 bp, Artificial Sequence
  Description of Artificial Sequence Synthetic oligonucleotide
ggtaaagagaaaccagtatc

SEQ ID NO: 280 - DNA, 20 bp, Artificial Sequence
  Description of Artificial Sequence Synthetic oligonucleotide
aaccagtatcgataaacttt

SEQ ID NO: 281 - DNA, 20 bp, Artificial Sequence
  Description of Artificial Sequence Synthetic oligonucleotide
gataaactttccatgaaata

SEQ ID NO: 282 - DNA, 20 bp, Artificial Sequence
  Description of Artificial Sequence Synthetic oligonucleotide
ccatgaaatagtgccatggc

SEQ ID NO: 283 - DNA, 20 bp, Artificial Sequence
  Description of Artificial Sequence Synthetic oligonucleotide
gtgccatggcaataaaacga

SEQ ID NO: 284 - DNA, 20 bp, Artificial Sequence
  Description of Artificial Sequence Synthetic oligonucleotide
aataaaacgaccaatgaaac

SEQ ID NO: 285 - DNA, 20 bp, Artificial Sequence
  Description of Artificial Sequence Synthetic oligonucleotide
ccaatgaaacagaagtatga

SEQ ID NO: 286 - DNA, 20 bp, Artificial Sequence
  Description of Artificial Sequence Synthetic oligonucleotide
agaagtatgaaagatggtct

SEQ ID NO: 287 - DNA, 20 bp, Artificial Sequence
  Description of Artificial Sequence Synthetic oligonucleotide
aagatggtctggattaatgg

SEQ ID NO: 288 - DNA, 20 bp, Artificial Sequence
  Description of Artificial Sequence Synthetic oligonucleotide
ggattaatggttgatgctgg

SEQ ID NO: 289 - DNA, 20 bp, Artificial Sequence
  Description of Artificial Sequence Synthetic oligonucleotide
ttgatgctggatttatctgc

SEQ ID NO: 290 - DNA, 20 bp, Artificial Sequence
  Description of Artificial Sequence Synthetic oligonucleotide
atttatctgcagacaatagt

SEQ ID NO: 291 - DNA, 20 bp, Artificial Sequence
  Description of Artificial Sequence Synthetic oligonucleotide
agacaatagttgttcttgcc

SEQ ID NO: 292 - DNA, 20 bp, Artificial Sequence
  Description of Artificial Sequence Synthetic oligonucleotide
tgttcttgcccgcatactca

SEQ ID NO: 293 - DNA, 20 bp, Artificial Sequence
  Description of Artificial Sequence Synthetic oligonucleotide
cgcatactcaaataagcaat

SEQ ID NO: 294 - DNA, 20 bp, Artificial Sequence
  Description of Artificial Sequence Synthetic oligonucleotide
aataagcaatacattgggtt

SEQ ID NO: 295 - DNA, 20 bp, Artificial Sequence
  Description of Artificial Sequence Synthetic oligonucleotide
acattgggttcaaaacttca

SEQ ID NO: 296 - DNA, 20 bp, Artificial Sequence
  Description of Artificial Sequence Synthetic oligonucleotide
caaaacttcatgtgaaagca

SEQ ID NO: 297 - DNA, 20 bp, Artificial Sequence
  Description of Artificial Sequence Synthetic oligonucleotide
tgtgaaagcaagaaaaacca

SEQ ID NO: 298 - DNA, 20 bp, Artificial Sequence
  Description of Artificial Sequence Synthetic oligonucleotide
agaaaaaccattctctcgct

SEQ ID NO: 299 - DNA, 20 bp, Artificial Sequence
  Description of Artificial Sequence Synthetic oligonucleotide
ttctctcgctaggccaccat

SEQ ID NO: 300 - DNA, 20 bp, Artificial Sequence
  Description of Artificial Sequence Synthetic oligonucleotide
aggccaccataatcaagtcc

SEQ ID NO: 301 - DNA, 20 bp, Artificial Sequence
  Description of Artificial Sequence Synthetic oligonucleotide
aatcaagtccttcttctcct

SEQ ID NO: 302 - DNA, 20 bp, Artificial Sequence
  Description of Artificial Sequence Synthetic oligonucleotide
ttcttctcctctaaatatta

SEQ ID NO: 303 - DNA, 20 bp, Artificial Sequence
  Description of Artificial Sequence Synthetic oligonucleotide
ctaaatattacatataagcg

SEQ ID NO: 304 - DNA, 20 bp, Artificial Sequence
  Description of Artificial Sequence Synthetic oligonucleotide
catataagcgcctcctcaag

SEQ ID NO: 305 - DNA, 20 bp, Artificial Sequence
  Description of Artificial Sequence Synthetic oligonucleotide
cctcctcaagtcatagggtt

SEQ ID NO: 306 - DNA, 20 bp, Artificial Sequence
  Description of Artificial Sequence Synthetic oligonucleotide
tcatagggttttaatgccat

SEQ ID NO: 307 - DNA, 20 bp, Artificial Sequence
  Description of Artificial Sequence Synthetic oligonucleotide
ttaatgccataatctgttgg

SEQ ID NO: 308 - DNA, 20 bp, Artificial Sequence
  Description of Artificial Sequence Synthetic oligonucleotide
aatctgttggaaggaatctt

SEQ ID NO: 309 - DNA, 20 bp, Artificial Sequence
  Description of Artificial Sequence Synthetic oligonucleotide
aaggaatcttcaaacaatgt

SEQ ID NO: 310 - DNA, 20 bp, Artificial Sequence
  Description of Artificial Sequence Synthetic oligonucleotide
caaacaatgtctgccgggac

SEQ ID NO: 311 - DNA, 20 bp, Artificial Sequence
  Description of Artificial Sequence Synthetic oligonucleotide
ctgccgggacacattgatct

SEQ ID NO: 312 - DNA, 20 bp, Artificial Sequence
  Description of Artificial Sequence Synthetic oligonucleotide
acattgatctttacatgact

SEQ ID NO: 313 - DNA, 20 bp, Artificial Sequence
  Description of Artificial Sequence Synthetic oligonucleotide
ttacatgactaggtagtgca

SEQ ID NO: 314 - DNA, 20 bp, Artificial Sequence
  Description of Artificial Sequence Synthetic oligonucleotide
aggtagtgcattagactggc

SEQ ID NO: 315 - DNA, 20 bp, Artificial Sequence
  Description of Artificial Sequence Synthetic oligonucleotide
ttagactggcacaaataacg

SEQ ID NO: 316 - DNA, 20 bp, Artificial Sequence
  Description of Artificial Sequence Synthetic oligonucleotide
acaaataacggaagtgagca

SEQ ID NO: 317 - DNA, 20 bp, Artificial Sequence
  Description of Artificial Sequence Synthetic oligonucleotide
gaagtgagcaagcttccacc

SEQ ID NO: 318 - DNA, 20 bp, Artificial Sequence
  Description of Artificial Sequence Synthetic oligonucleotide
agcttccacctaaagccgcg

SEQ ID NO: 319 - DNA, 20 bp, Artificial Sequence
  Description of Artificial Sequence Synthetic oligonucleotide
taaagccgcgttcataagca

SEQ ID NO: 320 - DNA, 20 bp, Artificial Sequence
  Description of Artificial Sequence Synthetic oligonucleotide
ttcataagcaatttgtggac

SEQ ID NO: 321 - DNA, 20 bp, Artificial Sequence
  Description of Artificial Sequence Synthetic oligonucleotide
atttgtggaccacctttagt

SEQ ID NO: 322 - DNA, 20 bp, Artificial Sequence
  Description of Artificial Sequence Synthetic oligonucleotide
cacctttagttacagatgac

SEQ ID NO: 323 - DNA, 20 bp, Artificial Sequence
  Description of Artificial Sequence Synthetic oligonucleotide
tacagatgacttcccattgc

SEQ ID NO: 324 - DNA, 20 bp, Artificial Sequence
  Description of Artificial Sequence Synthetic oligonucleotide
ttcccattgcgaggatcttt

SEQ ID NO: 325 - DNA, 20 bp, Artificial Sequence
  Description of Artificial Sequence Synthetic oligonucleotide
gaggatctttgaatgttgtt

SEQ ID NO: 326 - DNA, 20 bp, Artificial Sequence
  Description of Artificial Sequence Synthetic oligonucleotide
gaatgttgttgttcttgtgt

SEQ ID NO: 327 - DNA, 20 bp, Artificial Sequence
  Description of Artificial Sequence Synthetic oligonucleotide
gttcttgtgttatgatcaac

SEQ ID NO: 328 - DNA, 20 bp, Artificial Sequence
  Description of Artificial Sequence Synthetic oligonucleotide
tatgatcaacaaagtacctt

SEQ ID NO: 329 - DNA, 20 bp, Artificial Sequence
  Description of Artificial Sequence Synthetic oligonucleotide
aaagtaccttacaccttcac

SEQ ID NO: 330 - DNA, 20 bp, Artificial Sequence
  Description of Artificial Sequence Synthetic oligonucleotide
acaccttcacgagtatatct

SEQ ID NO: 331 - DNA, 20 bp, Artificial Sequence
  Description of Artificial Sequence Synthetic oligonucleotide
gagtatatctaatttcccag

SEQ ID NO: 332 - DNA, 20 bp, Artificial Sequence
  Description of Artificial Sequence Synthetic oligonucleotide
aatttcccagccttctggca

SEQ ID NO: 333 - DNA, 20 bp, Artificial Sequence
  Description of Artificial Sequence Synthetic oligonucleotide
ccttctggcaggggttcttc

SEQ ID NO: 334 - DNA, 20 bp, Artificial Sequence
  Description of Artificial Sequence Synthetic oligonucleotide
ggggttcttcattctgtaag

SEQ ID NO: 335 - DNA, 20 bp, Artificial Sequence
  Description of Artificial Sequence Synthetic oligonucleotide
attctgtaagccttgagttc

SEQ ID NO: 336 - DNA, 20 bp, Artificial Sequence
  Description of Artificial Sequence Synthetic oligonucleotide
ccttgagttcttggatcttc

SEQ ID NO: 337 - DNA, 20 bp, Artificial Sequence
  Description of Artificial Sequence Synthetic oligonucleotide
ttggatcttcccactgggtt

SEQ ID NO: 338 - DNA, 20 bp, Artificial Sequence
  Description of Artificial Sequence Synthetic oligonucleotide
ccactgggttgtttttgtgt

SEQ ID NO: 339 - DNA, 20 bp, Artificial Sequence
  Description of Artificial Sequence Synthetic oligonucleotide
gtttttgtgttatgattcac

SEQ ID NO: 340 - DNA, 20 bp, Artificial Sequence
  Description of Artificial Sequence Synthetic oligonucleotide
tatgattcacaaagtaaacc

SEQ ID NO: 341 - DNA, 20 bp, Artificial Sequence
  Description of Artificial Sequence Synthetic oligonucleotide
aaagtaaaccctgtctgttg

SEQ ID NO: 342 - DNA, 20 bp, Artificial Sequence
  Description of Artificial Sequence Synthetic oligonucleotide
ctgtctgttgaatccactct

SEQ ID NO: 343 - DNA, 20 bp, Artificial Sequence
  Description of Artificial Sequence Synthetic oligonucleotide
aatccactcttttttcccag

SEQ ID NO: 344 - DNA, 20 bp, Artificial Sequence
  Description of Artificial Sequence Synthetic oligonucleotide
tttttcccagcctggtggca

SEQ ID NO: 345 - DNA, 20 bp, Artificial Sequence
  Description of Artificial Sequence Synthetic oligonucleotide
cctggtggcaaaggtccata

SEQ ID NO: 346 - DNA, 20 bp, Artificial Sequence
  Description of Artificial Sequence Synthetic oligonucleotide
aaggtccataagggtcattt

SEQ ID NO: 347 - DNA, 20 bp, Artificial Sequence
  Description of Artificial Sequence Synthetic oligonucleotide
agggtcattttctgcagcta

SEQ ID NO: 348 - DNA, 20 bp, Artificial Sequence
  Description of Artificial Sequence Synthetic oligonucleotide
tctgcagctaacattgaagc

SEQ ID NO: 349 - DNA, 20 bp, Artificial Sequence
  Description of Artificial Sequence Synthetic oligonucleotide
acattgaagccgaatagagg

SEQ ID NO: 350 - DNA, 20 bp, Artificial Sequence
  Description of Artificial Sequence Synthetic oligonucleotide
cgaatagaggtatcgttggt

SEQ ID NO: 351 - DNA, 20 bp, Artificial Sequence
  Description of Artificial Sequence Synthetic oligonucleotide
tatcgttggttaaactgttg

SEQ ID NO: 352 - DNA, 20 bp, Artificial Sequence
  Description of Artificial Sequence Synthetic oligonucleotide
taaactgttgcatagctccc

SEQ ID NO: 353 - DNA, 20 bp, Artificial Sequence
  Description of Artificial Sequence Synthetic oligonucleotide
catagctccctgcaattggt

SEQ ID NO: 354 - DNA, 20 bp, Artificial Sequence
  Description of Artificial Sequence Synthetic oligonucleotide
tgcaattggttccgctgaga

SEQ ID NO: 355 - DNA, 20 bp, Artificial Sequence
  Description of Artificial Sequence Synthetic oligonucleotide
tccgctgagattgccactgt

SEQ ID NO: 356 - DNA, 20 bp, Artificial Sequence
  Description of Artificial Sequence Synthetic oligonucleotide
ttgccactgttcaaaatttc

SEQ ID NO: 357 - DNA, 20 bp, Artificial Sequence
  Description of Artificial Sequence Synthetic oligonucleotide
tcaaaatttcggacagattc

SEQ ID NO: 358 - DNA, 20 bp, Artificial Sequence
  Description of Artificial Sequence Synthetic oligonucleotide
ggacagattccatggtaggc

SEQ ID NO: 359 - DNA, 20 bp, Artificial Sequence
  Description of Artificial Sequence Synthetic oligonucleotide
catggtaggccgctgccacg

SEQ ID NO: 360 - DNA, 20 bp, Artificial Sequence
  Description of Artificial Sequence Synthetic oligonucleotide
cgctgccacgttgttgttct

SEQ ID NO: 361 - DNA, 20 bp, Artificial Sequence
  Description of Artificial Sequence Synthetic oligonucleotide
ttgttgttctggtgttatga

SEQ ID NO: 362 - DNA, 20 bp, Artificial Sequence
  Description of Artificial Sequence Synthetic oligonucleotide
ggtgttatgatccacataat

SEQ ID NO: 363 - DNA, 20 bp, Artificial Sequence
  Description of Artificial Sequence Synthetic oligonucleotide
tccacataataaactcttct

SEQ ID NO: 364 - DNA, 20 bp, Artificial Sequence
  Description of Artificial Sequence Synthetic oligonucleotide
aaactcttctacgatcatca

SEQ ID NO: 365 - DNA, 20 bp, Artificial Sequence
  Description of Artificial Sequence Synthetic oligonucleotide
acgatcatcaactcttcttt

SEQ ID NO: 366 - DNA, 20 bp, Artificial Sequence
  Description of Artificial Sequence Synthetic oligonucleotide
actcttctttcccaacctgg

SEQ ID NO: 367 - DNA, 20 bp, Artificial Sequence
  Description of Artificial Sequence Synthetic oligonucleotide
cccaacctggaggtaaaggt

SEQ ID NO: 368 - DNA, 20 bp, Artificial Sequence
  Description of Artificial Sequence Synthetic oligonucleotide
aggtaaaggttgtggtctct

SEQ ID NO: 369 - DNA, 20 bp, Artificial Sequence
  Description of Artificial Sequence Synthetic oligonucleotide
tgtggtctctcccatgtggt

SEQ ID NO: 370 - DNA, 20 bp, Artificial Sequence
  Description of Artificial Sequence Synthetic oligonucleotide
cccatgtggtagttcgagta

SEQ ID NO: 371 - DNA, 20 bp, Artificial Sequence
  Description of Artificial Sequence Synthetic oligonucleotide
agttcgagtattatgatcca

SEQ ID NO: 372 - DNA, 20 bp, Artificial Sequence
  Description of Artificial Sequence Synthetic oligonucleotide
ttatgatccacataataggt

SEQ ID NO: 373 - DNA, 20 bp, Artificial Sequence
  Description of Artificial Sequence Synthetic oligonucleotide
cataataggttctaccatga

SEQ ID NO: 374 - DNA, 20 bp, Artificial Sequence
  Description of Artificial Sequence Synthetic oligonucleotide
tctaccatgaggatcttttc

SEQ ID NO: 375 - DNA, 20 bp, Artificial Sequence
  Description of Artificial Sequence Synthetic oligonucleotide
ggatcttttctttgttccca

SEQ ID NO: 376 - DNA, 20 bp, Artificial Sequence
  Description of Artificial Sequence Synthetic oligonucleotide
tttgttcccaccctgatggc

SEQ ID NO: 377 - DNA, 20 bp, Artificial Sequence
  Description of Artificial Sequence Synthetic oligonucleotide
ccctgatggcaaggtttctg

SEQ ID NO: 378 - DNA, 20 bp, Artificial Sequence
  Description of Artificial Sequence Synthetic oligonucleotide
aaggtttctgtgttggcatt

SEQ ID NO: 379 - DNA, 20 bp, Artificial Sequence
  Description of Artificial Sequence Synthetic oligonucleotide
tgttggcattcccagactgc

SEQ ID NO: 380 - DNA, 20 bp, Artificial Sequence
  Description of Artificial Sequence Synthetic oligonucleotide
cccagactgctgccgtacag

SEQ ID NO: 381 - DNA, 20 bp, Artificial Sequence
  Description of Artificial Sequence Synthetic oligonucleotide
tgccgtacaggatccataca

SEQ ID NO: 382 - DNA, 20 bp, Artificial Sequence
  Description of Artificial Sequence Synthetic oligonucleotide
gatccatacacccatctggc

SEQ ID NO: 383 - DNA, 20 bp, Artificial Sequence
  Description of Artificial Sequence Synthetic oligonucleotide
cccatctggctgtcttgatt

SEQ ID NO: 384 - DNA, 20 bp, Artificial Sequence
  Description of Artificial Sequence Synthetic oligonucleotide
tgtcttgatttggctgcttc

SEQ ID NO: 385 - DNA, 20 bp, Artificial Sequence
  Description of Artificial Sequence Synthetic oligonucleotide
tggctgcttcaaaagcagaa

SEQ ID NO: 386 - DNA, 20 bp, Artificial Sequence
  Description of Artificial Sequence Synthetic oligonucleotide
aaaagcagaactacttctag

SEQ ID NO: 387 - DNA, 20 bp, Artificial Sequence
  Description of Artificial Sequence Synthetic oligonucleotide
ctacttctagaattagaggt

SEQ ID NO: 388 - DNA, 20 bp, Artificial Sequence
  Description of Artificial Sequence Synthetic oligonucleotide
aattagaggtgtcaggctct

SEQ ID NO: 389 - DNA, 20 bp, Artificial Sequence
  Description of Artificial Sequence Synthetic oligonucleotide
gtcaggctctaatatacttc

SEQ ID NO: 390 - DNA, 20 bp, Artificial Sequence
  Description of Artificial Sequence Synthetic oligonucleotide
aatatacttctagcttcaga

SEQ ID NO: 391 - DNA, 20 bp, Artificial Sequence
  Description of Artificial Sequence Synthetic oligonucleotide
tagcttcagattccaattct

SEQ ID NO: 392 - DNA, 20 bp, Artificial Sequence
  Description of Artificial Sequence Synthetic oligonucleotide
ttccaattctgcactggtag

SEQ ID NO: 393 - DNA, 20 bp, Artificial Sequence
  Description of Artificial Sequence Synthetic oligonucleotide
gcactggtagaaggaataca

SEQ ID NO: 394 - DNA, 20 bp, Artificial Sequence
  Description of Artificial Sequence Synthetic oligonucleotide
aaggaatacattcattgttt

SEQ ID NO: 395 - DNA, 20 bp, Artificial Sequence
  Description of Artificial Sequence Synthetic oligonucleotide
ttcattgttttctgaggaag

SEQ ID NO: 396 - DNA, 20 bp, Artificial Sequence
  Description of Artificial Sequence Synthetic oligonucleotide
tctgaggaagtcagtatttc

SEQ ID NO: 397 - DNA, 20 bp, Artificial Sequence
  Description of Artificial Sequence Synthetic oligonucleotide
tcagtatttcttgaactgga

SEQ ID NO: 398 - DNA, 20 bp, Artificial Sequence
  Description of Artificial Sequence Synthetic oligonucleotide
ttgaactggaggatcttcaa

SEQ ID NO: 399 - DNA, 20 bp, Artificial Sequence
  Description of Artificial Sequence Synthetic oligonucleotide
ggatcttcaacagtagtact

SEQ ID NO: 400 - DNA, 20 bp, Artificial Sequence
  Description of Artificial Sequence Synthetic oligonucleotide
cagtagtactagtgcaattt

SEQ ID NO: 401 - DNA, 20 bp, Artificial Sequence
  Description of Artificial Sequence Synthetic oligonucleotide
agtgcaatttggagacaagg

SEQ ID NO: 402 - DNA, 20 bp, Artificial Sequence
  Description of Artificial Sequence Synthetic oligonucleotide
ggagacaaggcattttcttc

SEQ ID NO: 403 - DNA, 20 bp, Artificial Sequence
  Description of Artificial Sequence Synthetic oligonucleotide
cattttcttcagacactact

SEQ ID NO: 404 - DNA, 20 bp, Artificial Sequence
  Description of Artificial Sequence Synthetic oligonucleotide
agacactactggagtacccg

SEQ ID NO: 405 - DNA, 20 bp, Artificial Sequence
  Description of Artificial Sequence Synthetic oligonucleotide
ggagtacccgtgacagacgc

SEQ ID NO: 406 - DNA, 20 bp, Artificial Sequence
  Description of Artificial Sequence Synthetic oligonucleotide
tgacagacgcattatcagtt

SEQ ID NO: 407 - DNA, 20 bp, Artificial Sequence
  Description of Artificial Sequence Synthetic oligonucleotide
attatcagttggtgcaaatg

SEQ ID NO: 408 - DNA, 20 bp, Artificial Sequence
  Description of Artificial Sequence Synthetic oligonucleotide
ggtgcaaatgaggatgattc

SEQ ID NO: 409 - DNA, 20 bp, Artificial Sequence
  Description of Artificial Sequence Synthetic oligonucleotide
aggatgattctccattaaca

SEQ ID NO: 410 - DNA, 20 bp, Artificial Sequence
  Description of Artificial Sequence Synthetic oligonucleotide
tccattaacagtgtcatcgg

SEQ ID NO: 411 - DNA, 20 bp, Artificial Sequence
  Description of Artificial Sequence Synthetic oligonucleotide
gtgtcatcggcaggctcaga

SEQ ID NO: 412 - DNA, 20 bp, Artificial Sequence
  Description of Artificial Sequence Synthetic oligonucleotide
caggctcagatgcgagtggt

SEQ ID NO: 413 - DNA, 20 bp, Artificial Sequence
  Description of Artificial Sequence Synthetic oligonucleotide
tgcgagtggttttggagctg

SEQ ID NO: 414 - DNA, 20 bp, Artificial Sequence
  Description of Artificial Sequence Synthetic oligonucleotide
tttggagctggtgtattttt

SEQ ID NO: 415 - DNA, 20 bp, Artificial Sequence
  Description of Artificial Sequence Synthetic oligonucleotide
gtgtatttttgggtctggca

SEQ ID NO: 416 - DNA, 20 bp, Artificial Sequence
  Description of Artificial Sequence Synthetic oligonucleotide
gggtctggcagcaacctgag

SEQ ID NO: 417 - DNA, 20 bp, Artificial Sequence
  Description of Artificial Sequence Synthetic oligonucleotide
gcaacctgagacggagatga

SEQ ID NO: 418 - DNA, 20 bp, Artificial Sequence
  Description of Artificial Sequence Synthetic oligonucleotide
acggagatgaaggtgtgttg

SEQ ID NO: 419 - DNA, 20 bp, Artificial Sequence
  Description of Artificial Sequence Synthetic oligonucleotide
aggtgtgttgtctccattaa

SEQ ID NO: 420 - DNA, 20 bp, Artificial Sequence
  Description of Artificial Sequence Synthetic oligonucleotide
tctccattaactacatacga

SEQ ID NO: 421 - DNA, 20 bp, Artificial Sequence
  Description of Artificial Sequence Synthetic oligonucleotide
ctacatacgagcagcatgag

SEQ ID NO: 422 - DNA, 20 bp, Artificial Sequence
  Description of Artificial Sequence Synthetic oligonucleotide
gcagcatgagttttggacta

SEQ ID NO: 423 - DNA, 20 bp, Artificial Sequence
  Description of Artificial Sequence Synthetic oligonucleotide
ttttggactagagtgcttgt

SEQ ID NO: 424 - DNA, 20 bp, Artificial Sequence
  Description of Artificial Sequence Synthetic oligonucleotide
gagtgcttgtaggtacatga

SEQ ID NO: 425 - DNA, 20 bp, Artificial Sequence
  Description of Artificial Sequence Synthetic oligonucleotide
aggtacatgattatctattc

SEQ ID NO: 426 - DNA, 20 bp, Artificial Sequence
  Description of Artificial Sequence Synthetic oligonucleotide
ttatctattccattcgtgcc

SEQ ID NO: 427 - DNA, 20 bp, Artificial Sequence
  Description of Artificial Sequence Synthetic oligonucleotide
cattcgtgccttcaacagcc

SEQ ID NO: 428 - DNA, 20 bp, Artificial Sequence
  Description of Artificial Sequence Synthetic oligonucleotide
ttcaacagccaacctggcag

SEQ ID NO: 429 - DNA, 20 bp, Artificial Sequence
  Description of Artificial Sequence Synthetic oligonucleotide
aacctggcagttgtccttgc

SEQ ID NO: 430 - DNA, 20 bp, Artificial Sequence
  Description of Artificial Sequence Synthetic oligonucleotide
ttgtccttgctgaaggctct

SEQ ID NO: 431 - DNA, 20 bp, Artificial Sequence
  Description of Artificial Sequence Synthetic oligonucleotide
tgaaggctctccattttcat

SEQ ID NO: 432 - DNA, 20 bp, Artificial Sequence
  Description of Artificial Sequence Synthetic oligonucleotide
ccattttcatgtaaggcatc

SEQ ID NO: 433 - DNA, 20 bp, Artificial Sequence
  Description of Artificial Sequence Synthetic oligonucleotide
gtaaggcatcaccattttcc

SEQ ID NO: 434 - DNA, 20 bp, Artificial Sequence
  Description of Artificial Sequence Synthetic oligonucleotide
accattttcctgtatttcta

SEQ ID NO: 435 - DNA, 20 bp, Artificial Sequence
  Description of Artificial Sequence Synthetic oligonucleotide
tgtatttctatggttggaga

SEQ ID NO: 436 - DNA, 20 bp, Artificial Sequence
  Description of Artificial Sequence Synthetic oligonucleotide
tggttggagatgagctgcag

SEQ ID NO: 437 - DNA, 20 bp, Artificial Sequence
  Description of Artificial Sequence Synthetic oligonucleotide
tgagctgcagtttgttatat

SEQ ID NO: 438 - DNA, 20 bp, Artificial Sequence
  Description of Artificial Sequence Synthetic oligonucleotide
tttgttatattttcttgctc

SEQ ID NO: 439 - DNA, 20 bp, Artificial Sequence
  Description of Artificial Sequence Synthetic oligonucleotide
tttcttgctcaatcaccaat

SEQ ID NO: 440 - DNA, 20 bp, Artificial Sequence
  Description of Artificial Sequence Synthetic oligonucleotide
aatcaccaatccatcaagca

SEQ ID NO: 441 - DNA, 20 bp, Artificial Sequence
  Description of Artificial Sequence Synthetic oligonucleotide
ccatcaagcacaactgtcaa

SEQ ID NO: 442 - DNA, 20 bp, Artificial Sequence
  Description of Artificial Sequence Synthetic oligonucleotide
caactgtcaattcaccagtt

SEQ ID NO: 443 - DNA, 20 bp, Artificial Sequence
  Description of Artificial Sequence Synthetic oligonucleotide
ttcaccagtttgtgctatgc

SEQ ID NO: 444 - DNA, 20 bp, Artificial Sequence
  Description of Artificial Sequence Synthetic oligonucleotide
tgtgctatgccattcttgtt

SEQ ID NO: 445 - DNA, 20 bp, Artificial Sequence
  Description of Artificial Sequence Synthetic oligonucleotide
cattcttgttttccaaggaa

SEQ ID NO: 446 - DNA, 20 bp, Artificial Sequence
  Description of Artificial Sequence Synthetic oligonucleotide
ttccaaggaaagttttaatt

SEQ ID NO: 447 - DNA, 20 bp, Artificial Sequence
  Description of Artificial Sequence Synthetic oligonucleotide
agttttaattgttctttcac

SEQ ID NO: 448 - DNA, 20 bp, Artificial Sequence
  Description of Artificial Sequence Synthetic oligonucleotide
gttctttcactctttccaat

SEQ ID NO: 449 - DNA, 20 bp, Artificial Sequence
  Description of Artificial Sequence Synthetic oligonucleotide
tctttccaattttctattgt

SEQ ID NO: 450 - DNA, 20 bp, Artificial Sequence
  Description of Artificial Sequence Synthetic oligonucleotide
tttctattgtgtatcaacag

SEQ ID NO: 451 - DNA, 20 bp, Artificial Sequence
  Description of Artificial Sequence Synthetic oligonucleotide
gtatcaacagagcttgtttc

SEQ ID NO: 452 - DNA, 20 bp, Artificial Sequence
  Description of Artificial Sequence Synthetic oligonucleotide
agcttgtttcaaatctatcg

SEQ ID NO: 453 - DNA, 20 bp, Artificial Sequence
  Description of Artificial Sequence Synthetic oligonucleotide
aaatctatcgttgcttttcc

SEQ ID NO: 454 - DNA, 19 bp, Artificial Sequence
  Description of Artificial Sequence Synthetic oligonucleotide
tgcttttcctaataaagca

SEQ ID NO: 455 - DNA, 20 bp, Artificial Sequence
  Description of Artificial Sequence Synthetic oligonucleotide
taataaagcatctgctttta

SEQ ID NO: 456 - DNA, 20 bp, Artificial Sequence
  Description of Artificial Sequence Synthetic oligonucleotide
tctgcttttaaagtgcgatg

SEQ ID NO: 457 - DNA, 20 bp, Artificial Sequence
  Description of Artificial Sequence Synthetic oligonucleotide
aagtgcgatggctccaaact

SEQ ID NO: 458 - DNA, 20 bp, Artificial Sequence
  Description of Artificial Sequence Synthetic oligonucleotide
gctccaaacttgaaattcca

SEQ ID NO: 459 - DNA, 20 bp, Artificial Sequence
  Description of Artificial Sequence Synthetic oligonucleotide
tgaaattccaatgtagtctg

SEQ ID NO: 460 - DNA, 20 bp, Artificial Sequence
  Description of Artificial Sequence Synthetic oligonucleotide
atgtagtctgtggcgtaaca

SEQ ID NO: 461 - DNA, 20 bp, Artificial Sequence
  Description of Artificial Sequence Synthetic oligonucleotide
tggcgtaacatttacagtta

SEQ ID NO: 462 - DNA, 20 bp, Artificial Sequence
  Description of Artificial Sequence Synthetic oligonucleotide
tttacagttagctgttcatc

SEQ ID NO: 463 - DNA, 20 bp, Artificial Sequence
  Description of Artificial Sequence Synthetic oligonucleotide
gctgttcatcccattttgga

SEQ ID NO: 464 - DNA, 20 bp, Artificial Sequence
  Description of Artificial Sequence Synthetic oligonucleotide
ccattttggattagaagaac

SEQ ID NO: 465 - DNA, 20 bp, Artificial Sequence
  Description of Artificial Sequence Synthetic oligonucleotide
ttagaagaactactggattt

SEQ ID NO: 466 - DNA, 20 bp, Artificial Sequence
  Description of Artificial Sequence Synthetic oligonucleotide
tactggattttgctgttttc

SEQ ID NO: 467 - DNA, 20 bp, Artificial Sequence
  Description of Artificial Sequence Synthetic oligonucleotide
tgctgttttcgtaatttctc

SEQ ID NO: 468 - DNA, 20 bp, Artificial Sequence
  Description of Artificial Sequence Synthetic oligonucleotide
gtaatttctccatctacaac

SEQ ID NO: 469 - DNA, 20 bp, Artificial Sequence
  Description of Artificial Sequence Synthetic oligonucleotide
catctacaactacttctgta

SEQ ID NO: 470 - DNA, 20 bp, Artificial Sequence
  Description of Artificial Sequence Synthetic oligonucleotide
tacttctgtatatattgctg

SEQ ID NO: 471 - DNA, 20 bp, Artificial Sequence
  Description of Artificial Sequence Synthetic oligonucleotide
tatattgctgttccgaacca

SEQ ID NO: 472 - DNA, 20 bp, Artificial Sequence
  Description of Artificial Sequence Synthetic oligonucleotide
ttccgaaccagttctttttt

SEQ ID NO: 473 - DNA, 20 bp, Artificial Sequence
  Description of Artificial Sequence Synthetic oligonucleotide
gttcttttttcttttaagtt

SEQ ID NO: 474 - DNA, 20 bp, Artificial Sequence
  Description of Artificial Sequence Synthetic oligonucleotide
cttttaagtttggcactaga

SEQ ID NO: 475 - DNA, 20 bp, Artificial Sequence
  Description of Artificial Sequence Synthetic oligonucleotide
tggcactagaaacagttacc

SEQ ID NO: 476 - DNA, 20 bp, Artificial Sequence
  Description of Artificial Sequence Synthetic oligonucleotide
aacagttacctgtaactgca

SEQ ID NO: 477 - DNA, 20 bp, Artificial Sequence
  Description of Artificial Sequence Synthetic oligonucleotide
tgtaactgcaaccttccact

SEQ ID NO: 478 - DNA, 20 bp, Artificial Sequence
  Description of Artificial Sequence Synthetic oligonucleotide
accttccactgtggttatta

SEQ ID NO: 479 - DNA, 20 bp, Artificial Sequence
  Description of Artificial Sequence Synthetic oligonucleotide
gtggttattactagtatcag

SEQ ID NO: 480 - DNA, 20 bp, Artificial Sequence
  Description of Artificial Sequence Synthetic oligonucleotide
ctagtatcagaccttggtga

SEQ ID NO: 481 - DNA, 20 bp, Artificial Sequence
  Description of Artificial Sequence Synthetic oligonucleotide
accttggtgaagcagtggcc

SEQ ID NO: 482 - DNA, 20 bp, Artificial Sequence
  Description of Artificial Sequence Synthetic oligonucleotide
agcagtggccatgtcccaaa

SEQ ID NO: 483 - DNA, 20 bp, Artificial Sequence
  Description of Artificial Sequence Synthetic oligonucleotide
atgtcccaaaattcagctaa

SEQ ID NO: 484 - DNA, 20 bp, Artificial Sequence
  Description of Artificial Sequence Synthetic oligonucleotide
attcagctaaaactatgtat

SEQ ID NO: 485 - DNA, 20 bp, Artificial Sequence
  Description of Artificial Sequence Synthetic oligonucleotide
aactatgtatcaggaactgc

SEQ ID NO: 486 - DNA, 20 bp, Artificial Sequence
  Description of Artificial Sequence Synthetic oligonucleotide
caggaactgctagacactgg

SEQ ID NO: 487 - DNA, 20 bp, Artificial Sequence
  Description of Artificial Sequence Synthetic oligonucleotide
tagacactggggatacaaag

SEQ ID NO: 488 - DNA, 20 bp, Artificial Sequence
  Description of Artificial Sequence Synthetic oligonucleotide
ggatacaaagatgagtaaga

SEQ ID NO: 489 - DNA, 20 bp, Artificial Sequence
  Description of Artificial Sequence Synthetic oligonucleotide
atgagtaagacacgattccc

SEQ ID NO: 490 - DNA, 20 bp, Artificial Sequence
  Description of Artificial Sequence Synthetic oligonucleotide
cacgattccctctttcaagg

SEQ ID NO: 491 - DNA, 20 bp, Artificial Sequence
  Description of Artificial Sequence Synthetic oligonucleotide
tctttcaaggagctcatagt

SEQ ID NO: 492 - DNA, 20 bp, Artificial Sequence
  Description of Artificial Sequence Synthetic oligonucleotide
agctcatagtctgtgaggag

SEQ ID NO: 493 - DNA, 20 bp, Artificial Sequence
  Description of Artificial Sequence Synthetic oligonucleotide
ctgtgaggagacaaccccgg

SEQ ID NO: 494 - DNA, 20 bp, Artificial Sequence
  Description of Artificial Sequence Synthetic oligonucleotide
acaaccccggcgcgaagctg

SEQ ID NO: 495 - DNA, 20 bp, Artificial Sequence
  Description of Artificial Sequence Synthetic oligonucleotide
cgcgaagctgtcggctcccg

SEQ ID NO: 496 - DNA, 20 bp, Artificial Sequence
  Description of Artificial Sequence Synthetic oligonucleotide
tcggctcccggcagccgggc

SEQ ID NO: 497 - DNA, 20 bp, Artificial Sequence
  Description of Artificial Sequence Synthetic oligonucleotide
gcagccgggcggccacggca

SEQ ID NO: 498 - DNA, 20 bp, Artificial Sequence
  Description of Artificial Sequence Synthetic oligonucleotide
ggccacggcagcggcacctc

SEQ ID NO: 499 - DNA, 20 bp, Artificial Sequence
  Description of Artificial Sequence Synthetic oligonucleotide
gcggcacctcctcctgcggc

SEQ ID NO: 500 - DNA, 20 bp, Artificial Sequence
  Description of Artificial Sequence Synthetic oligonucleotide
ctcctgcggcgggcgccctc

SEQ ID NO: 501 - DNA, 20 bp, Artificial Sequence
  Description of Artificial Sequence Synthetic oligonucleotide
gggcgccctcacccttccgc

SEQ ID NO: 502 - DNA, 20 bp, Artificial Sequence
  Description of Artificial Sequence Synthetic oligonucleotide
acccttccgcgcccaagatc

SEQ ID NO: 503 - DNA, 20 bp, Artificial Sequence
  Description of Artificial Sequence Synthetic oligonucleotide
gcccaagatcgccatctcgc

SEQ ID NO: 504 - DNA, 20 bp, Artificial Sequence
  Description of Artificial Sequence Synthetic oligonucleotide
gccatctcgcagcaggcgcg

SEQ ID NO: 505 - DNA, 20 bp, Artificial Sequence
  Description of Artificial Sequence Synthetic oligonucleotide
agcaggcgcggcggcagcag

SEQ ID NO: 506 - DNA, 20 bp, Artificial Sequence
  Description of Artificial Sequence Synthetic oligonucleotide
gcggcagcagccacccaggc

SEQ ID NO: 507 - DNA, 20 bp, Artificial Sequence
  Description of Artificial Sequence Synthetic oligonucleotide
ccacccaggcgtcggccccg

SEQ ID NO: 508 - DNA, 20 bp, Artificial Sequence
  Description of Artificial Sequence Synthetic oligonucleotide
gtcggccccggcagcccagg

SEQ ID NO: 509 - DNA, 20 bp, Artificial Sequence
  Description of Artificial Sequence Synthetic oligonucleotide
gcagcccaggccgccagcag

SEQ ID NO: 510 - DNA, 20 bp, Artificial Sequence
  Description of Artificial Sequence Synthetic oligonucleotide
ccgccagcagccactcggaa

SEQ ID NO: 511 - DNA, 20 bp, Artificial Sequence
  Description of Artificial Sequence Synthetic oligonucleotide
ccactcggaacccgcacgcg

SEQ ID NO: 512 - DNA, 20 bp, Artificial Sequence
  Description of Artificial Sequence Synthetic oligonucleotide
cccgcacgcggagctcgccg

SEQ ID NO: 513 - DNA, 20 bp, Artificial Sequence
  Description of Artificial Sequence Synthetic oligonucleotide
gagctcgccgacacccgacc

SEQ ID NO: 514 - DNA, 20 bp, Artificial Sequence
  Description of Artificial Sequence Synthetic oligonucleotide
acacccgaccctccccccac

SEQ ID NO: 515 - DNA, 20 bp, Artificial Sequence
  Description of Artificial Sequence Synthetic oligonucleotide
ctccccccacccccaccccg

SEQ ID NO: 516 - DNA, 20 bp, Artificial Sequence
  Description of Artificial Sequence Synthetic oligonucleotide
ccccaccccgtccacgcgcc

SEQ ID NO: 517 - DNA, 20 bp, Artificial Sequence
  Description of Artificial Sequence Synthetic oligonucleotide
tccacgcgccacccccagcc

SEQ ID NO: 518 - DNA, 20 bp, Artificial Sequence
  Description of Artificial Sequence Synthetic oligonucleotide
acccccagccgaccccacac

SEQ ID NO: 519 - DNA, 20 bp, Artificial Sequence
  Description of Artificial Sequence Synthetic oligonucleotide
gaccccacacctcccttcct

SEQ ID NO: 520 - DNA, 20 bp, Artificial Sequence
  Description of Artificial Sequence Synthetic oligonucleotide
ctcccttcctcgggagcgcg

SEQ ID NO: 521 - DNA, 20 bp, Artificial Sequence
  Description of Artificial Sequence Synthetic oligonucleotide
cgggagcgcgccgcgtggcc

SEQ ID NO: 522 - DNA, 20 bp, Artificial Sequence
  Description of Artificial Sequence Synthetic oligonucleotide
ccgcgtggccgcgtcgccgg

SEQ ID NO: 523 - DNA, 20 bp, Artificial Sequence
  Description of Artificial Sequence Synthetic oligonucleotide
gcgtcgccggcgccgcgccc

SEQ ID NO: 524 - DNA, 20 bp, Artificial Sequence
  Description of Artificial Sequence Synthetic oligonucleotide
cgccgcgccctaagcgcgcg

SEQ ID NO: 525 - DNA, 20 bp, Artificial Sequence
  Description of Artificial Sequence Synthetic oligonucleotide
taagcgcgcgcctccccgcc

SEQ ID NO: 526 - DNA, 20 bp, Artificial Sequence
  Description of Artificial Sequence Synthetic oligonucleotide
cctccccgccggccccggca

SEQ ID NO: 527 - DNA, 20 bp, Artificial Sequence
  Description of Artificial Sequence Synthetic oligonucleotide
ggccccggcaccgcgcgcta

SEQ ID NO: 528 - DNA, 20 bp, Artificial Sequence
  Description of Artificial Sequence Synthetic oligonucleotide
ccgcgcgctacgccgcggcc

SEQ ID NO: 529 - DNA, 20 bp, Artificial Sequence
  Description of Artificial Sequence Synthetic oligonucleotide
cgccgcggccgcccaaagcc

SEQ ID NO: 530 - DNA, 20 bp, Artificial Sequence
  Description of Artificial Sequence Synthetic oligonucleotide
gcccaaagcctccaactccg

SEQ ID NO: 531 - DNA, 20 bp, Artificial Sequence
  Description of Artificial Sequence Synthetic oligonucleotide
tccaactccggacccggaat

SEQ ID NO: 532 - DNA, 20 bp, Artificial Sequence
  Description of Artificial Sequence Synthetic oligonucleotide
ccatgtcccaaaattcagct

SEQ ID NO: 533 - DNA, 20 bp, Artificial Sequence
  Description of Artificial Sequence Synthetic oligonucleotide
gaccttggtgaagcagtggc

SEQ ID NO: 534 - DNA, 20 bp, Artificial Sequence
  Description of Artificial Sequence Synthetic oligonucleotide
gtatcagaccttggtgaagc

SEQ ID NO: 535 - DNA, 20 bp, Artificial Sequence
  Description of Artificial Sequence Synthetic oligonucleotide
cctgtaactgcaaccttcca

SEQ ID NO: 536 - DNA, 20 bp, Artificial Sequence
  Description of Artificial Sequence Synthetic oligonucleotide
ggcactagaaacagttacct

SEQ ID NO: 537 - DNA, 20 bp, Artificial Sequence
  Description of Artificial Sequence Synthetic oligonucleotide
gaactactggattttgctgt

SEQ ID NO: 538 - DNA, 20 bp, Artificial Sequence
  Description of Artificial Sequence Synthetic oligonucleotide
cagttagctgttcatcccat

SEQ ID NO: 539 - DNA, 20 bp, Artificial Sequence
  Description of Artificial Sequence Synthetic oligonucleotide
attccaatgtagtctgtggc

SEQ ID NO: 540 - DNA, 20 bp, Artificial Sequence
  Description of Artificial Sequence Synthetic oligonucleotide
gatggctccaaacttgaaat

SEQ ID NO: 541 - DNA, 2785 bp, Artificial Sequence
  Human WWP1 Sequence
agctgaattttgggacatggccactgcttcaccaaggtctgatactagtaataaccacagtggaaggttgcagttacaggtaactgtttctagtgccaaacttaaaagaaaaaagaactggttcggaacagcaatatatacagaagtagttgtagatggagaaattacgaaaacagcaaaatccagtagttcttctaatccaaaatgggatgaacagctaactgtaaatgttacgccacagactacattggaatttcaagtttggagccatcgcactttaaaagcagatgctttattaggaaaagcaacgatagatttgaaacaagctctgttgatacacaatagaaaattggaaagagtgaaagaacaattaaaactttccttggaaaacaagaatggcatagcacaaactggtgaattgacagttgtgcttgatggattggtgattgagcaagaaaatataacaaactgcagctcatctccaaccatagaaatacaggaaaatggtgatgccttacatgaaaatggagagccttcagcaaggacaactgccaggttggctgttgaaggcacgaatggaatagataatcatgtacctacaagcactctagtccaaaactcatgctgctcgtatgtagttaatggagacaacacaccttcatctccgtctcaggttgctgccagacccaaaaatacaccagctccaaaaccactcgcatctgagcctgccgatgacactgttaatggagaatcatcctcatttgcaccaactgataatgcgtctgtcacgggtactccagtagtgtctgaagaaaatgccttgtctccaaattgcactagtactactgttgaagatcctccagttcaagaaatactgacttcctcagaaaacaatgaatgtattccttctaccagtgcagaattggaatctgaagctagaagtatattagagcctgacacctctaattctagaagtagttctgcttttgaagcagccaaatcaagacagccagatgggtgtatggatcctgtacggcagcagtctgggaatgccaacacagaaaccttgccatcagggtgggaacaaagaaaagatcctcatggtagaacctattatgtggatcataatactcgaactaccacatgggagagaccacaacctttacctccaggttgggaaagaagagttgatgatcgtagaagagtttattatgtggatcataacaccagaacaacaacgtggcagcggcctaccatggaatctgtccgaaattttgaacagtggcaatctcagcggaaccaattgcagggagctatgcaacagtttaaccaacgatacctctattcggcttcaatgttagctgcagaaaatgacccttatggacctttgccaccaggctgggaaaaaagagtggattcaacagacagggtttactttgtgaatcataacacaaaaacaacccagtgggaagatccaagaactcaaggcttacagaatgaagaacccctgccagaaggctgggaaattagatatactcgtgaaggtgtaaggtactttgttgatcataacacaagaacaacaacattcaaagatcctcgcaatgggaagtcatctgtaactaaaggtggtccacaaattgcttatgaacgcggctttaggtggaagcttgctcacttccgttatttgtgccagtctaatgcactacctagtcatgtaaagatcaatgtgtcccggcagacattgtttgaagattccttccaacagattatggcattaaaaccctatgacttgaggaggcgcttatatgtaatatttagaggagaagaaggacttgattatggtggcctagcgagagaatggtttttcttgctttcacatgaagttttgaacccaatgtattgcttatttgagtatgcgggcaagaacaactattgtctgcagataaatccagcatcaaccattaatccagaccatctttcatacttctgtttcattggtcgttttattgccatggcactatttcatggaaagtttatcgatactggtttctctttaccattctacaagcgtatgttaagtaaaaaacttactattaaggatttggaatctattgatactgaattttataactcccttatctggataagagataacaacattgaagaatgtggcttagaaatgtacttttctgttgacatggagattttgggaaaagttacttcacatgacctgaagttgggaggttccaatattctggtgactgaggagaacaaagatgaatatattggtttaatgacagaatggcgtttttctcgaggagtacaagaacagaccaaagctttccttgatggttttaatgaagttgttcctcttcagtggctacagtacttcgatgaaaaagaattagaggttatgttgtgtggcatgcaggaggttgacttggcagattggcagagaaatactgtttatcgacattatacaagaaacagcaagcaaatcatttggttttggcagtttgtgaaagagacagacaatgaagtaagaatgcgactattgcagttcgtcactggaacctgccgtttacctctaggaggatttgctgagctcatgggaagtaatgggcctcaaaagttttgcattgaaaaagttggcaaagacacttggttaccaagaagccatacatgttttaatcgcttggatctaccaccatataagagttatgaacaactaaaggaaaaacttctttttgcaatagaagagacagagggatttggacaagaatga